SEQUENCE LISTING (491 sequences)

SEQ ID NO: 1 - DNA, 116 bp, Homo sapiens
gttcccggaacggcctcttgggggcgttccagccccacggacccgcagggagtccccgccgcaatttgcatggggctcatttgcatgaccccgccccgcgcgggagtcgggggcgc

SEQ ID NO: 2 - DNA, 116 bp, Artificial sequence
  Synthetic
gttttcggaacggttttttgggggcgttttagttttacggattcgtagggagttttcgtcgtaatttgtatggggtttatttgtatgatttcgtttcgcgcgggagtcgggggcgt

SEQ ID NO: 3 - DNA, 23 bp, Artificial sequence
  Synthetic
ggcgttttagttttacggattcg

SEQ ID NO: 4 - DNA, 27 bp, Artificial sequence
  Synthetic
acaaataaaccccatacaaattacgac

SEQ ID NO: 5 - DNA, 20 bp, Artificial sequence
  Synthetic
cgccgaggcgaaaactccct

SEQ ID NO: 6 - DNA, 126 bp, Homo sapiens
cggattcaacatgggcaatgtgcctacactttcattcttccagaacacgatggcaactgtcgtgagagtacgacagaccagtacaacacaaacgctctgcagagagatgctccacacgtggaaccg

SEQ ID NO: 7 - DNA, 126 bp, Artificial sequence
  Synthetic
cggatttaatatgggtaatgtgtttatatttttatttttttagaatacgatggtaattgtcgtgagagtacgatagattagtataatataaacgttttgtagagagatgttttatacgtggaatcg

SEQ ID NO: 8 - DNA, 26 bp, Artificial sequence
  Synthetic
ttttagaatacgatggtaattgtcgt

SEQ ID NO: 9 - DNA, 35 bp, Artificial sequence
  Synthetic
acatctctctacaaaacgtttatattatactaatc

SEQ ID NO: 10 - DNA, 20 bp, Artificial sequence
  Synthetic
cgccgaggctatcgtactct

SEQ ID NO: 11 - DNA, 109 bp, Homo sapiens
ggagctacgacgagcagctgcggctggcgatggaactgtcggcgcaggagcaggaggagaggcggcggcgcgcgcgccaggaggaggaggagctggagcgcatcctgag

SEQ ID NO: 12 - DNA, 109 bp, Artificial sequence
  Synthetic
ggagttacgacgagtagttgcggttggcgatggaattgtcggcgtaggagtaggaggagaggcggcggcgcgcgcgttaggaggaggaggagttggagcgtattttgag

SEQ ID NO: 13 - DNA, 20 bp, Artificial sequence
  Synthetic
agttacgacgagtagttgcg

SEQ ID NO: 14 - DNA, 18 bp, Artificial sequence
  Synthetic
tcctcctactcctacgcc

SEQ ID NO: 15 - DNA, 21 bp, Artificial sequence
  Synthetic
ccacggacgcgacaattccat

SEQ ID NO: 16 - DNA, 143 bp, Homo sapiens
ggtggcaacggctggagtgccgtcgcccgcgccactcaccccggcgcggcgccctgcgcggccgctcagcggaaggccagcaggaagatcagtacgacgttgatgagaaccaggagcgccagcacggcggagaccaccacgcg

SEQ ID NO: 17 - DNA, 143 bp, Artificial sequence
  Synthetic
ggtggtaacggttggagtgtcgtcgttcgcgttatttatttcggcgcggcgttttgcgcggtcgtttagcggaaggttagtaggaagattagtacgacgttgatgagaattaggagcgttagtacggcggagattattacgcg

SEQ ID NO: 18 - DNA, 23 bp, Artificial sequence
  Synthetic
cgttcgcgttatttatttcggcg

SEQ ID NO: 19 - DNA, 24 bp, Artificial sequence
  Synthetic
gctcctaattctcatcaacgtcgt

SEQ ID NO: 20 - DNA, 20 bp, Artificial sequence
  Synthetic
cgccgagggcggcgttttgc

SEQ ID NO: 21 - DNA, 100 bp, Homo sapiens
ggcccggggccgcctgggcccctaggggctggacgtcaacctgttagatagagggcgtgggaccccccgcaggcggctgctcggacgaccgcatccggag

SEQ ID NO: 22 - DNA, 100 bp, Artificial sequence
  Synthetic
ggttcggggtcgtttgggtttttaggggttggacgttaatttgttagatagagggcgtgggatttttcgtaggcggttgttcggacgatcgtattcggag

SEQ ID NO: 23 - DNA, 24 bp, Artificial sequence
  Synthetic
cgttaatttgttagatagagggcg

SEQ ID NO: 24 - DNA, 18 bp, Artificial sequence
  Synthetic
acgatcgtccgaacaacc

SEQ ID NO: 25 - DNA, 21 bp, Artificial sequence
  Synthetic
ccacggacgcgcctacgaaaa

SEQ ID NO: 26 - DNA, 17 bp, Artificial sequence
  Synthetic
tccgaacaaccgcctac

SEQ ID NO: 27 - DNA, 21 bp, Artificial sequence
  Synthetic
ccacggacgcgaaaaatccca

SEQ ID NO: 28 - DNA, 119 bp, Homo sapiens
gcttccagccgcgcgctccgtgccactgccgctctctgcagccccgcgtccccgcagcctccccatggccagcccgcttcgctccgctgcggcccttgcccgccaggtacctcgaaccc

SEQ ID NO: 29 - DNA, 119 bp, Artificial sequence
  Synthetic
gtttttagtcgcgcgtttcgtgttattgtcgttttttgtagtttcgcgttttcgtagtttttttatggttagttcgtttcgtttcgttgcggtttttgttcgttaggtatttcgaattt

SEQ ID NO: 30 - DNA, 27 bp, Artificial sequence
  Synthetic
gtgttattgtcgttttttgtagtttcg

SEQ ID NO: 31 - DNA, 18 bp, Artificial sequence
  Synthetic
cgcaacgaaacgaaacga

SEQ ID NO: 32 - DNA, 20 bp, Artificial sequence
  Synthetic
cgccgagggcgttttcgtag

SEQ ID NO: 33 - DNA, 140 bp, Homo sapiens
gcccgccgcacgccgcaatgctccgcgctccccgcggggtcgggcgactcagacagggaccggaaaagaaccacgcagaagaaagccctatttcttgtcgtctgttcctgtgcagccttgcagcctcgccgcccccgcgt

SEQ ID NO: 34 - DNA, 140 bp, Artificial sequence
  Synthetic
gttcgtcgtacgtcgtaatgtttcgcgtttttcgcggggtcgggcgatttagatagggatcggaaaagaattacgtagaagaaagttttattttttgtcgtttgtttttgtgtagttttgtagtttcgtcgttttcgcgt

SEQ ID NO: 35 - DNA, 21 bp, Artificial sequence
  Synthetic
cgtaatgtttcgcgtttttcg

SEQ ID NO: 36 - DNA, 27 bp, Artificial sequence
  Synthetic
actttcttctacgtaattcttttccga

SEQ ID NO: 37 - DNA, 20 bp, Artificial sequence
  Synthetic
cgccgagggcggggtcgggc

SEQ ID NO: 38 - DNA, 85 bp, Homo sapiens
gccggggagtcgagaagcaagtactagcgctccaggaccgcgcgcgccgccccgcgccgccccgcgccgcccctcggtccagagc

SEQ ID NO: 39 - DNA, 85 bp, Artificial sequence
  Synthetic
gtcggggagtcgagaagtaagtattagcgttttaggatcgcgcgcgtcgtttcgcgtcgtttcgcgtcgtttttcggtttagagt

SEQ ID NO: 40 - DNA, 23 bp, Artificial sequence
  Synthetic
agtattagcgttttaggatcgcg

SEQ ID NO: 41 - DNA, 21 bp, Artificial sequence
  Synthetic
actctaaaccgaaaaacgacg

SEQ ID NO: 42 - DNA, 21 bp, Artificial sequence
  Synthetic
ccacggacggcgaaacgacgc

SEQ ID NO: 43 - DNA, 74 bp, Homo sapiens
gccgggagcccgcacttcctcctcgggggcctcagaaaaccacagggcgcggggccagggcggcggcccccagg

SEQ ID NO: 44 - DNA, 74 bp, Artificial sequence
  Synthetic
gtcgggagttcgtatttttttttcgggggttttagaaaattatagggcgcggggttagggcggcggtttttagg

SEQ ID NO: 45 - DNA, 25 bp, Artificial sequence
  Synthetic
tcgggagttcgtatttttttttcgg

SEQ ID NO: 46 - DNA, 17 bp, Artificial sequence
  Synthetic
aaaaccgccgccctaac

SEQ ID NO: 47 - DNA, 20 bp, Artificial sequence
  Synthetic
cgccgaggccccgcgcccta

SEQ ID NO: 48 - DNA, 78 bp, Homo sapiens
cggggcctaccctcaggcagcgctcgctcgaggccagcttccgagctccaacccctgcccgaaacctcggcctcactg

SEQ ID NO: 49 - DNA, 78 bp, Artificial sequence
  Synthetic
cggggtttatttttaggtagcgttcgttcgaggttagttttcgagttttaatttttgttcgaaatttcggttttattg

SEQ ID NO: 50 - DNA, 24 bp, Artificial sequence
  Synthetic
gggtttatttttaggtagcgttcg

SEQ ID NO: 51 - DNA, 29 bp, Artificial sequence
  Synthetic
cgaaatttcgaacaaaaattaaaactcga

SEQ ID NO: 52 - DNA, 21 bp, Artificial sequence
  Synthetic
ccacggacggttcgaggttag

SEQ ID NO: 53 - DNA, 141 bp, Homo sapiens
tgtcctgacacgatggccacaggcacagtttgtggtgatgcccaggggcccgcgcggccccacggtggtccagtttacactcgggccccgcactcctgaagttccgcgcgggaggagaagggcgtccctttcgcagctcgg

SEQ ID NO: 54 - DNA, 141 bp, Artificial sequence
  Synthetic
tgttttgatacgatggttataggtatagtttgtggtgatgtttaggggttcgcgcggttttacggtggtttagtttatattcgggtttcgtatttttgaagtttcgcgcgggaggagaagggcgtttttttcgtagttcgg

SEQ ID NO: 55 - DNA, 19 bp, Artificial sequence
  Synthetic
tgatgtttaggggttcgcg

SEQ ID NO: 56 - DNA, 26 bp, Artificial sequence
  Synthetic
cgaaacttcaaaaatacgaaacccga

SEQ ID NO: 57 - DNA, 20 bp, Artificial sequence
  Synthetic
cgccgagggcggttttacgg

SEQ ID NO: 58 - DNA, 112 bp, Homo sapiens
ccggagcactcgccgctgcgcgccctgaagccgctggcggtaggcggccctcgaggccggcgggctgggcggctcggcagcctgcgccgcggcctccgcctcggccgccagc

SEQ ID NO: 59 - DNA, 112 bp, Artificial sequence
  Synthetic
tcggagtattcgtcgttgcgcgttttgaagtcgttggcggtaggcggttttcgaggtcggcgggttgggcggttcggtagtttgcgtcgcggttttcgtttcggtcgttagt

SEQ ID NO: 60 - DNA, 17 bp, Artificial sequence
  Synthetic
gtattcgtcgttgcgcg

SEQ ID NO: 61 - DNA, 18 bp, Artificial sequence
  Synthetic
cctcgaaaaccgcctacc

SEQ ID NO: 62 - DNA, 21 bp, Artificial sequence
  Synthetic
ccacggacgcgccaacgactt

SEQ ID NO: 63 - DNA, 134 bp, Homo sapiens
cgccgtgagtgttatagttcttaaaggcggcgtgtccggagtttcttccttctggtggggttcgtggtctcgccggctcaggagtgaagctgcagatcttcgcggtgagtgttacagctcctaaggcggcgcat

SEQ ID NO: 64 - DNA, 134 bp, Artificial sequence
  Synthetic
cgtcgtgagtgttatagtttttaaaggcggcgtgttcggagttttttttttttggtggggttcgtggtttcgtcggtttaggagtgaagttgtagattttcgcggtgagtgttatagtttttaaggcggcgtat

SEQ ID NO: 65 - DNA, 17 bp, Artificial sequence
  Synthetic
taaaggcggcgtgttcg

SEQ ID NO: 66 - DNA, 21 bp, Artificial sequence
  Synthetic
caacttcactcctaaaccgac

SEQ ID NO: 67 - DNA, 21 bp, Artificial sequence
  Synthetic
ccacggacgcgaaaccacgaa

SEQ ID NO: 68 - DNA, 107 bp, Homo sapiens
aactggccttctggctactccggaatcgccaagcagatgaggccagaccgccgccagcgctgatcacgcgcgctcccacaggtcctggcgcgcgtgttcagccgcgc

SEQ ID NO: 69 - DNA, 107 bp, Artificial sequence
  Synthetic
aattggttttttggttatttcggaatcgttaagtagatgaggttagatcgtcgttagcgttgattacgcgcgtttttataggttttggcgcgcgtgtttagtcgcgt

SEQ ID NO: 70 - DNA, 26 bp, Artificial sequence
  Synthetic
tggttttttggttatttcggaatcgt

SEQ ID NO: 71 - DNA, 19 bp, Artificial sequence
  Synthetic
gcgcgtaatcaacgctaac

SEQ ID NO: 72 - DNA, 20 bp, Artificial sequence
  Synthetic
cgccgaggcgacgatctaac

SEQ ID NO: 73 - DNA, 85 bp, Homo sapiens
ggagcgggcagaggaggagcccagcgccgaggcccaggcgcgccccgccctcgcccctccccgtgcccctcccccgctgctcccc

SEQ ID NO: 74 - DNA, 85 bp, Artificial sequence
  Synthetic
ggagcgggtagaggaggagtttagcgtcgaggtttaggcgcgtttcgttttcgttttttttcgtgtttttttttcgttgtttttt

SEQ ID NO: 75 - DNA, 19 bp, Artificial sequence
  Synthetic
gaggaggagtttagcgtcg

SEQ ID NO: 76 - DNA, 24 bp, Artificial sequence
  Synthetic
cacgaaaaaaaacgaaaacgaaac

SEQ ID NO: 77 - DNA, 20 bp, Artificial sequence
  Synthetic
cgccgaggcgcgcctaaacc

SEQ ID NO: 78 - DNA, 107 bp, Homo sapiens
gcggtctatcacgggcacccctaacacttggtgagtgcgcagtgctctcggcagtctctgggctccatacgatgcctaccgcacgccctagcagaggaggtctctgt

SEQ ID NO: 79 - DNA, 107 bp, Artificial sequence
  Synthetic
gcggtttattacgggtatttttaatatttggtgagtgcgtagtgttttcggtagtttttgggttttatacgatgtttatcgtacgttttagtagaggaggtttttgt

SEQ ID NO: 80 - DNA, 20 bp, Artificial sequence
  Synthetic
tgagtgcgtagtgttttcgg

SEQ ID NO: 81 - DNA, 27 bp, Artificial sequence
  Synthetic
ctcctctactaaaacgtacgataaaca

SEQ ID NO: 82 - DNA, 20 bp, Artificial sequence
  Synthetic
cgccgaggatcgtataaaac

SEQ ID NO: 83 - DNA, 21 bp, Artificial sequence
  Synthetic
atatttggtgagtgcgtagtg

SEQ ID NO: 84 - DNA, 26 bp, Artificial sequence
  Synthetic
acgtacgataaacatcgtataaaacc

SEQ ID NO: 85 - DNA, 20 bp, Artificial sequence
  Synthetic
cgccgagggttttcggtagt

SEQ ID NO: 86 - DNA, 121 bp, Homo sapiens
tactccactgccggcttggtgcccacgctcggcttccgcccacccatggactacgcctttagcgatctcatgcgtgaccgctcggccgccgctgctgcggcggtgcacaaggagccgacct

SEQ ID NO: 87 - DNA, 121 bp, Artificial sequence
  Synthetic
tattttattgtcggtttggtgtttacgttcggttttcgtttatttatggattacgtttttagcgattttatgcgtgatcgttcggtcgtcgttgttgcggcggtgtataaggagtcgattt

SEQ ID NO: 88 - DNA, 23 bp, Artificial sequence
  Synthetic
tggtgtttacgttcggttttcgt

SEQ ID NO: 89 - DNA, 17 bp, Artificial sequence
  Synthetic
ccgcaacaacgacgacc

SEQ ID NO: 90 - DNA, 20 bp, Artificial sequence
  Synthetic
cgccgaggcgaacgatcacg

SEQ ID NO: 91 - DNA, 106 bp, Homo sapiens
aggccggtcacgaacaaagcgctggcgagtgcgcgcccgcccacgcgcacaggtgcccgcgacaagacgccccgtccccgcccacgcggcccccgcgggctgagcc

SEQ ID NO: 92 - DNA, 106 bp, Artificial sequence
  Synthetic
aggtcggttacgaataaagcgttggcgagtgcgcgttcgtttacgcgtataggtgttcgcgataagacgtttcgttttcgtttacgcggttttcgcgggttgagtt

SEQ ID NO: 93 - DNA, 21 bp, Artificial sequence
  Synthetic
gttacgaataaagcgttggcg

SEQ ID NO: 94 - DNA, 20 bp, Artificial sequence
  Synthetic
aacgaaacgtcttatcgcga

SEQ ID NO: 95 - DNA, 21 bp, Artificial sequence
  Synthetic
ccacggacggagtgcgcgttc

SEQ ID NO: 96 - DNA, 85 bp, Homo sapiens
gccggccccgcagcatcctcctgctcgcggctctcccgccacctgtcccgctccctgccgcgccctggggcccgcacctacccac

SEQ ID NO: 97 - DNA, 85 bp, Artificial sequence
  Synthetic
gtcggtttcgtagtatttttttgttcgcggttttttcgttatttgtttcgttttttgtcgcgttttggggttcgtatttatttat

SEQ ID NO: 98 - DNA, 25 bp, Artificial sequence
  Synthetic
cggtttcgtagtatttttttgttcg

SEQ ID NO: 99 - DNA, 17 bp, Artificial sequence
  Synthetic
gaaccccaaaacgcgac

SEQ ID NO: 100 - DNA, 20 bp, Artificial sequence
  Synthetic
cgccgagggcggttttttcg

SEQ ID NO: 101 - DNA, 134 bp, Homo sapiens
cgcctcctgggctccccccggagtgggagggagccgcggtcccgcctccgcgcccgttccctcccaggcccctcggccgccgcgccgagctttccgcgcgtggacagactgcccggccgacggacggacgcagg

SEQ ID NO: 102 - DNA, 134 bp, Artificial sequence
  Synthetic
cgttttttgggtttttttcggagtgggagggagtcgcggtttcgttttcgcgttcgtttttttttaggtttttcggtcgtcgcgtcgagtttttcgcgcgtggatagattgttcggtcgacggacggacgtagg

SEQ ID NO: 103 - DNA, 19 bp, Artificial sequence
  Synthetic
gagtcgcggtttcgttttc

SEQ ID NO: 104 - DNA, 19 bp, Artificial sequence
  Synthetic
gacgcgacgaccgaaaaac

SEQ ID NO: 105 - DNA, 20 bp, Artificial sequence
  Synthetic
cgccgaggcgcgttcgtttt

SEQ ID NO: 106 - DNA, 108 bp, Homo sapiens
tccggcgccgcgttttctagagaaccgggtctcagcgatgctcatttcagccccgtcttaatgcaacaaacgaaaccccacacgaacgaaaaggaacatgtctgcgct

SEQ ID NO: 107 - DNA, 107 bp, Artificial sequence
  Synthetic
tcggcgtcgcgttttttagagaatcgggttttagcgatgtttattttagtttcgttttaatgtaataaacgaaattttatacgaacgaaaaggaatatgtttgcgtt

SEQ ID NO: 108 - DNA, 21 bp, Artificial sequence
  Synthetic
ggcgtcgcgttttttagagaa

SEQ ID NO: 109 - DNA, 28 bp, Artificial sequence
  Synthetic
ttccttttcgttcgtataaaatttcgtt

SEQ ID NO: 110 - DNA, 21 bp, Artificial sequence
  Synthetic
ccacggacgatcgggttttag

SEQ ID NO: 111 - DNA, 128 bp, Homo sapiens
gggcctgctggccggggacccgcgcgtcgagcgcctggtgcgcgacagcgcctcctactgccgcgagcgcttcgaccccgacgagtactccacggccgtgcgcgaggcgccagcggagctcgccgaag

SEQ ID NO: 112 - DNA, 128 bp, Artificial sequence
  Synthetic
gggtttgttggtcggggattcgcgcgtcgagcgtttggtgcgcgatagcgttttttattgtcgcgagcgtttcgatttcgacgagtattttacggtcgtgcgcgaggcgttagcggagttcgtcgaag

SEQ ID NO: 113 - DNA, 23 bp, Artificial sequence
  Synthetic
cgatagcgttttttattgtcgcg

SEQ ID NO: 114 - DNA, 22 bp, Artificial sequence
  Synthetic
gcacgaccgtaaaatactcgtc

SEQ ID NO: 115 - DNA, 21 bp, Artificial sequence
  Synthetic
ccacggacgcgaaatcgaaac

SEQ ID NO: 116 - DNA, 140 bp, Homo sapiens
tagcagcagccgcagccatggcggggatgaagacagcctccggggactacatcgactcgtcatgggagctgcgggtgtttgtgggagaggaggacccagaggccgagtcggtcaccctgcgggtcactggggagtcgcac

SEQ ID NO: 117 - DNA, 140 bp, Artificial sequence
  Synthetic
tagtagtagtcgtagttatggcggggatgaagatagttttcggggattatatcgattcgttatgggagttgcgggtgtttgtgggagaggaggatttagaggtcgagtcggttattttgcgggttattggggagtcgtat

SEQ ID NO: 118 - DNA, 24 bp, Artificial sequence
  Synthetic
gttttcggggattatatcgattcg

SEQ ID NO: 119 - DNA, 22 bp, Artificial sequence
  Synthetic
cccaataacccgcaaaataacc

SEQ ID NO: 120 - DNA, 20 bp, Artificial sequence
  Synthetic
cgccgaggcgactcgacctc

SEQ ID NO: 121 - DNA, 104 bp, Homo sapiens
gtcccagagacgccctagggtcagaggtcatctccgtggcaacggaaacttcccgcgctacggcggctccaacgggccgcttccgccgcattgcgtagcgaagc

SEQ ID NO: 122 - DNA, 104 bp, Artificial sequence
  Synthetic
gttttagagacgttttagggttagaggttattttcgtggtaacggaaatttttcgcgttacggcggttttaacgggtcgttttcgtcgtattgcgtagcgaagt

SEQ ID NO: 123 - DNA, 24 bp, Artificial sequence
  Synthetic
tttcgtggtaacggaaatttttcg

SEQ ID NO: 124 - DNA, 17 bp, Artificial sequence
  Synthetic
cgacgaaaacgacccgt

SEQ ID NO: 125 - DNA, 20 bp, Artificial sequence
  Synthetic
cgccgagggcgttacggcgg

SEQ ID NO: 126 - DNA, 107 bp, Homo sapiens
gcgccccggccgcaggcggaggacagggaggagcgcacacgagaaagctcccacgcgcccgcgcctcgcctccgacgggaaggcgcctcttccgaccgtcctggatg

SEQ ID NO: 127 - DNA, 107 bp, Artificial sequence
  Synthetic
gcgtttcggtcgtaggcggaggatagggaggagcgtatacgagaaagtttttacgcgttcgcgtttcgttttcgacgggaaggcgtttttttcgatcgttttggatg

SEQ ID NO: 128 - DNA, 25 bp, Artificial sequence
  Synthetic
gagcgtatacgagaaagtttttacg

SEQ ID NO: 129 - DNA, 17 bp, Artificial sequence
  Synthetic
aacgccttcccgtcgaa

SEQ ID NO: 130 - DNA, 21 bp, Artificial sequence
  Synthetic
ccacggacggcgttcgcgttt

SEQ ID NO: 131 - DNA, 108 bp, Homo sapiens
cgagagggcgcgagcacagccgaggccatggaggtgacggcggaccagccgcgctgggtgagccaccaccaccccgccgtgctcaacgggcagcacccggacacgcac

SEQ ID NO: 132 - DNA, 108 bp, Artificial sequence
  Synthetic
cgagagggcgcgagtatagtcgaggttatggaggtgacggcggattagtcgcgttgggtgagttattattatttcgtcgtgtttaacgggtagtattcggatacgtat

SEQ ID NO: 133 - DNA, 17 bp, Artificial sequence
  Synthetic
gggcgcgagtatagtcg

SEQ ID NO: 134 - DNA, 17 bp, Artificial sequence
  Synthetic
caacgcgactaatccgc

SEQ ID NO: 135 - DNA, 20 bp, Artificial sequence
  Synthetic
cgccgaggccgtcacctcca

SEQ ID NO: 136 - DNA, 141 bp, Homo sapiens
cgccccctcacctccccgatcatgccgttccagacgccatcgatcttctttccgtgcttgccattggtgaccaggtagaggtcgtagctgaagccgatggtatgcgccagccgcttcagaatgtcgatgcagaaacccttg

SEQ ID NO: 137 - DNA, 141 bp, Artificial sequence
  Synthetic
cgtttttttattttttcgattatgtcgttttagacgttatcgatttttttttcgtgtttgttattggtgattaggtagaggtcgtagttgaagtcgatggtatgcgttagtcgttttagaatgtcgatgtagaaatttttg

SEQ ID NO: 138 - DNA, 27 bp, Artificial sequence
  Synthetic
tcgattatgtcgttttagacgttatcg

SEQ ID NO: 139 - DNA, 28 bp, Artificial sequence
  Synthetic
tctacatcgacattctaaaacgactaac

SEQ ID NO: 140 - DNA, 21 bp, Artificial sequence
  Synthetic
ccacggacgcgcataccatcg

SEQ ID NO: 141 - DNA, 93 bp, Homo sapiens
cggcgaggcttcccgcctggcgcattacaacaagcgctcgaccatcacctccagggagatccagacggccgtgcgcctgctgcttcccgggga

SEQ ID NO: 142 - DNA, 93 bp, Artificial sequence
  Synthetic
cggcgaggtttttcgtttggcgtattataataagcgttcgattattatttttagggagatttagacggtcgtgcgtttgttgtttttcgggga

SEQ ID NO: 143 - DNA, 23 bp, Artificial sequence
  Synthetic
tggcgtattataataagcgttcg

SEQ ID NO: 144 - DNA, 18 bp, Artificial sequence
  Synthetic
aacaacaaacgcacgacc

SEQ ID NO: 145 - DNA, 21 bp, Artificial sequence
  Synthetic
ccacggacgcgtctaaatctc

SEQ ID NO: 146 - DNA, 101 bp, Homo sapiens
gggcgggccaggcgctgggcacggtgatggccaccactggggccctgggcaactactacgtggactcgttcctgctgggcgccgacgccgcggatgagctg

SEQ ID NO: 147 - DNA, 101 bp, Artificial sequence
  Synthetic
gggcgggttaggcgttgggtacggtgatggttattattggggttttgggtaattattacgtggattcgtttttgttgggcgtcgacgtcgcggatgagttg

SEQ ID NO: 148 - DNA, 24 bp, Artificial sequence
  Synthetic
ttgggtaattattacgtggattcg

SEQ ID NO: 149 - DNA, 16 bp, Artificial sequence
  Synthetic
actcatccgcgacgtc

SEQ ID NO: 150 - DNA, 21 bp, Artificial sequence
  Synthetic
ccacggacgcgacgcccaaca

SEQ ID NO: 151 - DNA, 95 bp, Homo sapiens
gggccattgccagaagacgtcttctcggggcgccaggattcacctttccttcccgacctcaacttcttcgcggccgactcctgtctccagctatc

SEQ ID NO: 152 - DNA, 95 bp, Artificial sequence
  Synthetic
gggttattgttagaagacgttttttcggggcgttaggatttatttttttttttcgattttaattttttcgcggtcgatttttgtttttagttatt

SEQ ID NO: 153 - DNA, 22 bp, Artificial sequence
  Synthetic
gttagaagacgttttttcgggg

SEQ ID NO: 154 - DNA, 20 bp, Artificial sequence
  Synthetic
aaaacaaaaatcgaccgcga

SEQ ID NO: 155 - DNA, 20 bp, Artificial sequence
  Synthetic
cgccgagggcgttaggattt

SEQ ID NO: 156 - DNA, 106 bp, Homo sapiens
ggccccggaagcccagctcccgggccctggagcccgccacggcggcagccctgcggcggcggctggacctgggcagttgcctggacgtgctggcctttgcccagca

SEQ ID NO: 157 - DNA, 106 bp, Artificial sequence
  Synthetic
ggtttcggaagtttagttttcgggttttggagttcgttacggcggtagttttgcggcggcggttggatttgggtagttgtttggacgtgttggtttttgtttagta

SEQ ID NO: 158 - DNA, 25 bp, Artificial sequence
  Synthetic
agttttcgggttttggagttcgtta

SEQ ID NO: 159 - DNA, 17 bp, Artificial sequence
  Synthetic
ccaaatccaaccgccgc

SEQ ID NO: 160 - DNA, 20 bp, Artificial sequence
  Synthetic
cgccgaggacggcggtagtt

SEQ ID NO: 161 - DNA, 106 bp, Homo sapiens
ggcggcgccggcggctgcgcggggggcgccaggccctgctgctgctgctgctgctgactgcggtagtaggcggcggcggccacggcggcaaagttgtgggtctgga

SEQ ID NO: 162 - DNA, 106 bp, Artificial sequence
  Synthetic
ggcggcgtcggcggttgcgcggggggcgttaggttttgttgttgttgttgttgttgattgcggtagtaggcggcggcggttacggcggtaaagttgtgggtttgga

SEQ ID NO: 163 - DNA, 19 bp, Artificial sequence
  Synthetic
ttgattgcggtagtaggcg

SEQ ID NO: 164 - DNA, 19 bp, Artificial sequence
  Synthetic
aacccacaactttaccgcc

SEQ ID NO: 165 - DNA, 20 bp, Artificial sequence
  Synthetic
cgccgaggcgtaaccgccgc

SEQ ID NO: 166 - DNA, 154 bp, Homo sapiens
ggtttccccccaccccggcctcggggtctctccacgtctccccgccgacgtgctcacctgctcagggggcgcccccgagccgcgccccgcgcccgcccccaggagggcctccgcgagccggctgcacaccccgaggcggtcccggctgcacaac

SEQ ID NO: 167 - DNA, 154 bp, Artificial sequence
  Synthetic
ggtttttttttatttcggtttcggggtttttttacgttttttcgtcgacgtgtttatttgtttagggggcgttttcgagtcgcgtttcgcgttcgtttttaggagggttttcgcgagtcggttgtatatttcgaggcggtttcggttgtataat

SEQ ID NO: 168 - DNA, 27 bp, Artificial sequence
  Synthetic
gtttcggggtttttttacgttttttcg

SEQ ID NO: 169 - DNA, 19 bp, Artificial sequence
  Synthetic
aaacgcgactcgaaaacgc

SEQ ID NO: 170 - DNA, 20 bp, Artificial sequence
  Synthetic
cgccgagggtcgacgtgttt

SEQ ID NO: 171 - DNA, 95 bp, Homo sapiens
ctccggttttcgcggttctcagcgatattaggcgcggccagtgtctgaaagctcctcggggttacgtcctggggcgactggaggcggctcacgac

SEQ ID NO: 172 - DNA, 95 bp, Artificial sequence
  Synthetic
tttcggttttcgcggtttttagcgatattaggcgcggttagtgtttgaaagtttttcggggttacgttttggggcgattggaggcggtttacgat

SEQ ID NO: 173 - DNA, 22 bp, Artificial sequence
  Synthetic
cggtttttagcgatattaggcg

SEQ ID NO: 174 - DNA, 18 bp, Artificial sequence
  Synthetic
cccaaaacgtaaccccga

SEQ ID NO: 175 - DNA, 20 bp, Artificial sequence
  Synthetic
cgccgagggcggttagtgtt

SEQ ID NO: 176 - DNA, 143 bp, Homo sapiens
cgacggccgcggaggaggaaggccagggggaaatttgcatttcgtaaaaccgcggttaagaaatgacgatgccacgtagacaagccagttgtgacgttcagcacaacgtgctactgaactaccgagatccgccaccaaatggc

SEQ ID NO: 177 - DNA, 143 bp, Artificial sequence
  Synthetic
cgacggtcgcggaggaggaaggttagggggaaatttgtatttcgtaaaatcgcggttaagaaatgacgatgttacgtagataagttagttgtgacgtttagtataacgtgttattgaattatcgagattcgttattaaatggt

SEQ ID NO: 178 - DNA, 25 bp, Artificial sequence
  Synthetic
gggaaatttgtatttcgtaaaatcg

SEQ ID NO: 179 - DNA, 27 bp, Artificial sequence
  Synthetic
acaactaacttatctacgtaacatcgt

SEQ ID NO: 180 - DNA, 21 bp, Artificial sequence
  Synthetic
ccacggacggcggttaagaaa

SEQ ID NO: 181 - DNA, 116 bp, Homo sapiens
ggcttggggtccagccgcccgcccctgccgccaccgcaccatgtcctgcctctactcccgcctcagcgccccctgcggggtccgcgccttcagctgcatctcggcctgcgggcccc

SEQ ID NO: 182 - DNA, 116 bp, Artificial sequence
  Synthetic
ggtttggggtttagtcgttcgtttttgtcgttatcgtattatgttttgtttttattttcgttttagcgttttttgcggggttcgcgtttttagttgtatttcggtttgcgggtttt

SEQ ID NO: 183 - DNA, 22 bp, Artificial sequence
  Synthetic
tcgttcgtttttgtcgttatcg

SEQ ID NO: 184 - DNA, 23 bp, Artificial sequence
  Synthetic
aaccgaaatacaactaaaaacgc

SEQ ID NO: 185 - DNA, 21 bp, Artificial sequence
  Synthetic
ccacggacgcgaaccccgcaa

SEQ ID NO: 186 - DNA, 108 bp, Homo sapiens
ggaaggctgcagcgagagatttacatattcatccgagcttaaggaagccgcgataatgcaggtacagcccgaaacccacgcccccagaccttatctgcgcgccccgcc

SEQ ID NO: 187 - DNA, 108 bp, Artificial sequence
  Synthetic
ggaaggttgtagcgagagatttatatatttattcgagtttaaggaagtcgcgataatgtaggtatagttcgaaatttacgtttttagattttatttgcgcgtttcgtt

SEQ ID NO: 188 - DNA, 19 bp, Artificial sequence
  Synthetic
ttcgagtttaaggaagtcg

SEQ ID NO: 189 - DNA, 23 bp, Artificial sequence
  Synthetic
tctaaaaacgtaaatttcgaact

SEQ ID NO: 190 - DNA, 21 bp, Artificial sequence
  Synthetic
ccacggacggcgataatgtag

SEQ ID NO: 191 - DNA, 137 bp, Homo sapiens
ggagttatttttaaccatcgcctcccagaacattacggagcttcctctctccaacacgcaggaaaccctacttggctgtgcttcctgctaacacgaggccctgcgattgctgagaacaacagccccgagactgcgcg

SEQ ID NO: 192 - DNA, 137 bp, Artificial sequence
  Synthetic
ggagttatttttaattatcgttttttagaatattacggagttttttttttttaatacgtaggaaattttatttggttgtgttttttgttaatacgaggttttgcgattgttgagaataatagtttcgagattgcgcg

SEQ ID NO: 193 - DNA, 30 bp, Artificial sequence
  Synthetic
tttaattatcgttttttagaatattacgga

SEQ ID NO: 194 - DNA, 26 bp, Artificial sequence
  Synthetic
actattattctcaacaatcgcaaaac

SEQ ID NO: 195 - DNA, 21 bp, Artificial sequence
  Synthetic
ccacggacgcctcgtattaac

SEQ ID NO: 196 - DNA, 117 bp, Homo sapiens
ggcgggcgcttggccaaacagcccaagactgcggaatcacactcgccactgtgtacctggacgccatctgcagacccagcgcctgcggggattccggaaacgggagagcgggcttcc

SEQ ID NO: 197 - DNA, 117 bp, Artificial sequence
  Synthetic
ggcgggcgtttggttaaatagtttaagattgcggaattatattcgttattgtgtatttggacgttatttgtagatttagcgtttgcggggatttcggaaacgggagagcgggttttt

SEQ ID NO: 198 - DNA, 27 bp, Artificial sequence
  Synthetic
agtttaagattgcggaattatattcgt

SEQ ID NO: 199 - DNA, 17 bp, Artificial sequence
  Synthetic
ttccgaaatccccgcaa

SEQ ID NO: 200 - DNA, 20 bp, Artificial sequence
  Synthetic
cgccgaggaacgctaaatct

SEQ ID NO: 201 - DNA, 156 bp, Homo sapiens
cgcaggctgaggccctcgggtccccagcgggtcctcgccatcagtcactctctacgggccaggcctgggggtcacggcctgcaggagcctccctgcgcggccccactccctcatctgcgaccccgtggggaggcgaccctgaccaccctcgttccg

SEQ ID NO: 202 - DNA, 156 bp, Artificial sequence
  Synthetic
cgtaggttgaggttttcgggtttttagcgggttttcgttattagttattttttacgggttaggtttgggggttacggtttgtaggagtttttttgcgcggttttatttttttatttgcgatttcgtggggaggcgattttgattattttcgtttcg

SEQ ID NO: 203 - DNA, 23 bp, Artificial sequence
  Synthetic
ggttgaggttttcgggtttttag

SEQ ID NO: 204 - DNA, 17 bp, Artificial sequence
  Synthetic
cctccccacgaaatcgc

SEQ ID NO: 205 - DNA, 20 bp, Artificial sequence
  Synthetic
cgccgagggcgggttttcgt

SEQ ID NO: 206 - DNA, 18 bp, Artificial sequence
  Synthetic
aggagtttttttgcgcgg

SEQ ID NO: 207 - DNA, 24 bp, Artificial sequence
  Synthetic
acgaaaataatcaaaatcgcctcc

SEQ ID NO: 208 - DNA, 20 bp, Artificial sequence
  Synthetic
cgccgaggcccacgaaatcg

SEQ ID NO: 209 - DNA, 114 bp, Homo sapiens
cgggggagggcggcatcagccagagcctcagccgacggcgctccccaggtccacttcccgctccgataccctccccctaagcacgatacccagggcccagggctgctcttggcg

SEQ ID NO: 210 - DNA, 114 bp, Artificial sequence
  Synthetic
cgggggagggcggtattagttagagttttagtcgacggcgttttttaggtttatttttcgtttcgatattttttttttaagtacgatatttagggtttagggttgtttttggcg

SEQ ID NO: 211 - DNA, 25 bp, Artificial sequence
  Synthetic
gcggtattagttagagttttagtcg

SEQ ID NO: 212 - DNA, 24 bp, Artificial sequence
  Synthetic
acaaccctaaaccctaaatatcgt

SEQ ID NO: 213 - DNA, 21 bp, Artificial sequence
  Synthetic
ccacggacggacggcgttttt

SEQ ID NO: 214 - DNA, 107 bp, Homo sapiens
ctccgctccccgcaggcctggccgcgcgacgggcacccagcgggttgttatcaattattcaggccccaagttcacgggcactgcatccatttccctcgcgtgcgccc

SEQ ID NO: 215 - DNA, 107 bp, Artificial sequence
  Synthetic
tttcgtttttcgtaggtttggtcgcgcgacgggtatttagcgggttgttattaattatttaggttttaagtttacgggtattgtatttatttttttcgcgtgcgttt

SEQ ID NO: 216 - DNA, 19 bp, Artificial sequence
  Synthetic
tttcgtaggtttggtcgcg

SEQ ID NO: 217 - DNA, 26 bp, Artificial sequence
  Synthetic
aacctaaataattaataacaacccgc

SEQ ID NO: 218 - DNA, 21 bp, Artificial sequence
  Synthetic
ccacggacggcgacgggtatt

SEQ ID NO: 219 - DNA, 88 bp, Homo sapiens
gtgggccgggcgtgacgcgcggtcaaagtgcaatgatttttcagttcggttggctaaacagggtcagagctgagagcgaagcagaagg

SEQ ID NO: 220 - DNA, 88 bp, Artificial sequence
  Synthetic
gtgggtcgggcgtgacgcgcggttaaagtgtaatgattttttagttcggttggttaaatagggttagagttgagagcgaagtagaagg

SEQ ID NO: 221 - DNA, 18 bp, Artificial sequence
  Synthetic
tggttcgggcgtgacgcg

SEQ ID NO: 222 - DNA, 23 bp, Artificial sequence
  Synthetic
tctaaccctatttaaccaaccga

SEQ ID NO: 223 - DNA, 20 bp, Artificial sequence
  Synthetic
cgccgagggcggttaaagtg

SEQ ID NO: 224 - DNA, 94 bp, Homo sapiens
ggacctcctcggccccgccccatccgccttcgggatgctgctgagccccgtcacctccacccccttctcggtcaaggacatcctgcgactggag

SEQ ID NO: 225 - DNA, 94 bp, Artificial sequence
  Synthetic
ggattttttcggtttcgttttattcgttttcgggatgttgttgagtttcgttatttttatttttttttcggttaaggatattttgcgattggag

SEQ ID NO: 226 - DNA, 25 bp, Artificial sequence
  Synthetic
gattttttcggtttcgttttattcg

SEQ ID NO: 227 - DNA, 24 bp, Artificial sequence
  Synthetic
caatcgcaaaatatccttaaccga

SEQ ID NO: 228 - DNA, 21 bp, Artificial sequence
  Synthetic
ccacggacggttttcgggatg

SEQ ID NO: 229 - DNA, 89 bp, Homo sapiens
ctgtcagtgctgaccgagcgccgcgccttccggccatacgggctccacggtgcgcggttccccagccctcgcggccctccccgcccccg

SEQ ID NO: 230 - DNA, 89 bp, Artificial sequence
  Synthetic
ttgttagtgttgatcgagcgtcgcgtttttcggttatacgggttttacggtgcgcggttttttagttttcgcggtttttttcgttttcg

SEQ ID NO: 231 - DNA, 22 bp, Artificial sequence
  Synthetic
cgtcgcgtttttcggttatacg

SEQ ID NO: 232 - DNA, 21 bp, Artificial sequence
  Synthetic
cgcgaaaactaaaaaaccgcg

SEQ ID NO: 233 - DNA, 21 bp, Artificial sequence
  Synthetic
ccacggacggcaccgtaaaac

SEQ ID NO: 234 - DNA, 114 bp, Homo sapiens
cggagtatggtgaggagcgcgggggacgggtgcgggaaggggacagcagggctgagcctggggcccgcaagacccagcagcccgagcgggcgcagagaccccacgccacgcaca

SEQ ID NO: 235 - DNA, 114 bp, Artificial sequence
  Synthetic
cggagtatggtgaggagcgcgggggacgggtgcgggaaggggatagtagggttgagtttggggttcgtaagatttagtagttcgagcgggcgtagagattttacgttacgtata

SEQ ID NO: 236 - DNA, 18 bp, Artificial sequence
  Synthetic
ggttgagtttggggttcg

SEQ ID NO: 237 - DNA, 23 bp, Artificial sequence
  Synthetic
cgtaacgtaaaatctctacgccc

SEQ ID NO: 238 - DNA, 21 bp, Artificial sequence
  Synthetic
ccacggacgcgctcgaactac

SEQ ID NO: 239 - DNA, 95 bp, Homo sapiens
ggagagcagcccgcagaacctggccgcgtactacacgcctttcccgtcctatggacactacagaaacagcctggccaccgtggaggaagacttcc

SEQ ID NO: 240 - DNA, 95 bp, Artificial sequence
  Synthetic
ggagagtagttcgtagaatttggtcgcgtattatacgtttttttcgttttatggatattatagaaatagtttggttatcgtggaggaagattttt

SEQ ID NO: 241 - DNA, 23 bp, Artificial sequence
  Synthetic
gagtagttcgtagaatttggtcg

SEQ ID NO: 242 - DNA, 31 bp, Artificial sequence
  Synthetic
ccacgataaccaaactatttctataatatcc

SEQ ID NO: 243 - DNA, 21 bp, Artificial sequence
  Synthetic
ccacggacggcgtattatacg

SEQ ID NO: 244 - DNA, 23 bp, Artificial sequence
  Synthetic
ggagagtagttcgtagaatttgg

SEQ ID NO: 245 - DNA, 35 bp, Artificial sequence
  Synthetic
ctatttctataatatccataaaacgaaaaaaacgt

SEQ ID NO: 246 - DNA, 21 bp, Artificial sequence
  Synthetic
ccacggacggtcgcgtattat

SEQ ID NO: 247 - DNA, 92 bp, Homo sapiens
gggaaggtgccctgcgcgcgcgcgctcaccagatgaagtcggtgcagtggctgcagaaggtgggctgcttgaagaagcgggcggtgaatttg

SEQ ID NO: 248 - DNA, 92 bp, Artificial sequence
  Synthetic
gggaaggtgttttgcgcgcgcgcgtttattagatgaagtcggtgtagtggttgtagaaggtgggttgtttgaagaagcgggcggtgaatttg

SEQ ID NO: 249 - DNA, 17 bp, Artificial sequence
  Synthetic
ggaaggtgttttgcgcg

SEQ ID NO: 250 - DNA, 21 bp, Artificial sequence
  Synthetic
cttctacaaccactacaccga

SEQ ID NO: 251 - DNA, 21 bp, Artificial sequence
  Synthetic
ccacggacggcgcgcgtttat

SEQ ID NO: 252 - DNA, 107 bp, Homo sapiens
gcctcggggcccggggactcacaattacgggcagagaacacatagtgaagagcacggtcatcagcgccagcagcaggaggtgatccagctcctccaggggctgaggg

SEQ ID NO: 253 - DNA, 107 bp, Artificial sequence
  Synthetic
gtttcggggttcggggatttataattacgggtagagaatatatagtgaagagtacggttattagcgttagtagtaggaggtgatttagtttttttaggggttgaggg

SEQ ID NO: 254 - DNA, 24 bp, Artificial sequence
  Synthetic
gggttcggggatttataattacgg

SEQ ID NO: 255 - DNA, 25 bp, Artificial sequence
  Synthetic
cctcctactactaacgctaataacc

SEQ ID NO: 256 - DNA, 21 bp, Artificial sequence
  Synthetic
ccacggacgcgtactcttcac

SEQ ID NO: 257 - DNA, 80 bp, Homo sapiens
ggcggctgcagcggcacccgcgctcctgcaccagggactgtgccgagccgcgcgcggacgggagggaagcgtcccctcag

SEQ ID NO: 258 - DNA, 80 bp, Artificial sequence
  Synthetic
ggcggttgtagcggtattcgcgtttttgtattagggattgtgtcgagtcgcgcgcggacgggagggaagcgtttttttag

SEQ ID NO: 259 - DNA, 18 bp, Artificial sequence
  Synthetic
gttgtagcggtattcgcg

SEQ ID NO: 260 - DNA, 18 bp, Artificial sequence
  Synthetic
cttctctcccgtccgcgc

SEQ ID NO: 261 - DNA, 20 bp, Artificial sequence
  Synthetic
cgccgaggcgcgactcgaca

SEQ ID NO: 262 - DNA, 143 bp, Homo sapiens
tccagaaacacgggtatctccgcgtggtgctttgcggtcgccgtcgttgtggccgtccggggtggggtgtgaggaggggacgaaggagggaaggaagggcaaggcggggggggctctgcgagagcgcgcccagccccgccttc

SEQ ID NO: 263 - DNA, 143 bp, Artificial sequence
  Synthetic
tttagaaatacgggtattttcgcgtggtgttttgcggtcgtcgtcgttgtggtcgttcggggtggggtgtgaggaggggacgaaggagggaaggaagggtaaggcggggggggttttgcgagagcgcgtttagtttcgttttt

SEQ ID NO: 264 - DNA, 21 bp, Artificial sequence
  Synthetic
agaaatacgggtattttcgcg

SEQ ID NO: 265 - DNA, 17 bp, Artificial sequence
  Synthetic
ccacaacgacgacgacc

SEQ ID NO: 266 - DNA, 21 bp, Artificial sequence
  Synthetic
ccacggacgcgcaaaacacca

SEQ ID NO: 267 - DNA, 120 bp, Homo sapiens
cggtcgggcaggcgggacggagattacctggctgtccaggggaccttatgcagggtttggcccgagcccaggggcagcgaggggcgtctgcggatgcggctccctgtgcggcacaacacc

SEQ ID NO: 268 - DNA, 120 bp, Artificial sequence
  Synthetic
cggtcgggtaggcgggacggagattatttggttgtttaggggattttatgtagggtttggttcgagtttaggggtagcgaggggcgtttgcggatgcggttttttgtgcggtataatatt

SEQ ID NO: 269 - DNA, 20 bp, Artificial sequence
  Synthetic
gttcgagtttaggggtagcg

SEQ ID NO: 270 - DNA, 17 bp, Artificial sequence
  Synthetic
ccgcacaaaaaaccgca

SEQ ID NO: 271 - DNA, 21 bp, Artificial sequence
  Synthetic
ccacggacgatccgcaaacgc

SEQ ID NO: 272 - DNA, 55 bp, Homo sapiens
ccggagcactcgccgctgcgcgccctgaagccgctggcggtaggcggccctcgag

SEQ ID NO: 273 - DNA, 55 bp, Artificial sequence
  Synthetic
tcggagtattcgtcgttgcgcgttttgaagtcgttggcggtaggcggttttcgag

SEQ ID NO: 274 - DNA, 18 bp, Artificial sequence
  Synthetic
ggagtattcgtcgttgcg

SEQ ID NO: 275 - DNA, 17 bp, Artificial sequence
  Synthetic
cgaaaaccgcctaccgc

SEQ ID NO: 276 - DNA, 20 bp, Artificial sequence
  Synthetic
cgccgagggcgttttgaagt

SEQ ID NO: 277 - DNA, 96 bp, Homo sapiens
cccgggcctacggtcctcccgccacctccacggggcggctgttggggccccaccaggcagagccgtgttctcaggcgttggctctcatggaggtgg

SEQ ID NO: 278 - DNA, 96 bp, Artificial sequence
  Synthetic
ttcgggtttacggttttttcgttatttttacggggcggttgttggggttttattaggtagagtcgtgtttttaggcgttggtttttatggaggtgg

SEQ ID NO: 279 - DNA, 25 bp, Artificial sequence
  Synthetic
acggttttttcgttatttttacggg

SEQ ID NO: 280 - DNA, 21 bp, Artificial sequence
  Synthetic
caacgcctaaaaacacgactc

SEQ ID NO: 281 - DNA, 20 bp, Artificial sequence
  Synthetic
cgccgaggggcggttgttgg

SEQ ID NO: 282 - DNA, 148 bp, Homo sapiens
gggcctgtcccgttccctgctccccatacaggcgaggctgcgtgcacacagcttcctgtaccccaggagggcctgcctggcacgcacccggtggctgcaccatccacacgcaagactgcaacttcagatgctccgcacgctggagatg

SEQ ID NO: 283 - DNA, 148 bp, Artificial sequence
  Synthetic
gggtttgtttcgttttttgttttttatataggcgaggttgcgtgtatatagttttttgtattttaggagggtttgtttggtacgtattcggtggttgtattatttatacgtaagattgtaattttagatgtttcgtacgttggagatg

SEQ ID NO: 284 - DNA, 20 bp, Artificial sequence
  Synthetic
ttatataggcgaggttgcgt

SEQ ID NO: 285 - DNA, 30 bp, Artificial sequence
  Synthetic
cttacgtataaataatacaaccaccgaata

SEQ ID NO: 286 - DNA, 21 bp, Artificial sequence
  Synthetic
ccacggacgacgtaccaaaca

SEQ ID NO: 287 - DNA, 88 bp, Homo sapiens
cggagctaggagggtggggctcggagggcgcaggaagagcggctctgcgaggaaagggaaaggagaggccgcttctgggaagggaccc

SEQ ID NO: 288 - DNA, 88 bp, Artificial sequence
  Synthetic
cggagttaggagggtggggttcggagggcgtaggaagagcggttttgcgaggaaagggaaaggagaggtcgtttttgggaagggattt

SEQ ID NO: 289 - DNA, 18 bp, Artificial sequence
  Synthetic
ttaggagggtggggttcg

SEQ ID NO: 290 - DNA, 18 bp, Artificial sequence
  Synthetic
ctttcctcgcaaaaccgc

SEQ ID NO: 291 - DNA, 21 bp, Artificial sequence
  Synthetic
ccacggacgggagggcgtagg

SEQ ID NO: 292 - DNA, 59 bp, Homo sapiens
gccccggcgggccccgaggcggccgcggcctgcaacgtcatcgtgaacggcacgcgcgg

SEQ ID NO: 293 - DNA, 59 bp, Artificial sequence
  Synthetic
gtttcggcgggtttcgaggcggtcgcggtttgtaacgttatcgtgaacggtacgcgcgg

SEQ ID NO: 294 - DNA, 17 bp, Artificial sequence
  Synthetic
tttcggcgggtttcgag

SEQ ID NO: 295 - DNA, 20 bp, Artificial sequence
  Synthetic
cgtaccgttcacgataacgt

SEQ ID NO: 296 - DNA, 20 bp, Artificial sequence
  Synthetic
cgccgaggggcggtcgcggt

SEQ ID NO: 297 - DNA, 20 bp, Artificial sequence
  Synthetic
cgccgaggttacaaaccgcg

SEQ ID NO: 298 - DNA, 89 bp, Homo sapiens
ctaggcgagatggtggaaggcgtgtccgtacgggggtgggctggggtccccgtgcagaagggcgcgcgaggacccaggctggttttccc

SEQ ID NO: 299 - DNA, 89 bp, Artificial sequence
  Synthetic
ttaggcgagatggtggaaggcgtgttcgtacgggggtgggttggggttttcgtgtagaagggcgcgcgaggatttaggttggttttttt

SEQ ID NO: 300 - DNA, 17 bp, Artificial sequence
  Synthetic
cgagatggtggaaggcg

SEQ ID NO: 301 - DNA, 17 bp, Artificial sequence
  Synthetic
gcgcccttctacacgaa

SEQ ID NO: 302 - DNA, 21 bp, Artificial sequence
  Synthetic
ccacggacggtgttcgtacgg

SEQ ID NO: 303 - DNA, 145 bp, Homo sapiens
gcgctgctgcgccgccaggcaaggcgagggtccgggagaaggctcggctccctcctaaacatgtggcccgtggcgtccccttgtcccctccgagcgatgctcctgcgcccttcgccgcctcccgcgctgctgcgccgccaggcaa

SEQ ID NO: 304 - DNA, 123 bp, Artificial sequence
  Synthetic
ggcgagggttcgggagaaggttcggtttttttttaaatatgtggttcgtggcgtttttttgtttttttcgagcgatgtttttgcgtttttcgtcgtttttcgcgttgttgcgtcgttaggtaa

SEQ ID NO: 305 - DNA, 21 bp, Artificial sequence
  Synthetic
aaatatgtggttcgtggcgtt

SEQ ID NO: 306 - DNA, 19 bp, Artificial sequence
  Synthetic
acgcaacaacgcgaaaaac

SEQ ID NO: 307 - DNA, 20 bp, Artificial sequence
  Synthetic
cgccgaggcgacgaaaaacg

SEQ ID NO: 308 - DNA, 137 bp, Homo sapiens
acgagaaagagatcgtgcagggggtgctgcaacagggcacggcgtggaggaggaaccagaccgcggccagagcgttcaggtactcctgccctcgcggctcctcccctctagcgtcctttcctccccgagtgcagagg

SEQ ID NO: 309 - DNA, 137 bp, Artificial sequence
  Synthetic
acgagaaagagatcgtgtagggggtgttgtaatagggtacggcgtggaggaggaattagatcgcggttagagcgtttaggtatttttgttttcgcggttttttttttttagcgttttttttttttcgagtgtagagg

SEQ ID NO: 310 - DNA, 21 bp, Artificial sequence
  Synthetic
ggggtgttgtaatagggtacg

SEQ ID NO: 311 - DNA, 19 bp, Artificial sequence
  Synthetic
ctaaacgctctaaccgcga

SEQ ID NO: 312 - DNA, 21 bp, Artificial sequence
  Synthetic
ccacggacgggcgtggaggag

SEQ ID NO: 313 - DNA, 117 bp, Homo sapiens
cgcgccgttggtcacctcgccggccgccagcgtcgaatggaagcccgacttgtaccaggactcgtacgggtgcgccatgcccacgcgcgggtacagcccgtcggctgccgtcgtgtg

SEQ ID NO: 314 - DNA, 117 bp, Artificial sequence
  Synthetic
cgcgtcgttggttatttcgtcggtcgttagcgtcgaatggaagttcgatttgtattaggattcgtacgggtgcgttatgtttacgcgcgggtatagttcgtcggttgtcgtcgtgtg

SEQ ID NO: 315 - DNA, 21 bp, Artificial sequence
  Synthetic
tagcgtcgaatggaagttcga

SEQ ID NO: 316 - DNA, 18 bp, Artificial sequence
  Synthetic
ggtcgttagcgtcgaatg

SEQ ID NO: 317 - DNA, 20 bp, Artificial sequence
  Synthetic
gcgcgtaaacataacgcacc

SEQ ID NO: 318 - DNA, 21 bp, Artificial sequence
  Synthetic
ccacggacgccgtacgaatcc

SEQ ID NO: 319 - DNA, 85 bp, Homo sapiens
ggttccttcccgtgggttcttaatcgtctcgctgacttccagaatgaaactgcagaccctcgcggtaaagatggcgtgaccagaa

SEQ ID NO: 320 - DNA, 85 bp, Artificial sequence
  Synthetic
ggttttttttcgtgggtttttaatcgtttcgttgatttttagaatgaaattgtagattttcgcggtaaagatggcgtgattagaa

SEQ ID NO: 321 - DNA, 22 bp, Artificial sequence
  Synthetic
tcgtgggtttttaatcgtttcg

SEQ ID NO: 322 - DNA, 18 bp, Artificial sequence
  Synthetic
tcacgccatctttaccgc

SEQ ID NO: 323 - DNA, 21 bp, Artificial sequence
  Synthetic
ccacggacgcgaaaatctaca

SEQ ID NO: 324 - DNA, 26 bp, Artificial sequence
  Synthetic
ggttttttttcgtgggtttttaatcg

SEQ ID NO: 325 - DNA, 21 bp, Artificial sequence
  Synthetic
ctaatcacgccatctttaccg

SEQ ID NO: 326 - DNA, 21 bp, Artificial sequence
  Synthetic
ccacggacggtttcgttgatt

SEQ ID NO: 327 - DNA, 115 bp, Homo sapiens
ggagtgagtgcctacaacgcgcaggccggactgatcccccgttgctgcaggttggtgccccaagctgcgggtgctcgggcgccaactaaagccagctctgtccagacgcggaaag

SEQ ID NO: 328 - DNA, 115 bp, Artificial sequence
  Synthetic
ggagtgagtgtttataacgcgtaggtcggattgatttttcgttgttgtaggttggtgttttaagttgcgggtgttcgggcgttaattaaagttagttttgtttagacgcggaaag

SEQ ID NO: 329 - DNA, 23 bp, Artificial sequence
  Synthetic
cgtaggtcggattgatttttcgt

SEQ ID NO: 330 - DNA, 30 bp, Artificial sequence
  Synthetic
tctaaacaaaactaactttaattaacgccc

SEQ ID NO: 331 - DNA, 21 bp, Artificial sequence
  Synthetic
ccacggacgcgaacacccgca

SEQ ID NO: 332 - DNA, 19 bp, Artificial sequence
  Synthetic
aggaaattgcgggttttcg

SEQ ID NO: 333 - DNA, 21 bp, Artificial sequence
  Synthetic
ggaaggaaattgcgggttttc

SEQ ID NO: 334 - DNA, 25 bp, Artificial sequence
  Synthetic
ccaaaaatcgtcgctaaaaatcaac

SEQ ID NO: 335 - DNA, 21 bp, Artificial sequence
  Synthetic
ccacggacgcgcgcattcact

SEQ ID NO: 336 - DNA, 100 bp, Homo sapiens
ggcggccgcgacccctccccgctgacctcactcgagccgccgcctggcgcagatataagcggcggcccatctgaagagggctcggcaggcgcccggggtc

SEQ ID NO: 337 - DNA, 100 bp, Artificial sequence
  Synthetic
ggcggtcgcgattttttttcgttgattttattcgagtcgtcgtttggcgtagatataagcggcggtttatttgaagagggttcggtaggcgttcggggtt

SEQ ID NO: 338 - DNA, 23 bp, Artificial sequence
  Synthetic
tttcgttgattttattcgagtcg

SEQ ID NO: 339 - DNA, 19 bp, Artificial sequence
  Synthetic
tcttcaaataaaccgccgc

SEQ ID NO: 340 - DNA, 20 bp, Artificial sequence
  Synthetic
cgccgagggtcgtttggcgt

SEQ ID NO: 341 - DNA, 118 bp, Homo sapiens
cgggtggtgaagctgccccacggcctgggagagccttatcgccgcggtcgctggacgtgtgtggatgtttatgagcgagacctggagccccacagcttcggcggactcctggagggaa

SEQ ID NO: 342 - DNA, 118 bp, Artificial sequence
  Synthetic
cgggtggtgaagttgttttacggtttgggagagttttatcgtcgcggtcgttggacgtgtgtggatgtttatgagcgagatttggagttttatagtttcggcggatttttggagggaa

SEQ ID NO: 343 - DNA, 22 bp, Artificial sequence
  Synthetic
gtttgggagagttttatcgtcg

SEQ ID NO: 344 - DNA, 18 bp, Artificial sequence
  Synthetic
cctccaaaaatccgccga

SEQ ID NO: 345 - DNA, 20 bp, Artificial sequence
  Synthetic
cgccgagggcggtcgttgga

SEQ ID NO: 346 - DNA, 70 bp, Homo sapiens
ggggcgggggccgacagcccacgctggcgcggcaggcgcgtgcgcccgccgttttcgtgagcccgagcag

SEQ ID NO: 347 - DNA, 70 bp, Artificial sequence
  Synthetic
ggggtcggggtcgatagtttacgttggcgcggtaggcgcgtgcgttcgtcgttttcgtgagttcgagtag

SEQ ID NO: 348 - DNA, 20 bp, Artificial sequence
  Synthetic
gtcggggtcgatagtttacg

SEQ ID NO: 349 - DNA, 19 bp, Artificial sequence
  Synthetic
actcgaactcacgaaaacg

SEQ ID NO: 350 - DNA, 20 bp, Artificial sequence
  Synthetic
cgccgagggacgaacgcacg

SEQ ID NO: 351 - DNA, 96 bp, Homo sapiens
ggagcccccagccccacgcgggcacacgcagggtgggtggtcacgcccgcagggtccgcgagcgcggcgcagagcgcgggccgtgggaagtttctc

SEQ ID NO: 352 - DNA, 96 bp, Artificial sequence
  Synthetic
ggagtttttagttttacgcgggtatacgtagggtgggtggttacgttcgtagggttcgcgagcgcggcgtagagcgcgggtcgtgggaagtttttt

SEQ ID NO: 353 - DNA, 18 bp, Artificial sequence
  Synthetic
gtagggtgggtggttacg

SEQ ID NO: 354 - DNA, 17 bp, Artificial sequence
  Synthetic
aacttcccacgacccgc

SEQ ID NO: 355 - DNA, 20 bp, Artificial sequence
  Synthetic
cgccgagggttcgtagggtt

SEQ ID NO: 356 - DNA, 127 bp, Homo sapiens
ggcgccgccattgcggtcctcattttgctgctggtgggttgggctacagcaggcctctggagccacaccagggcacgggagtgggtgcagggaccgtcaccgcgccttcacacgcaccatagtgccc

SEQ ID NO: 357 - DNA, 127 bp, Artificial sequence
  Synthetic
ggcgtcgttattgcggtttttattttgttgttggtgggttgggttatagtaggtttttggagttatattagggtacgggagtgggtgtagggatcgttatcgcgtttttatacgtattatagtgttt

SEQ ID NO: 358 - DNA, 23 bp, Artificial sequence
  Synthetic
tggagttatattagggtacggga

SEQ ID NO: 359 - DNA, 28 bp, Artificial sequence
  Synthetic
acactataatacgtataaaaacgcgata

SEQ ID NO: 360 - DNA, 21 bp, Artificial sequence
  Synthetic
ccacggacgaacgatccctac

SEQ ID NO: 361 - DNA, 140 bp, Homo sapiens
ggcggcgaggggcgcgtccgcgggtgggtttcacctgggtggtgggcatgtcgggcccgctagggcgagggtctggccaggggcgtagttctcctggtgggtggggacgctccgtggcgattggggtcactcctctgagg

SEQ ID NO: 362 - DNA, 140 bp, Artificial sequence
  Synthetic
ggcggcgaggggcgcgttcgcgggtgggttttatttgggtggtgggtatgtcgggttcgttagggcgagggtttggttaggggcgtagtttttttggtgggtggggacgtttcgtggcgattggggttatttttttgagg

SEQ ID NO: 363 - DNA, 17 bp, Artificial sequence
  Synthetic
ggtggtgggtatgtcgg

SEQ ID NO: 364 - DNA, 17 bp, Artificial sequence
  Synthetic
ccaatcgccacgaaacg

SEQ ID NO: 365 - DNA, 21 bp, Artificial sequence
  Synthetic
ccacggacgggttcgttaggg

SEQ ID NO: 366 - DNA, 117 bp, Homo sapiens
ccgtgggcgcggacagctgccgggagcggcaggcgtctcgatcggggacgcaggcacttccgtccctgcagagcatcagacgcgtctcgggacactggggacaacatctcctccgcg

SEQ ID NO: 367 - DNA, 117 bp, Artificial sequence
  Synthetic
tcgtgggcgcggatagttgtcgggagcggtaggcgtttcgatcggggacgtaggtattttcgtttttgtagagtattagacgcgtttcgggatattggggataatatttttttcgcg

SEQ ID NO: 368 - DNA, 18 bp, Artificial sequence
  Synthetic
gttgtcgggagcggtagg

SEQ ID NO: 369 - DNA, 21 bp, Artificial sequence
  Synthetic
ccaatatcccgaaacgcgtct

SEQ ID NO: 370 - DNA, 21 bp, Artificial sequence
  Synthetic
ccacggacggcgtttcgatcg

SEQ ID NO: 371 - DNA, 120 bp, Homo sapiens
aagctgcgcccggagacgtgggagcgttctcttgttttccgagtgcgcggactcatcgggtcacagtttatgcttttatgacgcggtgagtccagccactgattcctaacggtttagagt

SEQ ID NO: 372 - DNA, 120 bp, Artificial sequence
  Synthetic
aagttgcgttcggagacgtgggagcgtttttttgtttttcgagtgcgcggatttatcgggttatagtttatgtttttatgacgcggtgagtttagttattgatttttaacggtttagagt

SEQ ID NO: 373 - DNA, 23 bp, Artificial sequence
  Synthetic
cgtttttttgtttttcgagtgcg

SEQ ID NO: 374 - DNA, 23 bp, Artificial sequence
  Synthetic
tcaataactaaactcaccgcgtc

SEQ ID NO: 375 - DNA, 21 bp, Artificial sequence
  Synthetic
ccacggacggcggatttatcg

SEQ ID NO: 376 - DNA, 224 bp, Homo sapiens
ctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaaga

SEQ ID NO: 377 - DNA, 224 bp, Artificial sequence
  Synthetic
ttttgatttgagttttttttggaattttgtaggttttatttgtttttttttagatgagttttttttttggtgtttgtttttttgattaggtgtttaagatagtgttgtgggtgtaggtattaatattggtttgtgtgataaggttatgaggttggtgtaaagtggttttggagtgtgtattaagtaggtgtatagtaggtttgaatagattttttattttaaga

SEQ ID NO: 378 - DNA, 19 bp, Artificial sequence
  Synthetic
ccatgaggctggtgtaaag

SEQ ID NO: 379 - DNA, 24 bp, Artificial sequence
  Synthetic
ctactgtgcacctacttaatacac

SEQ ID NO: 380 - DNA, 20 bp, Artificial sequence
  Synthetic
cgccgagggcggccttggag

SEQ ID NO: 381 - DNA, 30 bp, Artificial sequence
  Synthetic
gtgtttgtttttttgattaggtgtttaaga

SEQ ID NO: 382 - DNA, 26 bp, Artificial sequence
  Synthetic
ctttacaccaacctcataaccttatc

SEQ ID NO: 383 - DNA, 21 bp, Artificial sequence
  Synthetic
gacgcggagatagtgttgtgg

SEQ ID NO: 384 - DNA, 139 bp, Homo sapiens
ggccacacaggcccactctggccctctgagcccccggcggacccagggcattcaaggagcggctctgggctgccagcgcaggcctccgcgcaaacacagcaggctggaagtggcgctcatcaccggcacgtcttcccag

SEQ ID NO: 385 - DNA, 139 bp, Artificial sequence
  Synthetic
ggttatataggtttattttggttttttgagttttcggcggatttagggtatttaaggagcggttttgggttgttagcgtaggttttcgcgtaaatatagtaggttggaagtggcgtttattatcggtacgtttttttag

SEQ ID NO: 386 - DNA, 28 bp, Artificial sequence
  Synthetic
ggtttattttggttttttgagttttcgg

SEQ ID NO: 387 - DNA, 24 bp, Artificial sequence
  Synthetic
tccaacctactatatttacgcgaa

SEQ ID NO: 388 - DNA, 21 bp, Artificial sequence
  Synthetic
ccacggacggcggatttaggg

SEQ ID NO: 389 - DNA, 171 bp, Artificial sequence
  Synthetic
tccacgtggtgcccactctggacaggtggagcagagggaaggtggtggcatggtggggagggtggcctggaggacccgattggctgagtgtaaaccaggagaggacatgactttcagccctgcagccagacacagctgagctggtgtgacctgtgtggagagttcatctgg

SEQ ID NO: 390 - DNA, 180 bp, Artificial sequence
  Synthetic
tttatcgtggtgtttattttggataggtggagtagagggaaggtggtgcgtatggtgggcgagcgcgtgcgtttggaggatttcgattggttgacgtgtaaattaggacgaggatatgatttttagttttgtagttagatatagttgagttggtgtgatttgtgtggagagtttatttgg

SEQ ID NO: 391 - DNA, 15 bp, Artificial sequence
  Synthetic
cgcatggtgggcgag

SEQ ID NO: 392 - DNA, 18 bp, Artificial sequence
  Synthetic
acacgtcagccaatcggg

SEQ ID NO: 393 - DNA, 20 bp, Artificial sequence
  Synthetic
gacgcggaggcgcgtgcgcc

SEQ ID NO: 394 - DNA, 17 bp, Artificial sequence
  Synthetic
tgcgtatggtgggcgag

SEQ ID NO: 395 - DNA, 26 bp, Artificial sequence
  Synthetic
cctaatttacacgtcaaccaatcgaa

SEQ ID NO: 396 - DNA, 21 bp, Artificial sequence
  Synthetic
gacgcggaggcgcgtgcgttt

SEQ ID NO: 397 - DNA, 21 bp, Artificial sequence
  Synthetic
ccacggacggcgcgtgcgttt

SEQ ID NO: 398 - DNA, 28 bp, Artificial sequence
  Synthetic
agccggttttccggctgagacctcggcg

SEQ ID NO: 399 - DNA, 28 bp, Artificial sequence
  Synthetic
agccggttttccggctgagacctcggcg

SEQ ID NO: 400 - DNA, 29 bp, Artificial sequence
  Synthetic
agccggttttccggctgagactccgcgtc

SEQ ID NO: 401 - DNA, 29 bp, Artificial sequence
  Synthetic
agccggttttccggctgagacgtccgtgg

SEQ ID NO: 402 - DNA, 28 bp, Artificial sequence
  Synthetic
agccggttttccggctgagaggacgcgc

SEQ ID NO: 403 - DNA, 108 bp, Homo sapiens
ggaaggaaattgcgggttcccgtctgccttgtctccagcttctctgctgaagcccggtagcagtgaatgcgcgctgactttcagcgacgactcctggaagcaacgcca

SEQ ID NO: 404 - DNA, 108 bp, Artificial sequence
  Synthetic
ggaaggaaattgcgggttttcgtttgttttgtttttagtttttttgttgaagttcggtagtagtgaatgcgcgttgatttttagcgacgatttttggaagtaacgtta

SEQ ID NO: 405 - DNA, 27 bp, Artificial sequence
  Synthetic
aggccacggacgcgaaaaatcccacgc

SEQ ID NO: 406 - DNA, 17 bp, Artificial sequence
  Synthetic
gtcgagcgtttggtgcg

SEQ ID NO: 407 - DNA, 19 bp, Artificial sequence
  Synthetic
ctcgtcgaaatcgaaacgc

SEQ ID NO: 408 - DNA, 32 bp, Artificial sequence
  Synthetic
cgcgccgagggcgatagcgttttttattgtcg

SEQ ID NO: 409 - DNA, 19 bp, Artificial sequence
  Synthetic
cgaggttatggaggtgacg

SEQ ID NO: 410 - DNA, 22 bp, Artificial sequence
  Synthetic
cgaatactacccgttaaacacg

SEQ ID NO: 411 - DNA, 27 bp, Artificial sequence
  Synthetic
aggccacggacgggcggattagtcgcg

SEQ ID NO: 412 - DNA, 330 bp, Homo sapiens
ggcggcgccgcgaccgccttccttcgctgcgtcccgcccgctccacgcctcgctcaccgccgccgcttctccctgccccgcagcgcgcagggaccatgtcggcggagaccgcgagcggccccacagaggaccaggtggaaatcctggagtacaacttcaacaaggtcgacaagcacccggattccaccacgctgtgcctcatcgcggccgaggcaggcctttccgaggaggagacccaggtgcgtccccacacgcgcccagcgcgccccgacccctgcctgggctgagccttctcgcggctgggcggtcctgttcgtccgcgcctcccgc

SEQ ID NO: 413 - DNA, 330 bp, Artificial sequence
  Synthetic
ggcggcgtcgcgatcgttttttttcgttgcgtttcgttcgttttacgtttcgtttatcgtcgtcgtttttttttgtttcgtagcgcgtagggattatgtcggcggagatcgcgagcggttttatagaggattaggtggaaattttggagtataattttaataaggtcgataagtattcggattttattacgttgtgttttatcgcggtcgaggtaggttttttcgaggaggagatttaggtgcgtttttatacgcgtttagcgcgtttcgatttttgtttgggttgagttttttcgcggttgggcggttttgttcgttcgcgtttttcgt

SEQ ID NO: 414 - DNA, 22 bp, Artificial sequence
  Synthetic
gcgtcgcgatcgttttttttcg

SEQ ID NO: 415 - DNA, 24 bp, Artificial sequence
  Synthetic
aacgacgacgataaacgaaacgta

SEQ ID NO: 416 - DNA, 30 bp, Artificial sequence
  Synthetic
aggccacggacggttgcgtttcgttcgttt

SEQ ID NO: 417 - DNA, 22 bp, Artificial sequence
  Synthetic
gtagcgcgtagggattatgtcg

SEQ ID NO: 418 - DNA, 27 bp, Artificial sequence
  Synthetic
tttccacctaatcctctataaaaccgc

SEQ ID NO: 419 - DNA, 26 bp, Artificial sequence
  Synthetic
aggccacggacgctcgcgatctccgc

SEQ ID NO: 420 - DNA, 19 bp, Artificial sequence
  Synthetic
acgttgtgttttatcgcgg

SEQ ID NO: 421 - DNA, 22 bp, Artificial sequence
  Synthetic
ctaaacgcgtataaaaacgcac

SEQ ID NO: 422 - DNA, 32 bp, Artificial sequence
  Synthetic
aggccacggacggtcgaggtaggttttttcga

SEQ ID NO: 423 - DNA, 16 bp, Artificial sequence
  Synthetic
caactcatccgcgacg

SEQ ID NO: 424 - DNA, 27 bp, Artificial sequence
  Synthetic
aggccacggacggtcgacgcccaacaa

SEQ ID NO: 425 - DNA, 37 bp, Artificial sequence
  Synthetic
cgcgccgagggcgttaggatttatttttttttttcga

SEQ ID NO: 426 - DNA, 72 bp, Homo sapiens
cgggacagagccgaccaatcaggcggctcggcagcggggcagaggtcagggggcgggccgaggggaagccaa

SEQ ID NO: 427 - DNA, 72 bp, Artificial sequence
  Synthetic
cgggatagagtcgattaattaggcggttcggtagcggggtagaggttagggggcgggtcgaggggaagttaa

SEQ ID NO: 428 - DNA, 24 bp, Artificial sequence
  Synthetic
cgggatagagtcgattaattaggc

SEQ ID NO: 429 - DNA, 18 bp, Artificial sequence
  Synthetic
taacttcccctcgacccg

SEQ ID NO: 430 - DNA, 24 bp, Artificial sequence
  Synthetic
cgcgccgaggcggttcggtagcgg

SEQ ID NO: 431 - DNA, 26 bp, Artificial sequence
  Synthetic
cgcgccgaggttacaaaccgcgaccg

SEQ ID NO: 432 - DNA, 26 bp, Artificial sequence
  Synthetic
ttttcgttgattttattcgagtcgtc

SEQ ID NO: 433 - DNA, 22 bp, Artificial sequence
  Synthetic
gaaccctcttcaaataaaccgc

SEQ ID NO: 434 - DNA, 30 bp, Artificial sequence
  Synthetic
cgcgccgaggcgtttggcgtagatataagc

SEQ ID NO: 435 - DNA, 33 bp, Artificial sequence
  Synthetic
aggccacggacggcggatttatcgggttatagt

SEQ ID NO: 436 - DNA, 35 bp, Artificial sequence
  Synthetic
acggacgcggaggcggatttagggtatttaaggag

SEQ ID NO: 437 - RNA, 1371 nt, Homo sapiens
aaucauuagagccugagucacucuccccaggagacccagaccuagaacuacccagagcaagaccacagcuggugaacaguccagccugucuccaguuggacuagccacaauucaagugcuugaaaaccacauguggagcagacaagauggagacaaauuccucucuccccacgaacaucucuggagggacaccugcuguaucugcuggcuaucucuuccuggauaucaucacuuaucugguauuugcagucaccuuuguccucgggguccugggcaacgggcuugugaucuggguggcuggauuccggaugacacacacagucaccaccaucaguuaccugaaccuggccguggcugacuucuguuucaccuccacuuugccauucuucauggucaggaaggccaugggaggacauuggccuuucggcugguuccugugcaaauucgucuuuaccauaguggacaucaacuuguucggaagugucuuccugaucgcccucauugcucuggaccgcuguguuugcguccugcauccagucuggacccagaaccaccgcaccgugagccuggccaagaaggugaucauugggcccugggugauggcucugcuccucacauugccaguuaucauucgugugacuacaguaccugguaaaacggggacaguagccugcacuuuuaacuuuucgcccuggaccaacgacccuaaagagaggauaaauguggccguugccauguugacggugagaggcaucauccgguucaucauuggcuucagcgcacccauguccaucguugcugucaguuaugggcuuauugccaccaagauccacaagcaaggcuugauuaaguccagucgucccuuacggguccucuccuuugucgcagcagccuuuuuucucugcugguccccauaucaggugguggcccuuauagccacagucagaauccgugaguuauugcaaggcauguacaaagaaauugguauugcaguggaugugacaagugcccuggccuucuucaacagcugccucaaccccaugcucuaugucuucaugggccaggacuuccgggagaggcugauccacgcccuucccgccagucuggagagggcccugaccgaggacucaacccaaaccagugacacagcuaccaauucuacuuuaccuucugcagagguggaguuacaggcaaagugaggagggagcugggggacacuuucgagcucccagcuccagcuucgucucaccuugaguuaggcugagccacaggcauuuccugcuuauuuuaggauuacccacucaucagaaaaaaaaaaaaaagccuuuguguccccugauuuggggagaauaaacagauaugaguuuauua

SEQ ID NO: 438 - RNA, 106 nt, Homo sapiens
guugccauguugacggugagaggcaucauccgguucaucauuggcuucagcgcacccauguccaucguugcugucaguuaugggcuuauugccaccaagauccaca

SEQ ID NO: 439 - DNA, 18 bp, Artificial sequence
  Synthetic
tgacggtgagaggcatca

SEQ ID NO: 440 - DNA, 22 bp, Artificial sequence
  Synthetic
ggtggcaataagcccataactg

SEQ ID NO: 441 - DNA, 23 bp, Artificial sequence
  Synthetic
cggttcatcattggcttcagcgc

SEQ ID NO: 442 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 443 - DNA, 25 bp, Artificial sequence
  Synthetic
ccagttggactagccacaattcaag

SEQ ID NO: 444 - DNA, 25 bp, Artificial sequence
  Synthetic
aggaatttgtctccatcttgtctgc

SEQ ID NO: 445 - DNA, 30 bp, Artificial sequence
  Synthetic
cgcgccgaggctccacatgtggttttcaag

SEQ ID NO: 446 - DNA, 32 bp, Artificial sequence
  Synthetic
aggccacggacgctccacatgtggttttcaag

SEQ ID NO: 447 - DNA, 20 bp, Artificial sequence
  Synthetic
gcccaagtctccatcatgac

SEQ ID NO: 448 - DNA, 19 bp, Artificial sequence
  Synthetic
ggtggtccacaaaggtgtc

SEQ ID NO: 449 - DNA, 32 bp, Artificial sequence
  Synthetic
cgcgccgaggcctacgatgaatttaagcactg

SEQ ID NO: 450 - DNA, 34 bp, Artificial sequence
  Synthetic
acggacgcggagcctacgatgaatttaagcactg

SEQ ID NO: 451 - DNA, 34 bp, Artificial sequence
  Synthetic
aggccacggacgcctacgatgaatttaagcactg

SEQ ID NO: 452 - DNA, 18 bp, Artificial sequence
  Synthetic
acgaccggtttggcttct

SEQ ID NO: 453 - DNA, 17 bp, Artificial sequence
  Synthetic
ccatcagcattgccgtc

SEQ ID NO: 454 - DNA, 29 bp, Artificial sequence
  Synthetic
cgcgccgaggcctgggtgtagagtctctc

SEQ ID NO: 455 - DNA, 31 bp, Artificial sequence
  Synthetic
acggacgcggagcctgggtgtagagtctctc

SEQ ID NO: 456 - DNA, 31 bp, Artificial sequence
  Synthetic
aggccacggacgcctgggtgtagagtctctc

SEQ ID NO: 457 - DNA, 21 bp, Artificial sequence
  Synthetic
tatcatttggctggcaaggag

SEQ ID NO: 458 - DNA, 23 bp, Artificial sequence
  Synthetic
tcaccaagggcgatttaatatgg

SEQ ID NO: 459 - DNA, 33 bp, Artificial sequence
  Synthetic
cgcgccgaggggtcattcatacttctcttggat

SEQ ID NO: 460 - DNA, 35 bp, Artificial sequence
  Synthetic
acggacgcggagggtcattcatacttctcttggat

SEQ ID NO: 461 - DNA, 22 bp, Artificial sequence
  Synthetic
agaacatggatgaccagtggat

SEQ ID NO: 462 - DNA, 18 bp, Artificial sequence
  Synthetic
ggcagcgttttgtgtggg

SEQ ID NO: 463 - DNA, 30 bp, Artificial sequence
  Synthetic
cgcgccgaggtgcaggatgaaatggagatc

SEQ ID NO: 464 - DNA, 32 bp, Artificial sequence
  Synthetic
acggacgcggagtgcaggatgaaatggagatc

SEQ ID NO: 465 - DNA, 26 bp, Artificial sequence
  Synthetic
tgcgaaaagatctgcaaaattttctc

SEQ ID NO: 466 - DNA, 24 bp, Artificial sequence
  Synthetic
aggtcctccatgatgtgttctatg

SEQ ID NO: 467 - DNA, 36 bp, Artificial sequence
  Synthetic
cgcgccgaggcaagaaggagaataagaatgaaaagg

SEQ ID NO: 468 - DNA, 38 bp, Artificial sequence
  Synthetic
acggacgcggagcaagaaggagaataagaatgaaaagg

SEQ ID NO: 469 - DNA, 38 bp, Artificial sequence
  Synthetic
aggccacggacgcaagaaggagaataagaatgaaaagg

SEQ ID NO: 470 - DNA, 21 bp, Artificial sequence
  Synthetic
ctgcttacaaaggagcttgca

SEQ ID NO: 471 - DNA, 26 bp, Artificial sequence
  Synthetic
gccttggaatatttcatcaatgacag

SEQ ID NO: 472 - DNA, 36 bp, Artificial sequence
  Synthetic
cgcgccgagggctttatctttgatattcttgatggt

SEQ ID NO: 473 - DNA, 38 bp, Artificial sequence
  Synthetic
aggccacggacggctttatctttgatattcttgatggt

SEQ ID NO: 474 - DNA, 22 bp, Artificial sequence
  Synthetic
ggattcaatgtcatccagagcc

SEQ ID NO: 475 - DNA, 20 bp, Artificial sequence
  Synthetic
tctgacccacgatacagcag

SEQ ID NO: 476 - DNA, 28 bp, Artificial sequence
  Synthetic
cgcgccgaggccagagcagatgcaagtg

SEQ ID NO: 477 - DNA, 30 bp, Artificial sequence
  Synthetic
aggccacggacgccagagcagatgcaagtg

SEQ ID NO: 478 - DNA, 24 bp, Artificial sequence
  Synthetic
acatgcaaaacttgcagattttgg

SEQ ID NO: 479 - DNA, 23 bp, Artificial sequence
  Synthetic
tgttcctatcactgtattccgct

SEQ ID NO: 480 - DNA, 32 bp, Artificial sequence
  Synthetic
acggacgcggagttggccatggtatctgtaag

SEQ ID NO: 481 - DNA, 32 bp, Artificial sequence
  Synthetic
aggccacggacgttggccatggtatctgtaag

SEQ ID NO: 482 - DNA, 18 bp, Artificial sequence
  Synthetic
gccggccgtctccttaac

SEQ ID NO: 483 - DNA, 21 bp, Artificial sequence
  Synthetic
tctccatcagaactctgcaac

SEQ ID NO: 484 - DNA, 28 bp, Artificial sequence
  Synthetic
acggacgcggagcaccgaacaccatgcc

SEQ ID NO: 485 - DNA, 19 bp, Artificial sequence
  Synthetic
gtgctgaggagtcggagtg

SEQ ID NO: 486 - DNA, 20 bp, Artificial sequence
  Synthetic
tccgagagaacagcatcacc

SEQ ID NO: 487 - DNA, 30 bp, Artificial sequence
  Synthetic
cgcgccgagggtgagagtgaagatggcatt

SEQ ID NO: 488 - DNA, 32 bp, Artificial sequence
  Synthetic
aggccacggacggtgagagtgaagatggcatt

SEQ ID NO: 489 - DNA, 24 bp, Artificial sequence
  Synthetic
tttgtaacctttgacgaccatgac

SEQ ID NO: 490 - DNA, 24 bp, Artificial sequence
  Synthetic
ggccattcacagtatggtatttct

SEQ ID NO: 491 - DNA, 34 bp, Artificial sequence
  Synthetic
aggccacggacgtgaatgacaatcttatccacgg

(19) United States
(12) Patent Application Publication
(10) Pub. No.: US 2022/0403471 A1
(43) Pub. Date: Dec. 22, 2022

(54) CHARACTERIZING METHYLATED DNA, RNA, AND PROTEINS IN SUBJECTS SUSPECTED OF HAVING LUNG NEOPLASIA

(71) Applicants: Exact Sciences Corporation, Madison, WI (US); Mayo Foundation for Medical Education and Research, Rochester, MN (US)

(72) Inventors: Scott Morris, Phoenix, AZ (US); David Mallery, Phoenix, AZ (US); Hatim T. Allawi, Middleton, WI (US); Graham P. Lidgard, Middleton, WI (US); Maria Giakoumopoulos, Middleton, WI (US); Michael W. Kaiser, Stoughton, WI (US); David A. Ahlquist, Rochester, MN (US); William R. Taylor, Lake City, MN (US); Douglas W. Mahoney, Rochester, MN (US)

(21) Appl. No.: 17/638,840

(22) PCT Filed: Aug. 27, 2020
(86) PCT No.: PCT/US2020/048270

§ 371 Date: Feb. 27, 2022

Related U.S. Application Data
(60) Provisional application No. 62/892,426, filed on Aug. 27, 2019.

(51) Int. Cl. C12Q 1/6886

(52) U.S. Cl. CPC C12Q 1/6886; C12Q 2600/158; C12Q 2600/154; C12Q 2600/16; C12Q 1/6806

(57) ABSTRACT

Provided herein is technology relating to detecting neoplasia and particularly, but not exclusively, to methods, compositions, and related uses for detecting neoplasms such as lung cancer.

The present application claims priority to U.S. Provisional Application Ser. No. 62/892,426, filed Aug. 27, 2019, which is incorporated herein by reference.

### FIELD OF THE INVENTION

Provided herein is technology relating to detecting neoplasia and particularly, but not exclusively, to methods, compositions, and related uses for detecting neoplasms such as lung cancer. Aspects of the invention relate to systems and methods for detecting lung cancer by assaying extracts from patient blood. In particular, embodiments include systems and methods for determining lung cancer progression at different stages by detecting immune cell RNA expression or circulating cell-free RNA levels.

### BACKGROUND OF THE INVENTION

Lung cancer remains the number one cancer killer in the US, and effective screening approaches are desperately needed. Lung cancer alone accounts for 221,000 deaths annually. Treatments exist, but are often not administered to patients until the disease has progressed to a point at which treatment efficacy is compromised.

A major challenge in cancer treatment is to identify patients early in the course of their disease. This is difficult under current methods because early cancerous or precancerous cell populations may be asymptomatic and may be located in regions which are difficult to access by biopsy. Thus, a robust, minimally invasive assay that may be used to identify all stages of the disease, including early stages which may be asymptomatic, would be of substantial benefit for the treatment of cancer.

### SUMMARY OF THE INVENTION

The systems, devices, kits, compositions, and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

The technology provides methods of characterizing a sample or combination of samples from a subject comprising analyzing the sample(s) for a plurality of different types of marker molecules. For example, in some embodiments, the technology provides a method comprising measuring an amount of at least one methylation marker gene in DNA from a sample obtained from a subject, and further comprises one or more of measuring an amount of at least one RNA marker in a sample obtained from the subject, and assaying for the presence or absence of at least one protein marker in a sample obtained from the subject. In some embodiments, a single sample from a subject is analyzed for methylation marker DNA(s), marker RNA(s), and marker protein(s).

Analyses of DNA, RNA and/or protein markers are not limited to use of any particular technologies. Methods for analyzing DNA and RNA include but are not limited to nucleic acid detection assays comprising amplification and probe hybridization, for example. Methods for analyzing proteins include but are not limited to enzyme-linked immunosorbent assay (ELISA) detection, protein immunoprecipitation, Western blot, immunostaining, etc.

One embodiment is a method of characterizing a sample from a subject, e.g., blood sampled from the subject, as a means of detecting lung cancer and/or determining lung cancer risk in a subject, e.g., a person. The method includes: providing a blood sample from the person; detecting target gene expression levels of target genes S100 Calcium Binding Protein A9 (S100A9), Selectin L (SELL), Peptidyl Arginine Deiminase 4 (PADI4), Apolipoprotein B MRNA Editing Enzyme Catalytic Subunit 3A (APOBE3CA), S100 Calcium Binding Protein A12 (S100A12), Matrix Metallopeptidase 9 (MMP9), Formyl Peptide Receptor 1 (FPR1), Thymidine Phosphorylase (TYMP), and/or Spermidine/spermine N1-acetyltransferase 1 (SAT1) in the blood sample; detecting a reference gene expression level of a reference gene in the blood sample; and determining the presence or absence of a lung neoplasia, or determining the person's risk of having lung cancer by comparing the detected target gene expression levels to the detected reference gene expression level.

In some embodiments, the technology provides a method for measuring amounts of one or more gene expression products in blood sampled from a subject, comprising:
a) extracting from blood sampled from a subject:
   i) at least one gene expression marker, wherein the at least one gene expression marker is product from expression of a marker gene selected from S100A9, SELL, PADI4, APOBE3CA, S100A12, MMP9, FPR1, TYMP, and SAT1; and
   ii) at least one reference marker;
b) measuring an amount of the at least one gene expression marker and an amount of at least one reference marker extracted in a);
c) calculating a value for the amount of the at least one gene expression marker as a percentage of the amount of the at least one reference marker, wherein the value indicates an amount of the at least one gene expression marker in the blood sampled from the subject.

In some embodiments, the extracting comprises extracting markers from a sample selected from whole blood, a blood product comprising white blood cells, and a blood product comprising plasma. In certain embodiments, the at least one gene expression marker comprises protein or RNA, and in certain preferred embodiments, RNA extracted from the blood sampled from the subject comprises circulating cell-free RNA. In some embodiments, RNA extracted from the blood sampled from the subject comprises RNA expressed by immune cells. In any of the embodiments, described hereinabove, the RNA extracted from the blood sampled from the subject may comprise mRNA.

The technology is not limited to measuring a single gene expression marker, and the technology encompasses measurement of multiple gene expression markers, e.g., such that measurement data may be analyzed in combination, as discussed in detail hereinbelow. In some embodiments, the technology is applied to measurement of a limited set of markers, e.g., for convenience or efficiency in applying the technology. For example, in any of the embodiments discussed above, the at least one gene expression marker may preferably consist of 2, 3, 4, 5, 6, 7, 8, or 9 gene expression markers.

In any of the embodiments discussed above, the at least one reference marker may comprise RNA or protein expressed from a gene selected from PLGLB2, GABARAP, NACA, EIF1, UBB, UBC, CD81, TMBIM6, MYL12B, HSP90BL, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACTB, HNRNPA1, CASC3, and SP. In certain preferred embodiments, the at least one reference marker comprises RNA. In certain embodiments, the reference marker comprises RNA selected from U1 snRNA and U6 snRNA.

As applied to any of the embodiments described above, the technology encompasses embodiments wherein measuring an amount of the at least one gene expression marker comprises using one or more of reverse transcription, polymerase chain reaction, nucleic acid sequencing, mass spectrometry, mass-based separation, and target cape, quantitative pyrosequencing, flap endonuclease assay, PCR-flap assay, enzyme-linked immunosorbent assay (ELISA) detection and protein immunoprecipitation. In certain embodiments, the measuring comprises multiplex amplification.

In some embodiments, DNA is also analyzed. Provided herein is a collection of methylation markers assayed on tissue or plasma that achieves extremely high discrimination for all types of lung cancer while remaining negative in normal lung tissue and benign nodules. Markers selected from the collection can be used alone or in a panel, for example, to characterize blood or bodily fluid, with applications in lung cancer screening and discrimination of malignant from benign nodules. In some embodiments, markers om the panel are used to distinguish one form of lung cancer from another, e.g., for distinguishing the presence of a lung adenocarcinoma or large cell carcinoma from the presence of a lung small cell carcinoma, or for detecting mixed pathology carcinomas. Provided herein is technology for screening markers that provide a high signal-to-noise ratio and a low background level when detected from samples taken from a subject.

Methylation markers and/or panels of markers (e.g., chromosomal region(s)) having an annotation selected from EMX1, GRIN2D, ANKRD13B, ZVF781, ZNF671, IFFO1, HOPX, BARX1, HOXA9, LOC100129726, SPOCA2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MA_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329 were identified in studies by comparing the methylation state of methylation markers from lung cancer samples to the corresponding markers in normal (non-cancerous) samples.

As described herein, the technology provides a number of methylation markers and subsets thereof (e.g., sets of 2, 3, 4, 5, 6, 7, 8, 9, 10, 11, 12 or more markers) with high discrimination for lung cancer and, in some embodiments, with discrimination between lung cancer types.

Accordingly, the technology of any of the embodiments described above measuring amounts of one or more gene expression products in blood sampled from a subject may further comprise:
d) extracting from blood sampled from the subject at least one methylation marker DNA and at least one reference marker DNA;
e) measuring an amount of at least one methylation marker DNA, wherein the at least one methylation marker DNA comprises a nucleotide sequence associated with at least one of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX.chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329;
f) measuring an amount of at least one reference marker DNA; and
g) calculating a value for the amount of the at least one methylation marker DNA as a percentage of the amount of the reference marker DNA, wherein the value indicates an amount of the at least one methylation marker DNA in the blood sampled from a subject.

The technology is not limited to measuring a methylation marker DNA, and the technology encompasses measurement of multiple methylation marker DNA, e.g., such that measurement data for different methylation marker DNAs may be analyzed in combination with each other, and/or in combination with measurement data for RNA and/or protein gene expression markers, as discussed in detail hereinbelow. In some embodiments, the technology is applied to measurement of a limited set of methylation marker DNAs, e.g., for convenience or efficiency in applying the technology. For example, in any of the embodiments discussed above, the at least one methylation marker DNA may preferably consist of 2, 3, 4, 5, 6, 7, 8, 9, 10, 11, 12, 13, 14, or 15 methylation marker DNAs. In certain embodiments, the at least one methylation marker DNA comprises a nucleotide sequence associated with at least one of BARX1, FLJ45983, HOPX, ZNF781, FAM59B, HOXA9, SOBP, and IFFO1. In certain of any of the embodiments described above, the at least one gene expression marker comprises a product from expression of a marker gene selected from FPR1, PADI4 and SELL.

In certain embodiments, the DNA extracted from the blood sampled from the subject comprises circulating cell-free DNA. In other embodiments the DNA comprises cellular DNA. In any of the embodiments discussed above, the at least one reference marker DNA used to calculate the value for the amount of the at least one methylation marker DNA is may preferably be selected from B3GALT6 DNA and β-actin DNA.

In any of the embodiments for measuring methylation marker DNA described above, included are embodiments in which the methylation marker DNA is treated with a reagent that selectively modifies DNA in a manner specific to the methylation status of the DNA. In some embodiments, the reagent comprises a bisulfite reagent, a methylation-sensitive restriction enzyme, or a methylation-dependent restriction enzyme, and in certain preferred embodiments, the bisulfite reagent comprises ammonium bisulfite.

While not limiting the technology to any particular method of measuring the amounts of methylation marker DNA, in some embodiments, measuring an amount of at least one methylation marker DNA comprises using one or more of polymerase chain reaction, nucleic acid sequencing, mass spectrometry, methylation-specific nuclease, mass-based separation, and target capture, and in certain preferred embodiments, measuring comprises multiplex amplification. In some embodiments measuring an amount of at least one methylation marker DNA comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

Embodiments of the technology provide a method of characterizing blood sampled from a subject, comprising:
i) treating blood sampled from a subject to produce extracted DNA and extracted RNA;
ii) measuring amounts of two or more marker RNAs in the extracted RNA, wherein the marker RNAs are selected from S100A9, SELL, PADI4, APOBE3CA, S100A12, MMP9, FPR1, TYMP, and SAT1 RNAs;
iii) measuring an amount of at least one reference RNA in the extracted RNA, wherein the reference RNA is selected from CASC3A, SKP1, and STK4;
iv) calculating a values for the amount of each of the two or more marker RNAs as a percentage of de amount of the at least one reference RNA, wherein the value for each marker RNA is indicative of the amount of the marker RNA in the blood sampled from the subject;
v) treating the extracted DNA with a bisulfite reagent to produce bisulfite-treated DNA;
vi) measuring amounts of two or more methylation marker DNAs in the bisulfite-treated DNA, wherein the methylation marker DNAs are selected from EMX1, GRIN2D, ANKRD13B, ZNF81, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXR2, MAX.chr12.526, BCL2L11, OPLAH, PARP15, KLHDC7B, SLC12A8, BHLHE23, CAPN2, FGF4, FLJ34208, BIN2_Z, DNMT3A, FERMT3, NFIX, SIPR4, SKI, SUCLG2, TBX15, and ZNF329 genes;
vii) measuring an amount of at least one reference DNA in the bisulfite-treated DNA wherein the at least one reference DNA is selected from B3GALT6 DNA and β-actin DNA; and
viii) calculating a value for the amount of each of the two or more methylation marker DNAs as a percentage of the amount of a reference DNA measured in the bisulfite-treated DNA, wherein the value for each methylation marker DNA is indicative of the amount of the methylation marker DNA in the blood sampled from the subject.

The embodiments comprising analysis of DNA and RNA described hereinabove encompass embodiments wherein DNA and RNA are isolated from blood collected in a single blood collection device, including but not limited to a single blood collection tube or blood collection bag.

Any of the embodiments described hereinabove comprise embodiments wherein the subject has or is suspected of having a lung neoplasm, and/or wherein the technology comprises assessing a risk of lung cancer in the subject based on values calculated using the measuring methods described above. For example, in some embodiments, an amount of the at least one gene expression marker and/or an amount of the at least one methylation marker DNA in the blood sampled from the subject is indicative of lung cancer risk of the subject.

In some embodiments, designs for assaying the methylation states of markers comprise analyzing background methylation at individual CpG loci in target regions of the markers to be interrogated by the assay technology. For example, in some embodiments, large numbers of individual copies of marker DNAs (e.g., >10,000, preferably >100,000 individual copies) from samples isolated from subjects diagnosed with disease, e.g., a cancer, are examined to determine frequency of methylation, and these data are compared to a similarly large numbers of individual copies of marker DNAs from samples isolated from subjects without disease. The frequencies of disease-associated methylation and of background methylation at individual CpG loci within the marker DNAs from the samples can be compared, such that CpG loci that having higher signal-to-noise, e.g., higher detectable methylation and/or reduced background methylation, may be selected for use in assay designs. See, e.g., U.S. Pat. Nos. 9,637,792 and 10,519,510, each of which is incorporated herein by reference in its entirety. In some embodiments a group of high signal-to-noise CpG loci (e.g., 2, 3, 4, 5, or more individual CpG loci in a marker region) are co-interrogated by an assay, such that all of the CpG loci must have a pre-determined methylation status (e.g., all must be methylated or none may be methylated) for the marker to be classified as “methylated” or “not methylated” on the basis of an assay result.

In some embodiments, a kit is provided comprising reagents or materials for assays are selected from measuring an amount of, or the presence or absence of at least one gene expression marker and/or at least one methylation marker DNA. The at least one gene expression marker may be an RNA marker or a protein marker.

For example, certain kit embodiments provide:
a) set of reagents for measuring an amount of at least one gene expression marker in blood sampled from a subject, wherein the at least one gene expression marker is produced from expression of a marker gene selected from S100A9, SELL, PADI4, APOBE3CA, S100A12, MMP9, FPR1, TYMP, and SAT1;
b) a set of reagents for measuring an amount of at least one reference marker in blood sampled from the subject.

In some embodiments, a kit further comprises a set of reagents for extracting the at least one gene expression marker and the at least one reference marker from blood. In some embodiments, the at least one gene expression marker comprises one or more of RNA and protein, and the at least one reference marker comprises one or more of RNA, DNA, and protein. In certain embodiments, a kit comprises:
i) at least one first oligonucleotide, wherein at least a portion of the at least one first oligonucleotide specifically hybridizes to a nucleic acid strand comprising a nucleotide sequence associated with a gene expression marker selected from S100A9, SELL, PADI4, APOBE3CA, S100A12, MMP9, FPR1, TYMP, and SAT1;
ii) at least one second oligonucleotide, wherein at least a portion of the at least one second oligonucleotide specifically hybridizes to a reference marker, wherein the reference marker is a reference nucleic acid.

In embodiments of kits described above, the nucleic acid strand comprising a nucleotide sequence associated with a gene expression marker is selected from RNA, cDNA, or amplified DNA. In certain embodiments, the reference nucleic acid comprises RNA or DNA, while in some embodiments, the reference gene expression marker preferably comprises RNA or protein expressed from a gene selected from PLGLB2, GABARAP, NACA, EIF1, UBB, UBC, CD81, TMBIM6, MYL12B, HSP90B1, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACTB, HNRNPA1, CASC3, and SKP1.

In any of the embodiments described above, a kit of the technology may further comprise;
c) a set of reagents for measuring an amount at least one methylation marker DNA in blood sampled from the subject, wherein the at least one methylation marker DNA comprises a nucleotide sequence associated with at least one of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX_chr16.55 PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX1, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329.

In some embodiments, the set of reagents for measuring an amount at least one methylation marker DNA comprises:
i) at least one third oligonucleotide, wherein at least a portion of the at least one third oligonucleotide specifically hybridizes to a nucleic acid strand comprising a nucleotide sequence associated with a methylation maker gene of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8S1A_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329.
   Embodiments of the kits described above may further comprise at least one fourth oligonucleotide, wherein at least a portion of the at least one fourth oligonucleotide specifically hybridizes to a reference marker DNA, preferably a reference marker DNA selected from B3GALT6 DNA and β-actin DNA. In some embodiments, at least one of the nucleic acid strand comprising a nucleotide sequence associated with a methylation maker gene and the reference marker DNA comprises bisulfite-treated DNA.

In some embodiments, a kit as described above further comprises a reagent that selectively modifies DNA in a manner specific to the methylation status of the DNA. In certain embodiments, the reagent that selectively modifies DNA in a manner specific to the methylation status of the DNA comprises a bisulfite reagent, a methylation-sensitive restriction enzyme, or a methylation-dependent restriction enzyme. In certain preferred embodiments, the bisulfite reagent comprises ammonium bisulfite.

Embodiments of kits provided above further encompass kits wherein one or more of the at least one first, second, third, and fourth oligonucleotides are selected from a capture oligonucleotide, a pair of nucleic acid primers, a nucleic acid probe, and an invasive oligonucleotide, and in certain embodiments, the capture oligonucleotide is attached to a solid support, e.g., covalently or through a non-covalent attachment (e.g., biotin-streptavidin binding or antigen-antibody binding). In preferred embodiments, the solid support is a magnetic bead.

Embodiments of any of the kits of the technology described hereinabove comprise kits comprising:
i) a first primer pair for producing a first amplified DNA from a gene expression marker product of expression of a marker gene selected from S100A9, SELL, PADI4, APOBE3CA, S100A12, MMP9, FPR1, TYMP, and SAT1;
ii) a first probe comprising a sequence complementary to a region of said first amplified DNA;
iii) a second primer pair for producing a second amplified DNA;
iv) a second probe comprising a sequence complementary to a region of said second amplified DNA;
v) reverse transcriptase; and
vi) a thermostable DNA polymerase.
   In some embodiments, the second amplified DNA is produced from a methylation marker gene or a reference marker nucleic acid.

In certain embodiments, the first probe further comprises a flap portion having a first flap sequence that is not substantially complementary to said first amplified DNA and in some embodiments, the second probe further comprises a flap portion having a second flap sequence that is not substantially complementary to said second amplified DNA. Kits of the technology may further comprise one or more of:
vii) a FRET cassette comprising a sequence complementary to said first flap sequence;
viii) a FRET cassette comprising a sequence complementary to said second flap sequence.
   Any of the kits described hereinabove may further comprise a flap endonuclease. In certain preferred embodiments, the flap endonuclease is a FEN-1 endonuclease, e.g., a thermostable FEN-1 endonuclease from a Archaeal organism.

Applications of the technology further provide compositions. For example, in some embodiments, the technology provides a composition comprising:
i) a first primer pair for producing a first amplified DNA from a gene expression marker product of expression of a gene selected from S100A9, SELL, PADI4, APOBE3CA, S100A12, MMP9, FPR1, TYMP, and SAT1;
ii) a first probe comprising a sequence complementary to a region of said first amplified DNA;
iii) a second primer pair for producing a second amplified DNA;
iv) a second probe comprising a sequence complementary to a region of said second amplified DNA;
v) reverse transciptase; and
vi) a thermostable DNA polymerase.

In some embodiments, the composition father comprises nucleic acid extracted from blood sampled from a subject, wherein the subject preferably has or Is suspected of having a lung neoplasm, or is a risk of having lung cancer. In some embodiments of the composition, the nucleic acid comprises one or more of:
cellular RNA;
circulating cell-fee RNA;
cellular DNA;
circulating cell-free DNA.

In some embodiments, the second primer pair produces a second amplified DNA from a methylation marker gene or a reference marker nucleic acid. In certain preferred embodiments, the second primer pair produces a second amplified DNA from a reference nucleic acid selected from:
RNA expressed from a gene selected from PLGLB2, GABARAP, NACA, EIF1, UBB, UBC, CD81, TMBIM6, MYL12B, HSP90B1, CLDN18, RAMP2, MFAP4, FABP4, MARCO, RGL1, ZBTB16, C10orf116, GRK5, AGER, SCGB1A1, HBR, TCF21, GMFG, HYAL1, TEK, GNG11, ADH1A, TGFBR3, INPP1, ADH1B, STK4, ACTB, HNRNPA1, CASC3, and SKP1;
RNA selected from U1 snRNA and U6 snRNA;
DNA selected from B3GALT6 DNA and β-actin DNA.

In certain embodiments, the second primer pair is selected to produce a second amplified DNA rom a methylation marker gene selected from EMX1, GRIN2D, ANKRD13B, ZNF781, ZNF67, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329.

The skilled artisan will recognize that the compositions above are not limited to two primer pairs, but encompass compositions that contain a number of different primer pairs for producing amplified DNA from a plurality of different gene expression markers and/or a number of different primer pairs for producing amplified DNA from a plurality of different methylation marker genes. Compositions may further comprise a number of different primer pairs for producing amplified DNA from a plurality of different reference marker nucleic acids.

In the compositions described above, the first probe and/or the second probe comprises a detection moiety comprising a fluorophore. In certain embodiments, probes of the technology may be labeled with a fluorphore and a quenching moiety, such that emission from the fluorophore is quenched when the probe is intact, e.g., when it has not been cleaved by a 5′ nuclease.

In some embodiments, the first probe further comprises a flap portion having a first flap sequence that is not substantially complementary to said first amplified DNA, and/or wherein the second probe further comprises a flap portion having a second flap sequence that is not substantially complementary to said second amplified DNA. In certain embodiments, the composition further comprises one or more of:
vii) a FRET cassette comprising a sequence complementary to the first flap sequence;
viii) a FRET cassette comprising a sequence complementary to the second flap sequence.

Any of the compositions described above may further comprise a flap endonuclease, preferably a FEN-1 endonuclease, e.g., a thermostable FEN-1 from an Archaeal organism.

In certain embodiments, the compositions described above comprise a buffer comprising Mg⁺⁺, e.g., MgCl₂. Preferably, the compositions comprise a PCR-flap assay buffer comprising having relatively high Mg⁺⁺ and low KCl compared to standard PCR buffers, (e.g., 6-10 mM, preferably 7.5 mM Mg⁺⁺, and 0.0 to 0.8 mM KCl).

Embodiments of the technology further comprise a reaction mixture comprising any one of the compositions described hereinabove.

In some embodiments, a kit comprises reagents or materials for at least two assays, wherein the assays are selected from measuring an amount of, or the presence or absence of 1) at least one methylated DNA marker; 2) at least one RNA marker; and/or 3) at least one protein marker. In preferred embodiments, the at least one methylated DNA marker is selected from the group consisting of BARX1, LOC100129726, SPOCK2, TSC22D4, MAX.chr8.124, RASSF1, ZNF671, ST8SIA1, NKX6_2, FAM59B, DIDO1, MAX_Chr1.110, AGRN, SOBP, MAX_chr10.226, ZWIZ1, MAX_chr8.145, MAX_chr10.225, PRDM14, ANGPT1, MAX.chr16.50, PTGDR_9, ANKRD13B, DOCK2, MAX_chr19.163, ZNF132, MAX chr19.372, HOXA9, TRH, SP9, DMRTA2, ARHGEF4, CYP26CL, ZNF781, PTGDR, GRIN2D, MATK, BCAT1, PRKCB_28, ST8SIA_22, FLJ45983, DLX4, SHOX2, EMX1, HOXB2, MAX.chr12.526, BCL2L11, OPLAH, PARP15, KLHDC7B, SLC12a, BHLHE23, CAPN2, FGF14, FLJ34208, B3GALT6, BIN2_Z, DNMT3A, FERMT3, NFIX, SIPR4, SKI, SUCLG2, TBX1S, ZDHHC1, ZNF329, IFFO1, and HOPX. In certain preferred embodiments, the at least RNA expression marker expressed from a gene selected from the group consisting of S100A9, SELL, PADI4, APOBE3CA, S100A12, MMP9, FPR1, TYMP, and SAT1. In some embodiments, the at least one protein comprises an antigen, e.g., a cancer-associated antigen, while in some embodiments, the at least one protein comprises an antibody, e.g., an autoantibody to a cancer-associated antigen.

In some embodiments, an oligonucleotide in said mixture comprises a reporter molecule, and in preferred embodiments, the reporter molecule comprises a fluorophore. In some embodiments the oligonucleotide comprises a flap sequence. In some embodiments the mixture further comprises one or more of a FRET cassette; a FEN-1 endonuclease and/or a thermostable DNA polymerase, preferably a bacterial DNA polymerase.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated. In some embodiments, a nucleic acid may be characterized as “unmethylated” if it is not methylated at a specific locus (e.g., the locus of a specific single CpG dinucleotide) or specific combination of loci, even if it is methylated at other loci in the same gene or molecule.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs. As used herein, the terms “marker gene” and “marker” are used interchangeably to refer to DNA, RNA, or protein (or other sample components) that is associated with a condition, e.g., cancer, regardless of whether the marker region is in a coding region of DNA. Markers may include, e.g., regulatory regions, flanking regions, intergenic regions, etc. Similarly, the term “marker” used in reference to any component of a sample, e.g., protein, RNA, carbohydrate, small molecule, etc., refers to a component that can be assayed in a sample (e.g., measured or otherwise characterized) and that is associated with a condition of a subject, or of the sample from a subject. The term “methylation marker” refers to a gene or DNA in which the methylation state of the gene or DNA is associated with a condition, e.g., cancer.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides is the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera, et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. An exemplary reagent is a bisulfite reagent.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivatives thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a nucleotide that is typically methylated and an unmethylated selected nucleotides refers specifically to a nucleotide that typically occurs in unmethylated form.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. Generally, the primer is sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence. As used in reference to a double-stranded nucleic acid, the term “target” is not limited to a particular strand of the duplexed target, e.g., a coding strand, but may be used in reference to either or both strands of, for example, a double-stranded gene or reference DNA.

As used herein, the terms “cell-free” and “circulating cell-free” as used in reference to nucleic acids from blood are used interchangeable and refer to nucleic acids, e.g., DNA and RNA species, that are found in blood but that are not within cells in the blood. The terms as used herein with respect to nucleic acid extracted from blood refer to the nature and location of the nucleic acid prior to collection of the sample from the subject and prior to extraction of the nucleic acid from the blood sample.

The term “marker”, as used herein, refers to a substance (e.g., a nucleic acid, or a region of a nucleic acid, or a protein) that may be used to distinguish non-normal cells (e.g., cancer cells) from normal cells (non-cancerous cells), e.g., based on presence, absence, or status (e.g., methylation state) of the marker substance. As used herein “normal” methylation of a marker refers to a degree of methylation typically found in normal cells, e.g., in non-cancerous cells.

The term “neoplasm” as used herein refers to any new and abnormal growth of tissue, including but not limited to a cancer. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

The term “sample” is used in its broadest sense. In one sense it can refer to an animal cell or tissue or fluid. In another sense, it refers to a specimen or culture obtained from any source, as well as biological and environmental samples. Biological samples may be obtained from plants or animals (including humans) and encompass, e.g., fluids, solids, tissues, and gases. Environmental samples include environmental material such as surface matter, soil, water, and industrial samples. These examples are not to be construed as limiting the sample types applicable to the present invention. As used herein in reference to samples, the term “a sample” collected from a source or subject, e.g., from a patient, is not limited to a single physical specimen but also encompasses a sample that is collected in multiple portions, e.g., “a sample” of blood may be collected in two, three, four or more different blood collection tubes or other blood collection devices (e.g., bags), or combinations of different blood collection devices.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (e.g., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

In some embodiments, target nucleic acid is amplified (e.g., by PCR) and amplified nucleic acid is detected simultaneously using an invasive cleavage assay. Assays configured for performing a detection assay (e.g., invasive cleavage assay) in combination with an amplification assay are described in U.S. Pat. No. 9,096,893, incorporated herein by reference in its entirety for all purposes. Additional amplification plus invasive cleavage detection configurations, termed the QuARTS method, are described in, e.g., in U.S. Pat. Nos. 8,361,720; 8,715,937; 8,916,344; 9,212,392, and U.S. patent application Ser. No. 15/841,006 each of which is incorporated herein by reference for all purposes. The term “invasive cleavage structure” as used herein refers to a cleavage structure comprising i) a target nucleic acid, ii) an upstream nucleic acid (e.g., an invasive or “INVADER” oligonucleotide), and iii) a downstream nucleic acid (e.g., a probe), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, e.g., as disclosed, for example, in U.S. Pat. No. 6,090,543, incorporated herein by reference in its entirety. In some embodiments, one or more of the nucleic acids may be attached to each other, e.g., through a covalent linkage such as nucleic acid stem-loop, or through a non-nucleic acid chemical linkage (e.g., a multi-carbon chain). As used herein, the term “flap endonuclease assay” includes “INVADER” invasive cleavage assays and QuARTS assays, as described above.

The term “probe oligonucleotide” or “flap oligonucleotide” when used in reference to flap assay, refers to an oligonucleotide that interacts with a target nucleic acid to form a cleavage structure in the presence of an invasive oligonucleotide.

The term “invasive oligonucleotide” refers to an oligonucleotide that hybridizes to a target nucleic acid at a location adjacent to the region of hybridization between a probe and the target nucleic acid, wherein the 3′ end of the invasive oligonucleotide comprises a portion (e.g., a chemical moiety, or one or more nucleotides) that overlaps with the region of hybridization between the probe and target. The 3′ terminal nucleotide of the invasive oligonucleotide may or may not base pair a nucleotide in the target. In some embodiments, the invasive oligonucleotide contains sequences at its 3′ end that are substantially the same as sequences located at the 5′ end of a portion of the probe oligonucleotide that anneals to the target strand.

The term “flap endonuclease” or “FEN,” as used herein, refers to a class of nucleolytic enzymes, typically 5′ nucleases, that act as structure-specific endonucleases on DNA structures with a duplex containing a single stranded 5′ overhang, or flap, on one of the strands that is displaced by another strand of nucleic acid (e.g., such that there are overlapping nucleotides at the junction between the single and double-stranded DNA). FENs catalyze hydrolytic cleavage of the phosphodiester bond at the junction of single and double stranded DNA, releasing the overhang, or the flap. Flap endonucleases are reviewed by Ceska and Savers (Trends Biochem. Sci. 1998 23:331-336) and Liu et al (Annu Rev. Biochem. 2004 73: 589-615; herein incorporated by reference in its entirety). FENs may be individual enzymes, multi-subunit enzymes, or may exist as an activity of another enzyme or protein complex (e.g., a DNA polymerase).

A flap endonuclease may be thermostable. For example, FEN-1 flap endonuclease from archival thermophiles organisms are typical thermostable. As used herein, the term “FEN-1” refers to a non-polymerase flap endonuclease from a eukaryote or archaeal organism. See, e.g., WO 02/070755, and U.S. Pat. No. 7,122,364, and Kaiser M. W., et al. (1999) J. Biol. Chem., 274:21387, which are all incorporated by reference herein in their entireties for all purposes.

As used herein, the term “cleaved flap” refers to a single-stranded oligonucleotide that is a cleavage product of a flap assay.

The term “cassette,” when used in reference to a flap cleavage reaction, refers to an oligonucleotide or combination of oligonucleotides configured to generate a detectable signal in response to cleavage of a flap or probe oligonucleotide, e.g., in a primary or first cleavage structure formed in a flap cleavage assay. In preferred embodiments, the cassette hybridizes to a non-target cleavage product produced by cleavage of a flap oligonucleotide to form a second overlapping cleavage structure, such that the cassette can then be cleaved by the same enzyme, e.g., a FEN-1 endonuclease.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label, e.g., a fluorophore. In particularly preferred embodiments, a cassette comprises labeled moieties that produce a FRET effect.

As used herein, the term “FRET” refers to fluorescence resonance energy transfer, a process in which moieties (e.g., fluorophores) transfer energy e.g., among themselves, or, from a fluorophore to a non-fluorophore (e.g., a quencher molecule). In some circumstances, FRET involves an excited donor fluorophore transferring energy to a lower-energy acceptor fluorophore via a short-range (e.g., about 10 nm or less) dipole-dipole interaction. In other circumstances, FRET involves a loss of fluorescence energy from a donor and an increase in fluorescence in an acceptor fluorophore. In still other forms of FRET, energy can be exchanged from an excited donor fluorophore to a non-fluorescing molecule (e.g., a “dark” quenching molecule, e.g., “BHQ” quenchers, Biosearch Technologies). FRET is known to those of skill in the art and has been described (See, e.g., Stryer et al., 1978, Ann. Rev. Biochem., 47:819; Selvin, 1995, Methods Enzymol., 246:300; Orpana, 2004 Biomol Eng 21, 45-50; Olivier, 2005 Mutant Res 573, 103-110, each of which is incorporated herein by reference in its entirety).

In an exemplary flap detection assay, an invasive oligonucleotide and flap oligonucleotide are hybridized to a target nucleic acid to produce a first complex having an overlap as described above. An unpaired “flap” is included on the 5′ end of the flap oligonucleotide. The first complex is a substrate for a flap endonuclease, e.g., a FEN-1 endonuclease, which cleaves the flap oligonucleotide to release the 5′ flap portion. In a secondary reaction, the released 5′ flap product serves as an invasive oligonucleotide on a FRET cassette to again create the structure recognized by the flap endonuclease, such that the FRET cassette is cleaved. When the fluorophore and the quencher are separated by cleavage of the FRET cassette, a detectable fluorescent signal above background fluorescence is produced.

As used herein, the term “PCR-flap assay” refers to an assay configuration combining PCR target amplification and detection of the amplified DNA by formation of a first overlap cleavage structure comprising amplified target DNA, and a second overlap cleavage structure comprising a cleaved 5′ flap from the first overlap cleavage structure and a labeled reporter oligonucleotide, e.g., a “FRET cassette” or 5′ hairpin FRET reporter oligonucleotide. In the PCR-flap assay as used herein, the assay reagents comprise a mixture containing DNA polymerase, FEN-1 endonuclease, a primary probe comprising a portion complementary to a target nucleic acid, and a FRET cassette or 5′ hairpin FRET reporter, and the target nucleic acid is amplified by PCR and the amplified nucleic acid is detected simultaneously (i.e., detection occurs during the course of target amplification). PCR-flap assays include the QuARTS assays described in U.S. Pat. Nos. 8,361,720; 8,715,937; and 8,916,344; flap assay using probe oligonucleotides having a longer target-specific region (Long probe Quantitative Amplified Signal, “LQAS”) is described in U.S. Pat. No. 10,648,025; and the amplification assays of U.S. Pat. No. 9,096,893 (for example, as diagrammed in FIG. 1 of that patent), each of which is incorporated herein by reference in its entirety.

As used herein, the term “PCR-flap assay reagents” refers to one or more reagents for detecting target sequences in a PCR-flap assay, the reagents comprising nucleic acid molecules capable of participating in amplification of a target nucleic acid and in formation of a flap cleavage structure in the presence of the target sequence, in a mixture containing DNA polymerase, FEN-1 endonuclease and a FRET cassette or 5′ hairpin FRET reporter.

The term “real time” as used herein in reference to detection of nucleic acid amplification or signal amplification refers to the detection or measurement of the accumulation of products or signal in the reaction while the reaction is in progress, e.g., during incubation or thermal cycling. Such detection or measurement may occur continuously, or it may occur at a plurality of discrete points during the progress of the amplification reaction, or it may be a combination. For example, in a polymerase chain reaction, detection (e.g., of fluorescence) may occur continuously during all or part of thermal cycling, or it may occur transiently, at one or more points during one or more cycles. In some embodiments, real time detection of PCR or QuARTS reactions is accomplished by determining a level of fluorescence at the same point (e.g., a time point in the cycle, or temperature step in the cycle) in each of a plurality of cycles, or in every cycle. Real time detection of amplification may also be referred to as detection “during” the amplification reaction.

As used herein, the term “quantitative amplification data set” refers to the data obtained during quantitative amplification of the target sample, e.g., target DNA. In the case of quantitative PCR or QuARTS assays, the quantitative amplification data set is a collection of fluorescence values obtained at during amplification, e.g., during a plurality of, or all of the thermal cycles. Data for quantitative amplification is not limited to data collected at any particular point in a reaction, and fluorescence may be measured at a discrete point in each cycle or continuously throughout each cycle.

The abbreviations “Ct” and “Cp” as used herein in reference to data collected during real time PCR and PCR+INVADER assays refer to the cycle at which signal (e.g., fluorescent signal) crosses a predetermined threshold value indicative of positive signal. Various methods have been used to calculate the threshold that is used as a determinant of signal verses concentration, and the value is generally expressed as either the “crossing threshold” (Ct) or the “crossing point” (Cp). Either Cp values or Ct values may be used in embodiments of the methods presented herein for analysis of real-time signal for the determination of the percentage of variant and/or non-variant constituents in an assay or sample.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides.

The term “system” as used herein refers to a collection of articles for use for a particular purpose. In some embodiments, the articles comprise instructions for use, as information supplied on e.g., an article, on paper, or on recordable media (e.g., DVD, CD, flash drive, etc.). In some embodiments, instructions direct a user to an online location, e.g., a website.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DESCRIPTION OF THE DRAWINGS

FIGS. 1-4 provide tables comparing Reduced Representation Bisulfite Sequencing (RRBS) results for selecting markers associated with lung carcinomas as described in Example 2, with each row showing the mean values for the indicated marker region (identified by chromosome and start and stop positions). The ratio of mean methylation for each tissue type (normal (Norm), adenocarcinoma (Ad), large cell carcinoma (LC), small cell carcinoma (SC), squamous cell carcinoma (SQ) and undefined cancer (UND)) is compared to the mean methylation of buffy coat samples from normal subjects (WBC or BC)) is shown for each region, and genes and transcripts identified with each region are indicated.

FIG. 1 provides a table comparing RRBS results for selecting markers associated with lung adenocarcinoma.

FIG. 2 provides a table comparing RRBS results for selecting markers associated with lung large cell carcinoma.

FIG. 3 provides a table comparing RRBS results for selecting markers associated with lung small cell carcinoma.

FIG. 4 provides a table comparing RRBS results for selecting markers associated with lung squamous cell carcinoma.

FIG. 5 provides a table of nucleic acid sequences of assay target regions in unconverted form and bisulfite-converted form, and detection oligonucleotides, with corresponding SEQ ID NOS. Target nucleic acids, in particular target DNAs (including bisulfite-converted DNAs) are shown for convenience as single strands but it is understood that embodiments of the technology encompass the complementary strands of the depicted sequences. For example, primers and flap oligonucleotides may be selected to hybridize to the target strands as shown, or to strands that are complementary to the target strands as shown.

FIG. 6 illustrates an exemplary workflow of one method of analyzing a blood sample to determine lung cancer risk in a person.

FIG. 7 shows data from experiments focused on the FPR1 gene expression by RNA detection. Panel A is a line chart of a training set of data showing the relationship of a true positive cancer rate to a false positive cancer rate. Panel B is a line chart of a validation data set showing the relationship of true positive cancer rate to a false positive cancer rates. Panel C is a dot plot showing the FPR1 RNA expression levels in white blood cells taken from nonsmokers, normal smokers, and patients with different stages of lung cancer, and indicating a slight sensitivity to tobacco in normal smokers.

FIG. 8 shows data from experiments focused on the S100A12 gene. Panel A is a line chart of a training set of data showing the relationship of a true positive cancer rate to a false positive cancer rate. Panel B is a line chart of a validation data set showing the relationship of true positive cancer rate to a false positive cancer rates. Panel C is a dot plot showing S100A12 RNA expression levels in white blood cells taken from nonsmokers, normal smokers, and patients with different stages of lung cancer.

FIG. 9 shows data from experiments focused on the MMP9 gene. Panel A is a line chart of a training set of data showing the relationship of a true positive cancer rate to a false positive cancer rate. Panel B is a line chart of a validation data set showing the relationship of true positive cancer rate to a false positive cancer rates, showing an improvement compared to FPR1. Panel C is a dot plot showing MMP9 RNA expression levels in white blood cells taken from nonsmokers, normal smokers, and patients with different stages of lung cancer.

FIG. 10 shows data from experiments focused on the SAT1 gene. Panel A is a line chart of a training set of data showing the relationship of a true positive cancer rate to a false positive cancer rate. Panel B is a line chart of a validation data set showing the relationship of true positive cancer rate to a false positive cancer rates. Panel C is a dot plot showing SAT1 RNA expression levels in white blood cells taken from nonsmokers, normal smokers, and patients with different stages of lung cancer.

FIG. 11 shows the results of experiments using FPR1 as a target gene and STK4 as a reference gene. Panel A is a dot plot showing the relationship between the FPR1 ratio and the FPR1 Fragments Per Kilobase Million normalization (FPKM). Panel B is a line graph showing the ratio of true positive rates and false positive rates of FPR1 as compared to STK4.

FIG. 12 shows an exemplary embodiment of a method using S100A12 as a target gene and STK4 as a reference gene. Panel A is a dot plot showing the relationship between the S100A12 ratio and the S100A12 FPKM. Panel B is a line graph showing the ratio of true positive rates and false positive rates of S100A12 as compared to STK4.

FIG. 13 shows an exemplary embodiment of a method using MMP9 as a target gene and STK4 as a reference gene. Panel A is a dot plot showing the relationship between the MMP9 ratio and the MMP9 FPKM. Panel B is a line graph showing the ratio of true positive rates and false positive rates of MMP9 as compared to STK4.

FIG. 14 is a scatter plot that shows data comparing RNA expression levels of both S100A12 and MMP9 as target genes in different stages of lung cancer. FPKM normalization was used and data includes all samples, both training and validation sets.

FIG. 15 is a scatter plot that shows data comparing RNA expression levels of both S100A12 and SAT1 as target genes in cancer, benign and normal patients. FPKM normalization was used. The dashed separating line is for visualization purposes only.

FIG. 16 is a scatter plot showing data comparing RNA expression levels of both S100A12 and TYMP as target genes in cancer, benign and normal patients. STK4 normalization was used. The dashed separating line is for visualization purposes only.

### DETAILED DESCRIPTION OF THE INVENTION

Provided herein are technologies relating to selection of marker analytes, and methods of characterizing a sample or combination of samples from a subject comprising analyzing the sample(s) for a plurality of different types of marker analytes, e.g., marker molecules such as DNAs, RNAs, and proteins. For example, in some embodiments, the technology provides a method comprising measuring an amount of at least one methylation marker gene in DNA having a particular methylation status (e.g., being methylated or unmethylated) from a sample obtained from a subject, and further comprises one or more of measuring an amount of at least one RNA marker in a sample obtained from the subject, and assaying for the presence or absence of, or an amount of, at least one protein marker in a sample obtained from the subject. In some embodiments, a single sample from a subject is analyzed for methylation marker DNA(s), marker RNA(s), and marker protein(s).

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference. The discussion below is divided into the following sections:
I. RNA Marker Analysis (including Quantitative RNA analysis and Quantitative Protein analysis); and
II. Methylation Marker Analysis

### I. RNA Marker Analysis

A. Quantitative RNA analysis

Embodiments relate to systems and methods of determining whether a patient at risk for cancer may have the disease by analyzing nucleic acid expression, particularly circulating cell-free nucleic acid or immune cell nucleic acid expression, in the blood. Determination of patients that may have cancer may be done on blood-derived specimens to assay RNA accumulation or expression levels, and such analysis may be conducted by expression microarray, nucleic acid sequencing, nCounter, or real-time PCR. In some embodiments, expression levels of a subset of reference nucleic acids are compared to expression levels of a subset of target nucleic acids that are known to be increased in patients having cancer. The subset of reference nucleic acids may be found by analyzing blood from many disease-free patients and selecting genes that are expressed at stable levels within those patients. Subsets of reference nucleic acids may also be found by analyzing solid tissue specimens taken from multiple tissue types (e.g., colon, lung, kidney, liver, etc.), and selecting genes that are expressed at stable levels in a patient's blood.

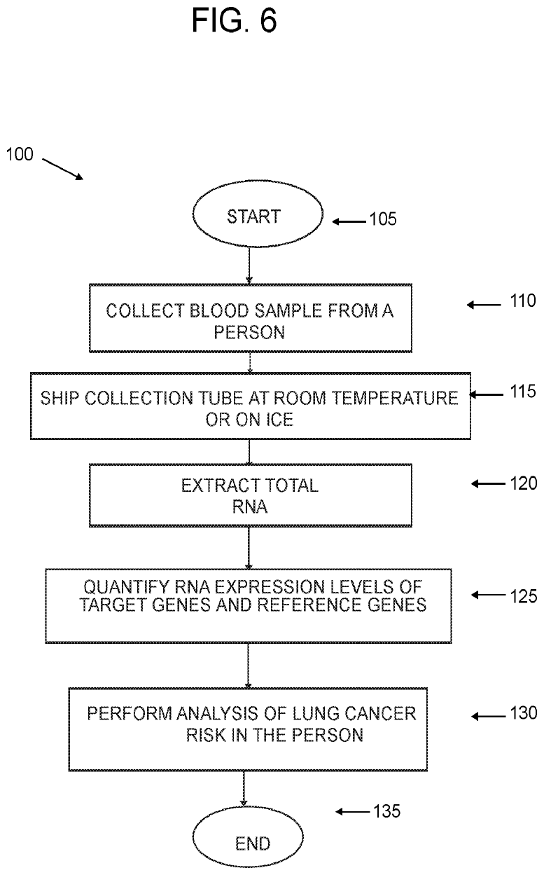

One embodiment is shown in the flow diagram of FIG. 6. As shown, the process 100 begins at a start state 105 and then moves to a state 110, wherein a blood sample is obtained from a person. The blood sample may be collected from a human patient suspected of having lung cancer, or where the patient is known to have lung cancer, but a more thorough analysis of the type or stage of cancer may be desired. The process 100 then moves to state 115 where the blood sample to be analyzed is shipped to a laboratory at room temperature or on ice in a blood collection tube, which ensures as little degradation of the sample as possible. Once the blood sample is received in the laboratory, the process 100 moves to state 120 where RNA is extracted from the blood, as discussed in more detail below. After the RNA is extracted, the process 100 moves to state 125 where the gene expression level of one or more target genes, and optionally one of more reference genes, is detected by measuring the levels of specific RNA in the sample. Methods of detecting gene expression and selecting the target genes and reference genes are discussed in more detail below. Once the gene expression levels for specific target genes are determined, the process 100 moves to state 130 where an analysis is performed to determine the patient's risk for having, or developing, lung cancer based on the measured levels of the target gene expression in the patient. The process 100 then terminates at an end state 135.

In some embodiments, subsets of target genes can be selected by analyzing genes whose transcript accumulation or expression levels increase in blood or in solid tumor specimens taken from individuals suffering from cancer.

In some embodiments, subsets of target genes include genes whose transcript accumulation or expression levels decrease in blood or in solid tumor specimens taken from individuals suffering from cancer.

In some embodiments, subsets of reference genes comprise genes whose transcript accumulation or expression levels are unchanged in normal individuals as compared to cancer patients. In these embodiments, subsets of target genes whose accumulation or expression levels increase in blood or in solid tumors specimens are selected in combination with one or more reference genes.

In some embodiment, aspects of the disclosed technology relate to the discovery that expression of RNA levels of formylpeptide receptor gene (FPR1), S100A12, MMP9, SAT1, and TYMP change in patients suffering from cancer. For example, RNA levels of FPR1, S100A12, MMP9, SAT1, and TYMP were found to increase in patients having lung cancer, as described below. Moreover, RNA levels of FPR1 were shown to increase in comparison to RNA levels of other reference genes, such as STK4, ACTB, and HNRNPA1.

In some embodiments, once the target gene is known, the reference gene can be selected by analyzing a large number of candidates from multiple specimens and selecting those for which the difference between the target gene and the reference gene is largest in gene expression from cancer patients. In some embodiments, the reference gene can be selected by surveying transcript accumulation or expression levels of many genes and finding which ones have the lowest variability. In some embodiments reference genes are selected not based on their individual accumulation or expression levels but on the lack of change in their relative accumulation or expression levels in cancer.

Once target genes (and reference genes in some embodiments) are known within a given cancer type, the expression profile can be measured in blood taken from cancer patients and patients for which a cancer is to be assayed. Because plasma or white blood cells can be collected and prepared within many primary care physician offices without posing any more risk than a standard blood draw, relative RNA accumulation or expression levels between target genes and reference genes in some embodiments may be a valuable cancer biomarker. Additionally, if target genes and reference genes in some embodiments may be assayed reliably, they may have a number of advantages over current cancer assays. For example, in some embodiments this method may detect cancer at an early stage of development, cancer that poses few symptoms, cancer that is difficult to distinguish from benign conditions or cancer that may be developing in an area of the body that may not be accessible to traditional biopsy assays.

Increased RNase activity is often present in tumors. This RNase activity may inhibit tumor growth, and may be part of the immune system's response to cancer. Cytotoxic T cells may lead to apoptosis of cancer cells via IFN-γ, and this apoptosis may result in activation of RNases, such as RNase L. Death of cells via necrosis, which may be caused by hypoxia due to tumor growth, may also contribute to the release of RNases. It is known that plasma of lung cancer patients has increased RNase activity (Marabella et al., (1976) “Serum ribonuclease in patients with lung carcinoma,” Journal of Surgical Oncology, 8(6):501-505; Reddi et al. (1976) “Elevated serum ribonuclease in patients with pancreatic cancer,” Proc. Nat'l. Acad. Sci. USA 73(7):2308-2310). It is also known that lung cells contain RNases similar to those found in plasma (Neuwelt et al., (1978) “Possible Sites of Origin of Human Plasma Ribonucleases as Evidenced by Isolation and Partial Characterization of Ribonucleases from Several Human Tissues,” Cancer Research 38:88-93).

When higher levels of RNase are present in plasma, any free RNA is susceptible to more rapid degradation. Thus, there may be less RNA detectable in plasma RNA preparations due to relates of RNases. While all RNA may be present at decreased levels, it may only be possible to detect this difference with a high level of accuracy when the normal variability of a gene is low. For example, if the normal range of a gene's expression is between 10 and 100 units, it may be difficult to accurately detect a decrease of 1 unit. However, if a gene's expression is normally between 10 and 11 units, a decrease of 1 unit is readily detectable (e.g., any number under 10 units would indicate a decrease).

In some embodiments, the target gene is FPR1. FPR1 plays multiple roles in the lungs and cancer. FPR1 is expressed in lung fibroblasts (VanCompemolle et al. (2003) J Immunol. 171(4):2050-6) and is necessary for wound repair in the lungs (Shao (2011) Am J Respir Cell Mol Biol 44:264-269). It is known that fibroblasts are important in both attracting immune cells that fight the tumor (Gemperle (2012) PLOSOne 7(11):1-7, e50195) and creation of stroma which protects the tumor (Wang (2009) Clin Cancer Res 15(21) 6630-6638). FPR1 may also exacerbate the activity of other oncogenes in tumors (Huang (2007) Cancer Res 67(12):5906-5913). There is no evidence that it is overexpressed in lung cancers, but FPR1 is known to be regulated by RNA stabilization (Mandal (2007) J Immunol 178:2542-2548, Mandal (2005) J Immunol 175:6085-6091). Given these roles, it is possible that FPR1 RNA is secreted deliberately by either tumor cells to enhance tumor growth (e.g., by activating wound-repair systems for growth or growing protective stroma) or immune cells to enhance the immune response (e.g., attracting additional immune cells).

In some embodiments, the target gene is S100 calcium binding protein A12 (S100A12), also known as calgranulin C and EN-RAGE (extracellular newly identified RAGE binding protein), which is specifically related to innate immune function. S100A12 is expressed by phagocytes and released at the site of tissue inflammation. It is an endogenous DAMP that turns pro-inflammatory after a release into the extracellular space following brain injury. The Receptor for Advanced Glycation End Products (RAGE) is a member of the immunoglobulin superfamily and is a specific cell surface reaction site for advanced glycation end products (AGEs) which increase with advancing age. Interaction between AGEs and RAGE has been linked to chronic inflammation. Once engaged RAGE interaction in inflammatory and vascular cells results in the increased expression of MMPs. The human s100A12 mRNA sequence is publicly available as GenBank Accession No. NM005621. The human S100A12 amino acid sequence is publicly available as GenPept Accession No. NP05612.

In some embodiments, the target gene comprises myeloid-related proteins (MRP), which play a role in the process of neutrophil migration to an inflammatory site. MRP proteins are a subfamily of S100 proteins in which three members of the MRP family have further been characterized, namely S100A8, S100A9 and S100A12, having molecular weight of 10.6, 13.5 and 10.4 kDa respectively, and are expressed abundantly in the cytosol of neutrophils and at lower levels in monocytes. S100A8 and S100A9 are also expressed by activated endothelial cells, certain epithelial cells, keratinocytes and neutrophilic and monocytic-differentiated HL-60 and THP-1. MRPs lack signal peptide sequences so they are not present in granules but rather in the cytosol where they account for up to 40% of the cytosolic proteins. The three MRPs exist as noncovalently-bonded homodimers. In addition, in the presence of calcium, S100A8 and S100A9 associate to form a noncovalent heterodimer called S100A8/A9; these are known as MRP-8/14 complex, calprotectin, p23 and cystic fibrosis antigen as well. S100A8 is also named MRP-8, L1 antigen light chain and calgranulin A and S100A9 is called MRP-14, L1 antigen heavy chain, cystic fibrosis antigen, calgranulin B and BEE22. Other names for S100A12 are p6, CAAF1, CGRP, MRP-6. EN-RAGE and calgranulin C.

The family of the S100 proteins comprises 19 members of small (10 to 14 kDa) acidic calcium-binding proteins. They are characterized by the presence of two EF-hand type calcium-binding motifs, one having two amino acids more than the other. These intracellular proteins are involved in the regulation of protein phosphorylation, enzymatic activities, Ca²⁺ homeostasis, and intermediate filaments polymerization. S100 proteins generally exist as homodimers, but some can form heterodimers. More than half of the S00 proteins are also found in the extracellular space where they exert cytokine-like activities through specific receptors; one being recently characterized as the receptor for advanced glycation end-products (RAGE). S100A8 and S100A9 belong to a subset of the S00 protein family called Myeloid Related Proteins (MRPs) because their expression is almost completely restricted to neutrophils and monocytes, which are products of the myeloid precursors.

High concentrations of MRP in serum may occur in pathologies associated with increased numbers of circulating neutrophils or their activity. Elevated levels of S100A8/A9 (more than 1 μg/ml) are observed in the serum of patients suffering from various infections and inflammatory pathologies such as cystic fibrosis, tuberculosis, and juvenile rheumatoid arthritis. They are also expressed at very high levels in the synovial fluid and plasma of patients suffering from rheumatoid arthritis and gout. High levels of MRPs (up to 13 μg/ml) are also known as being present in the plasma of chronic myeloid leukemia and chronic lymphoid leukemia patients. The presence of these proteins even preceded the appearance of leukemia cells in the blood of relapsing patients. The extracellular presence of S100A8/A9 suggests that the MRPs can be released either actively or during cell necrosis.

MRPs are expressed in the cytosol, implying that they are secreted via an alternative pathway. Once released in the extracellular environment, MRPs exert pro-inflammatory functions. These activities are shared by several other S100 proteins. For example, S100 stimulates the release of the pro-inflammatory cytokine IL-6 from neurons and promotes neurite extension. S100L (S100A2) is chemotactic towards eosinophils, while psoriasin (S100A7) is chemotactic for neutrophils and T lymphocytes, but not monocytes. S100A8, S100A9, and S100A8/A9 are chemotactic for neutrophils, with a maximal activity at 10⁻⁹ to 10⁻¹⁰ M. Murine S100A8, also called CP-10, is known to be a good potent chemotactic factor for murine myeloid cells with an activity of 10⁻¹² M.

In addition, S100A12 is chemotactic for monocytes and neutrophils and induces the expression of TNF-α and IL-1β from a murine macrophage cell line. MRPs also stimulate leukocyte adhesion to endothelium. S100A9 stimulates neutrophil adhesion to fibrinogen by activating the β₂ integrin Mac-1.

It was recently demonstrated that S100A8, S100A12 and S100A8/A9 also stimulate neutrophil adhesion to fibrinogen. Endothelial cells incubated with S100A12 had increased ICAM-1 and VCAM-1 surface expression, resulting in the adhesion of lymphocytes to endothelial cells. This induction follows activation of NF-κB. MRPs inhibit oxidative burst either directly or by reacting with oxygen metabolites. S100A9 reduces the levels of H₂O₂ released by peritoneal BCG-stimulated macrophages. This effect can be observed using human and murine S100A9, but not S100A8. Unlike S100A9, S100A8 can be efficiently oxidized by OCl⁻ anions, resulting in the formation of a covalently-linked S100A8 homodimer and loss of its chemotactic activity (demonstrated for murine S100A8).

Alternatively, since MRPs are cytosolic proteins, they could protect neutrophils from the harmful effects of its own oxidative burst. S100A9 is also known as being involved in the control of inflammatory pain by its nociceptive effect. The functions of the MRPs have also been explored in vivo. When injected interperitoneally into mice, murine S100A8 stimulated the accumulation of neutrophils and macrophages within 4 hours. Inhibition of S100A12 reduced the acute inflammation in murine models of delayed-type hypersensitivity and of chronic inflammation in colitis. All MRPs induce an inflammatory reaction when injected in the murine air pouch model.

In some embodiments, the target gene encodes proteins of the matrix metalloproteinase (MMP) family, which are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. The enzyme encoded by this gene degrades type IV and V collagens. Studies in rhesus monkeys suggest that the enzyme is involved in IL-8-induced mobilization of hematopoietic progenitor cells from bone marrow, and murine studies suggest a role in tumor-associated tissue remodeling.

MMPs, particularly MMP9, 2 and 3 have been implicated in cancer for more than 40 years. In addition to their role in ECM degradation, mounting evidence suggest their role in angiogenesis, lymphangiogenesis and vasculogenesis which are critical to cancer cell invasion and metastasis. For example, MMP9 increases the bioavailability of sequestered VEGF binding to its receptor in several cancers such as colon and pancreatic cancers. MMP9 also mediates the proteolytic activation of TGF-β which is an important grow factor in HCC. Matrix metalloproteinases (MMPs) are proteases to promoted cancer cells growth, migration, invasion and metastasis (Egeblad and Werb, 2002). Overexpression of MAN1A1 increased MMP9 mRNA expression level, and overexpression of MAN1C1 decreased MMP9 mRNA expression level. Due to MMPs are capable of degrading all kinds of extracellular matrix proteins, decreased MMP9 expression means that cell migration and invasion ability is inhibited. Genes that known to be involved in metastasis include MMP9 and C7TN. MMP9 is a member of a group of secreted zinc metalloproteases which, in mammals, degrade the collagens of the extracellular matrix. The elevated expression of MMP9 has been linked to metastasis in many different cancer types (Turner et al. 2000; Osman et al. 2002). C7TN has been shown to be the oncogene resided in the 11q13 region that is found to be frequently amplified in squamous cell carcinomas of the head and neck and breast cancer (Schuuring et al. 1992; Schuuring et al. 1998).

In some embodiments, the target gene may be genes that are involved in tumorigenesis, including BMP2 and EGFR. BMP2 is a member of the transforming growth factor-beta superfamily, which controls proliferation, differentiation, and other functions in many cell types. EGFR is one of the most frequently amplified and mutated gene in many different type of cancers, including head and neck SCC (Santani et al. 1991; Dassonville et al. 1993; Grandis and Tweardy 1993). Other identified candidate genes, that their roles in metastasis process have not been clearly defined, include GTSE1, EEF1A1, GTSE1 is a microtubule-localized protein. Its expression is cell cycle regulated and can induce G2/M-phase accumulation when overexpressed (Monte et al. 2000). It has been demonstrated that GTSE1 is able to down-regulate levels and activity of the p53 tumor suppressor protein and represses its ability to induce apoptosis after DNA damage (Monte et al. 2004). EEF1A1 gene codes for the alpha subunit of elongation factor-1 which is involved in the binding of aminoacyl-tRNAs to 80S ribosomes. The involvement of this gene with the tumorigenesis is not clear.

In some embodiments, the target gene is SAT1. The protein encoded by the SAT1 gene belongs to the acetyltransferase family, and is a rate-limiting enzyme in the catabolic pathway of polyamine metabolism. It catalyzes the acetylation of spermidine and spermine, and is involved in the regulation of the intracellular concentration of polyamines and their transport out of cells. Defects in this gene are associated with keratosis follicularis spinulosa decalvans (KFSD). Alternatively spliced transcripts have been found for this gene.

In some embodiments, the target gene is TYMP. The TYMP gene (previously known as ECGF1) provides instructions for making an enzyme called thymidine phosphorylase. Thymidine is a molecule known as a nucleoside, which (after a chemical modification) is used as a building block of DNA. Thymidine phosphorylase converts thymidine into two smaller molecules, 2-deoxyribose 1-phosphate and thymine. This chemical reaction is an important step in the breakdown of thymidine, which helps regulate the level of nucleosides in cells. Thymidine phosphorylase plays an important role in maintaining the appropriate amount of thymidine in cell structures called mitochondria. Mitochondria convert the energy from food into a form that cells can use. Although most DNA is packaged in chromosomes within the nucleus, mitochondria also have a small amount of their own DNA (called mitochondrial DNA or mtDNA). Mitochondria use nucleosides, including thymidine, to build new molecules of mtDNA as needed. About 50 mutations in the TYMP gene have been identified in people with mitochondrial neurogastrointestinal encephalopathy (MNGIE) disease. TYMP mutations greatly reduce or eliminate the activity of thymidine phosphorylase. A shortage of this enzyme allows thymidine to build up to very high levels in the body. An excess of thymidine appears to be damaging to mtDNA, disrupting its usual maintenance and repair. As a result, mutations can accumulate in mtDNA, causing it to become unstable. Mitochondria may also have less mtDNA than usual (mtDNA depletion). These genetic changes impair the normal function of mitochondria. Although mtDNA abnormalities underlie the digestive and neurological problems characteristic of MNGIE disease, it is unclear how defective mitochondria cause the specific features of the disorder.

In some embodiments, the reference gene is STK4. The protein encoded by the STK4 gene is a cytoplasmic kinase that is structurally similar to the yeast Ste20p kinase, which acts upstream of the stress-induced mitogen-activated protein kinase cascade. The encoded protein can phosphorylate myelin basic protein and undergoes autophosphorylation. A caspase-cleaved fragment of the encoded protein has been shown to be capable of phosphorylating histone H2B. The particular phosphorylation catalyzed by this protein has been correlated with apoptosis, and it's possible that this protein induces the chromatin condensation observed in this process.

In some embodiments, an assay may involve one or more of the following reference genes: PLGLB2, GABARAP, NACA, EIF1, UBB, UBC, CD81, TMBIM6, MYL12B, HSP90BL, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACTB, CASC3, SKP1, and HNRNPA1; and one or more of the following target genes: CTSS, FPR1, FPR2, FPRL1, FPRL2, CXCR2, NCF2, S100A12, MMP9, SAT1, TYMP, APOBEC3A, SELL, S100A9, and PADI4.

Regression may be used to fit data points generated from patient samples to the standard, such that results are expressed in standard units. In some embodiments, the standard consists of RNA created from one or more cell lines. In some embodiments, the standard may consist of synthetic RNAs. The number of fragments of each RNA within the standard may be known, and the standardized unit may be number of RNA molecules present for each target.

Assays may involve components of different sequence or with different detectable labels targeted to similar regions, components targeted to different regions of the same genes, or components targeting the regions of genes other than those listed in the R1a assay above.

The results may be evaluated using the Decision Rules for Viomics' Test for cancer such as Viomics' NSCLC Test. A plot may be created where one axis is the ratio of a particular target gene to a first reference gene, and the other axis is the ratio of the target gene to a second reference gene.

When a cell line control is used, NSCLC and Normal Sample results are significantly different from one another. Despite the presence of some overlap, NSCLC samples consistently show target gene expression to reference gene expression ratios that are significantly greater than non-cancer samples when fit to a cell line control.

When a synthetic RNA standard rather than a cell line control is used, similar results are obtained. A decreased overlap may be due to decreased variability in the standards resulting from reduced numbers of serial dilutions (from 6 to 3). Each step of the serial dilution may introduce error.

The results may also be interpreted as a single ratio between a linear combination of a first target gene expression and a linear combination of a second target gene expression. A decision rule may state that any score above a given threshold indicates cancer, while a score below the threshold indicates the lack of cancer. A synthetic standard may be designed such that the coefficient on each marker is 1, such that the score is calculated as: Score=Target gene/(Reference gene 1+Reference gene 2).

For example, gene expression values for genes selected from the lists above may be determined from a sample and compared to levels determined from a set of synthetic standards (e.g., in a serial dilution series) that span the range of values that are typically obtained. For each gene, the gene expression level determined from a patient sample is compared to the gene expression level determined by performing a regression analysis on a synthetic standard template to fit the accumulation level values for each gene. The regression and fitted values are obtained for each gene individually. Additional analysis (e.g., calculating ratios) may be done once fitted values are obtained.

These scores may be compared to threshold values, such that scores above a threshold are indicative of a heightened risk of lung cancer as indicated by a patient sample.

The correct concentrations for each standard, coefficients and threshold may be determined by collecting data on a small set of samples from both cancer and cancer-free patients, then using a linear model to separate them. The linear model may be generated via a statistical method such as logistic regression or support vector machines with a linear kernel function, or the linear model may be generated by inspection.

Exclusionary criteria may be implemented, such that any sample that meets the exclusionary criteria has no result reported. These exclusionary criteria may include other test preformed before or after one of the described embodiments. The exclusionary criteria may also be based on results of the test itself. For example, in some embodiments very low quantities of the markers indicate a degraded sample, and an unexpectedly large ratio between two reference genes' expression levels may indicate that there is contamination. In some embodiments a sample is excluded if the ratio of two reference genes differs by more than 10, 5, 4, 3, or 2-fold compared to the median ratio of the accumulation levels of the genes.

In some embodiments the method may involve a Statistical Distance Determination. In some embodiments, the method determines the assay outcome (e.g., positive or negative result) based on statistical distances between results as opposed to a fixed cutoff determined only through ROC curves.

Based on the specificity, the results may be divided into groups (high confidence, low confidence, etc.). This number may also be transformed by some simple formula to create a numerical score for confidence.

In some embodiments the method may involve Models and Derivations for predicting the type of cancer present in a patient based on results RNA expression in combination with demographic or lifestyle attribute(s).

### Methods of RNA Extraction

General methods for RNA extraction are disclosed in standard textbooks of molecular biology, including Ausubel et al. (1997) Current Protocols of Molecular Biology, John Wiley and Sons. In particular, RNA isolation can be performed using purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns. Numerous RNA isolation kits are commercially available and can be used in the methods of the disclosed technology.

In some embodiments, RNA in a whole blood sample may be extracted using the QIAamp® RNA Blood Mini Kit (Qiagen, Germantown, Md.). To purify total RNA from a biological material, e.g. whole blood, the biological material is contacted with the RNA Lysing/Binding Solution before it is contacted with the solid support. The RNA Lysing/Binding Solution is used to lyse the biological material and release the RNA before adding it to the solid support. Additionally, the RNA Lysing/Binding Solution prevents the deleterious effects of harmful enzymes such as RNases. The RNA Lysing/Binding Solution may be successfully used to lyse cultured cells or white blood cells in pellets, or to lyse cells adhering to or collected in culture plates, such as standard 96-well plates. If the biological material is composed of tissue chunks or small particles, the RNA Lysing/Binding Solution may be effectively used to grind such tissue chunks into a slurry because of its effective lysing capabilities. The RNA Lysing/Binding Solution volume may be scaled up or down depending on the cell numbers or tissue size. Once the biological material is lysed, the lysate may be added directly to the solid support or may be put through a pre-clear membrane to eliminate large particulates from the lysate. An example of an appropriate product is the Gentra Solid Phase RNA Pre-Clear Column (Gentra Systems, Inc., Minneapolis, Minn.).

Alternatively, the RNA Lysing/Binding Solution may be added directly to the solid support, thereby eliminating a step, and further simplifying the method. In this latter method, the RNA Lysing/Binding Solution may be applied to the solid support and then dried on the solid support before contacting the biological material with the treated solid support. For example, in one embodiment, a suitable volume of RNA Lysing/Binding Solution is directly added to a solid support placed in a Spin-X® basket (Costar, Corning N.Y.) which is further placed in a 2 ml spin tube. The solid support is heated until dry for at least 12 hours at a temperature of between 40-80° C., after which any excess unbound RNA Lysing/Binding Solution is removed, and is then stored under desiccation. The biological material may be directly added to the solid support pre-treated with the RNA Lysing/Binding Solution, and allowed to incubate for at least one minute, such as for at least 5 minutes, until it is suitably lysed and the nucleic acids are released, and bound to the solid support.

When the biological materials comprise cellular or viral materials, direct contact with the RNA Lysing/Binding Solution, or contact with the solid support pre-treated with the RNA Lysing/Binding Solution causes the cell and nuclear membranes, or viral coats, to solubilize and/or rupture, thereby releasing the nucleic acids as well as other contaminating substances such as proteins, phospholipids, etc. The released nucleic acids selectively bind to the solid support in the presence of the RNA-complexing lithium salt. Having the optional reducing agent helps provide for reduction in RNase activity, which may be necessary in high RNase-containing tissues.

After this incubation period, the remainder of the biological material is optionally removed by suitable means such as centrifugation, pipetting, pressure, vacuum, or by the combined use of these means with an RNA wash solution such that the nucleic acids are left bound to the solid support. The remainder of the non-nucleic acid biological material which includes proteins, phospholipids, etc., may be removed first by centrifugation. By doing this, the unbound contaminants in the lysate are separated from the solid support. The multiple wash steps rid the solid support of substantially all contaminants, and leave behind RNA preferentially bound to the solid support.

Subsequently, the bound RNA may be eluted using an adequate amount of an RNA Elution Solution known to those skilled in the art. The solid support may then be centrifuged, or subjected to pressure or vacuum, to release the RNA from the solid support and can then be collected in a suitable vessel.

In some embodiments the method can begin by extracting cfRNA from a patient's sample and assaying the extracted cfRNA. See. e.g., O'Driscoll, L. et al. (2008) “Feasibility and relevance of global expression profiling of gene transcripts in serum from breast cancer patients using whole genome microarrays and quantitative RT-PCR.” Cancer Genomics Proteomics 5:94-104, which is hereby incorporated by reference in its entirety. In some embodiments, a consistent, repeatable method is used to isolate cfRNA from plasma or other source of RNA to ensure the reliability of the data. To obtain cfRNA from blood, one may use the protocol listed below although other methods are also contemplated.

cfRNA molecules may be purified from plasma or other samples using, for example, Qiagen's QIAamp® circulating nucleic acid kit. The protocol in this kit is described in the document “QIAamp Circulating Nucleic Acid Handbook”, Second Edition, January 2011, which is hereby incorporated by reference in its entirety. This protocol provides an embodiment of a method to purify circulating total nucleic acid from 1 mL of plasma. In brief, lysis reagents and proteases are added along with inert carrier RNA. The total nucleic acid (DNA and RNA) is bound to a column, and the column is washed multiple times then eluted off the column.

For example the protocol may be performed by executing the steps as follows. Pipet 100 μl, 200 μl, or 300 μl QIAGEN® Proteinase K into a 50 ml centrifuge tube. Add 1 ml, 2 ml, or 3 ml of serum or plasma to the 50 ml tube. Add 0.8 ml, 1.6 ml, or 2.4 ml Buffer ACL (containing 1.0 μg carrier RNA). Close the cap and mix by pulse-vortexing for 30 s, making sure that a visible vortex forms in the tube. In order to ensure efficient lysis, mix the sample and Buffer ACL thoroughly to yield a homogeneous solution. The procedure should not be interrupted at this time.

To start the lysis incubation, incubate at 60° C. for 30 min. Place the tube back on the lab bench and add 1.8 ml, 3.6 ml, or 5.4 ml Buffer ACB to the lysate in the tube. Close the cap and mix thoroughly by pulse-vortexing for 15-30 seconds. Incubate the lysate-Buffer ACB mixture in the tube for 5 min on ice. Insert the QIAamp® Mini column into the VacConnector on the QIAvac® 24 Plus. Insert a 20 ml tube extender into the open QIAamp® Mini column. Make sure that the tube extender is firmly inserted into the QIAamp® Mini column in order to avoid leakage of sample.

Keep the collection tube for the dry spin, below. Apply the lysate-Buffer ACB mixture into the tube extender of the QIAamp® Mini column. Switch on the vacuum pump. When all lysates have been drawn through the columns completely, switch off the vacuum pump and release the pressure to 0 mbar. Carefully remove and discard the tube extender. Please note that large sample lysate volumes (about 11 ml when starting with 3 ml sample) may need up to 10 minutes to pass through the QIAamp® Mini membrane by vacuum force. For fast and convenient release of the vacuum pressure, the Vacuum Regulator should be used (part of the QIAvac® Connecting System). To avoid cross-contamination, be careful not to move the tube extenders over neighboring QIAamp® Mini Columns.

Apply 600 μl Buffer ACW1 to the QIAamp® Mini column. Leave the lid of the column open, and switch on the vacuum pump. After all of Buffer ACW1 has been drawn through the QIAamp® Mini column, switch off the vacuum pump and release the pressure to 0 mbar. Apply 750 μl Buffer ACW2 to the QIAamp® Mini column. Leave the lid of the column open, and switch on the vacuum pump. After all of Buffer ACW2 has been drawn through the QIAamp® Mini column, switch off the vacuum pump and release the pressure to 0 mbar. Apply 750 μl of ethanol (96-100%) to the QIAamp® Mini column. Leave the lid of the column open, and switch on the vacuum pump. After all of ethanol has been drawn through the spin column, switch off the vacuum pump and release the pressure to 0 mbar. Close the lid of the QIAamp® Mini column. Remove it from the vacuum manifold, and discard the VacConnector. Place the QIAamp® Mini column in a clean 2 ml collection tube, and centrifuge at full speed (20,000×g; 14,000 rpm) for 3 min.

Place the QIAamp® Mini Column into a new 2 ml collection tube. Open the lid, and incubate the assembly at 56° C. for 10 min to dry the membrane completely. Place the QIAamp® Mini column in a clean 1.5 ml elution tube (provided) and discard the 2 ml collection tube from step 14. Carefully apply 20-150 μl of Buffer AVE to the center of the QIAamp® Mini membrane. Close the lid and incubate at room temperature for 3 min. Ensure that the elution buffer AVE is equilibrated to room temperature (15-25° C.). If elution is done in small volumes (<50 μl) the elution buffer has to be dispensed onto the center of the membrane for complete elution of bound DNA. Elution volume is flexible and can be adapted according to the requirements of downstream applications. The recovered eluate volume will be up to 5 μl less than the elution volume applied to the QIAamp® Mini column. Centrifuge in a microcentrifuge at full speed (20,000×g; 14,000 rpm) for 1 min to elute the nucleic acids. The above example QIAamp® Circulating Nucleic Acid Handbook 1/2011 is representative on knowledge of one of skill in the art and it illustrative rather than limiting. Alternate embodiments, including variants on the methods above or distinct approaches to cfRNA purification, are contemplated herein, and the methods and compositions disclosed herein are not limited to any particular cfRNA purification method. Exemplary RNA methods are further discussed in Example 1, below.

i. Sequencing-Based Methods of Detecting Gene Expression Levels

In some embodiments, RNA levels may be assayed using sequencing technology. Examples of sequencing technology include but are not limited to one or more technologies such as pyrosequencing, e.g., ‘the’454′ method (Margulies et al., (2005) Genome sequencing in microfabricated high-density picolitre reactors. Nature 437:376-380; Ronaghi, et al. (1996) Real-time DNA sequencing using detection of pyrophosphate release. Anal. Biochem. 242:84-89), ‘Solexa’ or Illumina-type sequencing (Fedurco et al., (2006), BTA, a novel reagent for DNA attachment of glass and efficient generation of solid-phase amplified DNA colonies. Nucleic Acid Research 34, e22; Turcatti et al. (2008), A new class of cleavable 10 fluorescent nucleotides: synthesis and optimization as reversible terminators for DNA sequencing by synthesis. Nucleic Acid Research 36, e25), SOLiD sequencing technology (Shendure, J. et al. (2005) Accurate multiplex polony sequencing of an evolved bacterial genome. Science 309, 1728-1732; McKeman, K. et al, (2006) Reagents, methods, and libraries for bead-based sequencing. US patent application 20080003571), Heliscope Technology (Harris, T. D. et al. (2008) Single-molecule DNA sequencing of a viral genome. Science 320, 106-109), Ion Torrent Technology (Rothberg et al., (2011) An integrated semiconductor device enabling non-optical genome sequencing. Nature 475, 348-352), SMRT Sequencing Technology (Pacific Biosciences), or GridION nanopore-based sequencing (Oxford Nanopore Technologies; http://www.nanoporetech.com/technology/the-gridion-system/the-gridion-system). In some embodiments any number of so-called ‘next generation’ DNA sequencing methods may be used, as described in Shendure and Ji, “Next-generation DNA sequencing”, Nature Biotechnology 26(10):1135-1145 (2008) or in other art available to one of skill in the art. Other methods for the determination of DNA sequence are also applicable, and embodiments disclosed herein are not limited to any particular method of determining base identity at a particular locus to the exclusion of any other method.

In some embodiments, Next Generation Sequencing (NGS) techniques that allow for massively parallel sequencing of clonally amplified molecules and of single nucleic acid molecules are used. Non-limiting examples of NGS include sequencing-by-synthesis using reversible dye terminators, and sequencing-by-ligation.

In some embodiments, a ligation reaction composition is formed comprising at least one RNA molecule to be detected, at least one first adaptor, at least one second adaptor, and a double-strand specific RNA ligase. The first adaptor comprises a first oligonucleotide comprising at least two ribonucleosides on the 3′-end and a second oligonucleotide that comprises a single-stranded portion when the first oligonucleotide and the second oligonucleotide are hybridized together. The second adaptor comprises a third oligonucleotide that comprises a 5′ phosphate group and a fourth oligonucleotide that comprises a single-stranded portion when the third oligonucleotide and the fourth oligonucleotide are hybridized together. A first adaptor and a second adaptor are ligated to an RNA molecule in the ligation reaction composition by the double-strand specific RNA ligase to form a ligated product. The first adaptor and the second adaptor anneal with the RNA molecule in a directional manner due to their structure and each adaptor is ligated simultaneously or nearly simultaneously to the RNA molecule with which it is annealed, rather than sequentially (for example, when a second adaptor and the RNA molecule are combined with a ligase and the second adaptor is ligated to the 3′ end of the RNA molecule, then subsequently a first adaptor is combined with the ligated RNA molecule-second adaptor and the first adaptor is then ligated to the 5′ end of the RNA molecule-second adaptor, with an intervening purification step between ligating the second adaptor to the RNA molecule and ligating the first adaptor to the RNA molecule, see, e.g., Elbashir et al, Genes and Development 15: 188-200, 2001; Berezikov et al., Nat. Genet. Supp. 38: S2-S7, 2006). It is to be appreciated that the order in which components are added to the ligation reaction composition is not limiting and that the components may be added in any order. It is also to be appreciated that during the process of adding components, an adaptor may be ligated with a corresponding RNA molecule in the presence of a ligase before all of the components of the reaction composition are added, for example but without limitation, a second adaptor may be ligated with a corresponding RNA molecule in the presence of a ligase before the first adaptors are added, and that such reactions are within the intended scope of the current teachings, provided there is not a purification procedure between the time one adaptor is ligated to the RNA molecule and the time the other adaptor is ligated to the RNA molecule. An RNA-directed DNA polymerase (sometimes referred to as an RNA-dependent DNA polymerase) is combined with the ligated product to form reaction mixture, which is incubated under conditions suitable for a reverse transcribed product. The reverse transcribed product is combined with a ribonuclease, typically ribonuclease H (RNase H), and at least some of the ribonucleosides are digested from the reverse transcribed product to form an amplification template.

Next, the amplification template is combined with at least one forward primer, at least one reverse primer, and a DNA-directed DNA polymerase (sometimes referred to as a DNA-dependent DNA polymerase) to form an amplification reaction composition. The amplification reaction composition is thermocycled under conditions suitable to allow amplified products to be generated. In some embodiments, at least one species of amplified product is detected. In some embodiments, a reporter probe and/or a nucleic acid dye is used to indirectly detect the presence of at least one of the RNA species in the sample. In certain embodiments, an amplification reaction composition further comprises a reporter probe, for example but not limited to a TaqMan® probe, molecular beacon, Scorpion™ primer or the like, or a nucleic acid dye, for example but not limited to, SYBR® Green or other nucleic acid binding dye or nucleic acid intercalating dye. In certain embodiments of the current teachings, detecting comprises a real-time or end-point detection technique, including without limitation, quantitative PCR. In some embodiments, the sequence of at least part of the amplified product is determined, which allows the corresponding RNA molecule to be identified. In some embodiments, a library of amplified products comprising a library-specific nucleotide sequence is generated from the RNA molecules in a starting material, wherein at least some of the amplified product species share a library-specific identifier, for example but not limited to a library-specific nucleotide sequence, including without limitation, a barcode sequence or a hybridization tag, or a common marker or affinity tag. In some embodiments, two or more libraries are combined and analyzed, then the results are deconvoluted based on the library-specific identifier.

In some embodiments, only one polymerase, a DNA polymerase comprising both DNA-directed DNA polymerase activity and RNA-directed DNA polymerase activity, is employed in the reverse transcription reaction composition and no additional polymerase is used. In other method embodiments, both an RNA-directed DNA polymerase and a DNA-directed DNA polymerase are added to the reverse transcription reaction composition and no additional polymerase is added to the amplification reaction composition.

In some embodiments, a method for detecting a RNA molecule in a sample comprises combining the sample with at least one first adaptor, at least one second adaptor, and a polypeptide comprising double-strand specific RNA ligase activity to form a ligation reaction composition in which the at least one first adaptor and the at least one second adaptor are ligated to the RNA molecule of the sample to form a ligated product in the same ligation reaction composition, and detecting the RNA molecule of the ligated product or a surrogate thereof. In some embodiments, the at least one first adaptor comprises a first oligonucleotide having a length of 10 to 60 nucleotides and comprising at least two ribonucleosides on the 3′-end, and a second oligonucleotide comprising a nucleotide sequence substantially complementary to the first oligonucleotide and further comprising a single-stranded 5′ portion of 1 to 8 nucleotides when the first oligonucleotide and the second oligonucleotide are duplexed. In some embodiments, the at least one second adaptor comprises a third oligonucleotide having a length of 10 to 60 nucleotides and comprising a 5′ phosphate group, and a fourth oligonucleotide comprising a nucleotide sequence substantially complementary to the third oligonucleotide and further comprising a single-stranded 3′ portion of 1 to 8 nucleotides when the third oligonucleotide and the fourth oligonucleotide are duplexed. In some embodiments, the single-stranded portions independently have a degenerate nucleotide sequence, or a sequence that is complementary to a portion of the RNA molecule. In some embodiments, the first and third oligonucleotides have a different nucleotide sequence. In the ligation reaction composition, the RNA molecule to be detected hybridizes with the single-stranded portion of the at least one first adaptor and the single-stranded portion of the at least one second adaptor.

In some embodiments, detecting the RNA molecule or a surrogate thereof comprises combining the ligated product with i) a RNA-directed DNA polymerase, ii) a DNA polymerase comprising DNA dependent DNA polymerase activity and RNA dependent DNA polymerase activity, or iii) a RNA-directed DNA polymerase and a DNA-directed DNA polymerase; reverse transcribing the ligated product to form a reverse transcribed product; digesting at least some of the ribonucleosides from the reverse transcribed product with ribonuclease H to form an amplification template; combining the amplification template with at least one forward primer, at least one reverse primer, and a DNA-directed DNA polymerase when the ligated product is combined as in i), to form an amplification reaction composition; cycling the amplification reaction composition to form at least one amplified product, and determining the sequence of at least part of the amplified product, thereby detecting the RNA molecule.

In some embodiments, a method for generating an RNA library comprises combining a multiplicity of different RNA molecules with a multiplicity of first adaptor species, a multiplicity of second adaptor species, and a double-strand specific RNA ligase to form a ligation reaction composition, wherein the at least one first adaptor comprises a first oligonucleotide comprising at least two ribonucleosides on the 3′-end and a second oligonucleotide that comprises a single-stranded portion when the first oligonucleotide and the second oligonucleotide are hybridized together, and wherein the at least one second adaptor comprises a third oligonucleotide that comprises a 5′ phosphate group and a fourth oligonucleotide that comprises a single-stranded portion when the third oligonucleotide and the fourth oligonucleotide are hybridized together and ligating the at least one first adaptor and the at least one second adaptor to the RNA molecule to form a multiplicity of different ligated product species, wherein the first adaptor and the second adaptor are ligated to the RNA molecule in the same ligation reaction composition. The method further comprises combining the multiplicity of ligated product species with an RNA-directed DNA polymerase, reverse transcribing at least some of the multiplicity of ligated product species to form a multiplicity of reverse transcribed product species, digesting at least some of the ribonucleosides from at least some of the multiplicity of reverse transcribed products with a ribonuclease H (RNase H) to form a multiplicity of amplification template species, combining the multiplicity of amplification template species with at least one forward primer, at least one reverse primer, and a DNA-directed DNA polymerase to form an amplification reaction composition, and cycling the amplification reaction composition to form a library comprising a multiplicity of amplified product species, wherein at least some of the amplified product species comprise an identification sequence that is common to at least some of the other amplified product species in the library.

In some embodiments, the sequence of at least part of the amplified product is determined thereby detecting the RNA molecule of interest. The term “sequencing” is used in a broad sense herein and refers to any technique known in the art that allows the order of at least some consecutive nucleotides in at least part of a RNA to be identified, including without limitation at least part of an extension product or a vector insert. Some non-limiting examples of sequencing techniques include Sanger's dideoxy terminator method and the chemical cleavage method of Maxam and Gilbert, including variations of those methods; sequencing by hybridization, for example but not limited to, hybridization of amplified products to a microarray or a bead, such as a bead array; pyrosequencing (see, e.g., Ronaghi et al., Science 281:363-65, 1998); and restriction mapping. Some sequencing methods comprise electrophoreses, including without limitation capillary electrophoresis and gel electrophoresis; mass spectrometry; and single molecule detection. In some embodiments, sequencing comprises direct sequencing, duplex sequencing, cycle sequencing, single-base extension sequencing (SBE), solid-phase sequencing, or combinations thereof. In some embodiments, sequencing comprises detecting the sequencing product using an instrument, for example but not limited to an ABI PRISM® 377 DNA Sequencer, an ABI PRISM® 310, 3100, 3100-Avant, 3730, or 3730xl Genetic Analyzer, an ABI PRISM® 3700 DNA Analyzer, or an Applied Biosystems SOLiD.® System (all from Applied Biosystems), a Genome Sequencer 20 System (Roche Applied Science), or a mass spectrometer. In certain embodiments, sequencing comprises emulsion PCR (see, e.g., Williams et al., Nature Methods 3(7):545-50, 2006.) In certain embodiments, sequencing comprises a high throughput sequencing technique, for example but not limited to, massively parallel signature sequencing (MPSS). Descriptions of MPSS can be found, among other places, in Zhou et al., Methods of Molecular Biology 331:285-311, Humana Press Inc.; Reinartz et al., Briefings in Functional Genomics and Proteomics, 1:95-104, 2002; Jongeneel et al., Genome Research 15:1007-14, 2005. In some embodiments, sequencing comprises incorporating a dNTP, including without limitation a dATP, a dCTP, a dGTP, a dTTP, a dUTP, a dITP, or combinations thereof and including dideoxyribonucleotide versions of dNTPs, into an amplified product.

Further exemplary techniques that are useful for determining the sequence of at least a portion of a nucleic acid molecule include, without limitation, emulsion-based PCR followed by any suitable massively parallel sequencing or other high-throughput technique. In some embodiments, determining the sequence of at least a part of an amplified product to detect the corresponding RNA molecule comprises quantitating the amplified product. In some embodiments, sequencing is carried out using the SOLiD® System (Applied Biosystems) as described in, for example, PCT patent application publications WO 06/084132 entitled “Reagents, Methods, and Libraries For Bead-Based Sequencing and WO07/121489 entitled “Reagents, Methods, and Libraries for Gel-Free Bead-Based Sequencing.” In some embodiments, quantitating the amplified product comprises real-time or end-point quantitative PCR or both. In some embodiments, quantitating the amplified product comprises generating an expression profile of the RNA molecule to be detected, such as an mRNA expression profile or a miRNA expression profile. In certain embodiments, quantitating the amplified product comprises one or more Y-nuclease assays, for example but not limited to, TaqMan® Gene Expression Assays and TaqMan® miRNA Assays, which may comprise a microfluidics device including without limitation, a low density array. Any suitable expression profiling technique known in the art may be employed in various embodiments of the disclosed methods.

Those in the art will appreciate that the sequencing method employed is not typically a limitation of the present methods. Rather, any sequencing technique that provides the order of at least some consecutive nucleotides of at least part of the corresponding amplified product or RNA to be detected or at least part of a vector insert derived from an amplified product can typically be used in the current methods. Descriptions of sequencing techniques can be found in, among other places, McPherson, particularly in Chapter 5; Sambrook and Russell; Ausubel et al.; Siuzdak, The Expanding Role of Mass Spectrometry in Biotechnology, MCC Press, 2003, particularly in Chapter 7; and Rapley. In some embodiments, unincorporated primers and/or dNTPs are removed prior to a sequencing step by enzymatic degradation, including without limitation exonuclease I and shrimp alkaline phosphatase digestion, for example but not limited to the ExoSAP-IT® reagent (USB Corporation). In some embodiments, unincorporated primers, dNTPs, and/or ddNTPs are removed by gel or column purification, sedimentation, filtration, beads, magnetic separation, or hybridization-based pull out, as appropriate (see, e.g., ABI PRISM® Duplex™ 384 Well F/R Sequence Capture Kit, Applied Biosystems P/N 4308082).

Those in the art will appreciate that, in certain embodiments, the read length of the sequencing/resequencing technique employed may be a factor in the size of the RNA molecules that can effectively be detected (see, e.g., Kling, Nat. Biotech. 21(12):1425-27). In some embodiments, the amplified products generated from the RNA molecules from a first sample are labeled with a first identification sequence (sometimes referred to as a “barcode” herein) or other marker, the amplified products generated from the RNA molecules from a second sample are labeled with a second identification sequence or second marker, and the amplified products comprising the first identification sequence and the amplified products comprising the second identification sequence are pooled prior to determining the sequence of the corresponding RNA molecules in the corresponding samples. In certain embodiments, three or more different RNA libraries, each comprising a identifier sequence that is specific to that library, are combined. In some embodiments, a first adaptor, a second adaptor, a forward primer, a reverse primer, or combinations thereof, comprise an identification sequence or the complement of an identification sequence.

In some embodiments, sequencing comprises using technologies that are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies include, but are not limited to, the SMRT® technology of Pacific Biosciences, the ION TORRENT® technology, and nanopore sequencing developed for example, by Oxford Nanopore Technologies.

In some embodiments, the method comprises creating a complimentary DNA (cDNA) library representing a particular strand of a RNA molecule in an RNA sample, by: (a) hybridizing a plurality of first primers to an RNA sample under conditions wherein complexes are formed between a 3′ region of two or more first primers in the plurality of first primers and two or more RNA molecules in the RNA sample, wherein the 3′ region of the first primers include a random nucleotide sequence and a first nucleotide sequence tag; (b) extending the plurality of first primers of the complexes by reverse transcription, thereby generating complementary DNA (cDNA) molecules of the two or more RNA molecules; (c) hybridizing a plurality of double stranded polynucleotide molecules including a second nucleotide sequence tag to the two or more cDNA molecules under conditions wherein: (i) a complex is formed between a 3′ overhang of a double stranded polynucleotide molecule in the plurality of double stranded polynucleotide molecules and a 3′ region of the cDNA molecule, wherein the 3′ overhang includes a second random nucleotide sequence, and (ii) a 5′ end of a complementary second strand of the double stranded polynucleotide molecule in the plurality of double stranded polynucleotide molecules is adjacent to a 3′ end of the cDNA molecule; (d) attaching the 5′ end of the complementary second strand of the double stranded polynucleotide molecule to the 3′ end of the two or more cDNA molecules, thereby generating unattached strands of the double stranded polynucleotide molecules; (e) removing the unattached strands the double stranded polynucleotide molecules, thereby forming a plurality of single stranded cDNA molecules including a first and a second nucleotide sequence tag; and (f) converting the plurality of single stranded cDNA molecules to double stranded cDNA molecules, thereby creating a cDNA library representing a particular strand of a RNA molecule of in an RNA sample.

In other embodiments, the method comprises creating a cDNA library representing a particular strand of a RNA molecule in an RNA sample, by: (a) hybridizing a plurality of first primers to an RNA sample under conditions wherein complexes are formed between a 3′ region of two or more first primers in the plurality of first primers and two or more RNA molecules in the RNA sample, wherein the 3′ region of the single stranded primers include a random nucleotide sequence and a first nucleotide sequence tag; (b) extending the first primers of the complexes by reverse transcription, thereby generating complementary DNA (cDNA) molecules of the two or more RNA molecules; (c) attaching double stranded polynucleotide molecules to the cDNA molecules under conditions wherein the (c) attaching double stranded polynucleotide molecules to the cDNA molecules under conditions wherein the 5′ end of the double stranded polynucleotide molecules are attached to the cDNA molecules and the RNA molecules are not attached to the 3′ end of the double stranded polynucleotide molecules, wherein the double stranded DNA molecules include a second nucleotide sequence tag; (d) removing said RNA molecules; and (e) synthesizing complementary second strand DNA molecules from said cDNA molecules, thereby forming a cDNA library representing a particular strand of an RNA molecule in an RNA sample.

In some embodiments, the primer may hybridize to the polynucleotide using a non-random sequence, e.g. a poly T or poly A sequence which, in some forms of this embodiment, may end in a random or non-random non-poly-T or non-poly-T sequence that hybridizes with the target. As another example, a primer may include a sequence corresponding to either substantially complementing or substantially the same as the exon sequence. When multiple polynucleotides are targeted simultaneously, the primers may be the same or different that target the multiple polynucleotides.

In some embodiments, massively parallel sequencing uses Illumina's sequencing-by-synthesis and reversible terminator-based sequencing chemistry (e.g. as described in Bentley et al., Nature 6:53-59 [2009]). In some embodiments, Illumina's sequencing technology relies on the attachment of complimentary DNA (cDNA) of the RNA transcripts to a planar, optically transparent surface on which oligonucleotide anchors are bound. Template cDNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchors. Under limiting-dilution conditions, adapter-modified, single-stranded template DNA is added to the flow cell and immobilized by hybridization to the anchors. Attached DNA fragments are extended and bridge amplified to create an ultra-high density sequencing flow cell with hundreds of millions of clusters, each containing about 1,000 copies of the same template. In one embodiment, the complementary DNA (cDNA) is amplified using PCR before it is subjected to cluster amplification.

In some embodiments, the templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about 20-40 bp, e.g., 36 bp, are aligned against a repeat-masked reference genome and unique mapping of the short sequence reads to the reference genome are identified using specially developed data analysis pipeline software. Non-repeat-masked reference genomes can also be used. Whether repeat-masked or non-repeat-masked reference genomes are used, only reads that map uniquely to the reference genome are counted. After completion of the first read, the templates can be regenerated in situ to enable a second read from the opposite end of the fragments. Thus, either single-end or paired end sequencing of the DNA fragments can be used. Partial sequencing of DNA fragments present in the sample is performed, and sequence tags comprising reads of predetermined length, e.g., 36 bp, are mapped to a known reference genome are counted. In one embodiment, one end of the clonally expanded copies of the cDNA molecules is sequenced and processed by bioinformatic alignment analysis for the Illumina Genome Analyzer, which uses the Efficient Large-Scale Alignment of Nucleotide Databases (ELAND) software.

ii. PCR-Based Methods of Detecting RNA Expression Levels

Samples produced by RNA extraction methods may be highly pure and free of PCR inhibitors, and may be suitable for qPCR as used in some embodiments to assay RNA relative expression as an assay of, for example, various types of cancer.

In some embodiments the methods include performing PCR or qPCR in order to generate an amplicon. PCR and qPCR protocols are exemplified herein below and can be directly applied or adapted for use using the presently described compositions for the detection and/or identification of target genes and reference genes.

Some embodiments provide methods including Quantitative PCR (qPCR) (also referred as real-time PCR). qPCR can provide quantitative measurements, and also provide the benefits of reduced time and contamination. As used herein, “quantitative PCR” (“qPCR” or more specifically “real time qPCR”) refers to the direct monitoring of the progress of a PCR amplification as it is occurring without the need for repeated sampling of the reaction products. In qPCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence (herein referred to as cycle threshold or “CT”) varies directly with the concentration of amplifiable targets at the beginning of the PCR process, enabling a measure of signal intensity to provide a measure of the amount of target nucleic acid in a sample in real time.

To set up PCR and qPCR reactions, the reaction mixture minimally comprises template nucleic acid (e.g., as present in test samples, except in the case of a negative control as described below) and oligonucleotide primers and/or probes in combination with suitable buffers, salts, and the like, and an appropriate concentration of a nucleic acid polymerase. As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-3′ direction along the template until synthesis terminates. An appropriate concentration includes one that catalyzes this reaction in the presently described methods. Known DNA polymerases useful in the methods disclosed herein include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase, FASTSTART™ Taq DNA polymerase, APTATAQ™ DNA polymerase (Roche), KLENTAQ 1™ DNA polymerase (AB peptides Inc.), HOTGOLDSTAR™ DNA polymerase (Eurogentec), KAPATAQ™ HotStart DNA polymerase, KAPA2G™ Fast HotStart DNA polymerase (Kapa Biosystemss), PHUSION™ Hot Start DNA Polymerase (Finnzymes), or the like.

In addition to the above components, the reaction mixture of the present methods includes primers, probes, and deoxyribonucleoside triphosphates (dNTPs).

Usually the reaction mixture will further comprise four different types of dNTPs corresponding to the four naturally occurring nucleoside bases, e.g., dATP, dTTP, dCTP, and dGTP. In some embodiments, each dNTP will typically be present in an amount ranging from about 10 to 5000 μM, usually from about 20 to 1000 μM, about 100 to 800 μM, or about 300 to 600 μM.

The reaction mixture can further include an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like. In some embodiments, the reaction mixture can include BSA, or the like. In addition, in some embodiments, the reactions can include a cryoprotectant, such as trehalose, particularly when the reagents are provided as a master mix, which can be stored over time.

In preparing a reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods disclosed herein, according to the manufacturer's instructions, or modified to improve reaction conditions (e.g., modification of buffer concentration, cation concentration, or dNTP concentration, as necessary), including, for example, Quantifast PCR mixes (Qiagen), TAQMAN® Universal PCR Master Mix (Applied Biosystems), OMNIMIX® or SMARTMIX® (Cepheid), IQ&#8482; Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

The reaction mixture can be subjected to primer extension reaction conditions (“conditions sufficient to provide polymerase-based nucleic acid amplification products”), e.g., conditions that permit for polymerase-mediated primer extension by addition of nucleotides to the end of the primer molecule using the template strand as a template. In many embodiments, the primer extension reaction conditions are amplification conditions, which conditions include a plurality of reaction cycles, where each reaction cycle comprises: (1) a denaturation step, (2) an annealing step, and (3) a polymerization step. As discussed below, in some embodiments, the amplification protocol does not include a specific time dedicated to annealing, and instead comprises only specific times dedicated to denaturation and extension. The number of reaction cycles will vary depending on the application being performed, but will usually be at least 15, more usually at least 20, and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture will usually be raised to, and maintained at, a temperature ranging from about 85 to 100° C., usually from about 90 to 98° C., and more usually from about 93 to 96° C., for a period of time ranging from about 3 to 120 sec, usually from about 3 sec.

Following denaturation, the reaction mixture can be subjected to conditions sufficient for primer annealing to template nucleic acid present in the mixture (if present), and for polymerization of nucleotides to the primer ends in a manner such that the primer is extended in a 5′ to 3′ direction using the nucleic acid to which it is hybridized as a template, e.g., conditions sufficient for enzymatic production of primer extension product. In some embodiments, the annealing and extension processes occur in the same step. The temperature to which the reaction mixture is lowered to achieve these conditions will usually be chosen to provide optimal efficiency and specificity, and will generally range from about 50 to 85° C., usually from about 55 to 70° C., and more usually from about 60 to 68° C. In some embodiments, the annealing conditions can be maintained for a period of time ranging from about 15 sec to 30 min, usually from about 20 sec to 5 min, or about 30 sec to 1 minute, or about 30 seconds.

This step can optionally comprise one of each of an annealing step and an extension step with variation and optimization of the temperature and length of time for each step. In a two-step annealing and extension, the annealing step is allowed to proceed as above. Following annealing of primer to template nucleic acid, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65 to 75° C., usually from about 67 to 73° C., and maintained for a period of time ranging from about 15 sec to 20 min, usually from about 30 sec to 5 min. In some embodiments, the methods disclosed herein do not include a separate annealing and extension step. Rather, the methods include denaturation and extension steps, without any step dedicated specifically to annealing.

The above cycles of denaturation, annealing, and extension may be performed using an automated device, typically known as a thermal cycler. Thermal cyclers that may be employed are described elsewhere herein as well as in U.S. Pat. Nos. 5,612,473; 5,602,756; 5,538,871; and 5,475,610; the disclosures of which are herein incorporated by reference.

The methods described herein can also be used in non-PCR based applications to detect a target nucleic acid sequence, where such target may be immobilized on a solid support. Methods of immobilizing a nucleic acid sequence on a solid support are described in Ausubel et al, eds. (1995) Current Protocols in Molecular Biology (Greene Publishing and Wiley-Interscience, NY), and in protocols provided by the manufacturers, e.g., for membranes: Pall Corporation, Schleicher &amp; Schuell; for magnetic beads: Dynal; for culture plates: Costar, Nalgenunc; for bead array platforms: Luminex and Becton Dickinson; and, for other supports useful according to the embodiments provided herein, CPG, Inc.

Variations on the exact amounts of the various reagents and on the conditions for the PCR or other suitable amplification procedure (e.g., buffer conditions, cycling times, etc.) that lead to similar amplification or detection/quantification results are considered to be equivalents. In one embodiment, the subject qPCR detection has a sensitivity of detecting fewer than 50 copies (preferably fewer than 25 copies, more preferably fewer than 15 copies, still more preferably fewer than 10 copies, e.g., 5, 4, 3, 2, or 1 copy) of target nucleic acid in a sample.

In some embodiments the method may involve PCR amplification of template RNA. A DNase treatment may be conducted to remove DNA contamination from RNA samples. Target RNA may be converted to cDNA with a reverse transcriptase and this step may use one or more of the same primers used within a PCR reaction. Target cDNAs may be amplified by, for example, a consistent, repeatable method to amplify cDNA from plasma or other cDNA. In some embodiments, one or more targets in cDNA may be amplified and quantified via Taqman® chemistry. This protocol may not be the only suitable protocol to detect RNA quantity. However, it may be important to use a consistent protocol for cDNA synthesis and amplification, as variations in protocol may have a large effect on the eventual results.

In some embodiments, Qiagen assay #QF00119602 may be used for the qPCR, using the primers/probes provided accorded to the manufacturer's protocol. Agilent's Universal RNA may be used as a standard in qPCR.

An RNA standard may be used to standardize result across multiple runs. This standard may be run at different dilutions. In some embodiments a synthetic standard may be used. For example, the normal ranges and cut-offs for one or more markers may be examined, and synthetic standards may be obtained and used directly, or diluted or combined such that they are at levels similar to predicted levels, such as predicted levels of the markers. In some embodiments the synthetic standards are present at levels that are at or within an order of magnitude of (e.g., 10-fold higher or 10-fold lower than) predicted levels in a patient sample. In some embodiments the synthetic standards are present at or within a difference of 5× (either 5-fold higher or five-fold lower) than levels predicted for a patient sample. In some embodiments the synthetic standards are present at or within a difference of 2× (either 2-fold higher or 2-fold lower) than levels predicted for a patient sample.

Many methods may be used to determine the appropriate level of each synthetic RNA in the synthetic standard. In one embodiment, one may run some number of samples representative of those and record the results (e.g., Ct value or fitted value to a standard). Each synthetic RNA may then be run on the same assay and the results may be measured on the same scale as the samples (e.g., Ct score or fitted value to a standard). Upon examination, one can determine which standards should be used. For example, 50 samples may be run and Ct scores ranging from 33-38 are obtained for a given gene. Standards of 10⁷, 10⁶, 10⁵, 10⁴, 10³, 10² copies per μL may yield Ct scores of 24, 28, 32, 36, 40, or 44. Thus, it may be decided to use the 10⁵ standard, with dilutions to 10⁴ and 10³ conducted during assay setup. Using this strategy, only the original standard and two dilutions are needed to cover future samples. A similar method could be used to select appropriate concentrations for other standards in the same multiplex. Using this method, different concentrations may be used for each transcript to be assayed so a single standard can be used even if there are large discrepancies between different genes in the multiplex. By using the method disclosed herein, transcripts of widely ranging accumulation levels may be assayed with a reduced number of amplification reactions on standard templates.

For example, if one expects gene A to be in the range of 100 to 10,000 copies/μl and gene B to be in the range of 1,000,000 to 100,000,000 copies, one may create a mixed synthetic standard of 10,000 copies gene A and 100,000,000 copies gene B, thereby only requiring three standards in a 10-fold dilution series to cover the whole range expected for a sample. Using such a synthetic standard may in some embodiments dramatically reduce the number of standard or control samples that need to be run in a qPCR reaction plate to generate a standard curve that covers the expected ranges of both gene a and gene B. This method will also minimize risk of small errors introduced by pipetting from compounding during serial dilutions.

In some embodiments, Reverse Transcriptase PCR (RT-PCR) can be used to determine RNA levels, e.g., mRNA or miRNA levels, of the biomarkers. RT-PCR can be used to compare such RNA levels of the biomarkers in different sample populations, in normal and tumor tissues, with or without drug treatment, to characterize patterns of gene expression, to discriminate between closely related RNAs, and to analyze RNA structure.

Typically, a first step is the isolation of RNA, e.g., mRNA, from a sample. The starting material can be total RNA isolated from a human sample, e.g., human tumors or tumor cell lines, and corresponding normal tissues or cell lines, respectively. Thus RNA can be isolated from a sample, e.g., tumor cells or tumor cell lines, and compared with pooled DNA from healthy donors. If the source of mRNA is a primary tumor, mRNA can be extracted.

Whether the RNA comprises mRNA, miRNA or other types of RNA, gene expression profiling by RT-PCR can include reverse transcription of the RNA template into cDNA, followed by amplification in a PCR reaction. Commonly used reverse transcriptases include, but are not limited to, avian myeloblastosis virus reverse transcriptase (AMV-RT) and Moloney murine leukemia virus reverse transcriptase (MMLV-RT). A reverse transcription step is typically primed using specific primers, random hexamers, stem-loop primers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

In some embodiments, the PCR step employs the Taq DNA polymerase, which has a 5′-3′ nuclease activity but lacks a 3′-5′ proofreading endonuclease activity. TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

In some embodiments, TaqMan™ RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. TaqMan data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

In some embodiments, to minimize errors and the effect of sample-to-sample variation, RT-PCR is performed using an internal standard. An ideal internal standard is expressed at a constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

In some embodiments, real time quantitative PCR can measure PCR product accumulation using a dual-labeled FRET fluorigenic probe (e.g., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. See, e.g. Held et al. (1996) Genome Research 6:986-994.

In some embodiments, PCR flap assays can be used to measure RNA in a sample. As discussed in detail in Example 1, QuARTS and LQAS/TELQAS flap assay technologies combine a polymerase-based target DNA amplification process with an invasive cleavage-based signal amplification process. Described hereinbelow are assays that combine reverse transcription and these flap assay technologies for quantitation of RNAs from a sample.

iii. Alternative Methods of Detecting Ene Expression Levels

In some embodiments, the RNA levels may be assayed via hybridization to a microarray, nCounter or similar. For example, one class of arrays commonly used in differential expression studies includes microarrays or oligonucleotide arrays. These arrays utilize a large number of probes that are synthesized directly on a substrate and are used to interrogate complex RNA or message populations based on the principle of complementary hybridization. Typically, these microarrays provide sets of 16 to 20 oligonucleotide probe pairs of relatively small length (20mers-25mers) that span a selected region of a gene or nucleotide sequence of interest. The probe pairs used in the oligonucleotide array may also include perfect match and mismatch probes that are designed to hybridize to the same RNA or message strand. The perfect match probe contains a known sequence that is fully complementary to the message of interest while the mismatch probe is similar to the perfect match probe with respect to its sequence except that it contains at least one mismatch nucleotide which differs from the perfect match probe. During expression analysis, the hybridization efficiency of messages from a sample nucleotide population are assessed with respect to the perfect match and mismatch probes in order to validate and quantitate the levels of expression for many messages simultaneously. In some embodiments an entire gene array is printed to a microarray. In some embodiments a subset of genes comprising at least one of a target gene and at least one of a reference gene is included on a microarray.

In some embodiments, a device such as an nCounter, offered by Nanostring technologies, for example, may be used to facilitate analysis. An nCounter Analysis System is an integrated system comprising a fully automated prep station, a digital analyzer, the CodeSet (molecular barcodes) and all of the reagents and consumables needed to perform the analysis. Analysis on the nCounter system consists of in-solution hybridization, post-hybridization processing, digital data acquisition, and normalization in one simple workflow. In some embodiments the process is automated. In some embodiments custom or pre-designed sets of barcoded probes may be pre-mixed with a comprehensive set of system controls as part of the analysis.

Some embodiments use an in situ hybridization assay to detect gene expression levels. In an in situ hybridization assay, cells are fixed to a solid support, typically a glass slide. In some embodiments, the cells may be denatured with heat or alkali. The cells are then contacted with a hybridization solution at a moderate temperature to permit annealing of specific probes that are labeled. The probes are preferably labeled with radioisotopes or fluorescent reporters.

In some embodiments, FISH (fluorescence in situ hybridization) uses fluorescent probes that bind to only those parts of a sequence with which they show a high degree of sequence similarity. FISH is a cytogenetic technique used in some embodiments to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

In some embodiments, Comparative Genomic Hybridization (CGH) employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. The copy number information originates from comparisons of the intensities of the hybridization signals among the different locations on the reference genome. The methods, techniques and applications of CGH are described in U.S. Pat. No. 6,335,167, and in U.S. App. Ser. No. 60/804,818, the relevant parts of which are herein incorporated by reference.

B. Quantitative Protein Analysis

In some embodiments, the level of gene expression is determined by detecting the protein expression level. Protein-based detection techniques include immunoaffinity assays. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells.

In other embodiments, alternative antibody-based techniques can also be used, including enzyme-linked immunosorbent assay (ELISA), radioimmunoassay (RIA), immunoradiometric assays (IRMA) and immunoenzymatic assays (IEMA), and sandwich assays using monoclonal or polyclonal antibodies. See, e.g., U.S. Pat. Nos. 4,376,110 and 4,486,530, both of which are incorporated herein by reference.

In some embodiments, Immunohistochemistry is used to detect protein levels. Immunohistochemistry (IHC) is a process of localizing antigens (e.g., proteins) in cells of a tissue binding antibodies specifically to antigens in the tissues. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avidin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag. Using IHC, one or more proteins may be detected. The expression of a gene product can be related to its staining intensity compared to control levels.

In some embodiments, liquid chromatography or mass spectrometry can be used to detect protein levels. In the HPLC-microscopy tandem a spectrometry technique, proteolytic digestion is performed on a protein, and the resulting peptide mixture is separated by reversed-phase chromatographic separation. Tandem mass spectrometry is then performed and the data collected therefrom is analyzed. See Gatlin et al, Anal. Chem., 72:757-763 (2000).

A number of methods of and devices for obtaining the gene expression level data necessary to perform the methods and for use with the compositions and kits disclosed herein, and no single data accumulation method or device should be seen as limiting.

### II. Methylation Marker Analysis

In some embodiments, a marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample, sputum, a blood sample (e.g., plasma, serum, whole blood), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

The technology provides reagents for detecting a differentially methylated region (DMR). In some embodiments an oligonucleotide is provided, the oligonucleotide comprising a sequence complementary to a chromosomal region having an annotation selected from EMX1, GRIN2D, ANKRD13BD, ZNF781, ZNF671, IFFO1, HOPX, BARX1, HOXA9, LOC100129726, SPOCK2, =322D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr19.50, PTGDR_9, DOCK2, MAX_chr19.163, ZNF132, MAX chr19.372, TRH, SP9, DMRTA2, ARNGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329; or a marker selected from any of the subsets of markers defining the group consisting of ZNF781, BARX1, and EMX1; the group consisting of SHOX2, SOBP, ZNF781, CYP26C1, SUCLG2, and SKI; the group consisting of SLC1248, KLHDC7B, PARP15, OPLAH, BCL2L11, MAX.chr12.526, HOXB2, and EMX1; the group consisting of SHOX2, SOBP, 22F781, BTACT, CYP26C1, and DLX4; the group consisting of SHOX2, SOBP, ZNF781, CYP26C1, SUCLG2, and SKI; the group consisting of ZNF781, BARX1, and EMX1, with SOBP and/or HOXA9; the group consisting of BARX1, FLJ45983, SOBP, HOPX, IFFO1, and ZNF781; and the group consisting of BARX1, FAM9B, HOXA9, SOBP, and IFFO1.

Kit embodiments are provided, e.g., a kit comprising a bisulfite reagent; and a control nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above, and having a methylation state associated with a subject who does not have a cancer (e.g., lung cancer). In some embodiments, kits comprise a bisulfite reagent and an oligonucleotide as described herein. In some embodiments, kits comprise a bisulfite reagent; and a control nucleic acid comprising a sequence from such a chromosomal region and having a methylation state associated with a subject who has lung cancer.

The technology is related to embodiments of compositions (e.g., reaction mixtures). In some embodiments are provided a composition comprising a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above, and a bisulfite reagent. Some embodiments provide a composition comprising a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.223, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK4, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above, and an oligonucleotide as described herein. Some embodiments provide a composition comprising a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM39B, DIDO1, MAX_Chr1.110 AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A, BHLHE23, CAPN2, FPF14, FLJ34208, R2_Z, DNMT3A, FERMT3, NFIX, SIPR4, SM SUCLG2, TBX13, and ZNF329, preferably Qom any of the subsets of markers as recited above, and a methylation-specific restriction enzyme.

Some embodiments provide a composition comprising a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX.chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF4, FLJ34208, BIN2_Z, DNMT3A, FERMT3, NFIX, SIPR4, SKI, SUCLG2, TBX15, and ZNF329, preferably from any of the subsets of markers as recited above, and a polymerase.

Additional related method embodiments are provided for screening for a neoplasm (e.g., lung carcinoma) in a sample obtained from a subject, e.g., a method comprising determining a methylation state of a marker in the sample comprising a base in a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM9B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK BCAT1, PRKCB_28, ST8SIA_22, FLJ45983, DLX4, SHOD, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above; comparing the methylation state of the marker from the subject sample to a methylation state of the marker from a normal control sample from a subject who does not have lung cancer; and determining a confidence interval and/or a p value of the difference in the methylation state of the subject sample and the normal control sample. In some embodiments, the confidence interval is 90%, 95%, 97.5%, 9%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above, with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the chromosomal region from a subject who does not have lung cancer to identify differences In the two sequences, and identifying the subject as having a neoplasm when a difference is present.

Systems for screening for lung cancer in a sample obtained from a subject are provided by the technology. Exemplary embodiments of systems include, e.g., a system for screening for lung cancer in a sample obtained from a subject, the system comprising an analysis component configured to determine the methylation state of a sample, a software component configured to compare the methylation state of the sample with a control sample or a reference sample methylation state recorded in a database, and an alert component configured to alert a user of a cancer-associated methylation state. An alert is determined in some embodiments by a software component that receives the results from multiple assays (e.g., determining the methylation states of multiple makers, e.g., a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE3, CAPN2, FGF14, FLJ34208, BIN2_Z, DNMT34, FERMT3, NFIX, SIPR4, SKI, SUCLG2, TBX15, and ZNF329, preferably from any of the subsets of markers as recited above, and calculating a value or result to report based on the multiple results. Some embodiments provide a database of weighted parameters associated with each a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM39B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX13, and ZNF329, preferably from any of the subset of markers as recited above, provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays that is indicative of a lung cancer risk in a subject.

In some embodiments of systems, a sample comprises a nucleic acid comprising a chromosomal region having an annotation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above. In some embodiments the system further comprises a component for Isolating a nucleic acid, a component for collecting a sample such as a component for collecting a stool sample. In some embodiments, the system comprises nucleic acid sequences comprising a chromosomal region having an annotation selected from NA, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6, FAM59, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above. In some embodiments the database comprises nucleic acid sequences from subjects who do not have lung cancer. Also provided are nucleic acids, e.g., a set of nucleic acids, each nucleic acid having a sequence comprising a chromosomal region having an notation selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM39B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX13, and ZNF329, preferably from ay of the subsets of markers as recited above.

Related system embodiments comprise a set of nucleic acids as described, and a database of nucleic acid sequences associated with the set of nucleic acids. Some embodiments further comprise a bisulfite reagent. And, some embodiments further comprise a nucleic acid sequencer.

In certain embodiments, methods for characterizing a sample obtained from a human subject are provided, comprising a) obtaining a sample from a human subject; b) assaying a methylation state of one or more markers in the sample, wherein the marker comprises a base in a chromosomal region having an annotation selected from the following groups of markers: EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX.chr19.163, ZNF132, MAX chr19.372, TRH, SP9, DMRTA2, ARHGEF4, CYP26C, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329, preferably from any of the subsets of markers as recited above; and c) comparing the methylation state of the assayed marker to the methylation state of the marker assayed in a subject that does not have a neoplasm.

In some embodiments, the technology is related to assessing the presence of and methylation state of one or more of the markers identified herein in a biological sample. These markers comprise one or more differentially methylated regions (DMR) as discussed herein. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-specific arbitrarily primed PCR (Gonzalgo et al. (1997) Cancer Res. 57: 594-599). In some embodiments, changes in methylation patterns at specific CpG sites are monitored by digestion of genomic DNA with methylation-specific restriction enzymes, particularly methylation-sensitive enzymes, followed by Southern analysis of the regions of interest (digestion-Southern method). In some embodiments, analyzing changes in methylation patterns involves a process comprising digestion of genomic DNA with one or more methylation-specific restriction enzymes, and analyzing regions for cleavage or non-cleavage indicating the methylation status of analyzed regions. In some embodiments, analysis of the treated DNA comprises PCR amplification, with the amplification result indicating whether the DNA was or was not cleaved by the restriction enzyme. In some embodiments, one or more of the presence, absence, amount, size, and sequence of an amplification product produced is assessed to analyze the methylation status of a DNA of interest. See, e.g., Melnikov, et al., (2005) Nucl. Acids Res, 33(10):e93; Hua, et al. (2011) Exp. Mol. Pathol. 91(1):455-60; and Singer-Sam et al. (1990) Nucl. Acids Res. 18: 687. In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments.

In some embodiments, designs for assaying the methylation states of markers comprise analyzing background methylation at individual CpG loci in target regions of the markers to be interrogated by the assay technology. For example, in some embodiments, large numbers of individual copies of marker DNAs (e.g., >10,000, preferably >100,000 individual copies) from samples isolated from subjects diagnosed with disease, e.g., a cancer, are examined to determine frequency of methylation, and these data are compared to a similarly large numbers of individual copies of marker DNAs from samples isolated from subjects without disease. The frequencies of disease-associated methylation and of background methylation at individual CpG loci within the marker DNAs from the samples can be compared, such that CpG loci that having higher signal-to-noise, e.g., higher detectable methylation and/or reduced background methylation, may be selected for use in assay designs. See, e.g., U.S. Pat. Nos. 9,637,792 and 10,519,510, each of which is incorporated herein by reference in its entirety. In some embodiments a group of high signal-to-noise CpG loci (e.g., 2, 3, 4, 5, or more individual CpG loci in a marker region) are co-interrogated by an assay, such that all of the CpG loci must have a pre-determined methylation status (e.g., all must be methylated or none may be methylated) for the marker to be classified as “methylated” or “not methylated” on the basis of an assay result.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QuARTS assay (e.g., as provided by U.S. Pat. Nos. 8,361,720; 8,715,937; 8,916,344; and 9,212,392, and U.S. patent application Ser. No. 15/841,006).

For example, in some embodiments, methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amounts of methylated DNAs relative to the total amount of DNAs in the population, e.g., (number of methylated DNAs)/(the number of methylated DNAs+number of unmethylated DNAs)×100.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, or other assays). In some embodiments, the kits containing one or more reagent necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

Genomic DNA may be isolated by any means, including the use of commercially available kits. Briefly, wherein the DNA of interest is encapsulated by a cellular membrane the biological sample generally is disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The technology relates to the analysis of any sample that may be associated with lung cancer, or that may be examined to establish the absence of lung cancer. For example, in some embodiments the sample comprises a tissue and/or biological fluid obtained from a patient. In some embodiments, the sample comprises a secretion. In some embodiments, the sample comprises sputum, blood, serum, plasma, gastric secretions, lung tissue samples, lung cells or lung DNA recovered from stool. In some embodiments, the subject is human. Such samples can be obtained by any number of means known in the art, such as will be apparent to the skilled person.

A. Methylation Assays to Detect Lung Cancer

Candidate methylated DNA markers were identified by unbiased whole methylome sequencing of selected lung cancer case and lung control tissues. The top marker candidates were further evaluated in 255 independent patients with 119 controls, of which 37 were from benign nodules, and 136 cases inclusive of all lung cancer subtypes. DNA extracted from patient tissue samples was bisulfite treated and then candidate markers and β-actin (ACTB) as a normalizing gene were assayed by Quantitative Allele-Specific Real-time Target and Signal amplification (QuARTS amplification). QuARTS assay chemistry yields high discrimination for methylation marker selection and screening.

On receiver operator characteristics analyses of individual marker candidates, areas under the curve (AUCs) ranged from 0.512 to 0.941. At 100% specificity, a combined panel of 8 methylation markers (SLC12A8, KLHDC7B, PARP15, OPLAH, BCL2L11, MAX.12.526, HOXB2, and EMX1) yielded a sensitivity of 98.5% across all subtypes of lung cancer. Furthermore, using the 8 markers panel, benign lung nodules yielded no false positives.

B. Methylation Detection Assays and Kits

The markers described herein find use in a variety of methylation detection assays. The most frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Homsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-specific restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides.

Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophore per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

In some embodiments, the bisulfite-treated DNA is purified prior to the quantification. This may be conducted by any means known in the art, such as but not limited to ultrafiltration, e.g., by means of Microcon™ columns (manufactured by Millipore™). The purification is carried out according to a modified manufacturer's protocol (see, e.g., PCT/EP2004/011715, which is incorporated by reference in its entirety). In some embodiments, the bisulfite treated DNA is bound to a solid support, e.g., a magnetic bead, and desulfonation and washing occurs while the DNA is bound to the support. Examples of such embodiments are provided, e.g., in WO 2013/116375 and U.S. Pat. No. 9,315,853, and in U.S. Pat. Appl. Ser. No. 63/058,179, each of which is incorporated herein by reference in its entirety. In certain preferred embodiments, support-bound DNA is ready for a methylation assay immediately after desulfonation and washing on the support. In some embodiments, the desulfonated DNA is eluted from the support prior to assay.

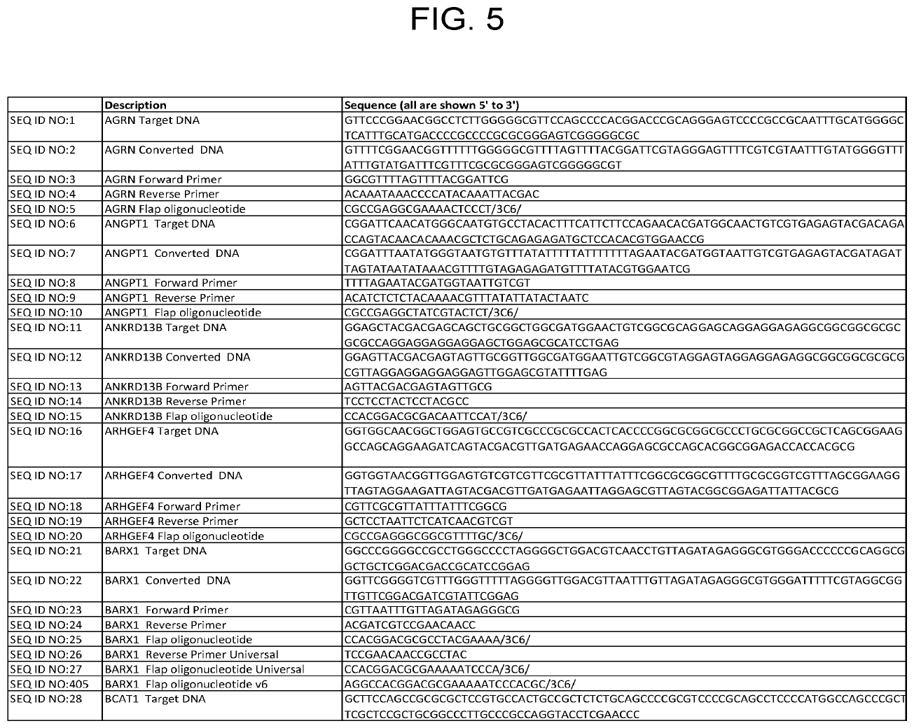

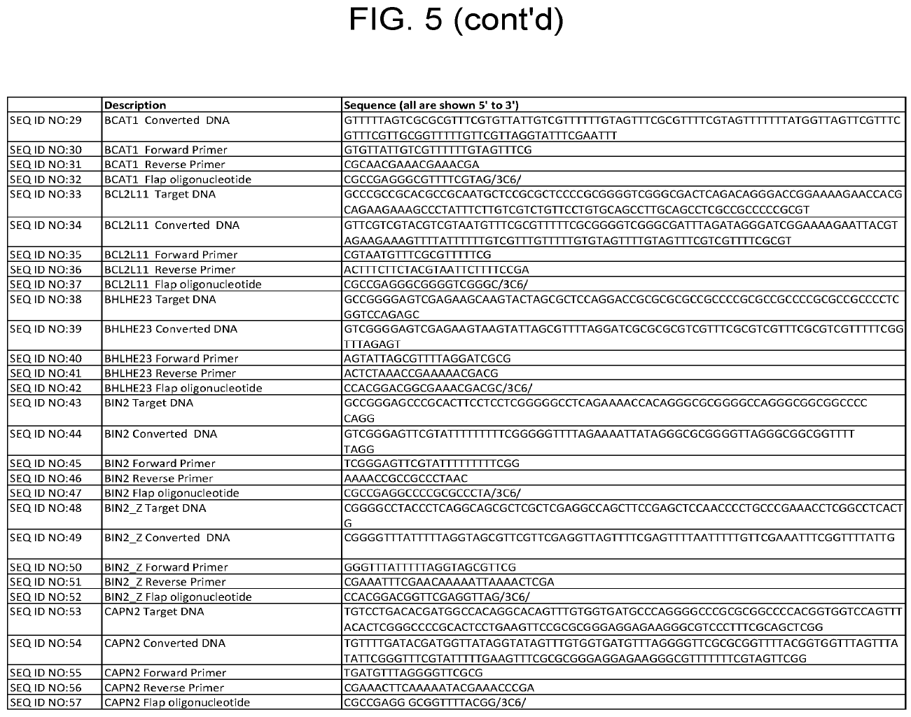

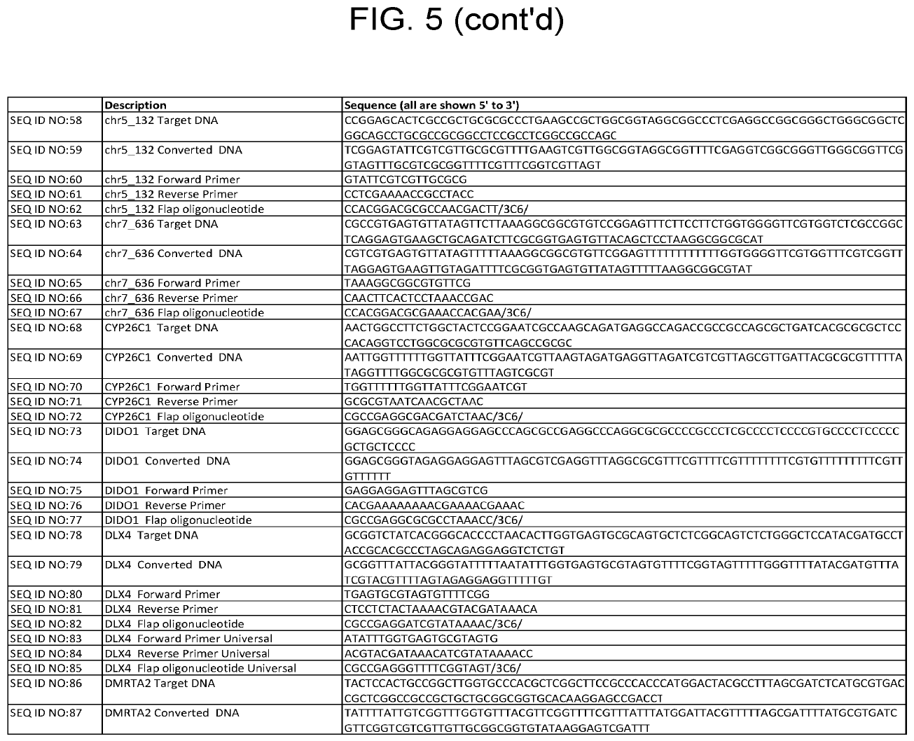

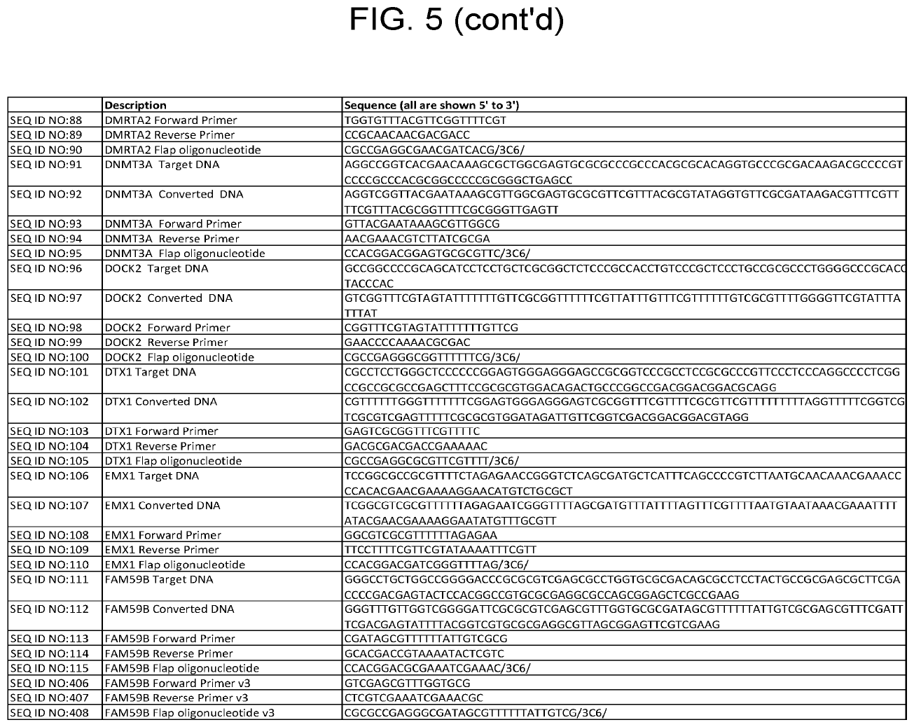

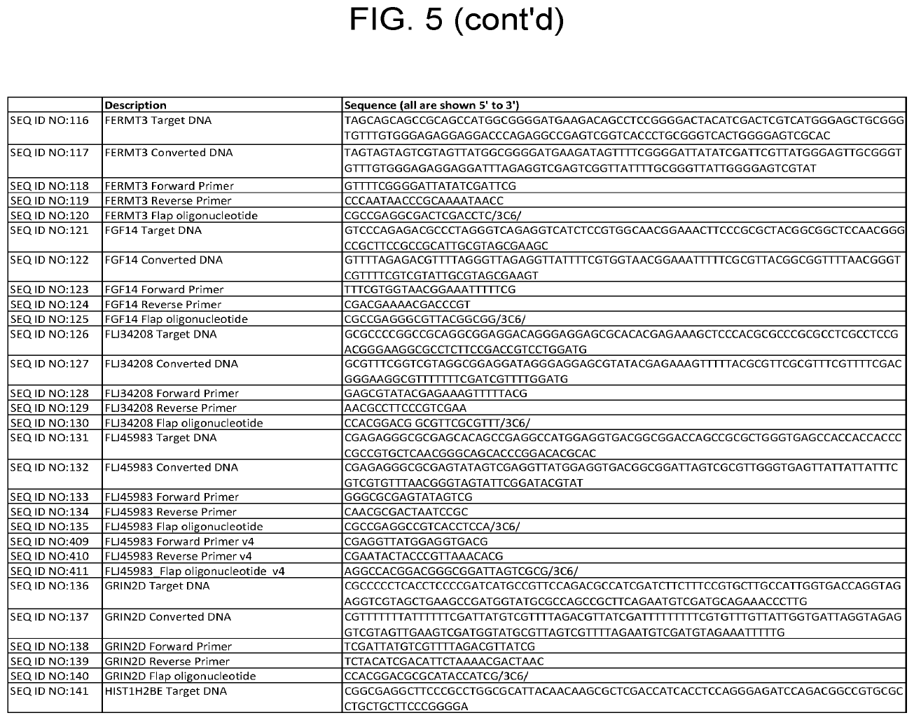

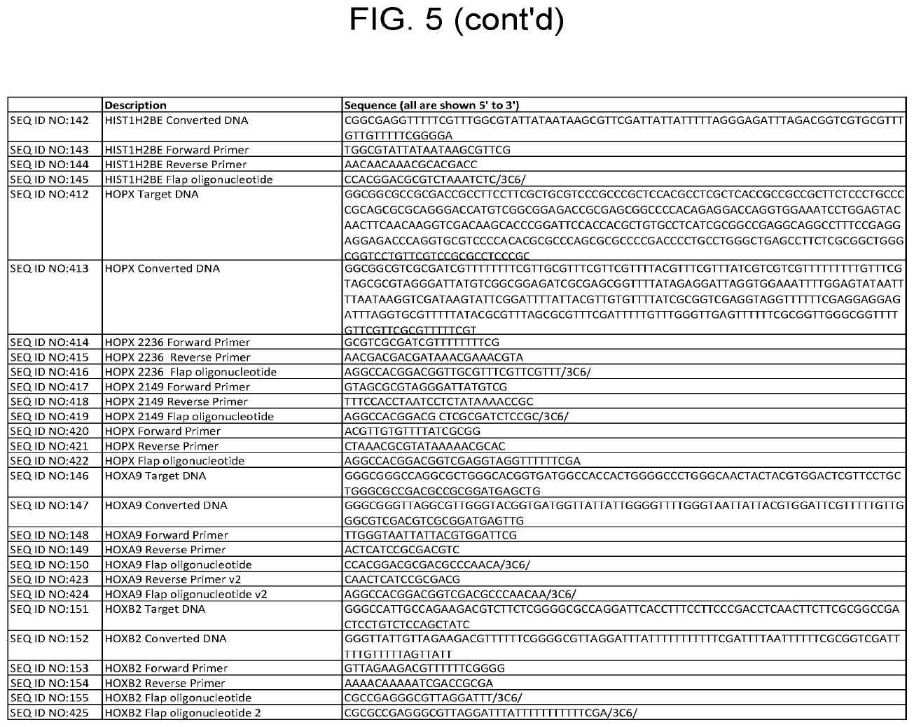

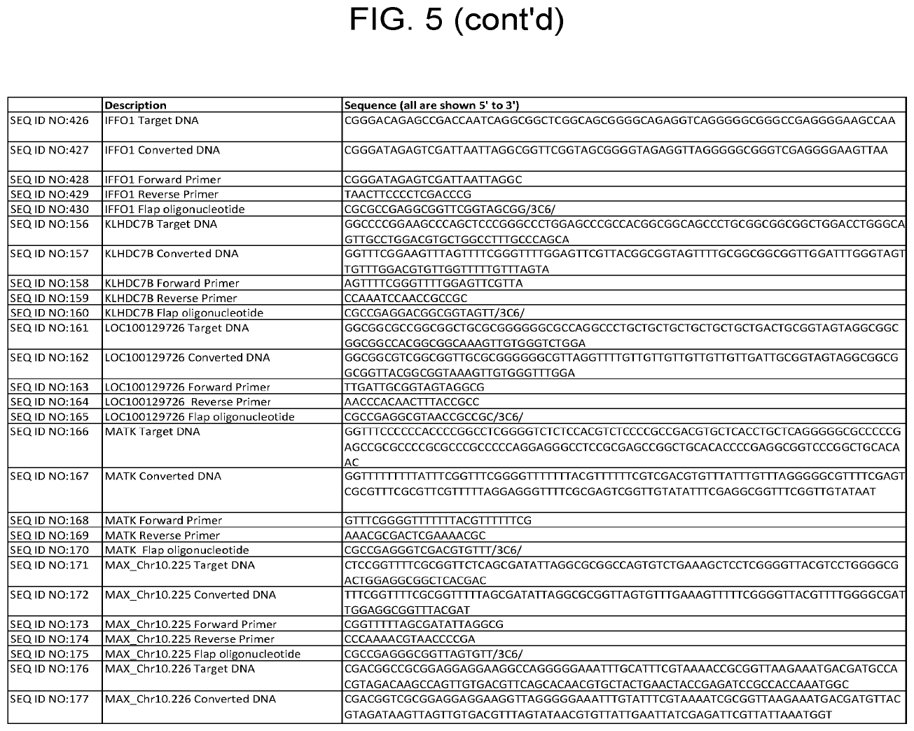

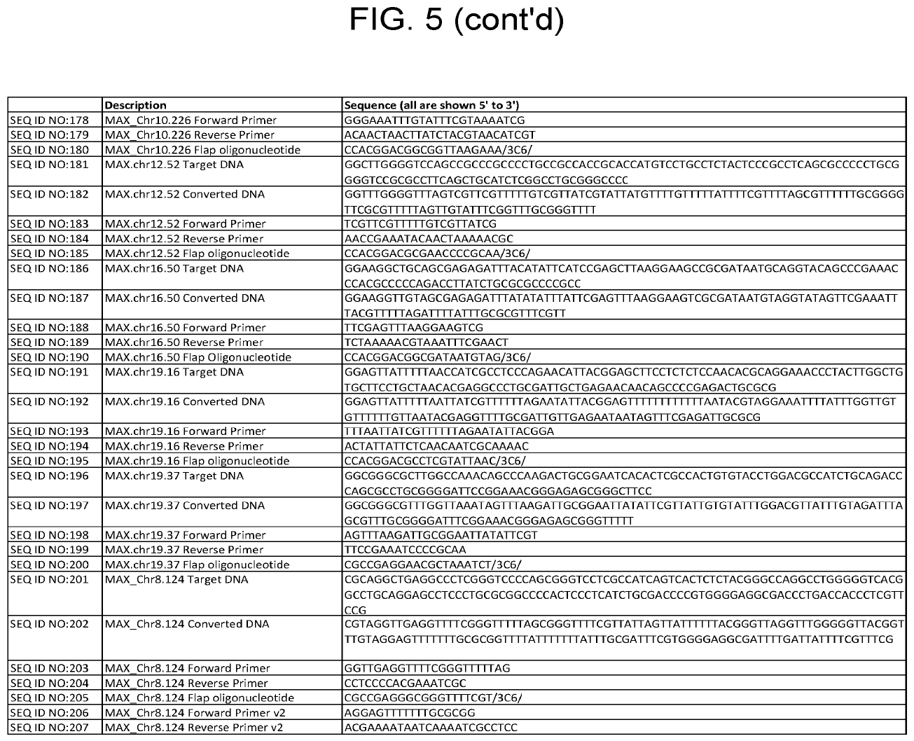

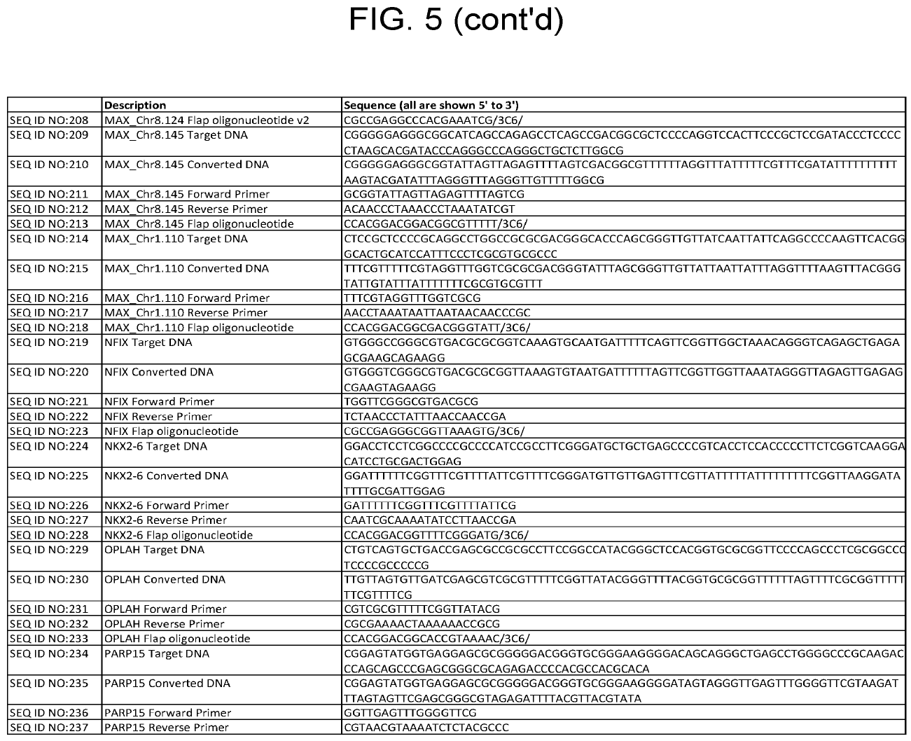

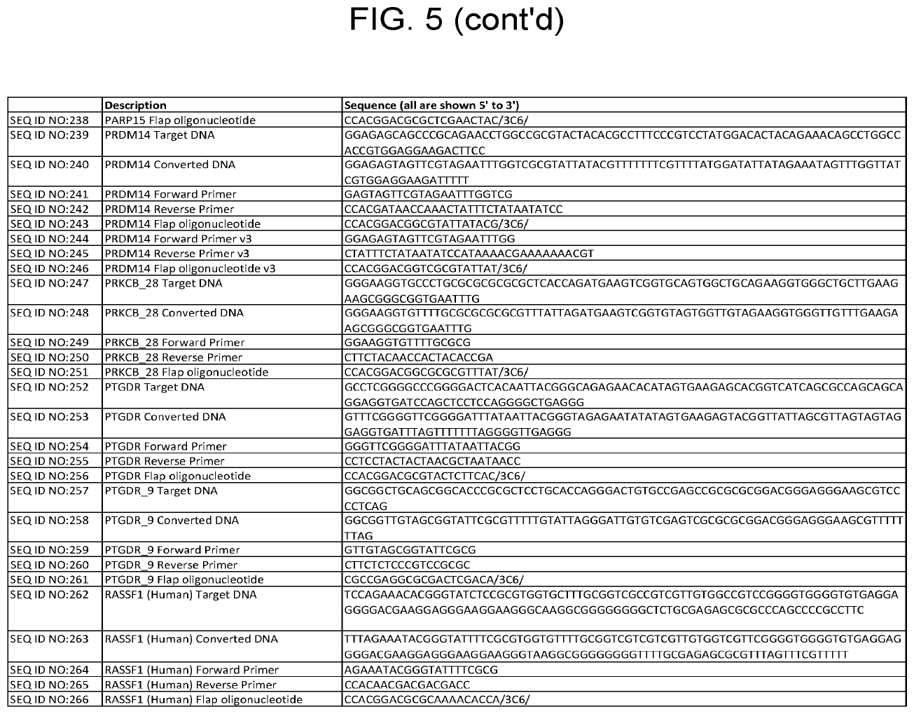

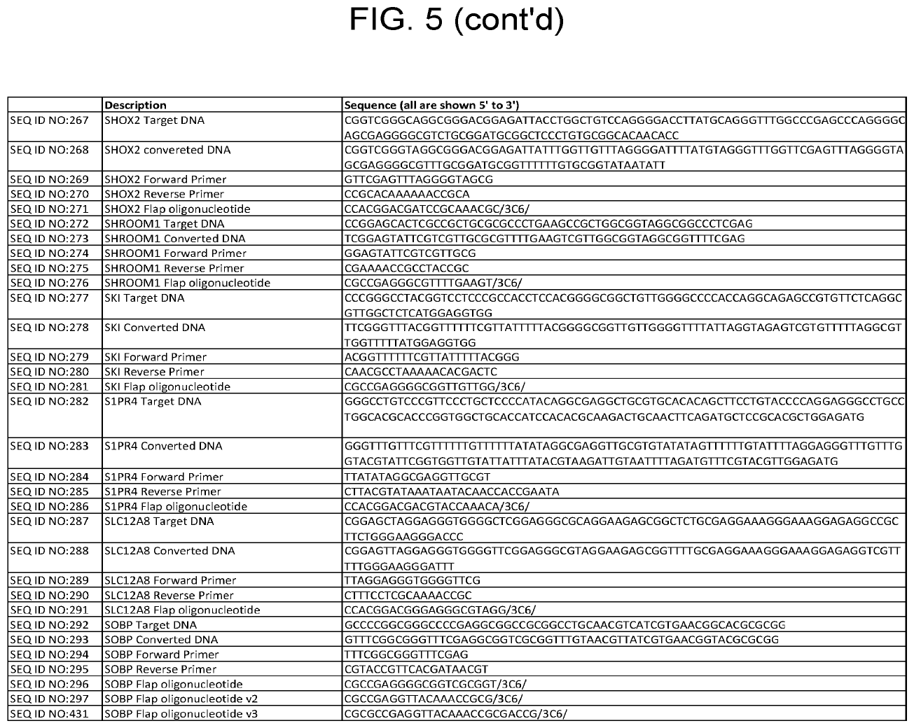

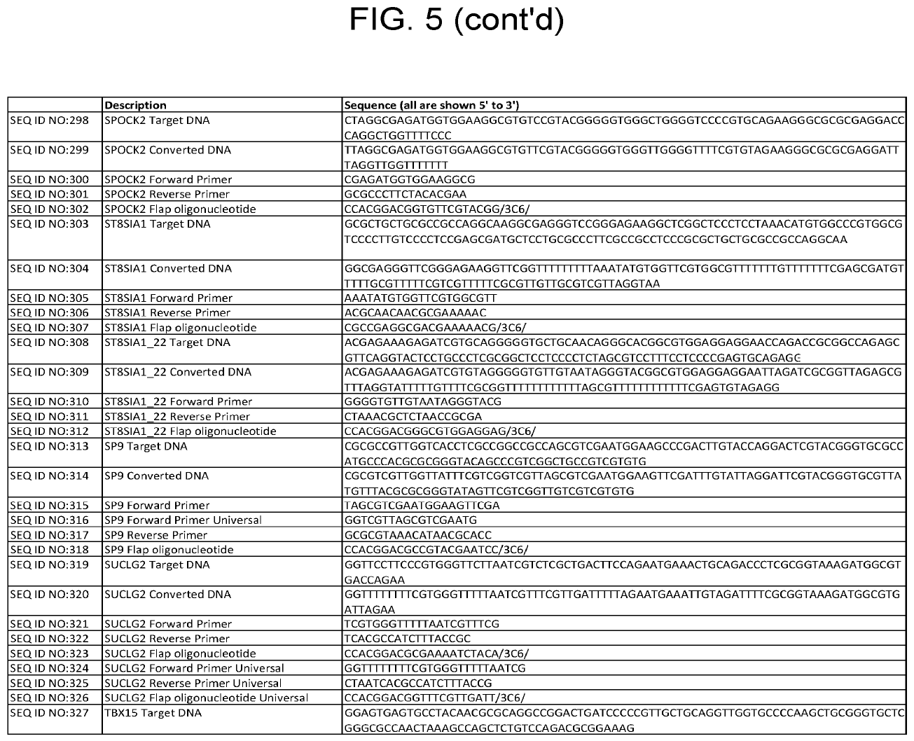

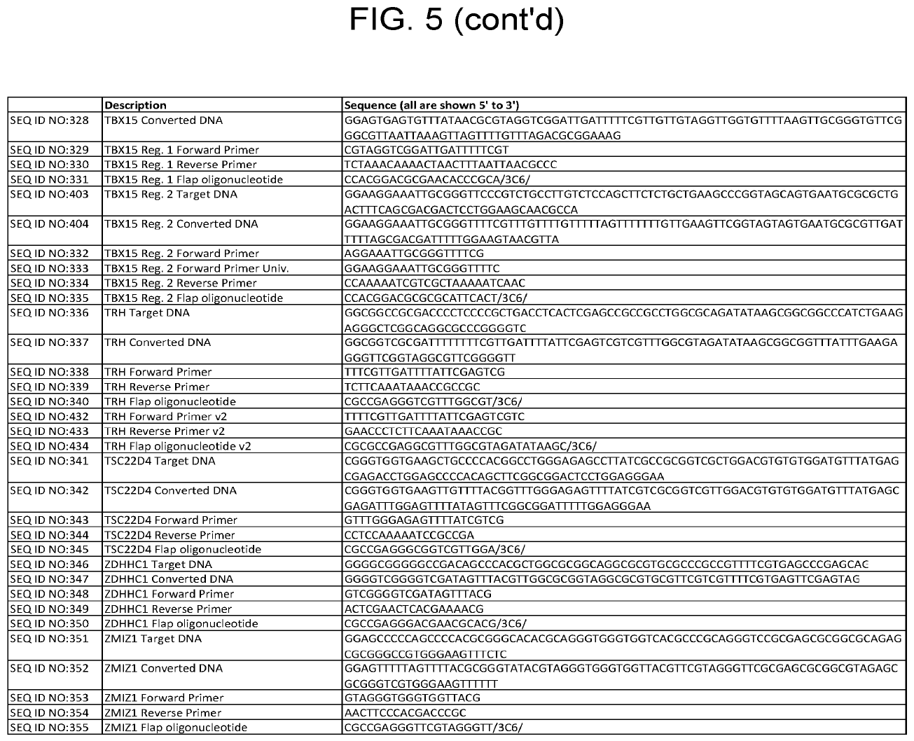

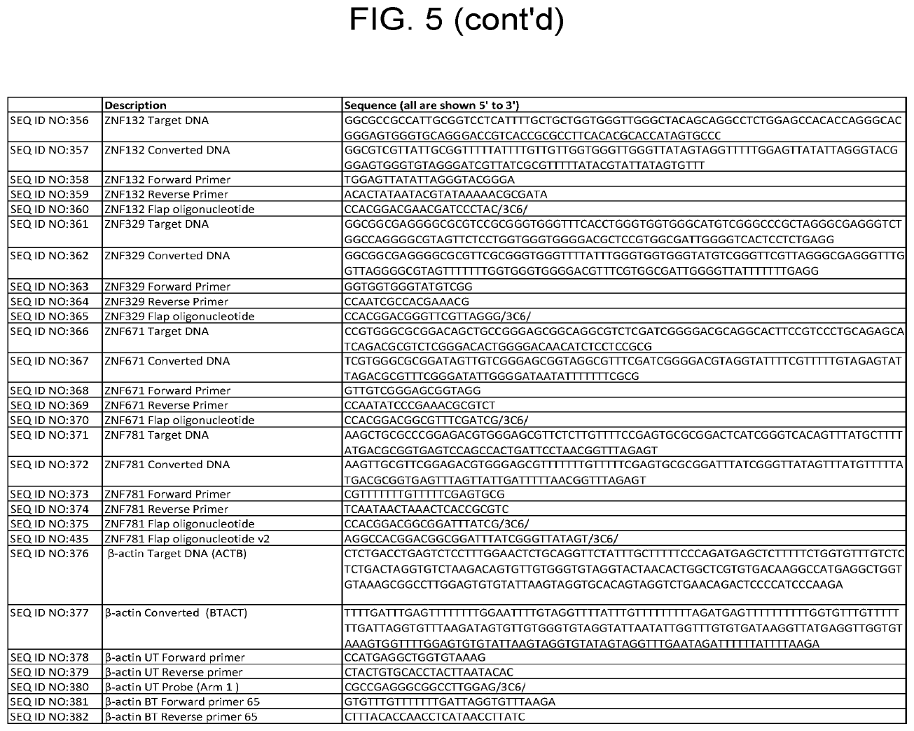

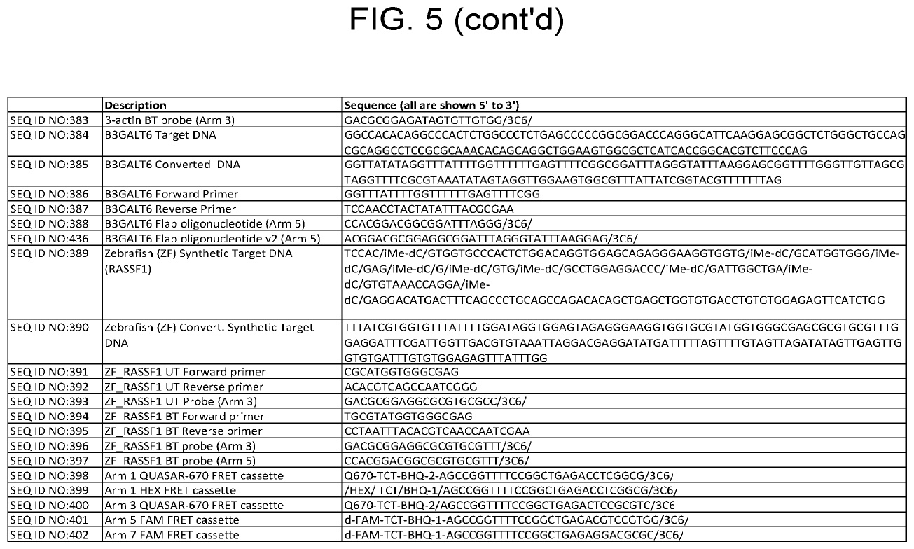

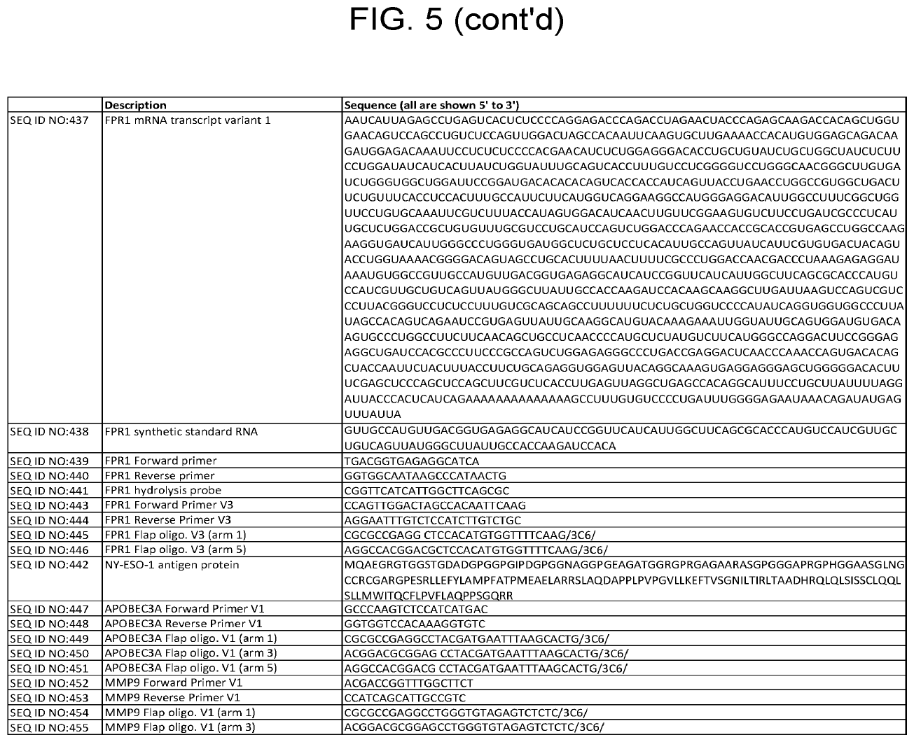

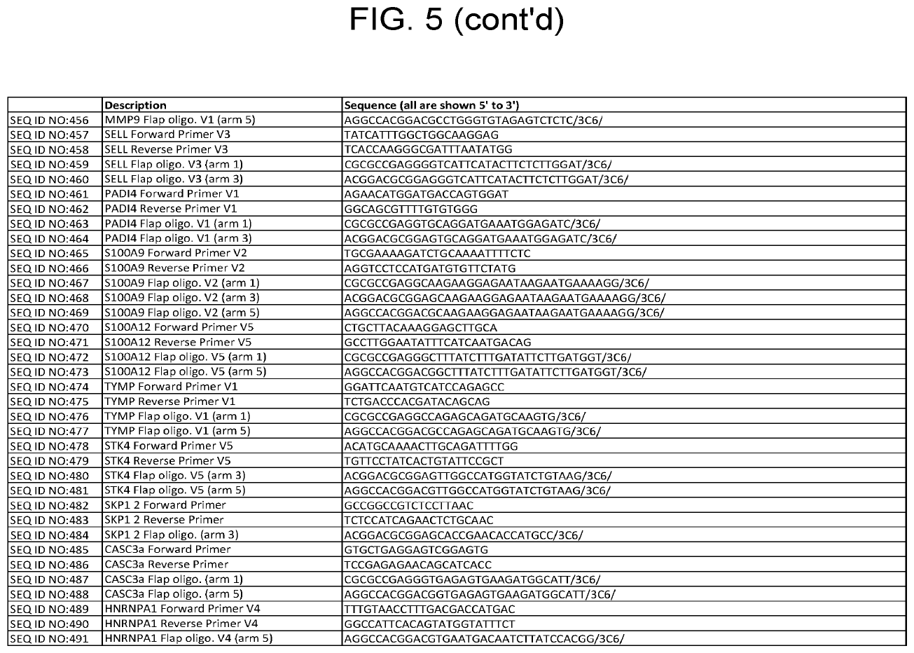

In some embodiments, fragments of the treated DNA are amplified using sets of primer oligonucleotides according to the present invention (e.g., see FIG. 5) and an amplification enzyme. The amplification of several DNA segments can be carried out simultaneously in one and the same reaction vessel. Typically, the amplification is carried out using a polymerase chain reaction (PCR).

Methods for isolating DNA suitable for these assay technologies are known in the art. In particular, some embodiments comprise isolation of nucleic acids as described in U.S. Pat. Nos. 9,000,146; 9,163,278; and 10,704,081, each incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QUARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises blood, serum, plasma, or saliva. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

It is contemplated that embodiments of the technology are provided in the form of a kit. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiment, certain component of the system are provided by the user.

### III. Applications

In some embodiments, diagnostic assays Identify the presence of a disease or condition in an individual. In some embodiments, the disease is cancer (e.g., lung cancer).

In some embodiments, markers whose aberrant methylation is associated with a lung cancer (e.g., one or more markers selected from de markers listed in Table 1, or preferably one or more of EMX1, GRIN2D, ANKRD13B, ZNF781, ZNF671, IFFO1, HOPX, BARX1, HOXA9, LOC100129726, SPOCA2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC1248, BHLHE23, CAPN2, FGF14, FLJ34208, BIN2_Z, DNMT3A, FERMT3, NFIX, SIPR4, SKI, SUCLG2, TBX15, and ZNF329) are used. In some embodiments, an assay further comprises detection of a reference gene (e.g., β-actin, ZDHHC1, B3GALT26. See, e.g., U.S. Pat. No. 10,465,248, and WO 2018/017740, each of which is incorporated herein by reference for all purposes).

In some embodiments, markers whose aberrant expression is associated with a lung cancer (preferably one or more markers listed in Table 3: S100A9, SELL, PADI4, APOBE3CA, S100A12, MMP9, FPR1, TYMP, and SAT1) are used, and are detected by measurement of one or more of RNA (e.g., an mRNA) or protein in a sample. In some embodiments, an assay further comprises detection of a reference gene (e.g., as shown in Table 3.)

In some embodiments, the technology finds application in treating a patient (e.g., a patient with lung cancer, with early stage lung cancer, or who may develop lung cancer), the method comprising determining the methylation state of one or more markers as provided herein and administering a treatment the patient based on the results of determining the methylation state. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, the technology finds application in methods for diagnosing lung cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker, the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of making determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers disclosed herein.

Further, in some embodiments of the technology, multiple determinations of the biomarkers over time can be made to facilitate diagnosis and/or prognosis. A temporal change in the biomarker can be used to predict a clinical outcome, monitor the progression of lung cancer, and/or monitor the efficacy of appropriate therapies directed against the cancer.

In such an embodiment for example, one might expect to see a change in the methylation state of one or more biomarkers disclosed herein (and potentially one or more additional biomarker(s), if monitored) in a biological sample over time during the course of an effective therapy.

The technology further finds application in methods for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with risk for developing lung, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted above, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition, the chance of a given outcome (e.g., suffering from lung cancer) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with lung cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having lung cancer if, when compared to a control methylation state, there is a measurable difference in the methylation state of at least one biomarker in the sample. Conversely, when no change in methylation state is identified in the biological sample, the subject can be identified as not having lung cancer, not being at risk for the cancer, or as having a low risk of the cancer. In this regard, subjects having lung cancer or risk thereof can be differentiated from subjects having low to substantially no cancer or risk thereof. Those subjects having a risk of developing lung cancer can be placed on a more intensive and/or regular screening schedule. On the other hand, those subjects having low to substantially no risk may avoid being subjected to screening procedures, until such time as a future screening, for example, a screening conducted in accordance with the present technology, indicates that a risk of lung cancer has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, lung cancer indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

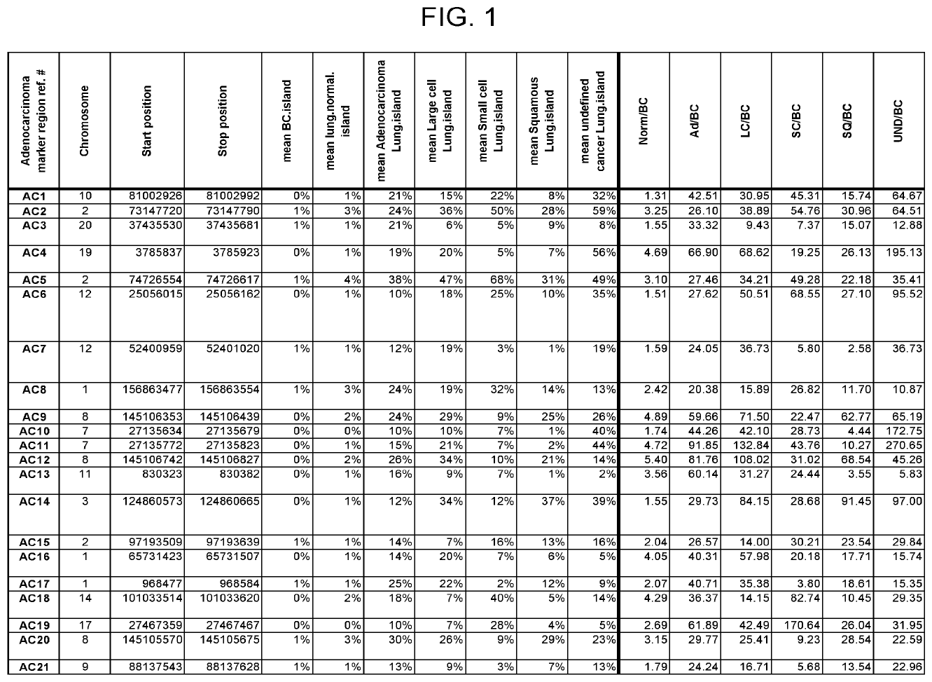

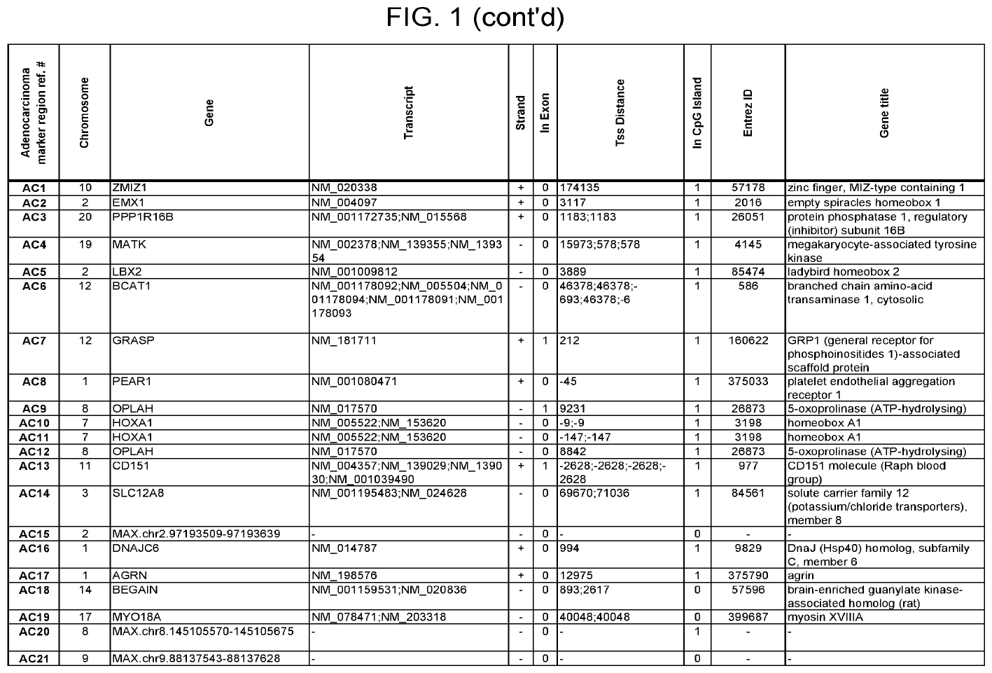

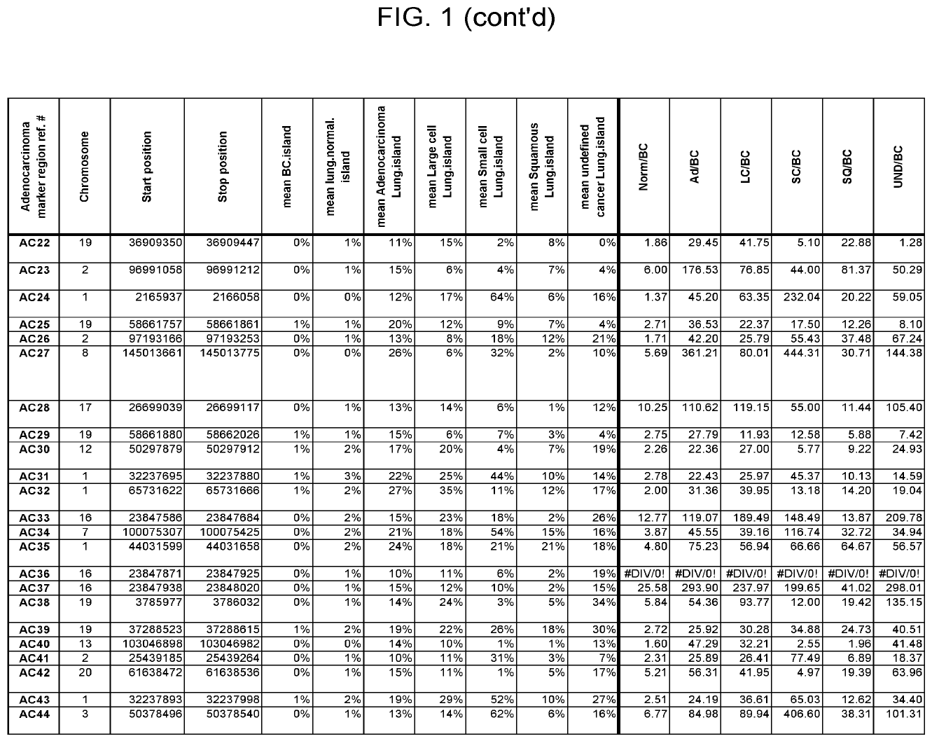

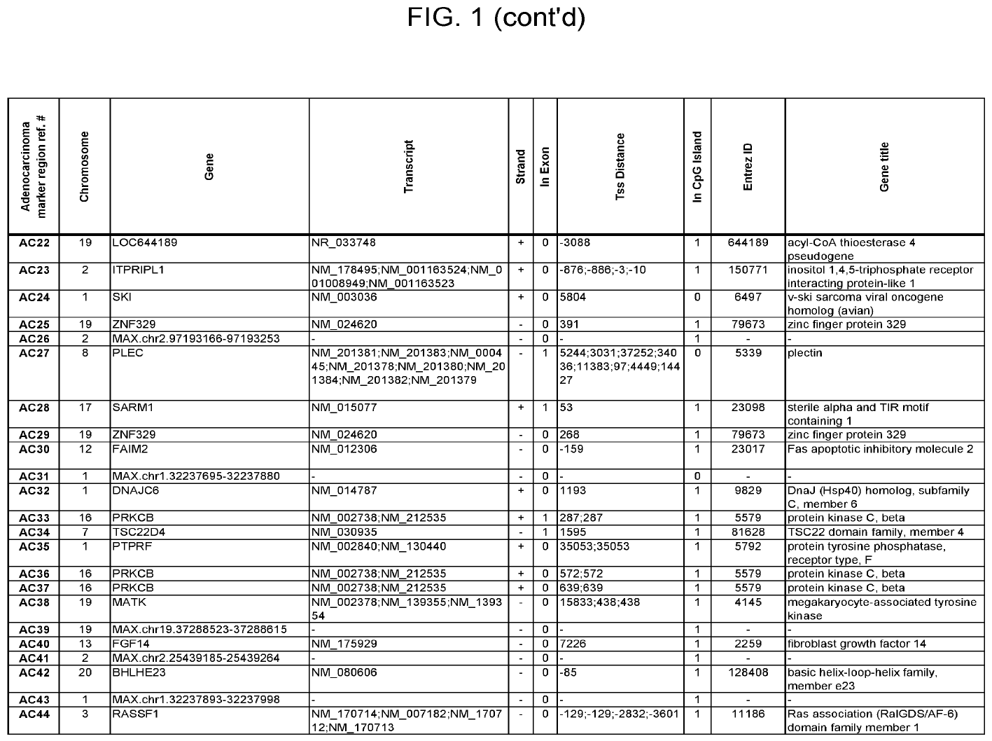

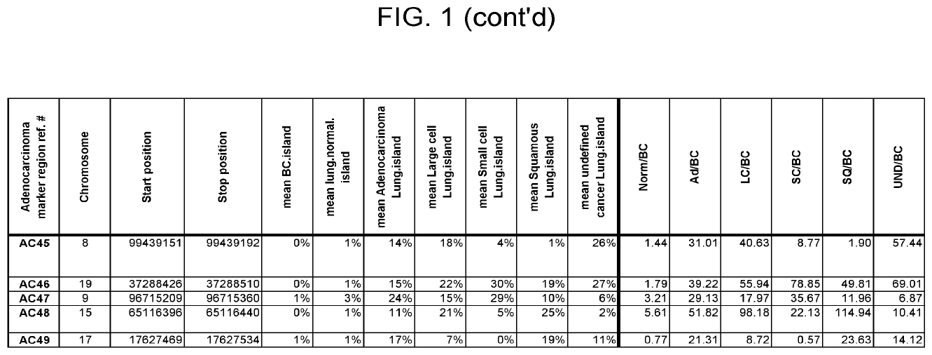

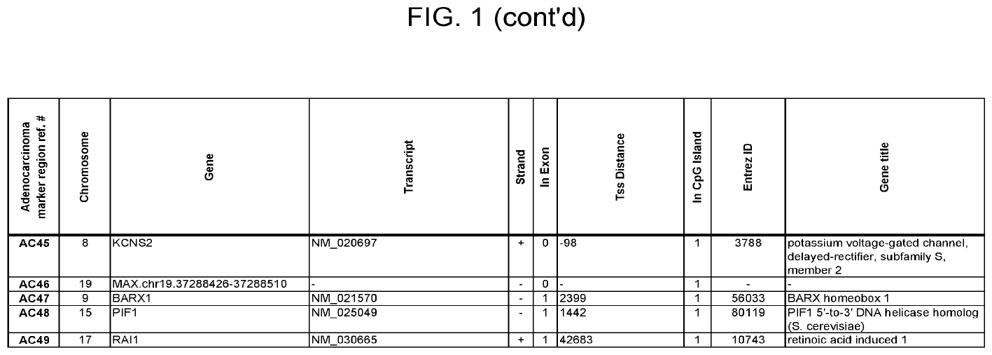

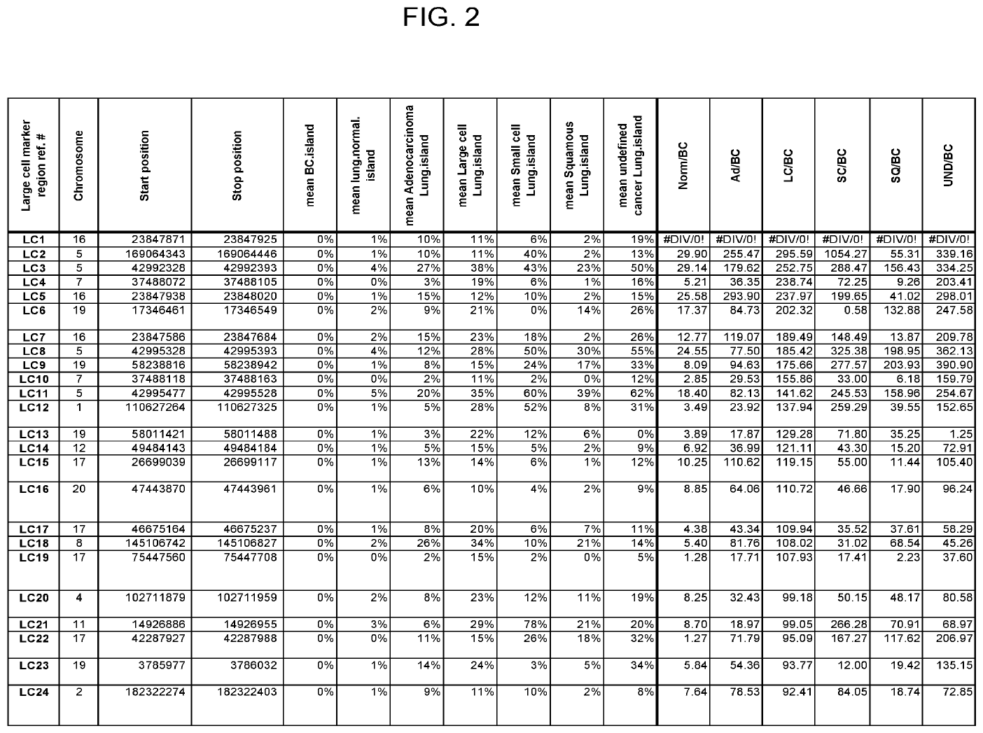

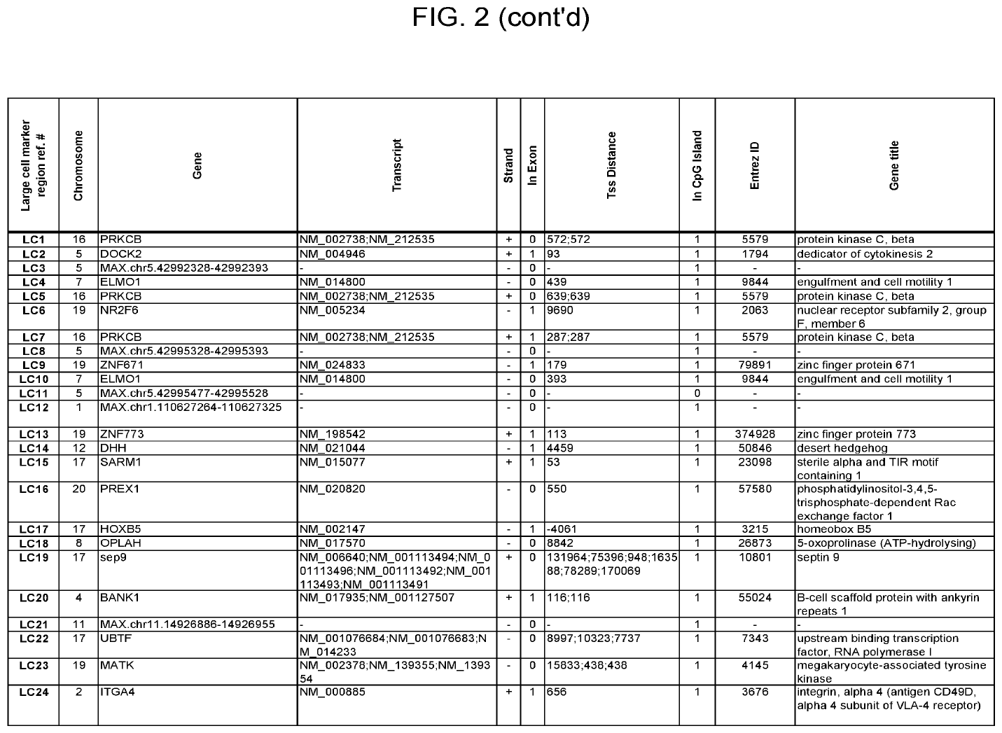

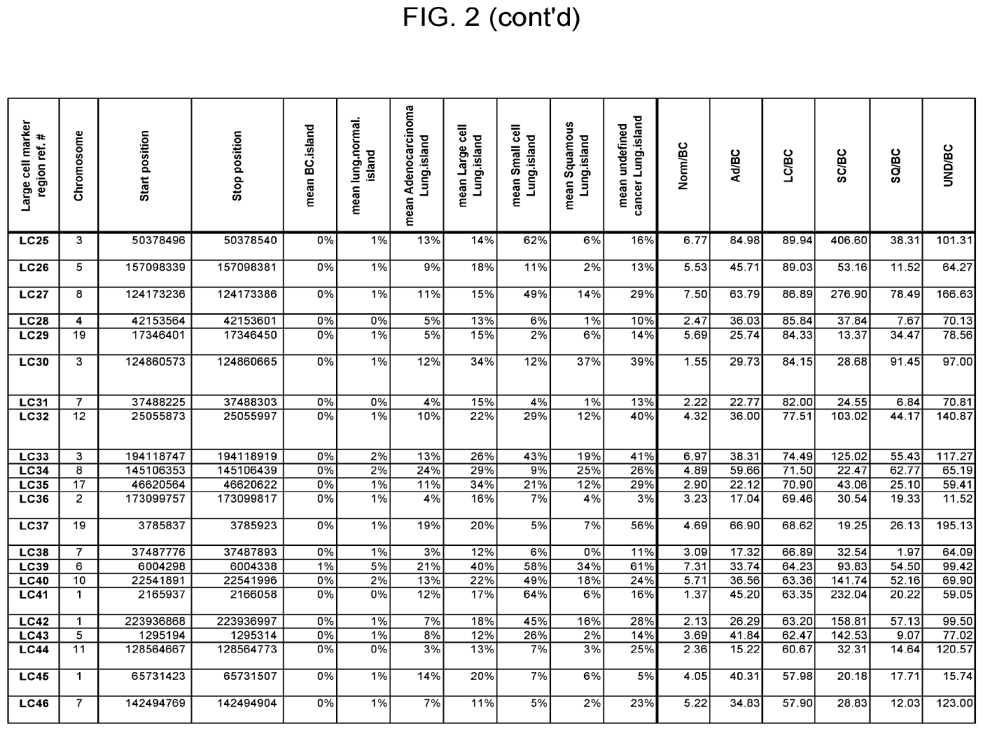

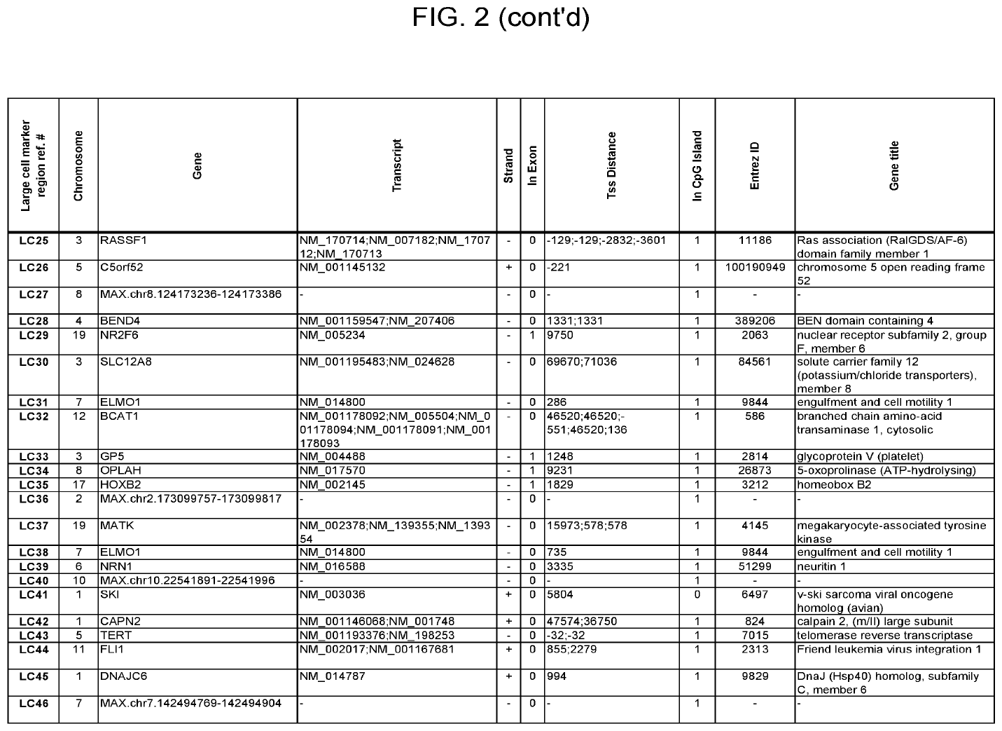

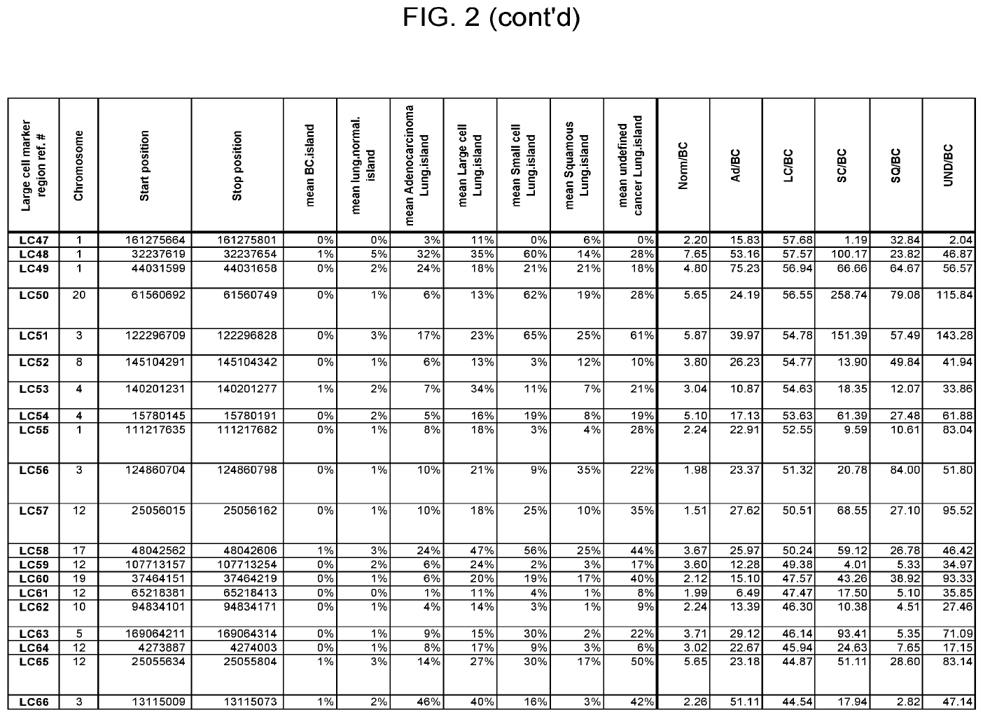

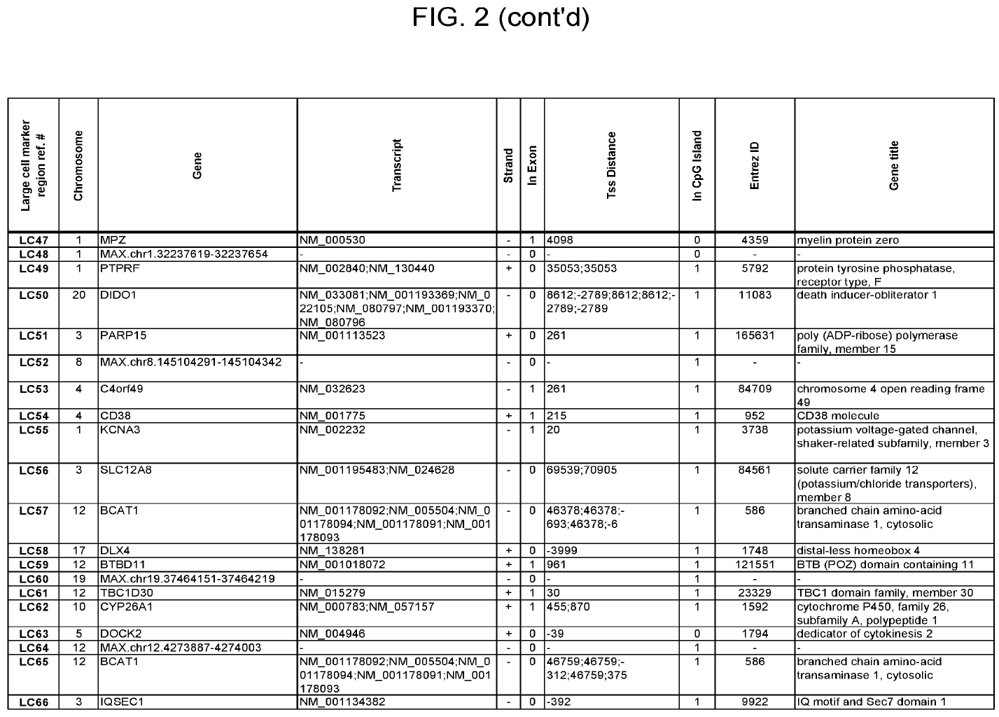

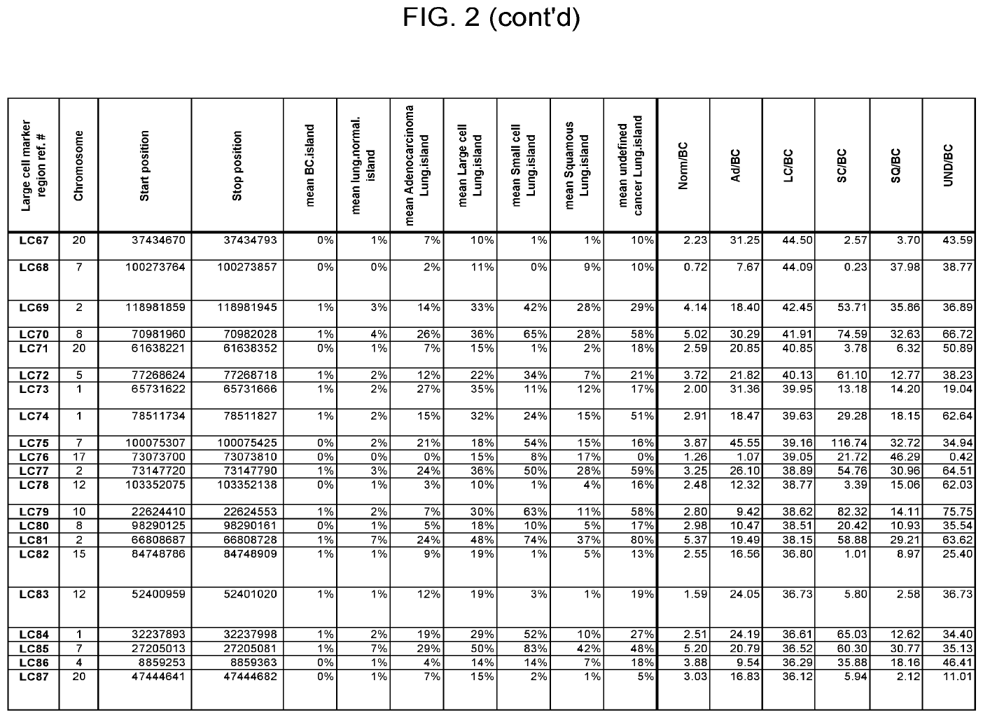

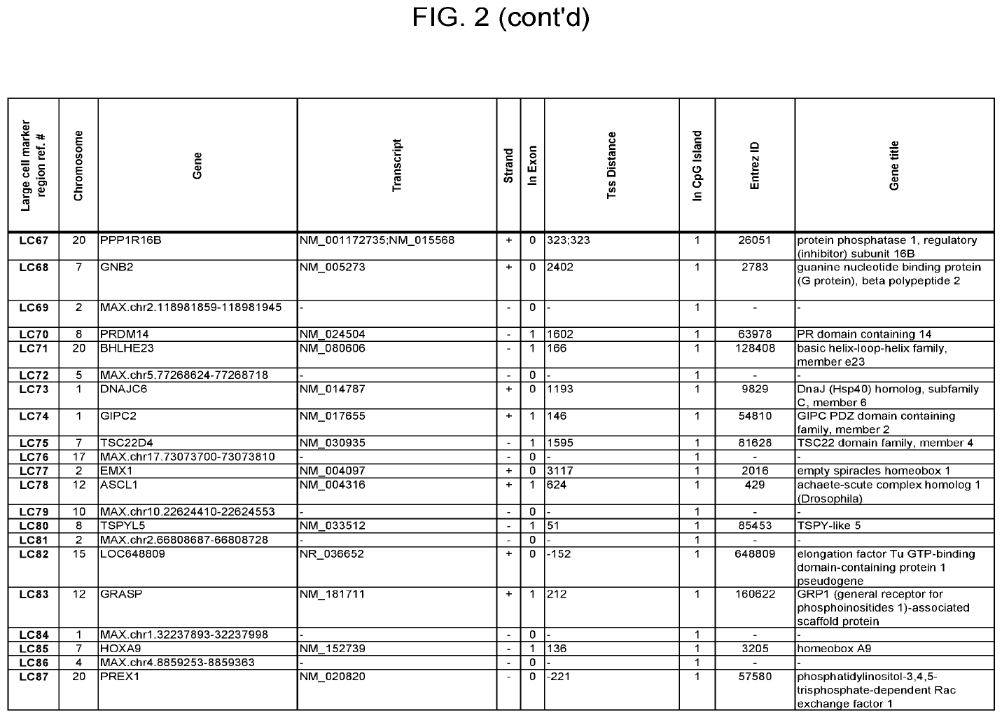

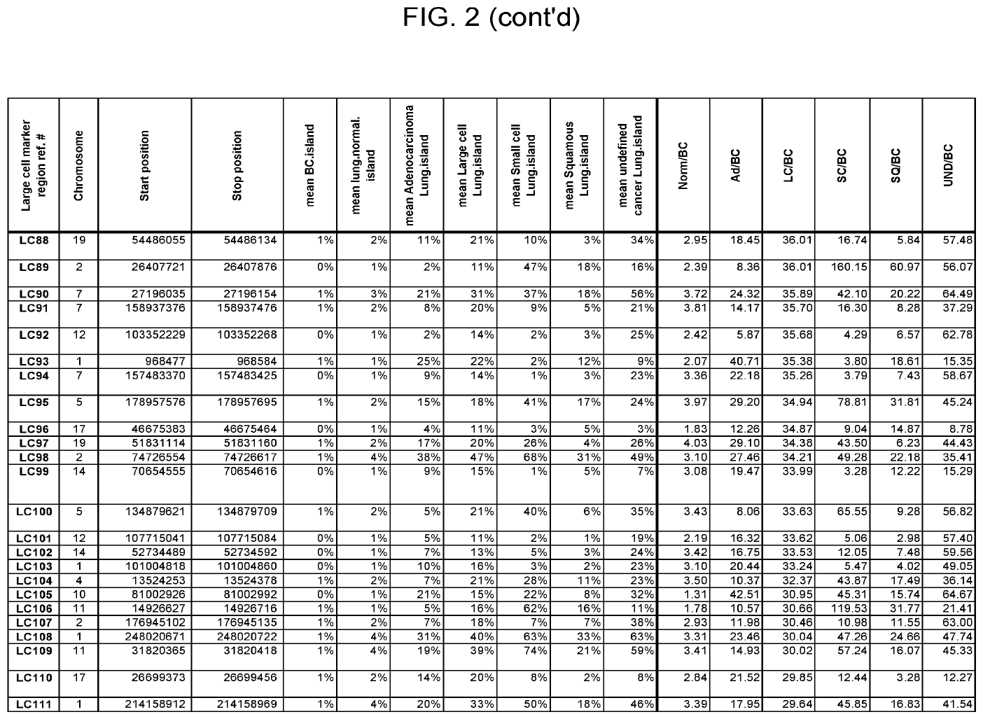

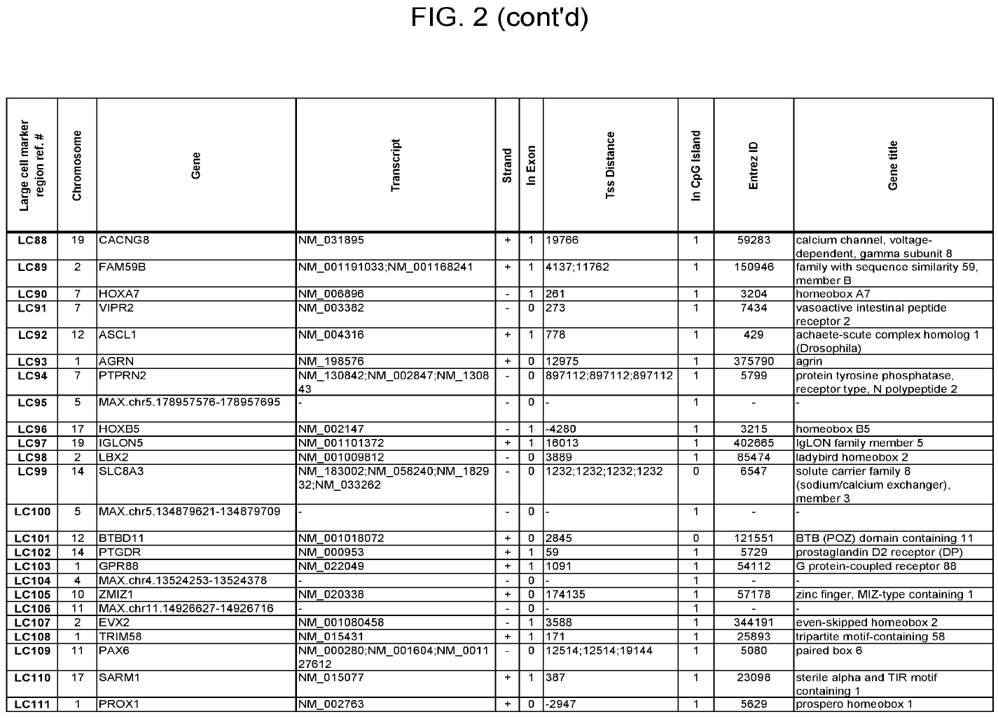

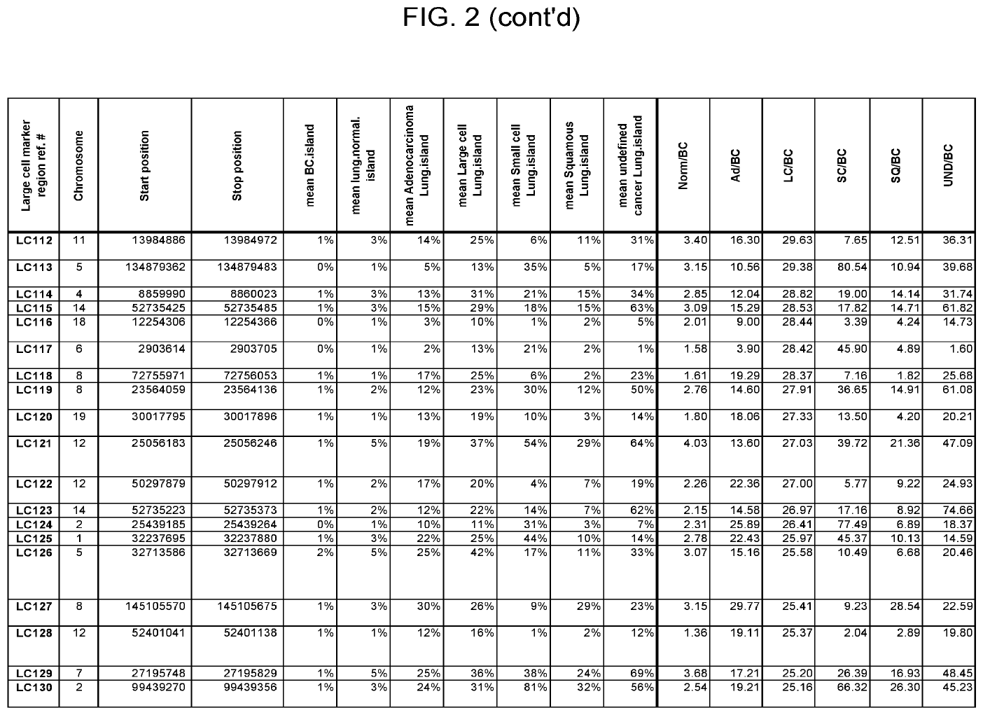

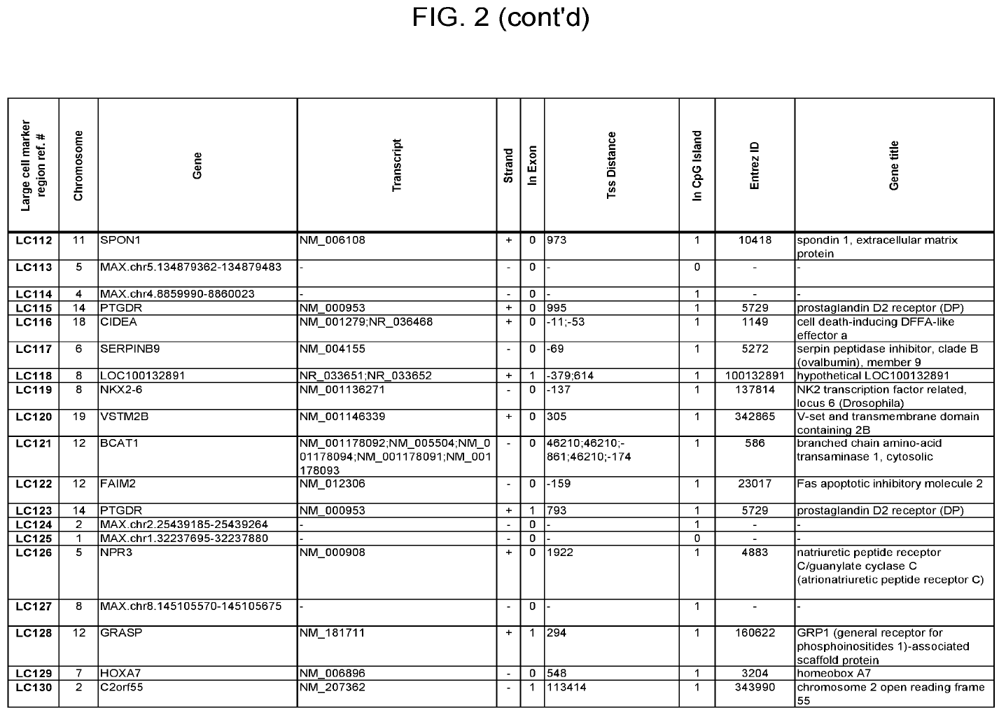

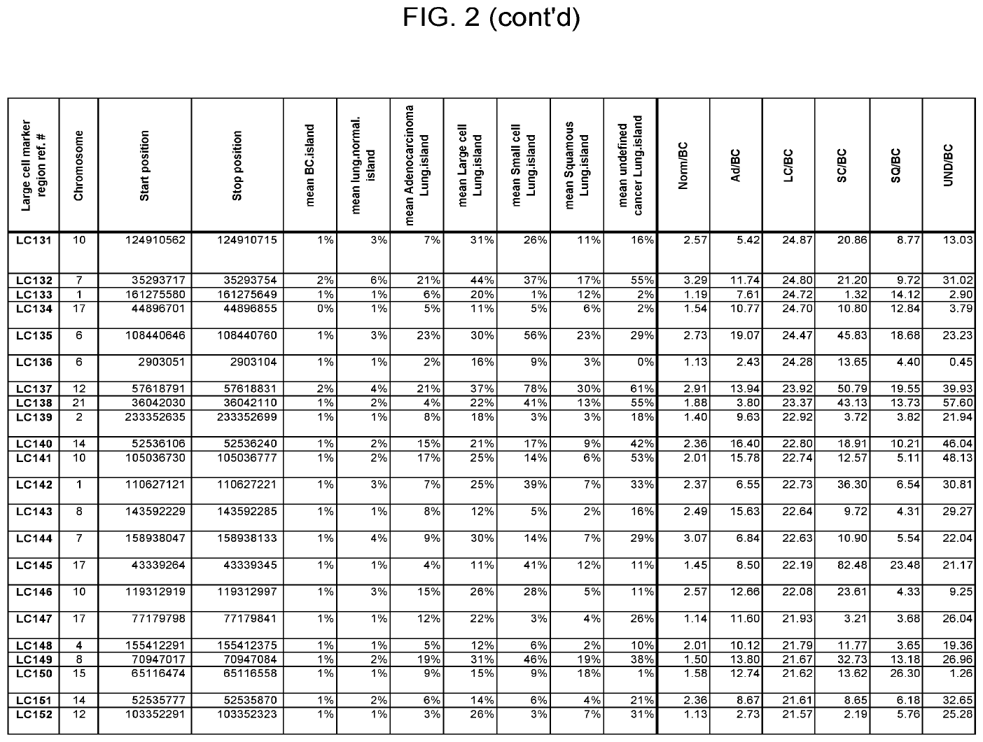

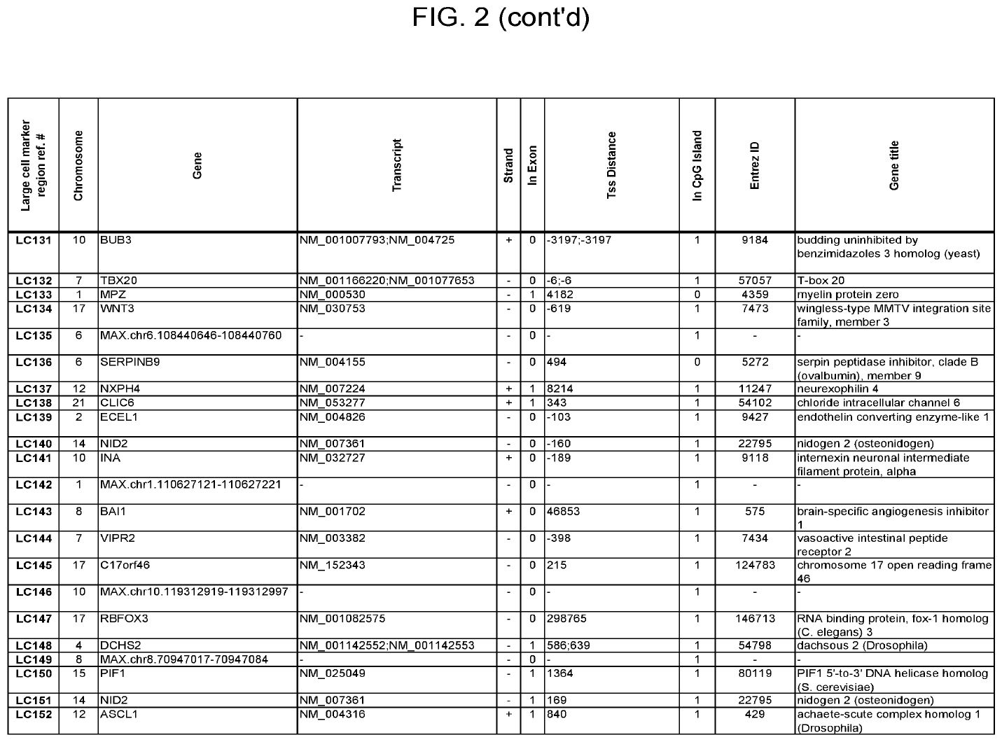

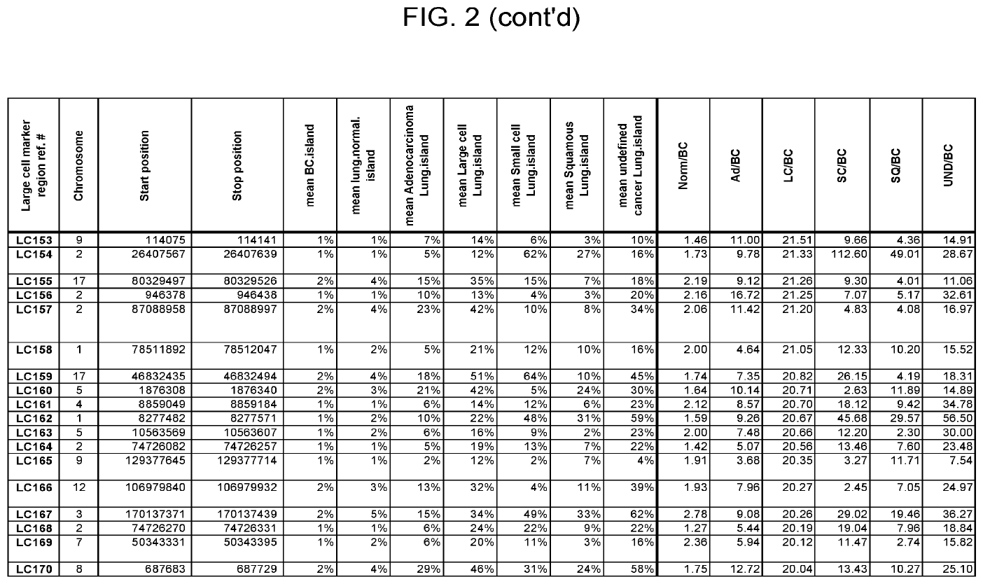

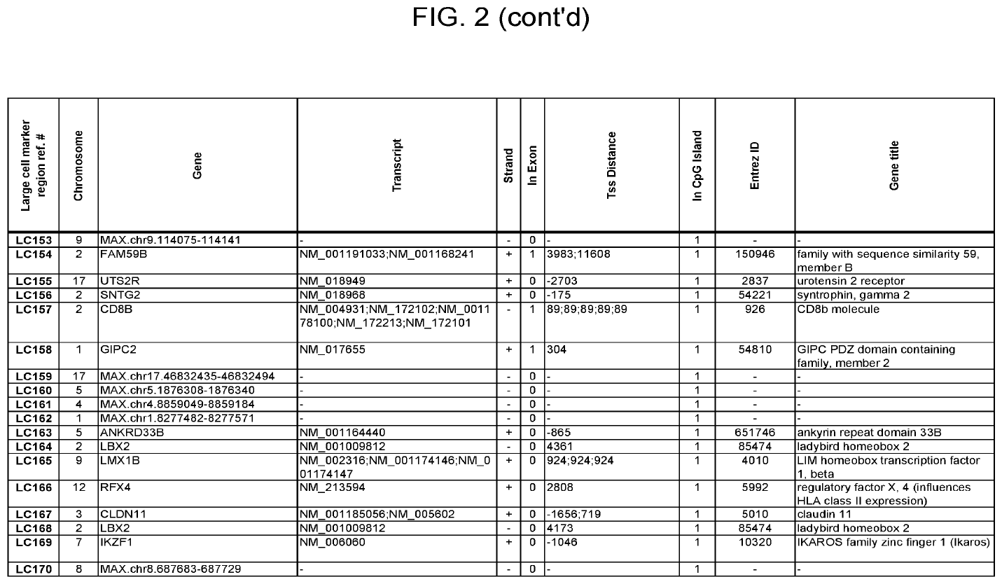

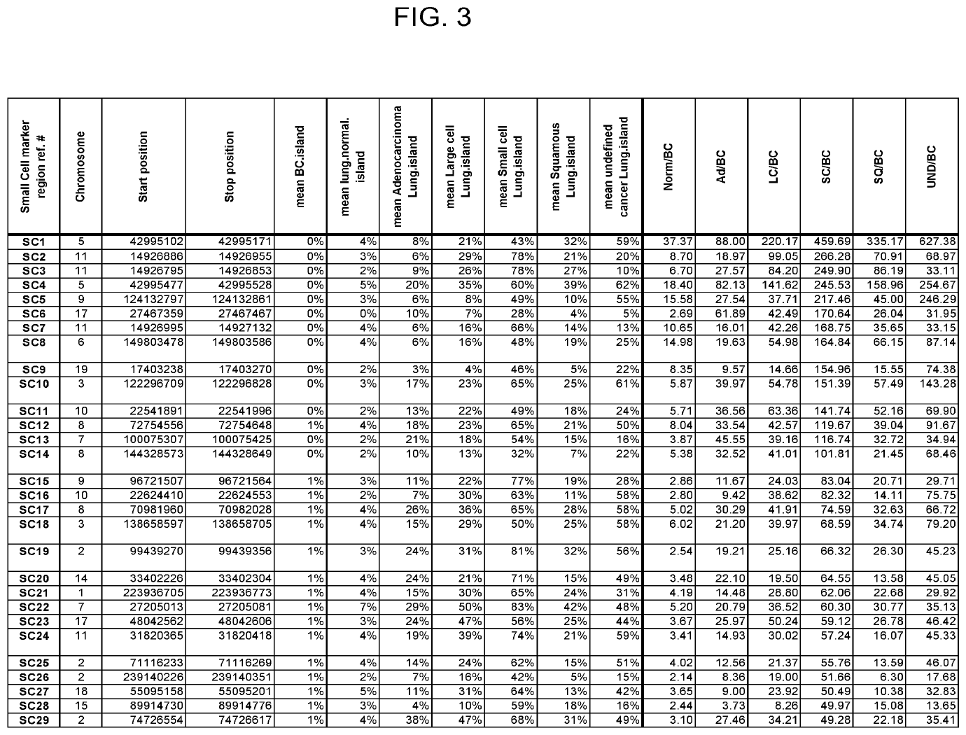

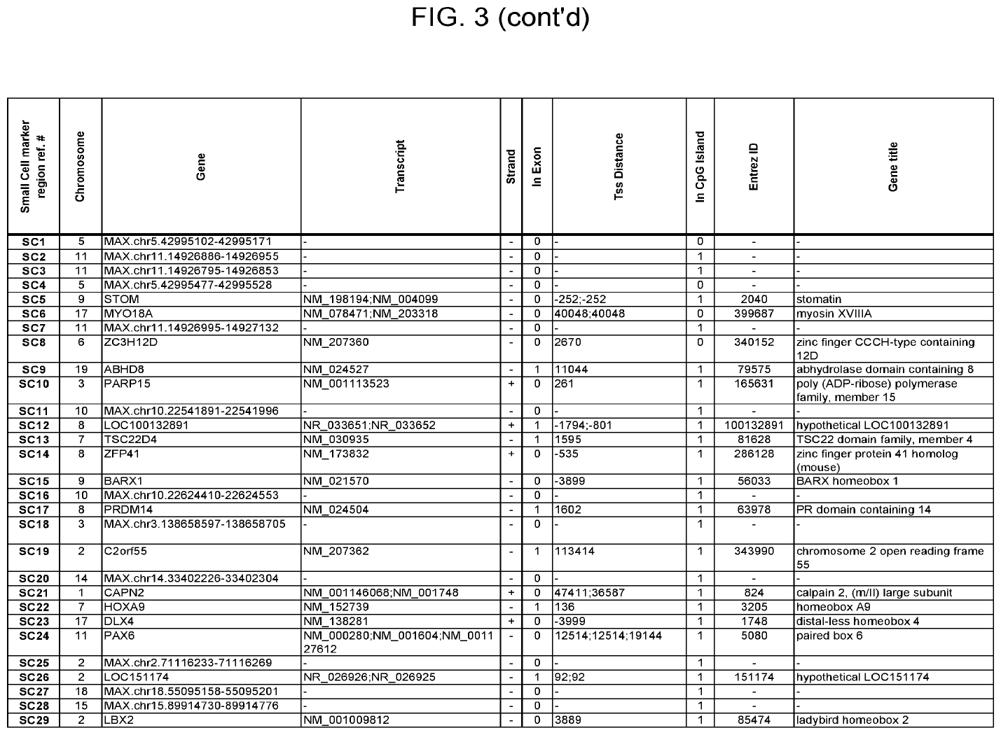

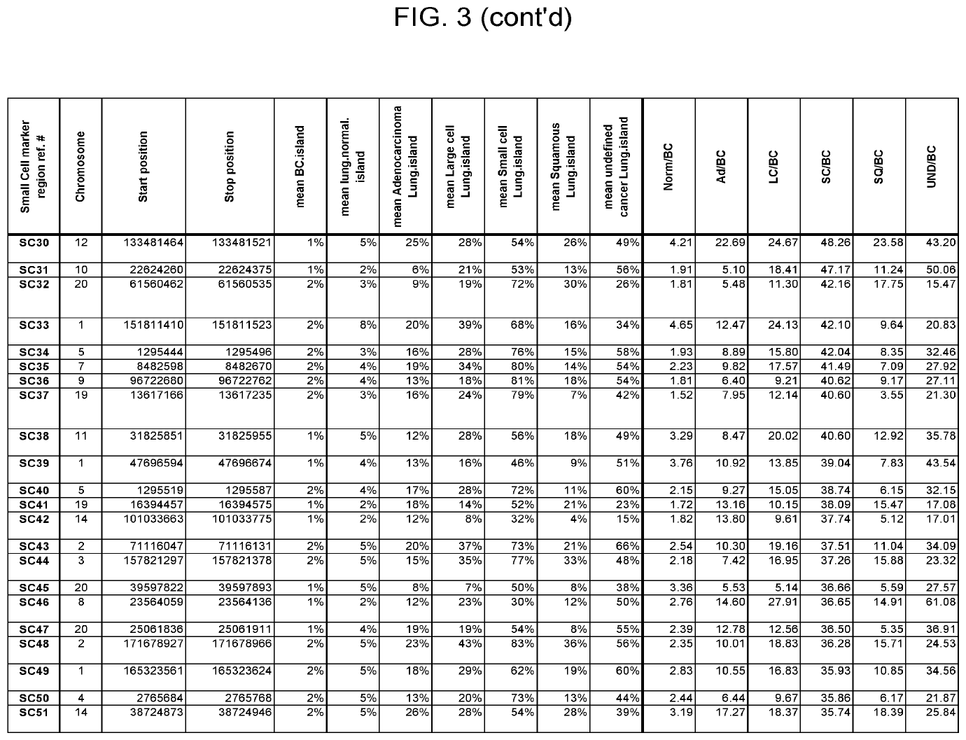

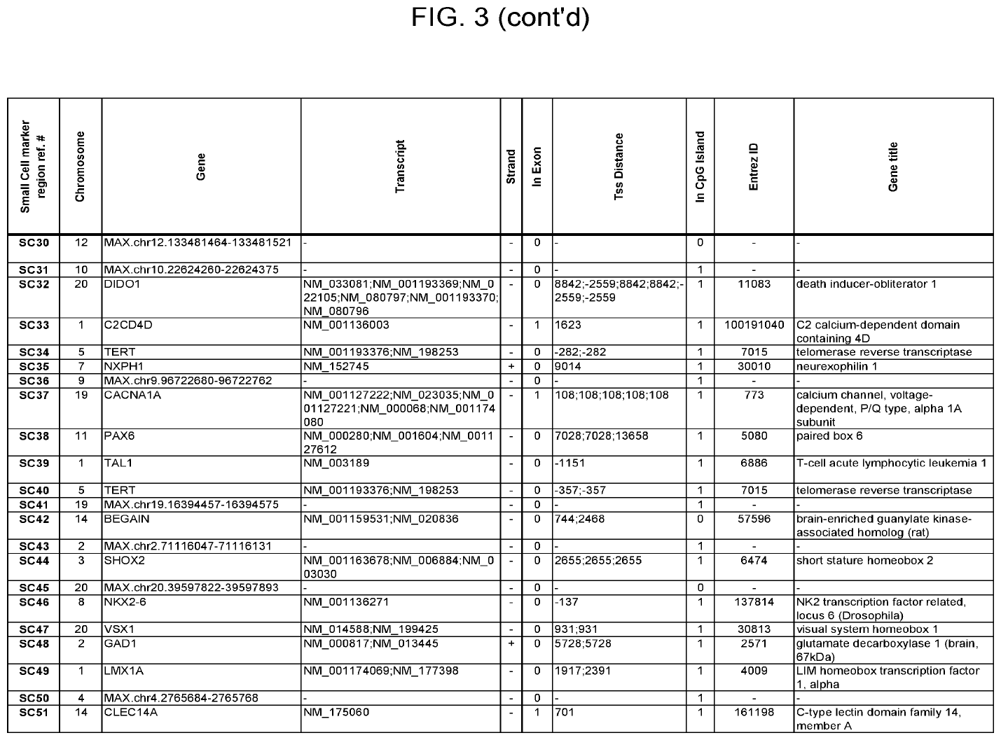

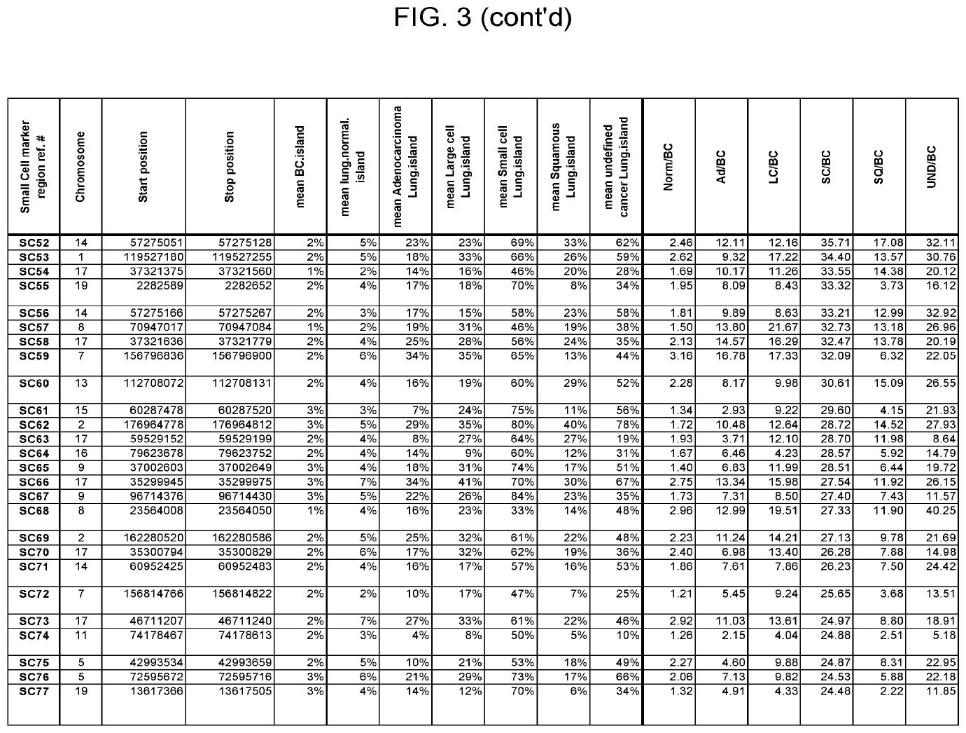

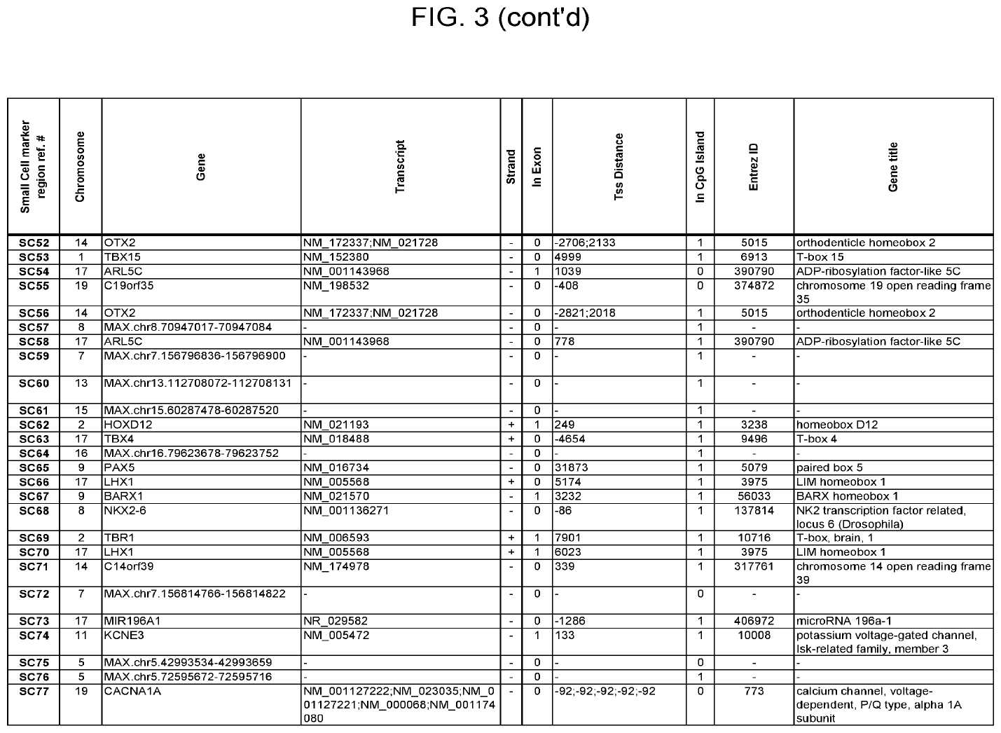

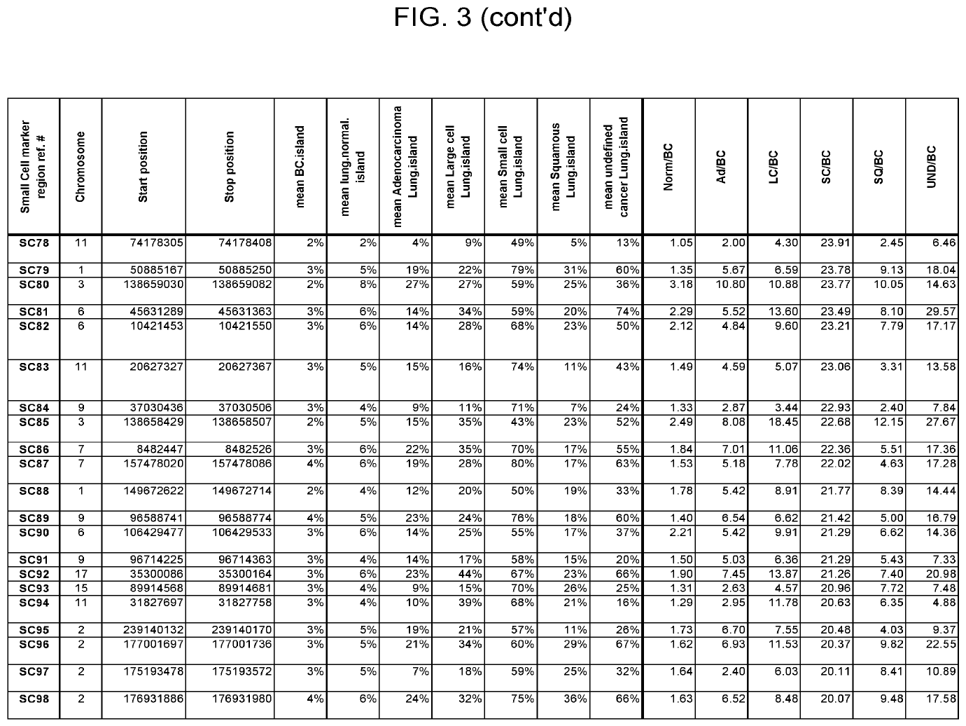

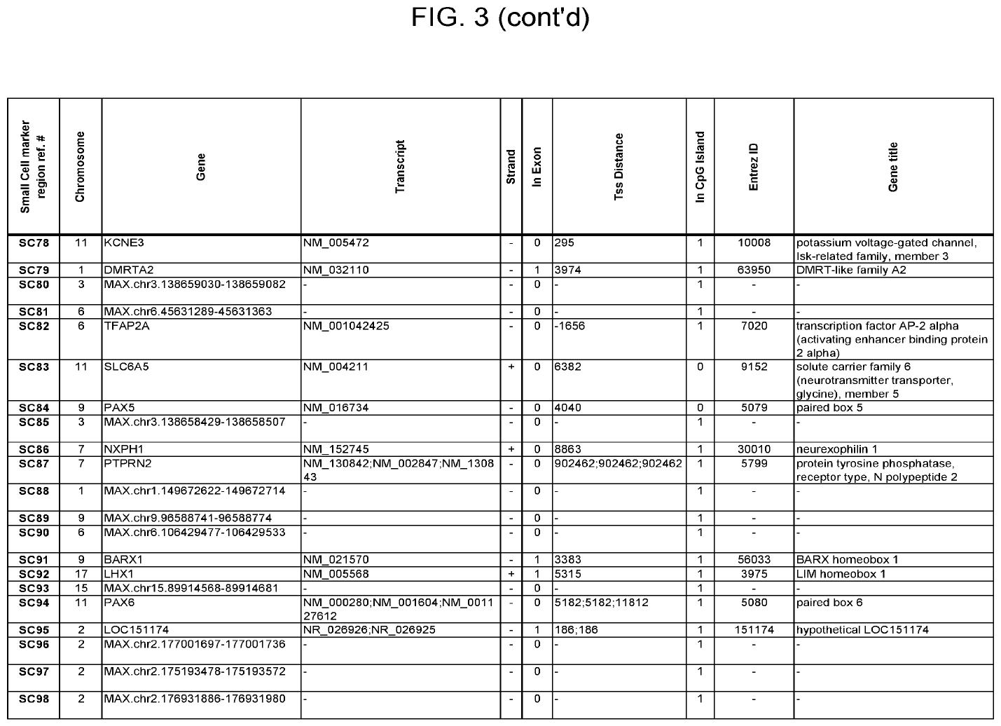

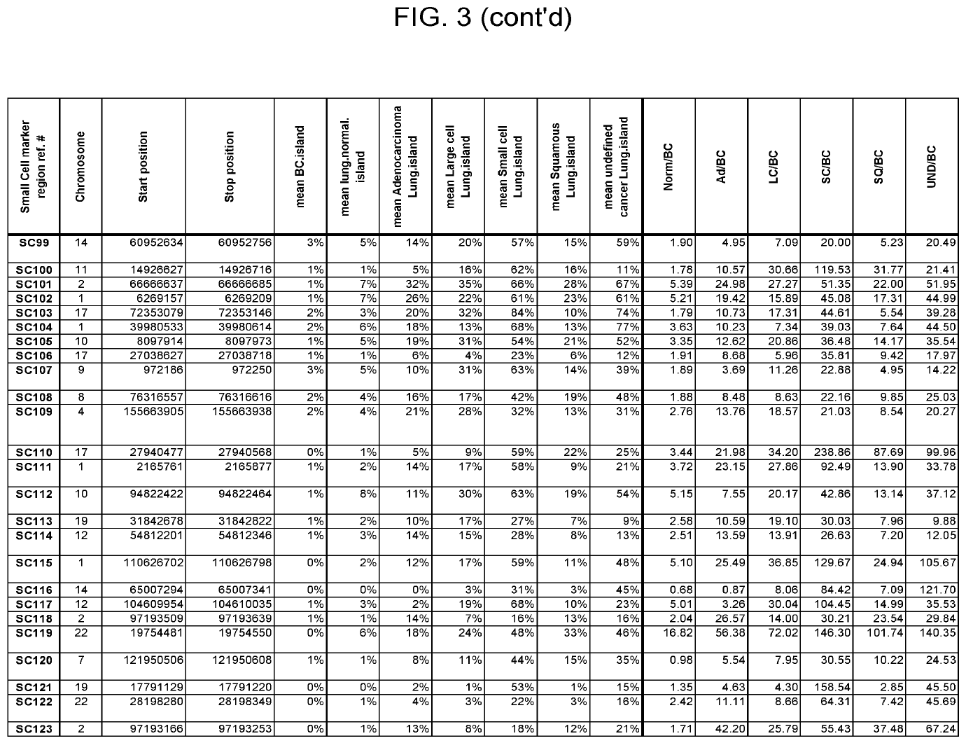

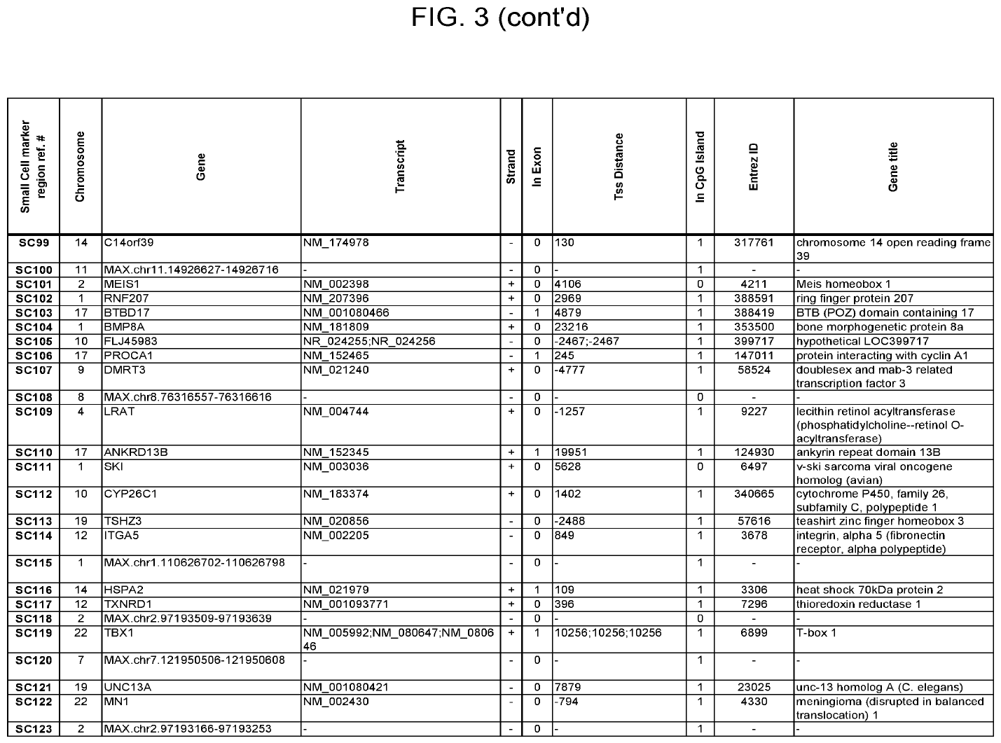

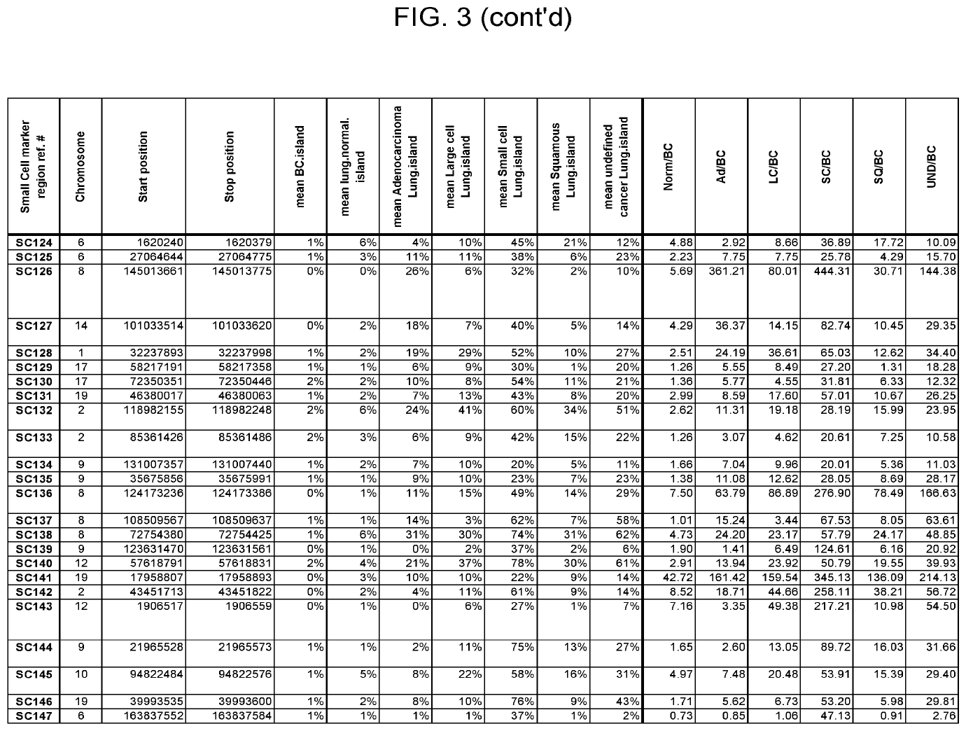

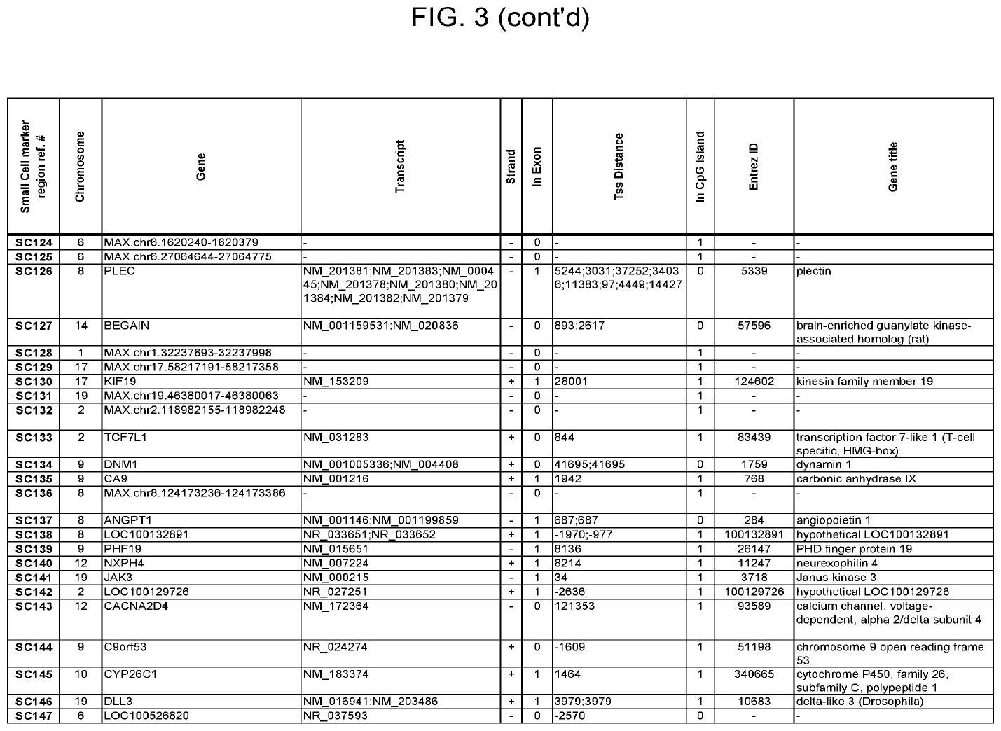

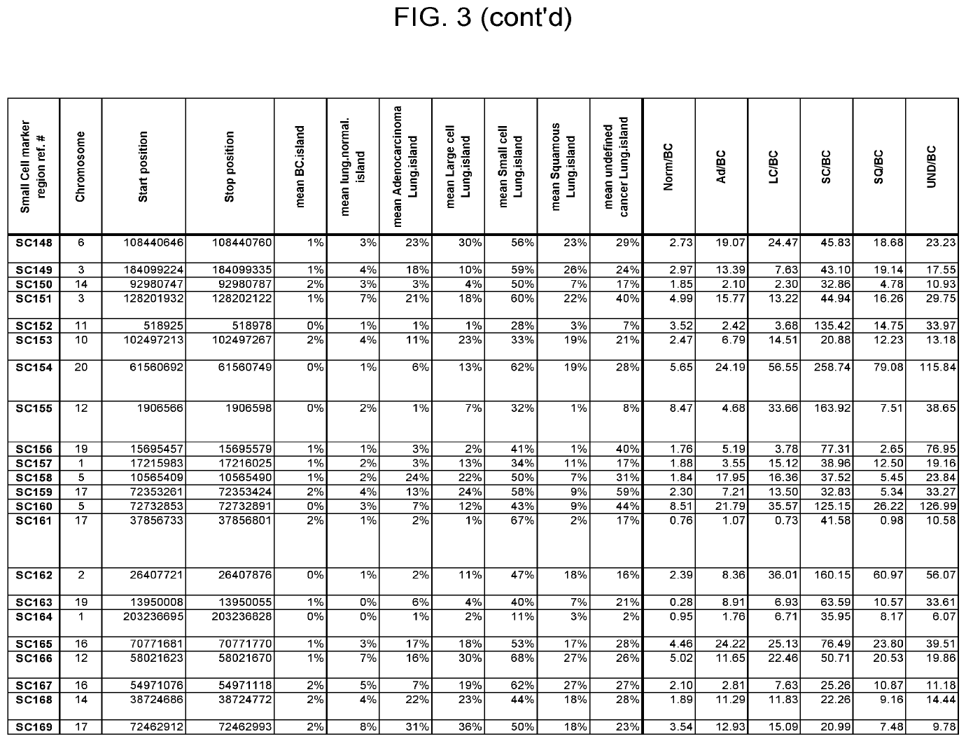

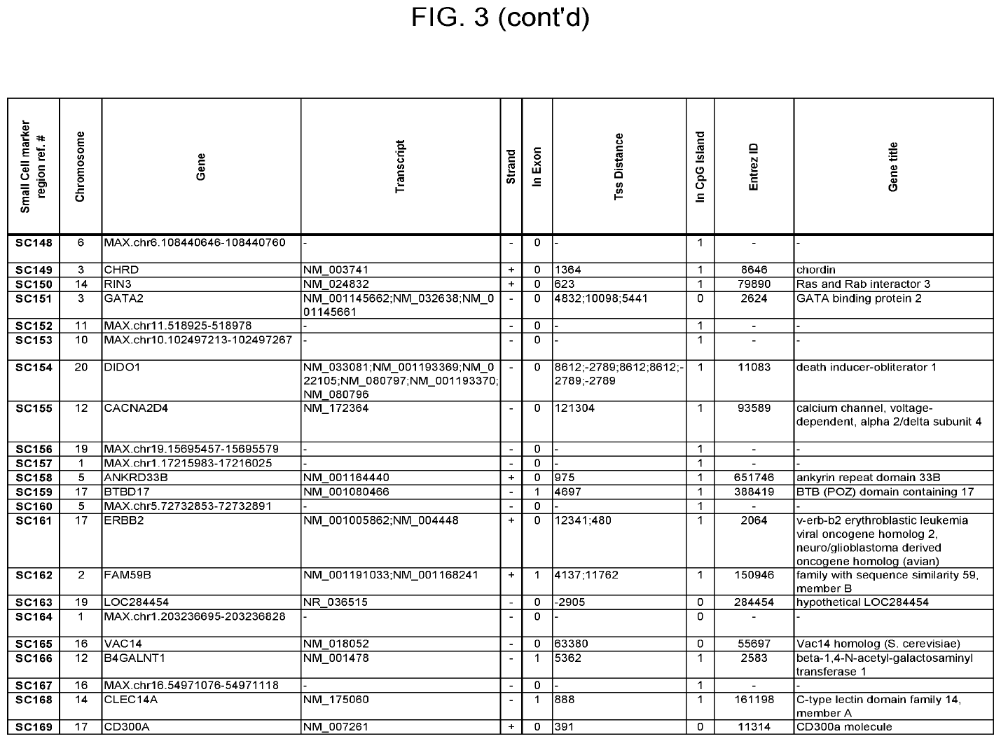

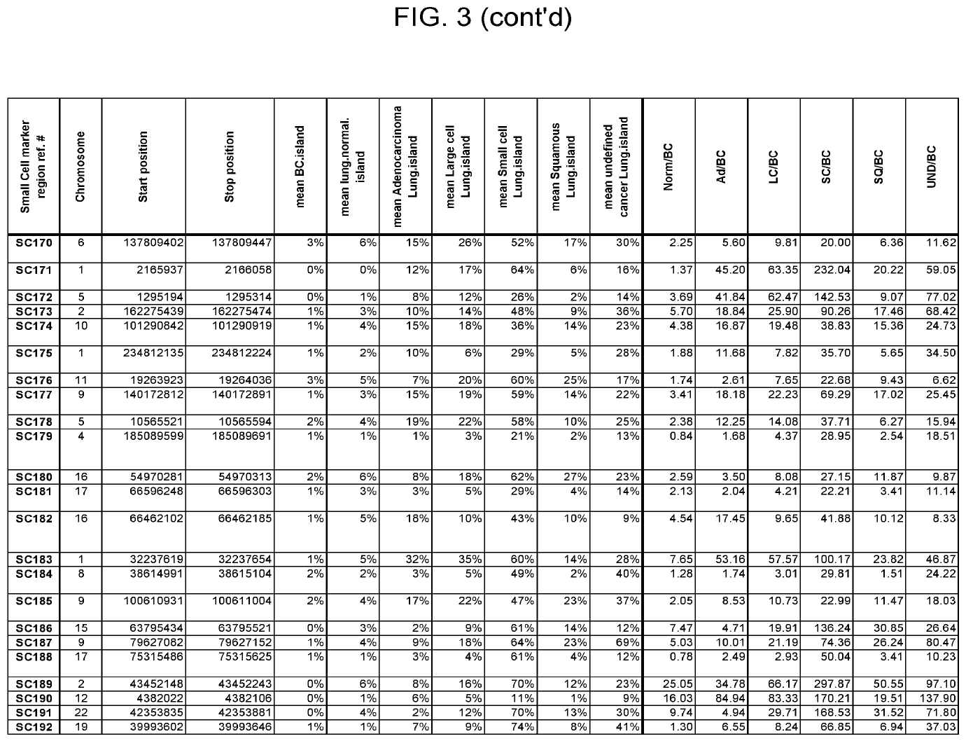

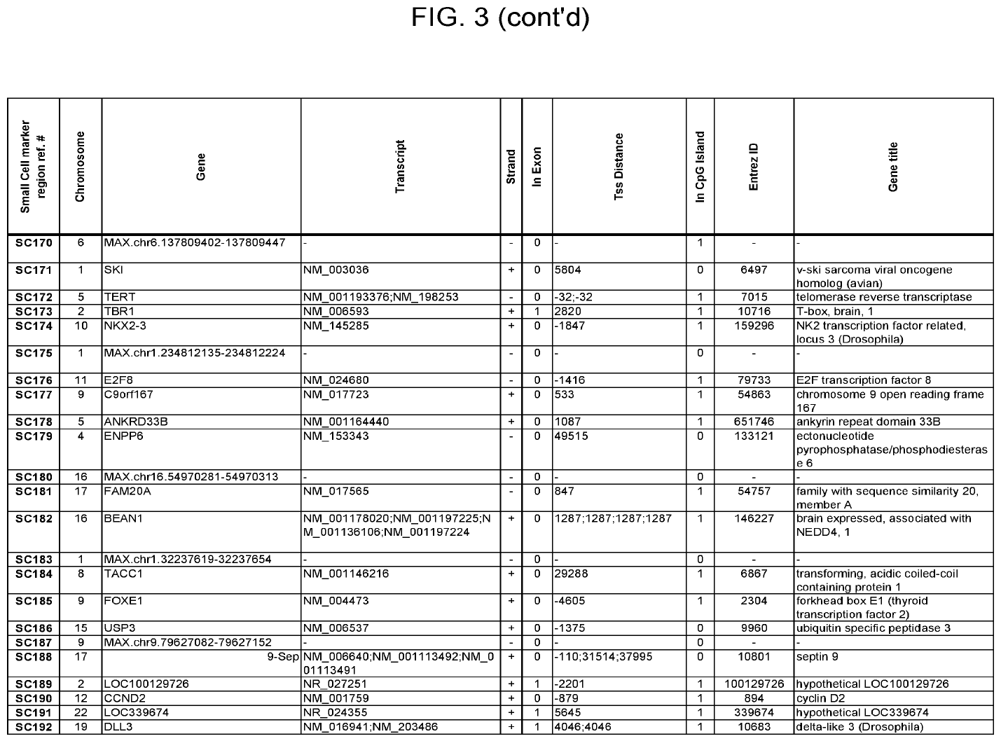

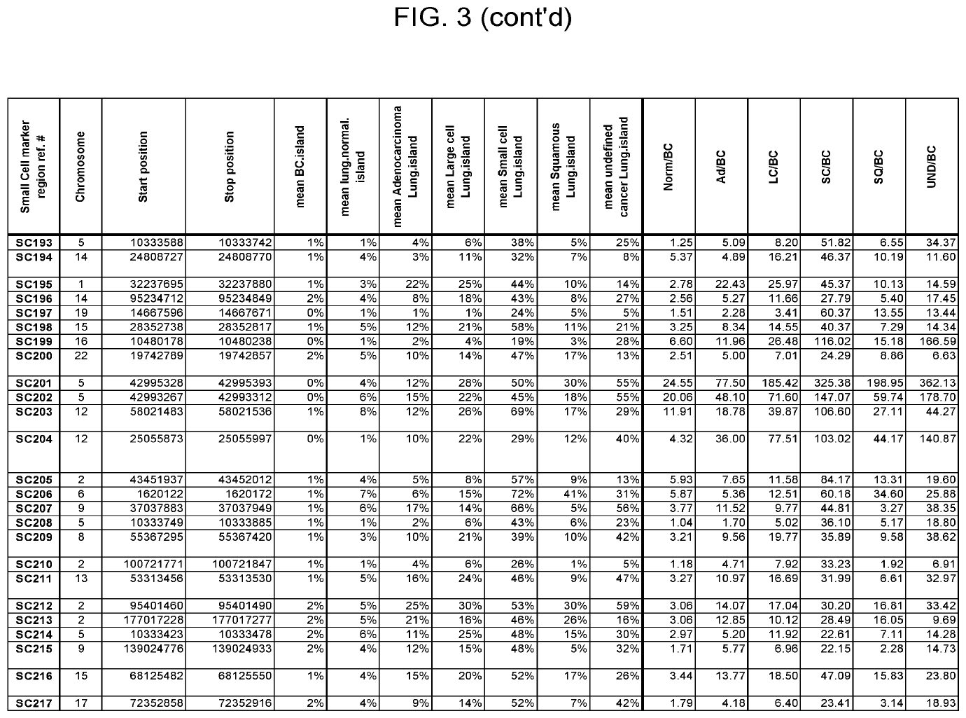

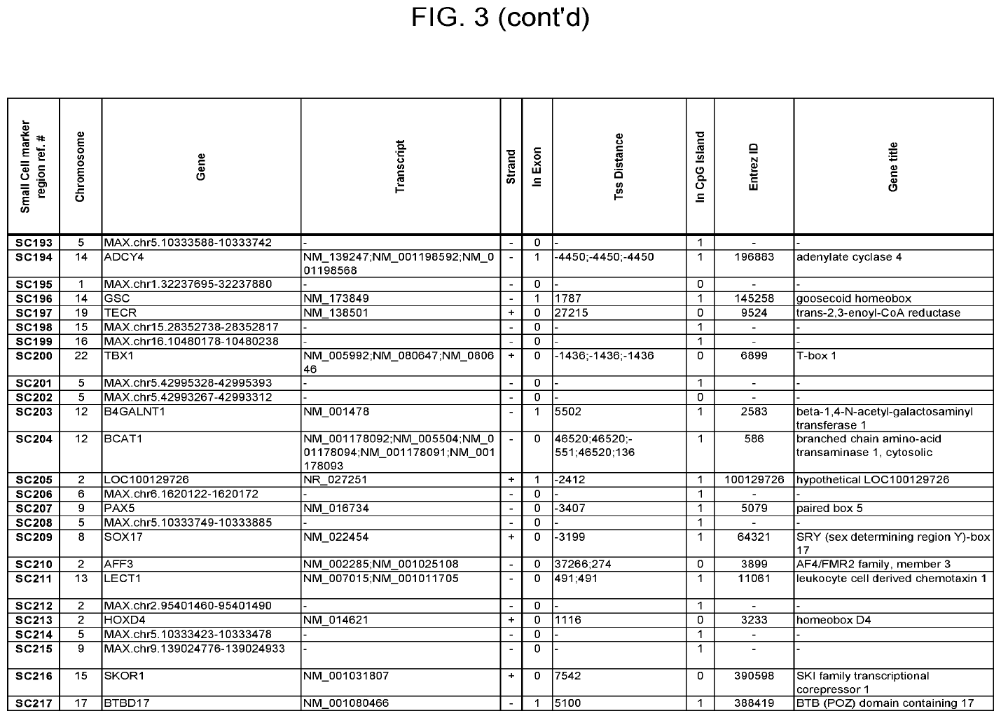

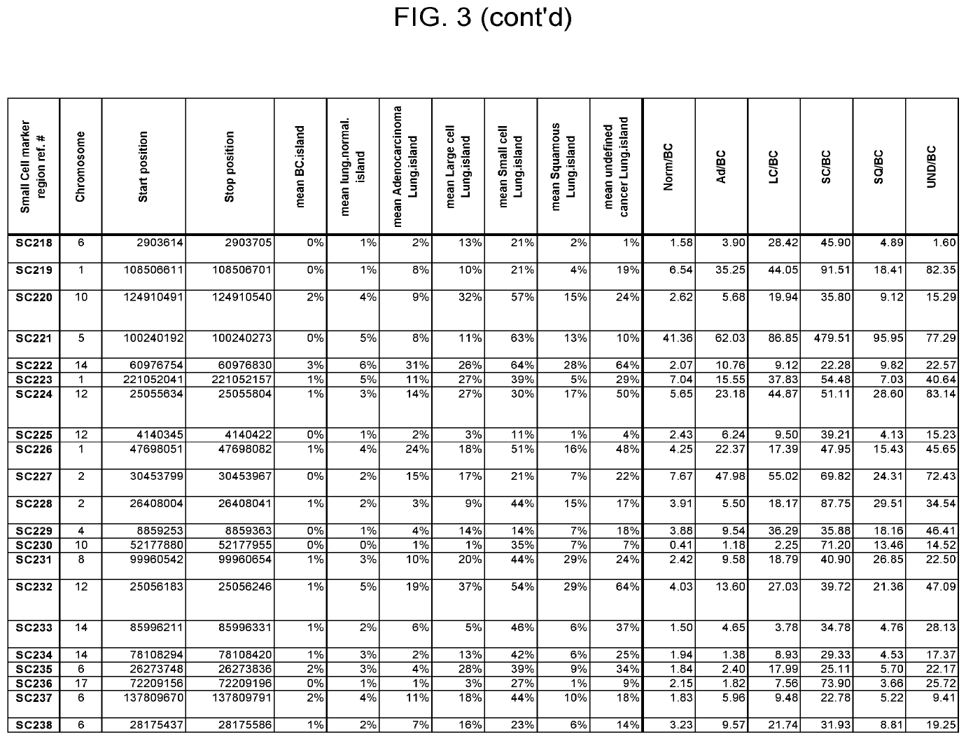

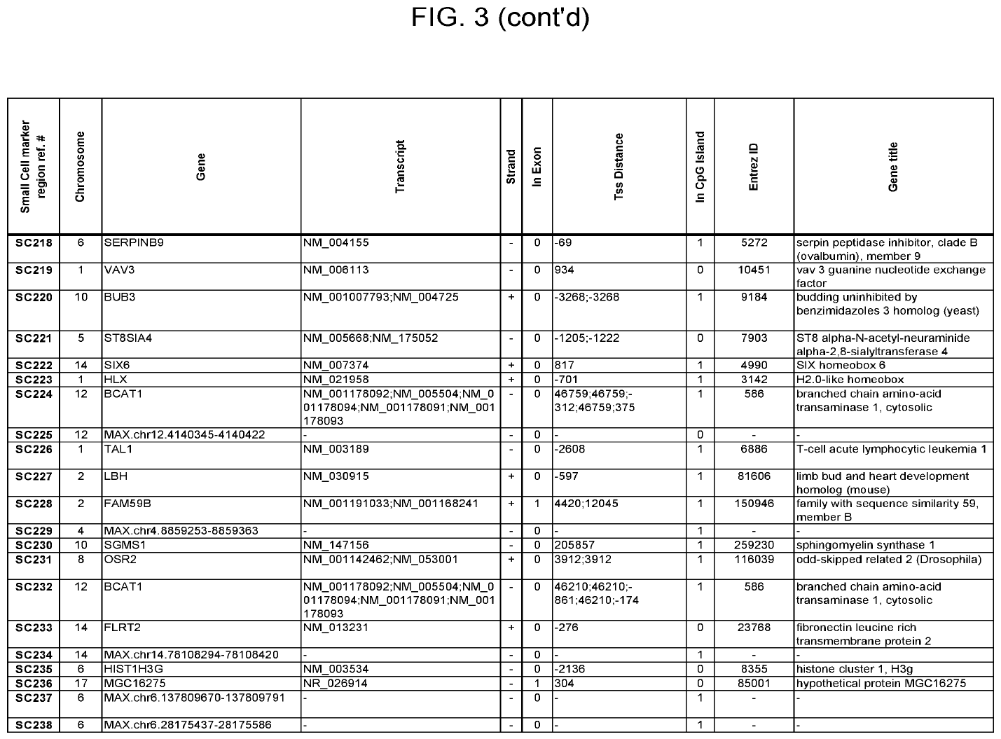

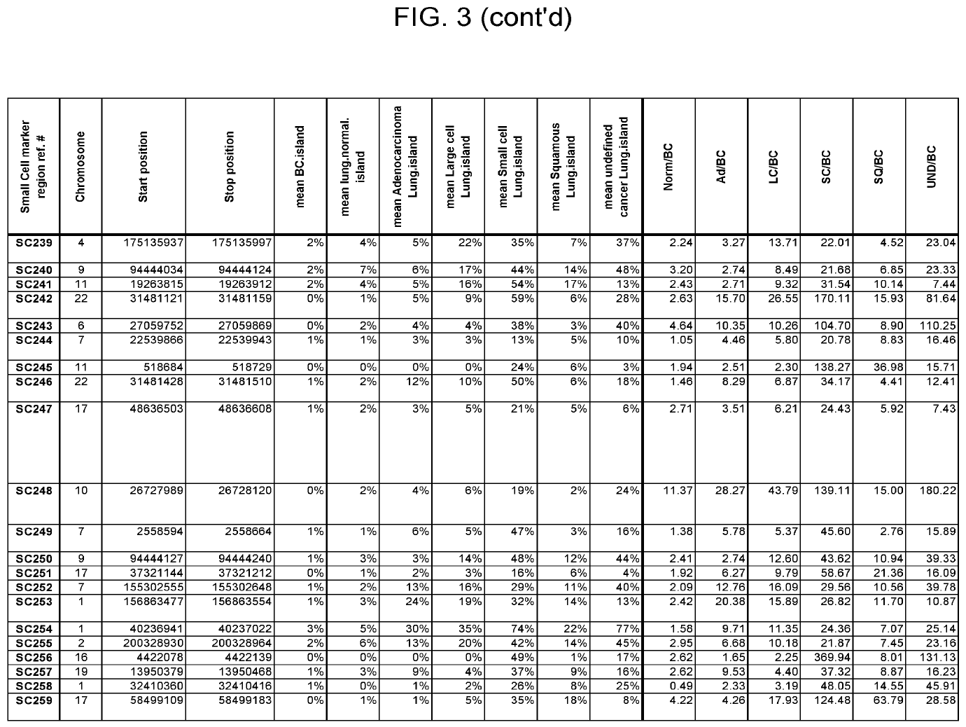

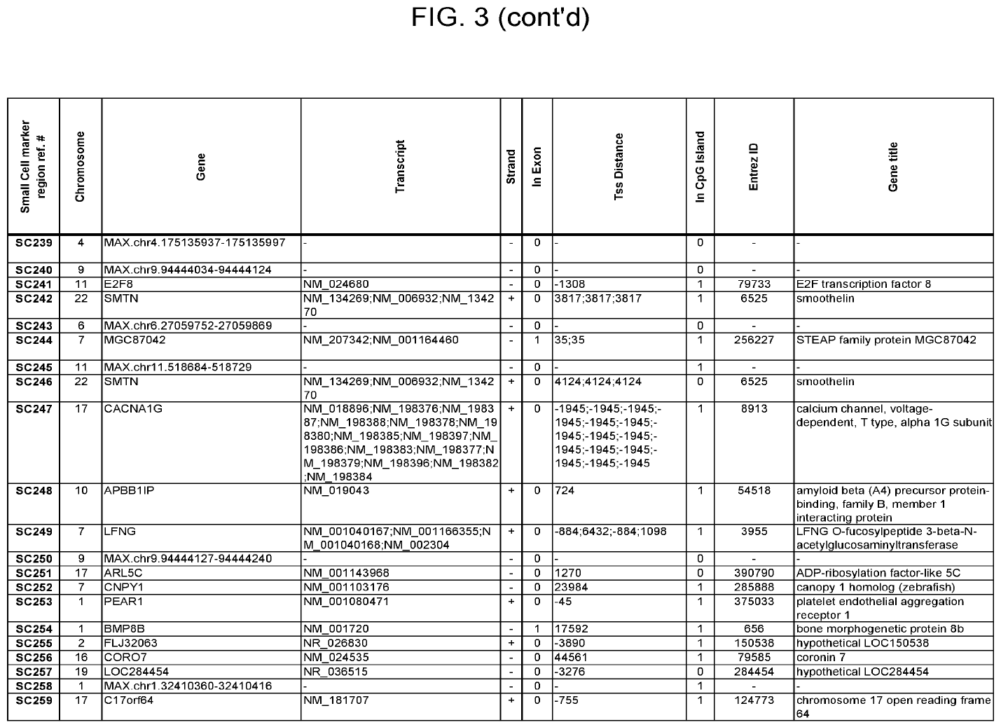

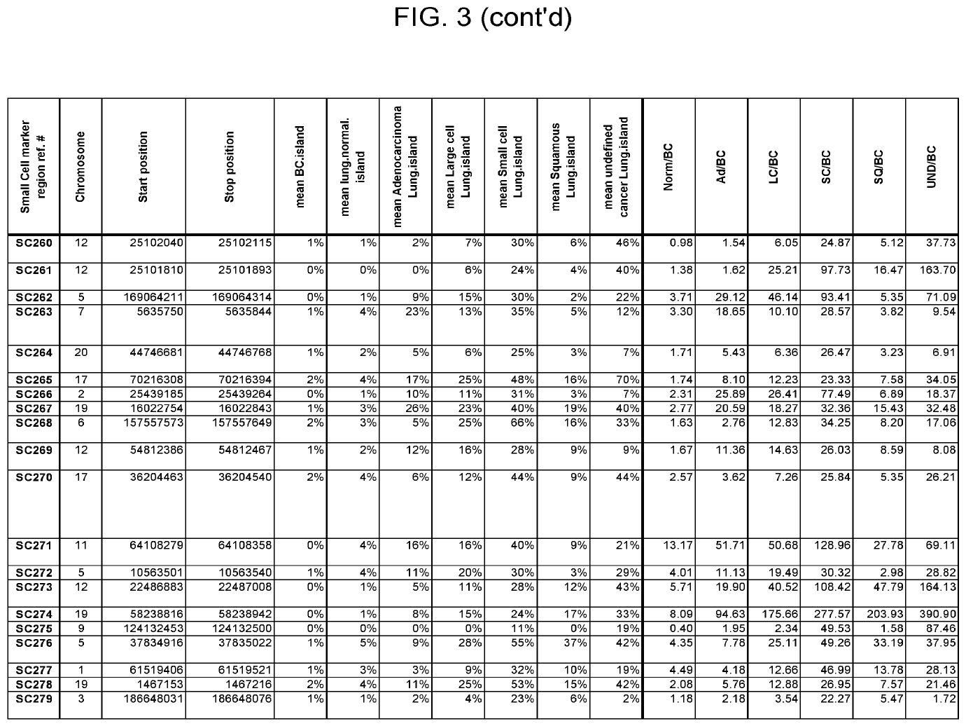

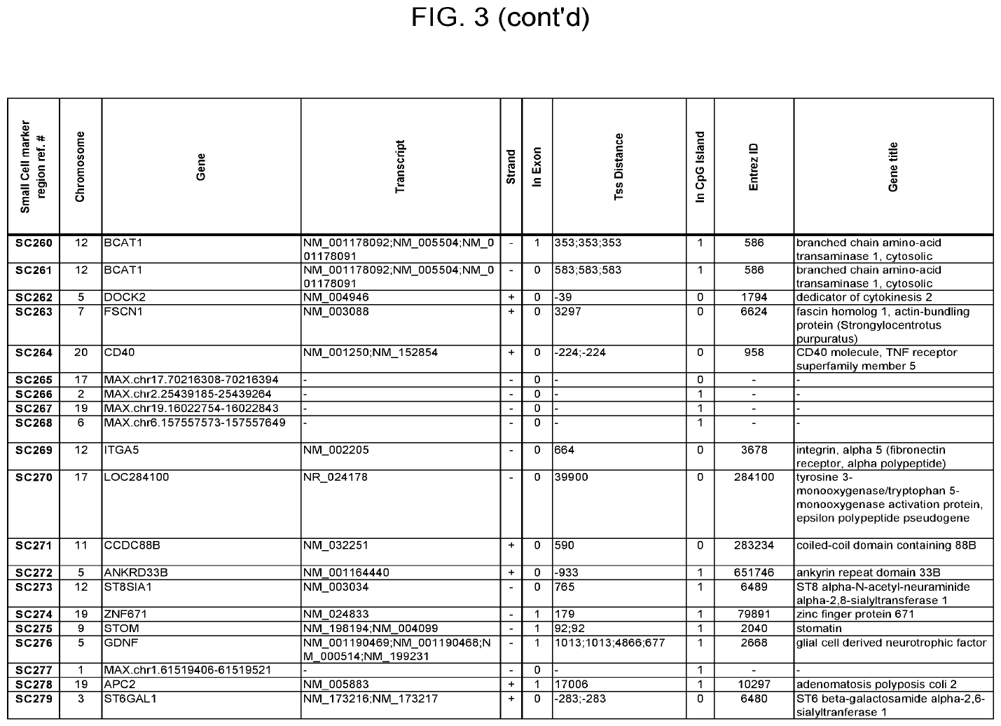

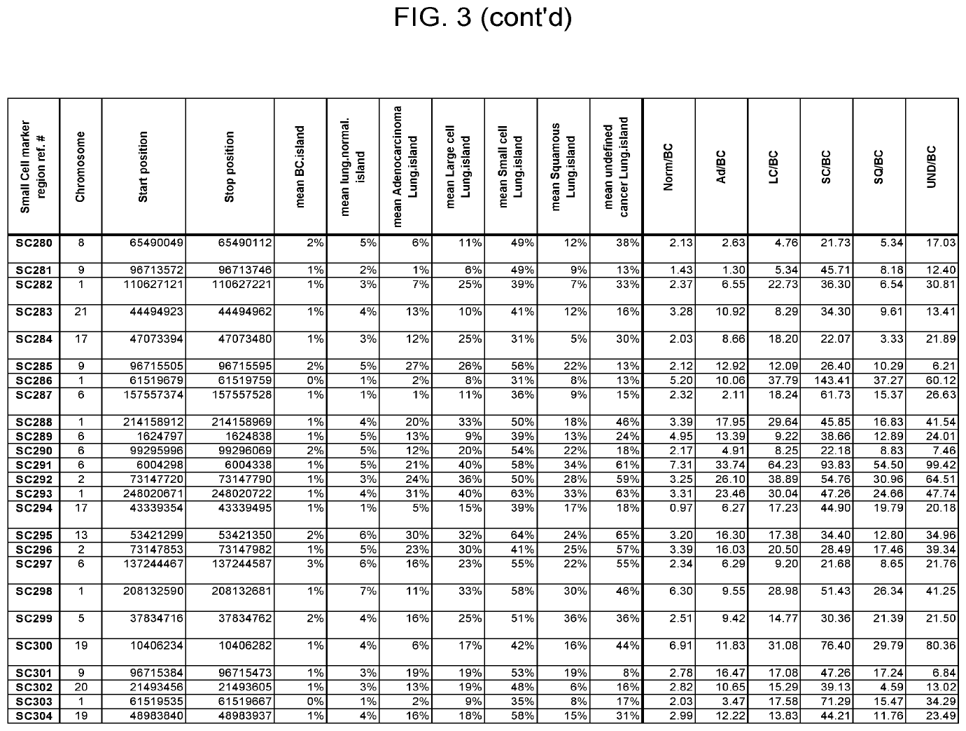

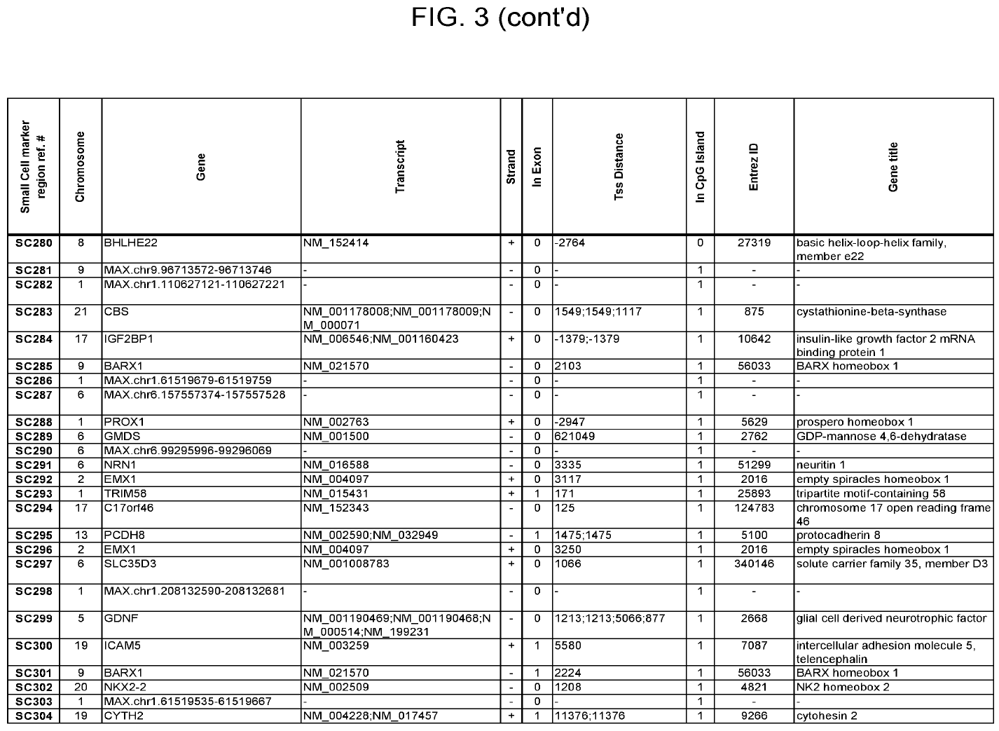

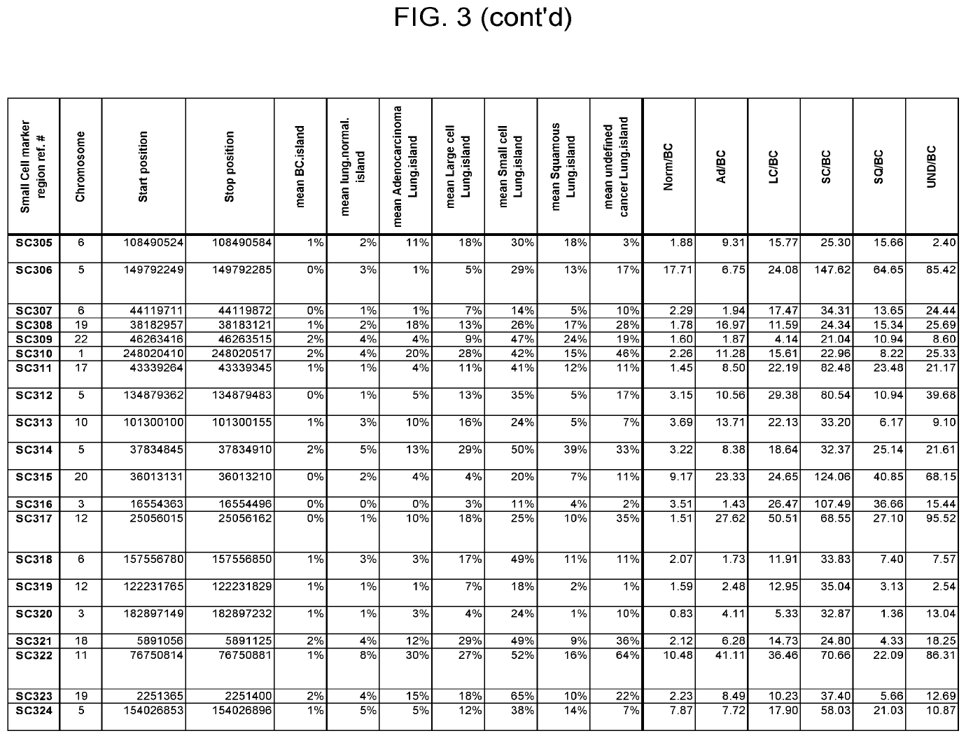

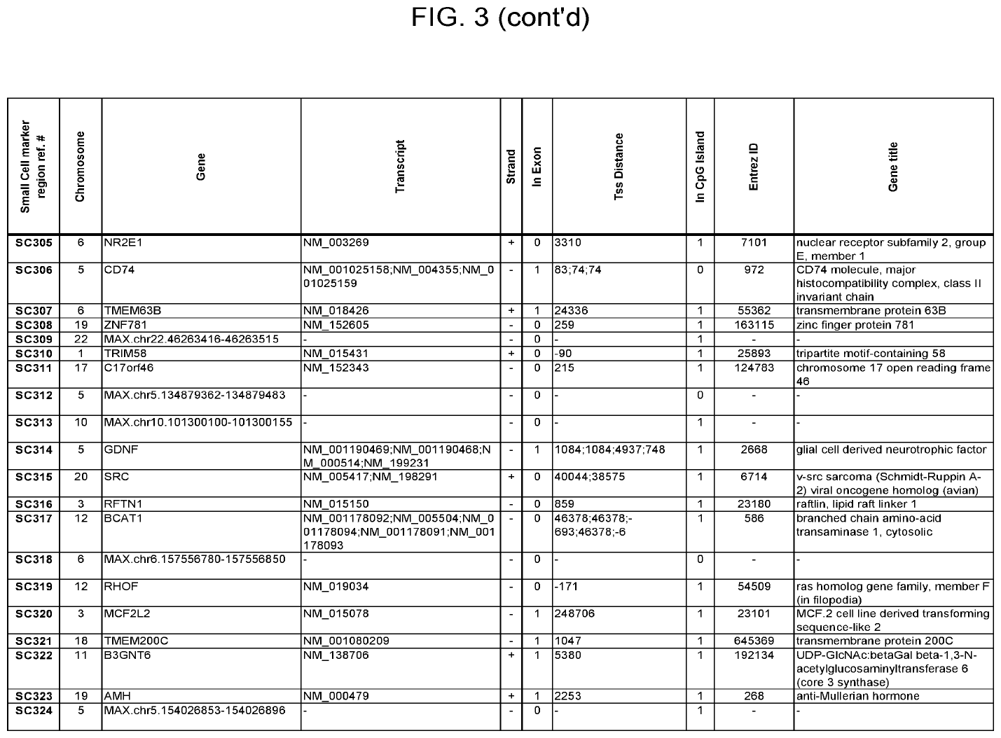

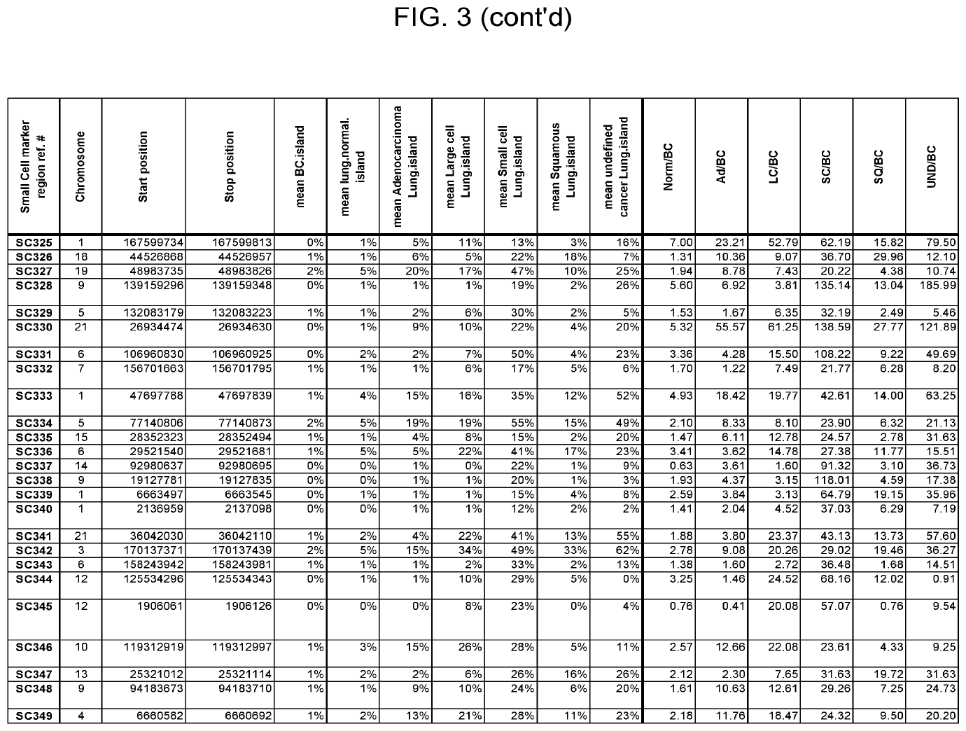

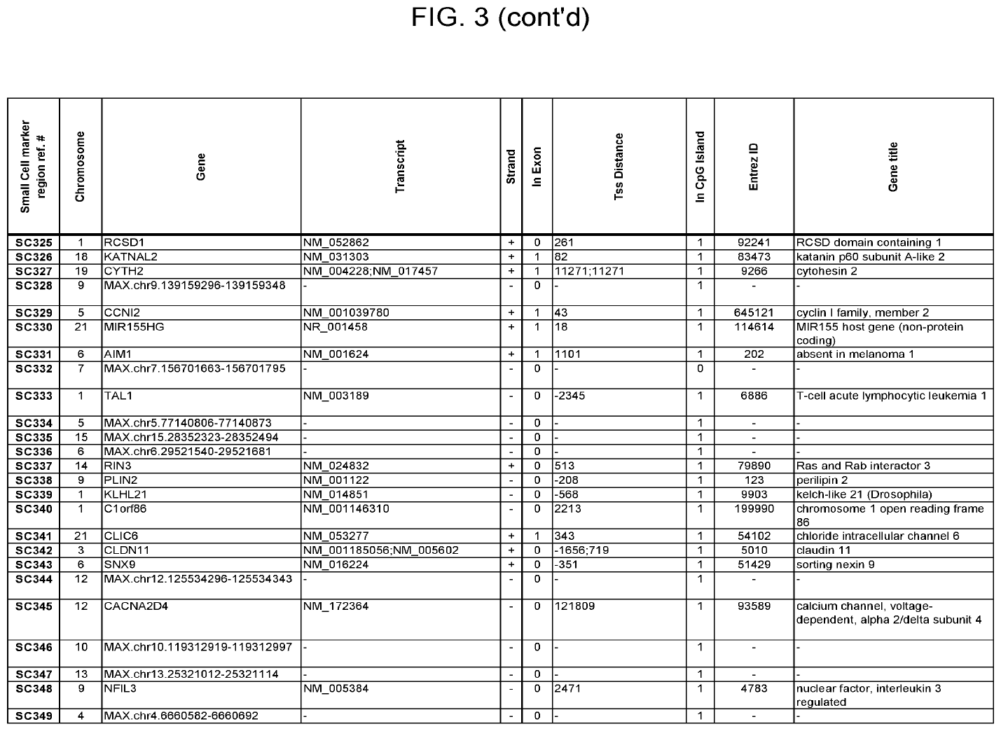

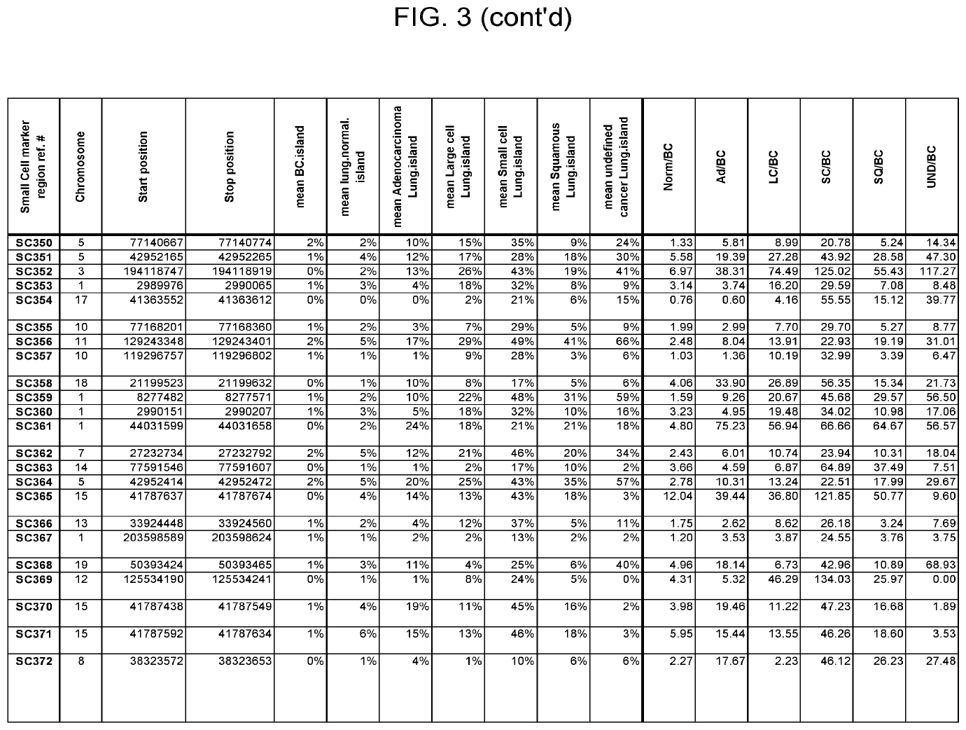

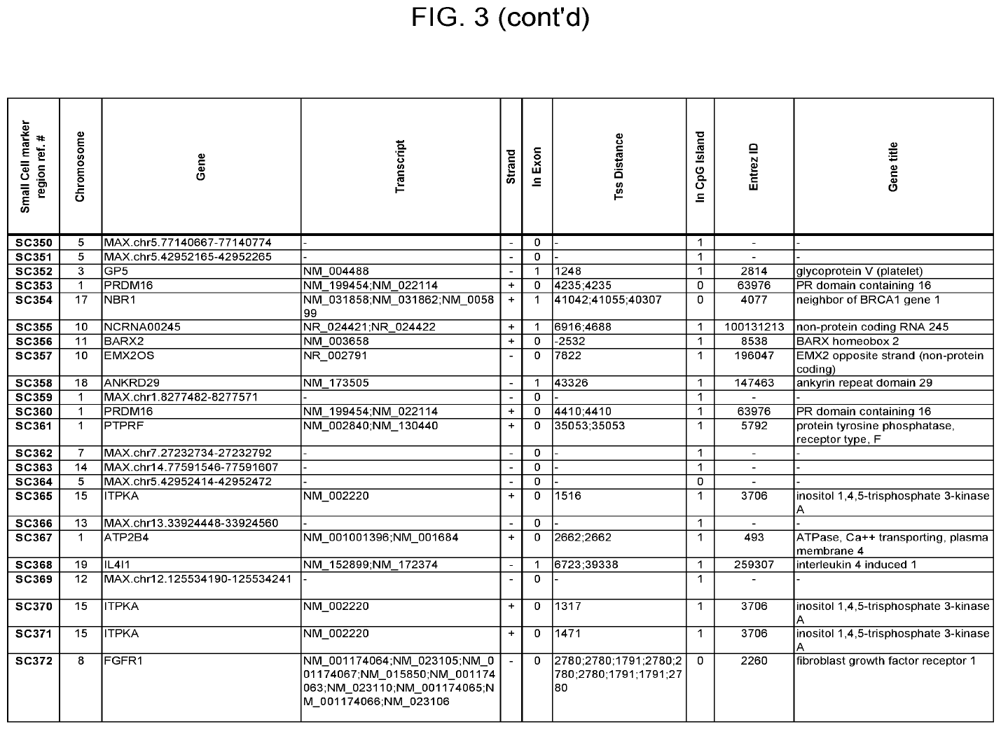

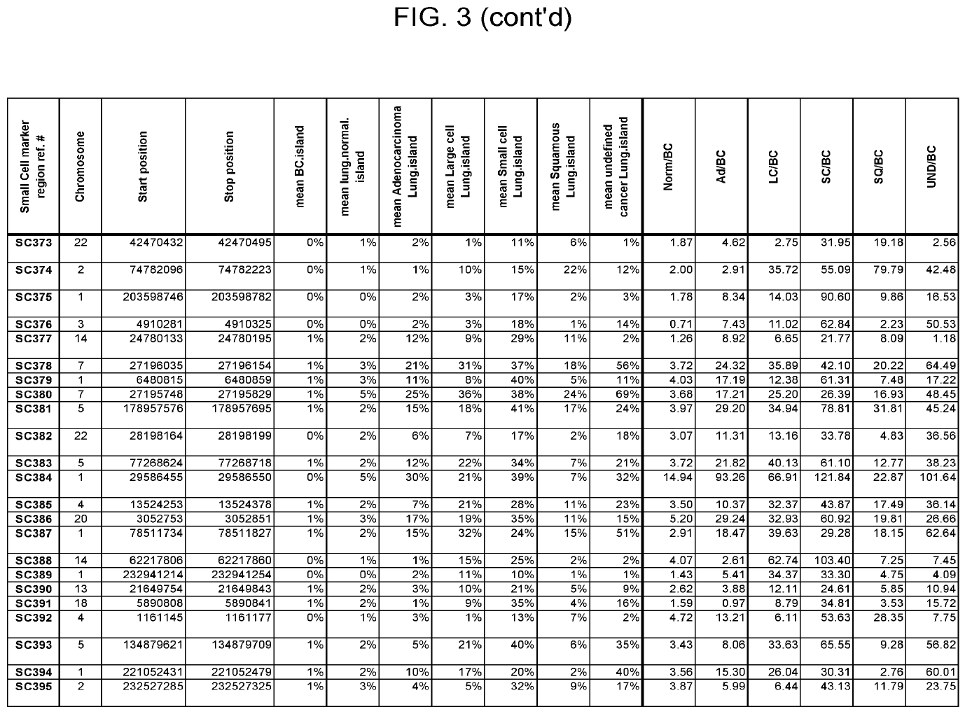

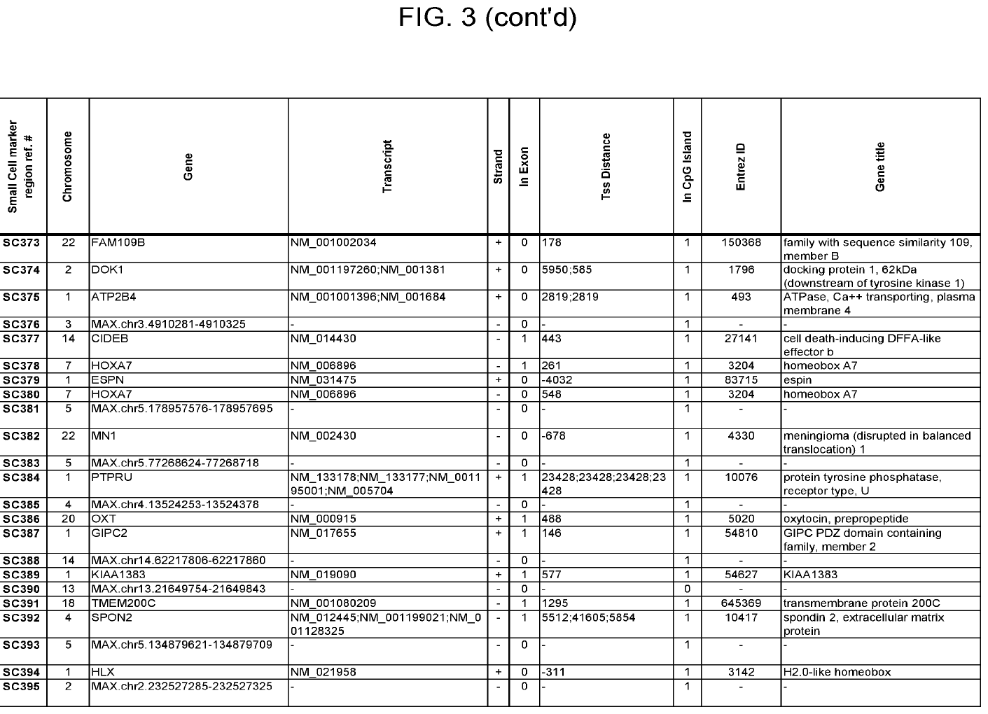

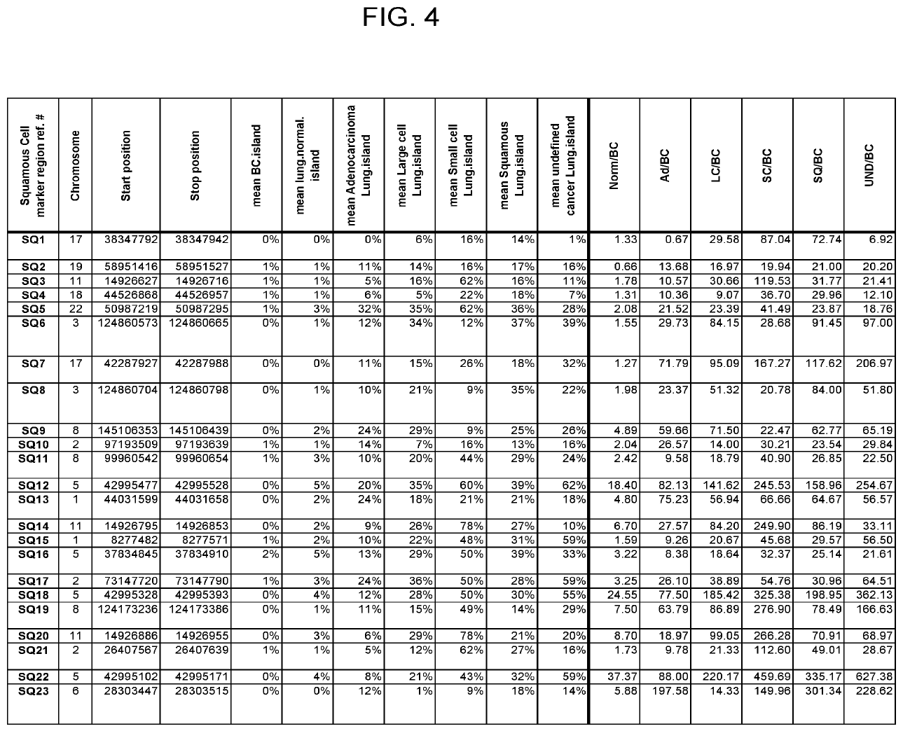

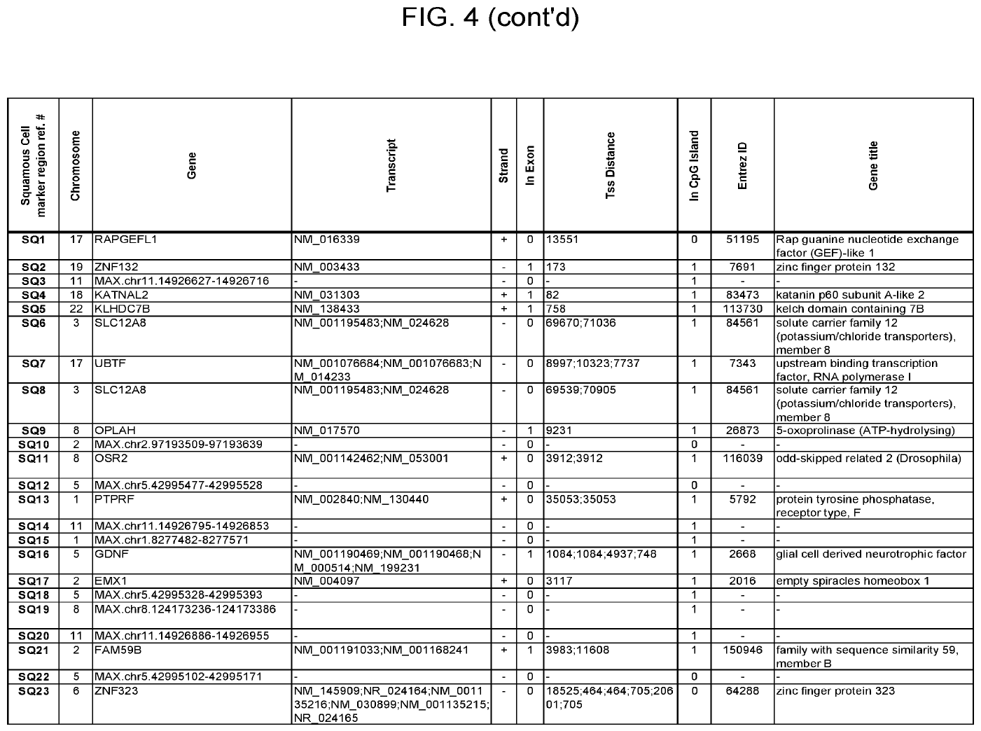

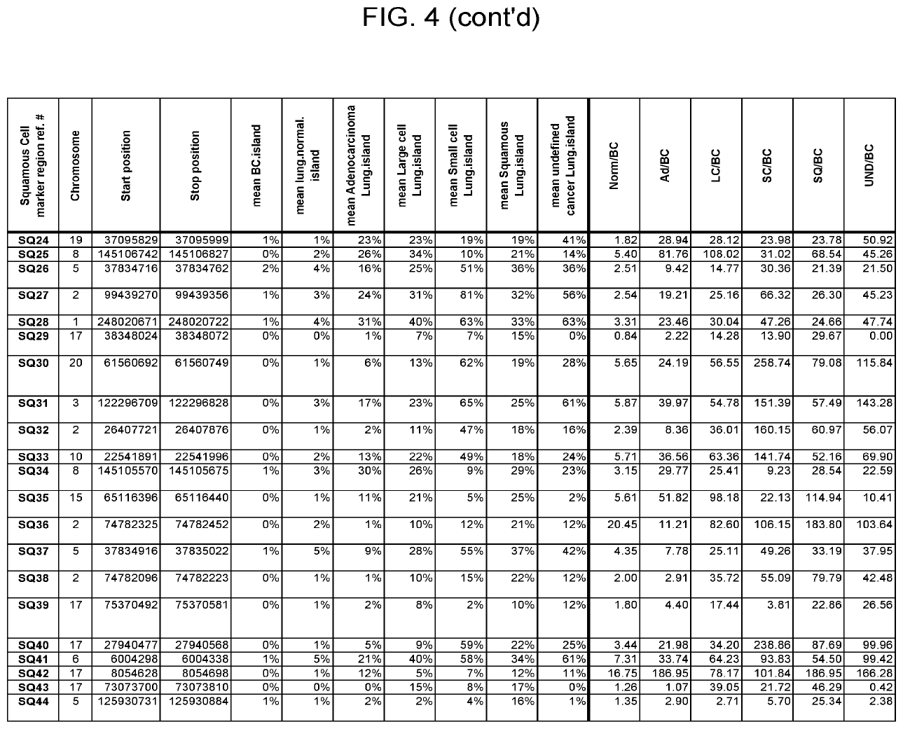

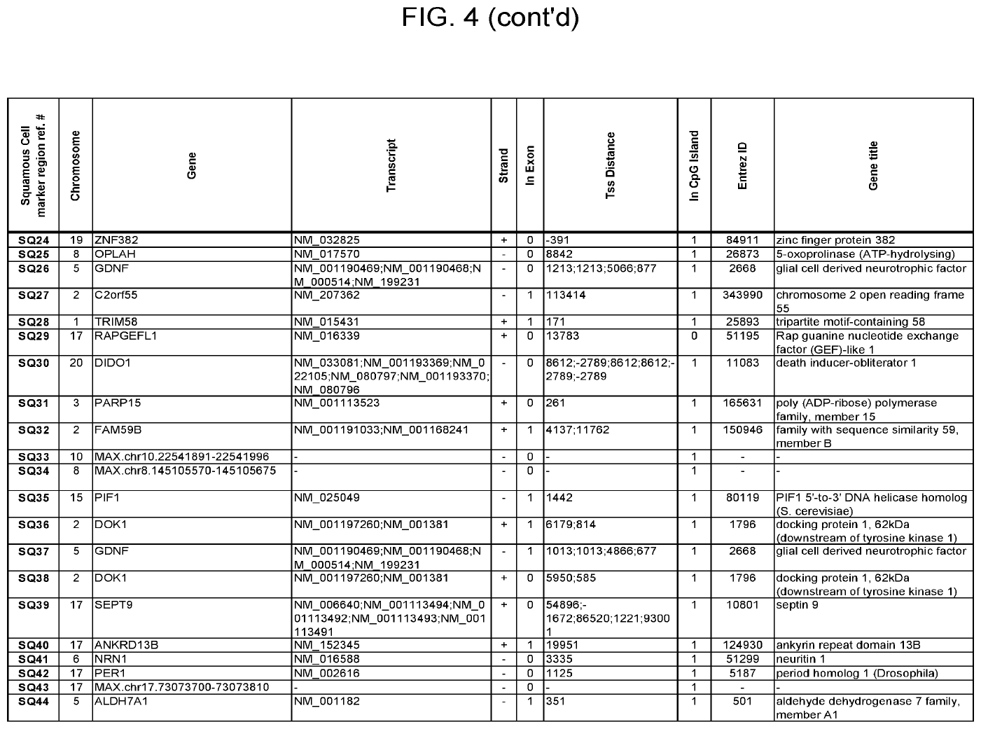

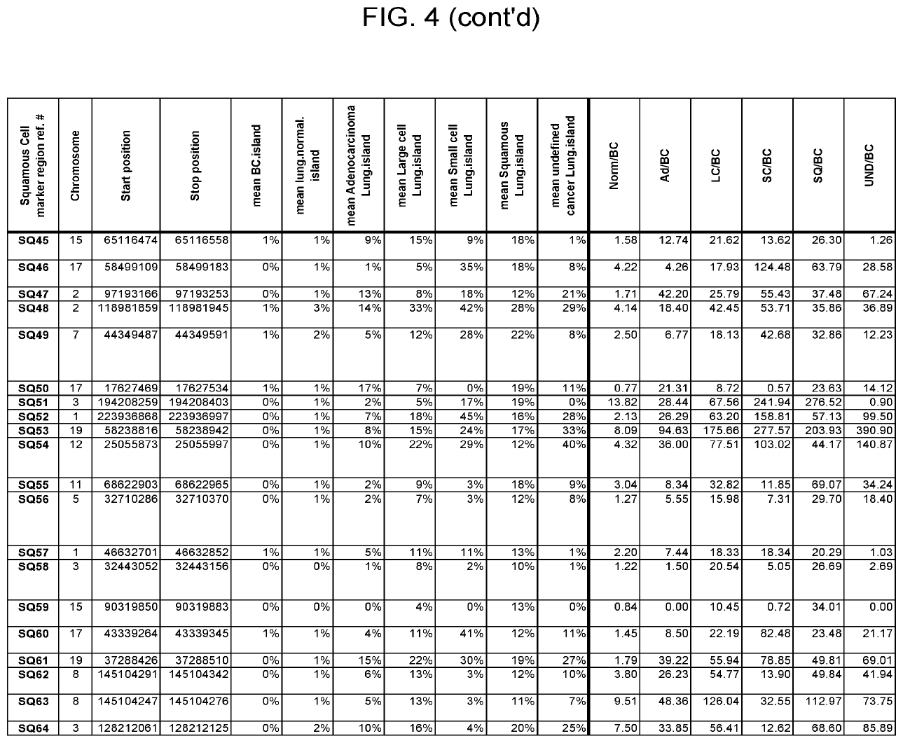

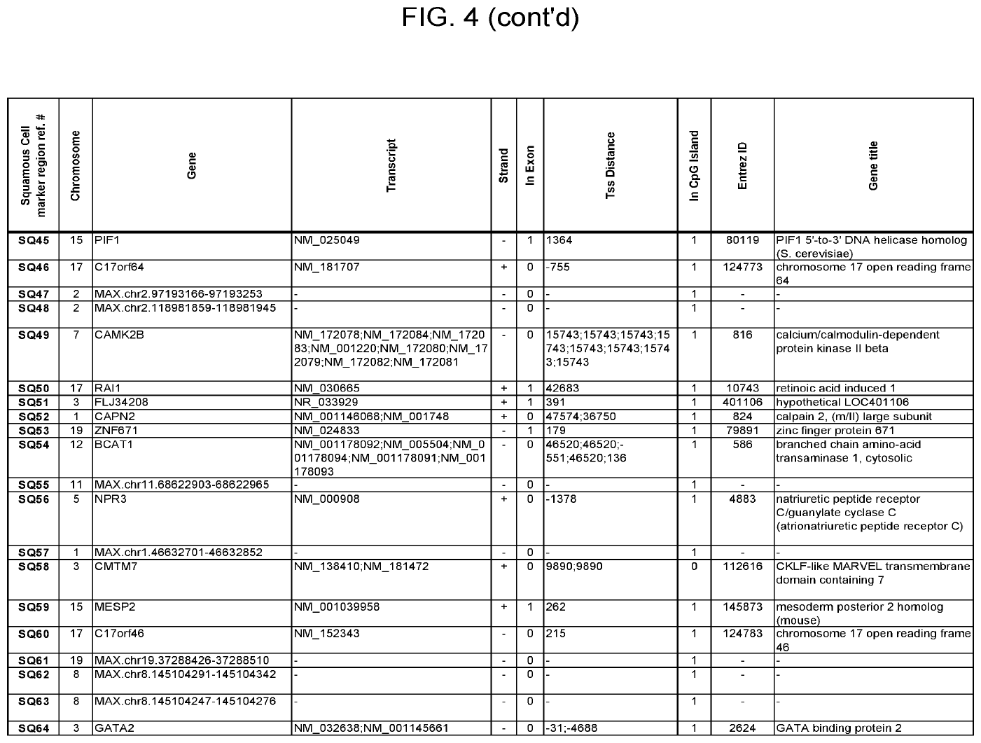

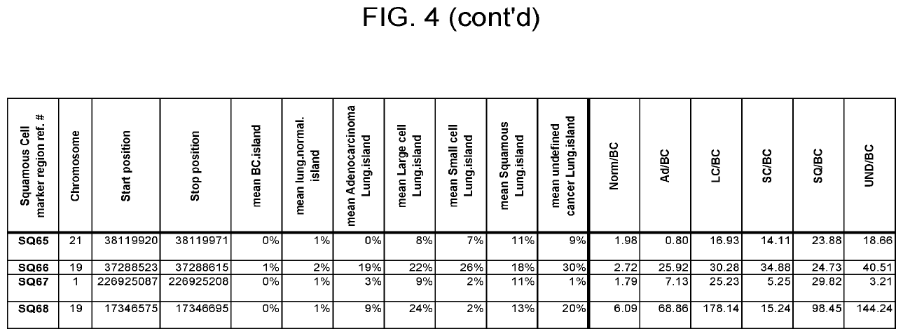

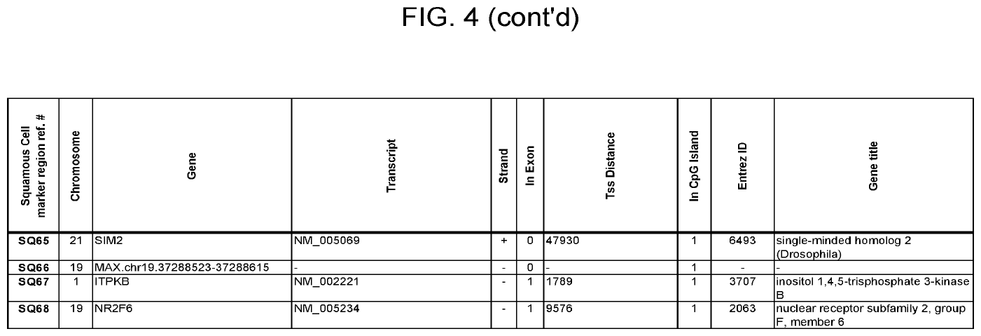

In some embodiments, a sample from a subject having or suspected of having lung cancer is screened using one or more methylation markers and suitable assay methods that provide data that differentiate between different types of lung cancer, e.g., non-small cell (adenocarcinoma, large cell carcinoma, squamous cell carcinoma) and small cell carcinomas. See, e.g., marker ref. #AC27 (FIG. 2; PLEC), which is highly methylated (shown as mean methylation compared to mean methylation at that locus in normal buffy coat) in adenocarcinoma and small cell carcinomas, but not in large cell or squamous cell carcinoma; marker ref. #AC23 (FIG. 1; ITPRIPL1), which is more highly methylated in adenocarcinoma than in any other sample type; marker ref. #LC2 (FIG. 2; DOCK2)), which is more highly methylated in large cell carcinomas than in any other sample type; marker ref #SC221 (FIG. 3; ST8SIA4), which is more highly methylated in small cell carcinomas than in any other sample type; and marker ref. #SQ36 (FIG. 4, DOK1), which is more highly methylated in squamous cell carcinoma than in than in any other sample type.

Methylation markers selected as described herein may be used alone or in combination (e.g., in panels) such that analysis of a sample from a subject reveals the presence of a lung neoplasm and also provides sufficient information to distinguish between lung cancer type, e.g., small cell carcinoma vs. non-small cell carcinoma. In preferred embodiments, a marker or combination of markers further provide data sufficient to distinguish between adenomcarcinomas, large cell carcinomas, and squamous cell carcinomas; and/or to characterize carcinomas of undetermined or mixed pathologies. In other embodiments, methylation markers or combinations thereof are selected to provide a positive result (e.g., a result indicating the presence of lung neoplasm) regardless of the type of lung carcinoma present, without differentiating data.

Over recent years, it has become apparent that circulating epithelial cells, representing metastatic tumor cells, can be detected in the blood of many patients with cancer. Molecular profiling of rare cells is important in biological and clinical studies. Applications range from characterization of circulating epithelial cells (CEpCs) in the peripheral blood of cancer patients for disease prognosis and personalized treatment (See e.g., Cristofanilli M, et al. (2004) N Engl J Med 351:781-791; Hayes D F, et al. (2006) Clin Cancer Res 12:4218-4224; Budd G T, et al., (2006) Clin Cancer Res 12:6403-6409; Moreno J G, et al. (2005) Urology 65:713-718; Pantel et al., (2008) Nat Rev 8:329-340; and Cohen S J, et al. (2008) J Clin Oncol 26:3213-3221). Accordingly, embodiments of the present disclosure provide compositions and methods for detecting the presence of metastatic cancer in a subject by identifying the presence of methylation markers in plasma or whole blood.

Also described herein are assays comprising multiplex reverse transcription and pre-amplification, followed by LQAS PCR-flap assays (A combined reverse transcription and pre-amplification with an LQAS assay is referred to as the RT-TELQAS assay (for “Reverse Transcription—Target Enrichment Long probe Quantitative Amplified Signal”). In RT-TELQAS assays, target RNAs, e.g., total RNA from a sample, is treated in an RT-pre-amplification reaction containing, e.g., 20U of MMLV reverse transcriptase, 1.5 U of GoTaq® DNA Polymerase, 10 mM MOPS buffer, pH7.5, 7.5 mM MgCl₂, 250 μM each dNTP, and oligonucleotide primers (e.g., for 12 targets, 12 primer pairs/24 primers, in equimolar amounts (e.g., 200 nM each primer) or in amounts modified to adjust amplification efficiencies of different target RNAs, and is incubated at a moderate temperature (e.g., 42° C.) for reverse transcription, followed by a limited number of thermal cycles (e.g., 10 cycles of 95° C., 63° C., 70° C.) to provide preamplification of target sequences corresponding to the included primers pairs. After thermal cycling, aliquots of the RT-pre-amplification reaction (e.g., 10 μL) are used in LQAS PCR-flap assays, as described below. RNAs suitable for detection in RT-TELQAS and RT-LQAS assays are not limited to any particular types of RNA targets. For example all manner of RNAs from tissues, cells or circulating cell-free RNAs from blood, such as protein-coding messenger RNAs (mRNA), microRNAs (miRNAs), piRNAs, tRNAs, and other non-coding RNA molecules (ncRNAs) (see, e.g., SU Umu, et al. “A comprehensive profile of circulating RNAs in human serum,” RNA Biology 15(2):242-250 (2018), which is incorporated herein by reference in its entirety) may be assayed using the RT-TELQAS and RT-LQAS methods described hereinbelow.

In preferred embodiments, the methods are conducted in reaction mixtures that comprise a PCR-flap assay buffer comprising having relatively high Mg⁺⁺ and low KCl compared to standard PCR buffers, (e.g., 6-10 mM, preferably 7.5 mM Mg⁺⁺, and 0.0 to 0.8 mM KCl). A typical PCR buffer is 1.5 mM MgCl₂, 20 mM Tris-HCl, pH 8, and 50 mM KCl, and PCR-flap assay buffer comprises 7.5 mM MgCl₂, 10 mM MOPS, 0.3 mM Tris-HCl, pH 8.0, 0.8 mM KCl, 0.1 μg/μL BSA, 0.0001% Tween-20, and 0.0001% IGEPAL CA-630. Surprisingly, in RT-LQAS and RT-TELQAS methods described hereinbelow, all amplification steps, including the reverse transcription of RT-LQAS flap assay and the RT-preamplification of the TELQAS method are conducted in the same PCR-flap assay buffer. When multiplex pre-amplification is used, the same primer pairs may be used for the pre-amplification target enrichment and the quantitative PCR-flap assay, i.e., the primers need not be nested primers. See, e.g., U.S. Pat. No. 10,704,081, which is incorporated herein by reference.

### EXPERIMENTAL EXAMPLES

The following examples are offered to illustrate but not to limit the invention. In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1

### Methods for RNA Isolation, DNA Isolation, Protein Isolation

The following provides exemplary method for RNA Isolation, DNA isolation, and protein sample preparation prior to analysis

RNA Isolation from Blood

Blood samples are collected in a blood collection tube suitable for subsequent RNA detection (e.g., PAXgene Blood RNA Tube; Qiagen, Inc.). Samples may be assayed immediately or frozen until future analysis. RNA is extracted from a sample by standard methods, e.g., Qiasymphony PAXgene blood RNA kit. (Prod. ID: 762635) per manufacturer's instructions. Prior to testing in RT-LQAS, RNA samples may be diluted (e.g., 1:50 in 10 mM Tris-HCl, pH 8.0, 0.1 mM EDTA)

DNA Isolation from Cells and Plasma

For cell lines, genomic DNA may be isolated from cell conditioned media using, for example, the “Maxwell® RSC ccfDNA Plasma Kit (Promega Corp., Madison, Wis.). Following the kit protocol, 1 mL of cell conditioned media (CCM) is used in place of plasma, and processed according to the kit procedure. The elution volume is 100 μL, of which 70 μL are generally used for bisulfite conversion.

An exemplary procedure for isolating DNA from a 4 mL sample of plasma is as follows:
To a 4 mL sample of plasma, 300 μL of Proteinase K (20 mg/mL) is added and mixed.
Add 3 μL of 1 μg/μL of Fish DNA to the plasma-proteinase K mixture.
Add 2 mL of plasma lysis buffer to plasma.
   Plasma lysis buffer is:
      4.3M guanidine thiocyanate
      10% IGEPAL CA-630 (Octylphenoxy poly(ethyleneoxy)ethanol, branched)
      (5.3 g of IGEPAL CA-630 combined with 45 mL of 4.8 M guanidine thiocyanate)
Incubate mixtures at 55° C. for 1 hour with shaking at 500 rpm.
Add and mix:
   3 mL of plasma lysis buffer
   2 mL of 100% isopropanol
   200 μL magnetic silica binding beads (16 μg of beads/μL)
   (optionally mix after each addition and/or optionally pre-mix the lysis buffer and isopropanol before adding to the mixture)
Incubate at 30° C. for 30 minutes with shaking at 500 rpm.
Place tube(s) on magnet and let the beads collect. Aspirate and discard the supernatant.
Add 750 μL GuHCl-EtOH to vessel containing the binding beads and mix.
   GuHCl-EtOH wash buffer is:
      3M GuHCl (guanidine hydrochloride)
      57% EtOH (ethyl alcohol)
Shake at 400 rpm for 1 minute.
Transfer samples to a deep well plate or 2 mL microcentrifuge tubes.
Place tubes on magnet and let the beads collect for 10 minutes. Aspirate and discard the supernatant.
Add 1000 μL wash buffer (10 mM Tris HCl, 80% EtOH) to the beads, and incubate at 30° C. for 3 minutes with shaking.
Place tubes on magnet and let the beads collect. Aspirate and discard the supernatant.
Add 500 μL wash buffer to the beads and incubate at 30° C. for 3 minutes with shaking.
Place tubes on magnet and let the beads collect. Aspirate and discard the supernatant.
Add 250 μL wash buffer and incubate at 30° C. for 3 minutes with shaking.
Place tubes on magnet and let the beads collect. Aspirate and discard the remaining buffer.
Add 250 μL wash buffer and incubate at 30° C. for 3 minutes with shaking.
Place tubes on magnet and let the beads collect. Aspirate and discard the remaining buffer.
Dry the beads at 70° C. for 15 minutes, with shaking.
Add 125 μL elution buffer (10 mM Tris HCl, pH 8.0, 0.1 mM EDTA) to the beads and incubate at 65° C. for 25 minutes with shaking.
Place tubes on magnet and let the beads collect for 10 minutes.
Aspirate and transfer the supernatant containing the DNA to a new vessel or tube.

### Bisulfite Conversion

### I. Sulfonation of DNA Using Ammonium Hydrogen Sulfite

1. In each tube, combine 64 μL DNA, 7 μL 1 N NaOH, and 9 μL of carrier solution containing 0.2 mg/mL BSA and 0.25 mg/mL of fish DNA.
2. Incubate at 42° C. for 20 minutes.
3. Add 120 μL of 45% ammonium hydrogen sulfite and incubate at 66° for 75 minutes.
4. Incubate at 4° C. for 10 minutes.

### II. Desulfonation Using Magnetic Beads

### Materials

Magnetic beads (Promega MagneSil Paramagnetic Particles, Promega catalogue number AS1050, 16 μg/μL).
Binding buffer: 6.5-7 M guanidine hydrochoride.
Post-conversion Wash buffer: 80% ethanol with 10 mM Tris HCl (pH 8.0).
Desulfonation buffer: 70% isopropyl alcohol, 0.1 N NaOH was selected for the desulfonation buffer.

Samples are mixed using any appropriate device or technology to mix or incubate samples at the temperatures and mixing speeds essentially as described below. For example, a Thermomixer (Eppendorf) can be used for the mixing or incubation of samples. An exemplary desulfonation is as follows:
1. Mix bead stock thoroughly by vortexing bottle for 1 minute.
2. Aliquot 50 μL of beads into a 2.0 mL tube (e.g., from USA Scientific).
3. Add 750 μL of binding buffer to the beads.
4. Add 150 μL of sulfonated DNA from step I.
5. Mix (e.g., 1000 RPM at 30° C. for 30 minutes).
6. Place tube on the magnet stand and leave in place for 5 minutes. With the tubes on the stand, remove and discard the supernatant.
7. Add 1,000 μL of wash buffer. Mix (e.g., 1000 RPM at 30° C. for 3 minutes).
8. Place tube on the magnet stand and leave in place for 5 minutes. With the tubes on the stand, remove and discard the supernatant.
9. Add 250 μL of wash buffer. Mix (e.g., 1000 RPM at 30° C. for 3 minutes).
10. Place tube on magnetic rack; remove and discard supernatant after 1 minute.
11. Add 200 μL of desulfonation buffer. Mix (e.g., 1000 RPM at 30° C. for 5 minutes).
12. Place tube on magnetic rack; remove and discard supernatant after 1 minute.
13. Add 250 μL of wash buffer. Mix (e.g., 1000 RPM at 30° C. for 3 minutes).
14. Place tube on magnetic rack; remove and discard supernatant after 1 minute.
15. Add 250 μL of wash buffer to the tube. Mix (e.g., 1000 RPM at 30° C. for 3 minutes).
16. Place tube on magnetic rack; remove and discard supernatant after 1 minute.
17. Incubate all tubes at 30° C. with the lid open for 15 minutes.
18. Remove tube from magnetic rack and add 70 μL of elution buffer directly to the beads.
19. Incubate the beads with elution-buffer (e.g., 1000 RPM at 40° C. for 45 minutes).
20. Place tubes on magnetic rack for about one minute; remove and save the supernatant.

The converted DNA is then used in a detection assay, e.g., a pre-amplification and/or flap endonuclease assays, as described below.

For additional embodiments of bisulfite treatment of nucleic acids, also U.S. Pat. No. 10,704,081, and U.S. Patent Appl. Ser. Nos. 63/058,179, filed Jul. 29, 2020, each of which is incorporated herein by reference in its entirety, for all purposes, and which may be applied in the technology described herein.

In some embodiments, RNA and DNA are isolated from different samples of blood from a subject. For example, blood may be collected in a first collection tube configured for optimal preservation and/or isolation of RNA and in a second collection tube configured to optimal preservation and isolation of DNA, and the RNA and DNA may be extracted from portions of blood collected in this fashion. IN other embodiments, RNA and DNA are both extracted from a single collected blood sample, using, e.g., a collection tube configured to optimal preservation and isolation of both DNA and RNA (e.g., cf-DNA/cf-RNA Preservative Tubes (Cat. 63950) from NORGEN Biotek Corp., for preservation and isolation of both cell-free DNA and cell-free RNA).

In some embodiments, RNA and DNA are assayed together, e.g., in an RT-LQAS/RT-TELQAS reaction. In some embodiments, the RNA and DNA are separately isolated and/or separately treated, e.g., with bisulfite, as described above, while in some embodiments, RNA and DNA are processed together, e.g., both being present during bisulfite treatment and subsequent purification, and added together to the assay reactions.

### Flap Endonuclease Assays

The QuARTS and LQAS/TELQAS flap assay technologies combine a polymerase-based target DNA amplification process with an invasive cleavage-based signal amplification process. The QuARTS technology is described, e.g., in U.S. Pat. Nos. 8,361,720; 8,715,937; 8,916,344; and 9,212,392, and a flap assay using probe oligonucleotides having a longer target-specific region (Long probe Quantitative Amplified Signal, “LQAS”) is described in U.S. Pat. No. 10,648,025, each of which is incorporated herein by reference in its entirety for all purposes. In the QuARTS assays described herein, the flap oligonucleotides have a target specific region of 12 bases, while the LQAS assays use flap oligonucleotides have a target specific region of at least 13 bases, and use different thermal cycling procedures for amplification. Fluorescence signal generated by the QuARTS and LQAS reactions are monitored in a fashion similar to real-time PCR, permitting quantitation of the amount of a target nucleic acid in a sample.

An exemplary QuARTS reaction typically comprises approximately 200-600 nmol/L (e.g., 500 nmol/L) of each primer and detection probe, approximately 100 nmol/L of the invasive oligonucleotide, approximately 600-700 nmol/L of each FRET cassette (FAM, e.g., as supplied commercially by Hologic, Inc.; HEX, e.g., as supplied commercially by BioSearch Technologies; and Quasar 670, e.g., as supplied commercially by BioSearch Technologies, and comprising a “black hole” quencher, e.g., BHQ-1, BHQ-2, or BHQ-3, BioSearch Technologies), 6.675 ng/μL FEN-1 endonuclease (e.g., Cleavase® 2.0, Hologic, Inc.), 1 unit Taq DNA polymerase in a 30 μL reaction volume (e.g., GoTaq® DNA polymerase, Promega Corp., Madison, Wis.), 10 mmol/L 3-(n-morpholino) propanesulfonic acid (MOPS), 7.5 mmol/L MgCl₂, and 250 μmol/L of each dNTP. Exemplary QuARTS cycling conditions are as shown in the table below. In some applications, analysis of the quantification cycle (C_{q}) provides a measure of the initial number of target DNA strands (e.g., copy number) in the sample.

| Stage | Temp/Time | # of Cycles |
|---|---|---|
| Denaturation | 95° C./3′ | 1 |
| Amplification 1 | 95° C./20″ | 10 |
| | 67° C./30″ | |
| | 70° C./30″ | |
| Amplification 2 | 95° C./20″ | 37 |
| | 53° C./1′ | |
| | 70° C./30″ | |
| Cooling | 40° C./30″ | 1 |

An exemplary LQAS reaction typically comprises approximately 200-600 nmol/L of each primer, approximately 100 nmol/L of the invasive oligonucleotide, approximately 500 nmol/L of each flap oligonucleotide probe and FRET cassette. LQAS reactions may, for example, be subjected to the following thermocycling conditions:

| Stage | Temp/Time | # of Cycles |
|---|---|---|
| Denaturation | 95° C./3′ | 1 |
| Amplification | 95° C./20″ | 40 |
| | 63° C./1′ | |
| | 70° C./30″ | |
| Cooling | 40° C./30″ | 1 |

### Multiplex Targeted Pre-Amplification for QuARTS and LQAS Assays

### Multiplex Targeted Pre-Amplification of Bisulfite-Converted DNA

To pre-amplify most or all of the bisulfite-treated DNA from an input sample, a large volume of the treated DNA may be used in a single, large-volume multiplex amplification reaction. For example, DNA is extracted from a cell lines (e.g., DFCI032 cell line (adenocarcinoma); H1755 cell line (neuroendocrine), using, for example, the Maxwell Promega blood kit #AS1400, as described above. The DNA is bisulfite converted, e.g., as described above.

A pre-amplification is conducted, for example, in a reaction mixture containing 7.5 mM MgCl₂, 10 mM MOPS, 0.3 mM Tris-HCl, pH 8.0, 0.8 mM KCl, 0.1 μg/μL BSA, 0.0001% Tween-20, 0.0001% IGEPAL CA-630, 250 μM each dNTP, oligonucleotide primers, (e.g., for 12 targets, 12 primer pairs/24 primers, in equimolar amounts (including but not limited to the ranges of, e.g., 200-500 nM each primer), or with individual primer concentrations adjusted to balance amplification efficiencies of the different target regions), 0.025 units/μL HotStart GoTaq concentration, and 20 to 50% by volume of bisulfite-treated target DNA (e.g., 10 μL of target DNA into a 50 μL reaction mixture, or 50 μL of target DNA into a 125 μL reaction mixture). Thermal cycling times and temperatures are selected to be appropriate for the volume of the reaction and the amplification vessel. For example, the reactions may be cycled as follows:

| Stage | Temp/Time | #of Cycles |
|---|---|---|
| Pre-incubation | 95° C./5′ | 1 |
| Amplification 1 | 95° C./30″ | 10-12 |
| | 64° C./30″ | |
| | 72° C./30″ | |
| Cooling | 4° C./Hold | 1 |

After thermal cycling, aliquots of the pre-amplification reaction (e.g., 10 μL) are diluted to 500 μL in 10 mM Tris, 0.1 mM EDTA, with or without fish DNA. Aliquots of the diluted pre-amplified DNA (e.g., 10 μL) are used in a QuARTS PCR-flap assay, e.g., as described above. See also U.S. Patent Appl. Ser. No. 62/249,097, filed Oct. 30, 2015; application Ser. No. 15/335,096, filed Oct. 26, 2016, and PCT/US16/58875, filed Oct. 26, 2016, each of which is incorporated herein by reference in its entirety for all purposes.

A combined pre-amplification and LQAS assay is referred to as the TELQAS assay (for “Target Enrichment Long probe Quantitative Amplified Signal”).

Using the pre-amplified sample, QuARTS and TELQAS reactions are set up as follows:

Volume per
reaction (μL)

Mastermix (per reaction)

| | |
|---|---|
| Water (mol. biol. grade) | 15.50 |
| 10X Oligo Mix* | 3.00 |
| 20X QuARTS/LQAS | 1.50 |
| Enzyme Mix** | |
| Total Mastermix volume | 20.0 |

Reaction Mix

| | |
|---|---|
| Mastermix | 20 |
| Pre-amplified Sample | 10 |
| Final Reaction volume | 10 |

*10X oligonucleotide mix = 2 μM each primer and 5 μM each probe and FRET oligonucleotide
**20X enzyme mix contains 1 unit/μL GoTaq Hot start polymerase (Promega), 292 ng/μL Cleavase 2.0 flap endonuclease(Hologic).

As noted above, the flap oligonucleotides in the QuARTS assays have a target specific region of 12 bases, while the LQAS assays use flap oligonucleotides have a target specific region of at least 13 bases and are subjected to different thermal cycling conditions.

QuARTS reactions are subjected to the following thermocycling conditions:

QuARTS Assay Reaction Cycle:

| | | Ramp Rate | | |
|---|---|---|---|---|
| | | (° C. per | Number of | Signal |
| Stage | Temp/Time | second) | Cycles | Acguisition |
| Pre-incubation | 95° C./3 min | 4.4 | 1 | No |
| Amplification 1 | 95° C./20 sec | 4.4 | 5 | No |
| | 63° C./30 sec | 2.2 | | No |
| | 70° C./30 sec | 4.4 | | No |
| Amplification 2 | 95° C./20 sec | 4.4 | 40 | No |
| | 53° C./1 min | 2.2 | | Yes |
| | 70° C./30 sec | 4.4 | | No |
| Cooling | 40° C./30 sec | 2.2 | 1 | No |

TELQAS reactions are subjected to the following thermocycling conditions:

TELQAS Assay Reaction Cycle:

| | | Ramp Rate | | |
|---|---|---|---|---|
| | | (° C. per | Number of | Signal |
| Stage | Temp/Time | second) | Cycles | Acguisition |
| Pre-incubation | 95° C./3 min | 4.4 | 1 | No |
| Amplification | 95° C./20 sec | 4.4 | 40 | No |
| | 63° C./1 min | 2.2 | | Yes |
| | 70° C./30 sec | 4.4 | | No |
| Cooling | 40° C./30 sec | 2.2 | 1 | No |

### LQAS/TELQAS for RNA Detection (“RT-LQAS” or “RT-TELQAS”)

An exemplary RT-LQAS reaction contains 20U of MMLV reverse transcriptase (MMLV-RT), 219 ng of Cleavase®) 2.0, 1.5 U of GoTaq® DNA Polymerase, 200 nM of each primer, 500 nM each of probe and FRET oligonucleotides, 10 mM MOPS buffer, pH 7.5, 7.5 mM MgCl₂, and 250 μM each nNTP. An exemplary protocol is as follows:
1. Remove the required oligonucleotide mixes needed from the −20° C. freezer and allow to thaw.
2. Thaw controls from the −80° C. for a brief time at room temperature, then place on ice.
3. Thaw sample plate from the −80° C. for a brief time at room temperature, then place on ice.
4. Prepare master mix for the oligo mixtures in an appropriately sized tube.
5. Dilute MMLV-RT 1:20 in H₂O

mRNA Reverse Transcription 10X Master Mix Formulation

| Component | μL/reaction |
|---|---|
| Nuclease Free-H₂O (Promega) | 14.5 |
| MMLV_RT Diluted in NF H₂O | 1.0 |
| 10X Oligo Mix | 3.00 |
| 20X Enzyme Mix | 1.5 |
| Total Volume Master Mix (μL) | 20.0 |
| Sample Vol. (μL) | 10 |
| Final RT- LQAS Reaction Vol. (μL) | 30 |

6. Pipette 20 μL of master mix into a 96-well RT-LQAS plate, using a matrix pipet OR an eight-channel P20 pipet, per the plate layout.
7. Load 10 μL of samples, controls, calibrators (per plate layout).
8. Seal plate and briefly centrifuge.
9. Run plates with following reaction conditions on the

Reactions are typically run on a thermal cycler configured to collect fluorescence data in real time (e.g., continuously, or at the same point in some or all cycles). For example, a Roche LightCycler 480 instrument or an Applied Biosystem QuantStudioDX Real-Time PCR instrument may be used under the following conditions:

RT-LQAS Assay Reaction Cycle:

| | | Ramp Rate | Number | |
|---|---|---|---|---|
| | | (° C. per | of | Signal |
| Stage | Temp/Time | second) | Cycles | Acquisition |
| Reverse | 42° C./30 min | 4.4 | 1 | No |
| Transcription | | | | |
| Pre-incubation | 95° C./3 min | 4.4 | 1 | No |
| Amplification | 95° C./20 sec | 4.4 | 45 | No |
| | 63° C./1 min | 2.2 | | Single |
| | 70° C./30 sec | 4.4 | | No |
| Cooling | 40° C./30 sec | 2.2 | 1 | No |

In some embodiments, RT-LQAS assays may comprise a step of multiplex reverse transcription and pre-amplification, e.g., to pre-amplify 2, 5, 10, 12, or more targets in a sample (or any number of targets greater than 1 target), as described above, and may be referred to as “RT-TELQAS.” In preferred embodiments, an RT-pre-amplification is conducted in a reaction mixture containing, e.g., 20U of MMLV reverse transcriptase, 1.5 U of GoTaq® DNA Polymerase, 10 mM MOPS buffer, pH7.5, 7.5 mM MgCl₂, 250 μM each dNTP, and oligonucleotide primers, (e.g., for 12 targets, 12 primer pairs/24 primers, in equimolar amounts (e.g., 200 nM each primer), or with individual primer concentrations adjusted to balance amplification efficiencies of the different targets). Thermal cycling times and temperatures are selected to be appropriate for the volume of the reaction and the amplification vessel. For example, the reactions may be cycled as follows:

| | | # of |
|---|---|---|
| Stage | Temp/Time | Cycles |
| RT | 42° C./30′ | 1 |
| | 95° C./3′ | 1 |
| Amplification | 95° C./20″ | 10 |
| | 63° C./30″ | |
| | 70° C./30″ | |
| Cooling | 4° C./Hold | 1 |

After thermal cycling, aliquots of the RT-pre-amplification reaction (e.g., 10 μL) are diluted to 500 μL in 10 mM Tris, 0.1 mM EDTA, with or without fish DNA. Aliquots of the diluted pre-amplified DNA (e.g., 10 μL) are used in LQAS/TELQAS PCR-flap assays, as described above. In some embodiments, LQAS/TELQAS PCR flap assays are performed using additional amounts of the same primer pairs

### Example 2

### Selection and Testing of Methylation Markers

### Marker Selection Process:

Reduced Representation Bisulfite Sequencing (RRBS) data was obtained on tissues from 16 adenocarcinoma lung cancer, 11 large cell lung cancer, 14 small cell lung cancer, 24 squamous cell lung cancer, and 18 non-cancer lung as well as RRBS results of buffy coat samples obtained from 26 healthy patients.

After alignment to a bisulfite-converted form of the human genome sequence, average methylation at each CpG island was computed for each sample type (i.e., tissue or buffy coat) and marker regions were selected based on the following criteria:
Regions were selected to be 50 base pairs or longer.
For QuARTS flap assay designs, regions were selected to have a minimum of 1 methylated CpG under each of: a) the probe region, b) the forward primer binding region, and c) the reverse primer binding region. For the forward and reverse primers, it is preferred that the methylated CpGs are close to the 3′-ends of the primers, but not at the 3′terminal nucleotide. Exemplary flap endonuclease assay oligonucleotides are shown in FIG. 5.
Preferably, buffy coat methylation at any CpG in a region of interest is no more than >0.5%.
Preferably, cancer tissue methylation in a region of interest is >10%.
For assays designed for tissue analysis, normal tissue methylation in a region of interest is preferably <0.5%.

RRBS data for different lung cancer tissue types is shown in FIGS. 2-5. Based on the criteria above, the markers shown in the table below were selected and QuARTS flap assays were designed for them, as shown in FIG. 5.

TABLE 1

| Marker Name | Genomic coordinates |
|---|---|
| AGRN | chr1: 968467-968582, strand = + |
| ANGPT1 | chr8: 108509559-108509684, strand = − |
| ANKRD13B | chr17: 27940470-27940578, strand = + |
| ARHGEF4 | chr2: 131792758-131792900, strand = − |
| B3GALT6 | chr1: 1163595-1163733, strand = + |
| BARX1 | chr9: 96721498-96721597, strand = − |
| BCAT1 | chr12: 25055868-25055986, strand = − |
| BCL2L11 | chr2: 111876620-111876759, strand = − |
| BHLHE23 | chr20: 61638462-61638546, strand = − |
| BIN2 | chr12: 51717898-51717971, strand = − |
| BIN2_Z | chr12: 51718088-51718165, strand = + |
| CAPN2 | chr1: 223936858-223936998, strand = + |
| chrl7_737 | chr17: 73749814-73749919, strand = − |
| chr5_132 | chr5: 132161371-132161482,Strand = + |
| chr7_636 | chr7: 104581684-104581817, Strand = − |
| CYP26C1 | chr10: 94822396-94822502, strand = + |
| DIDO1 | chr20: 61560669-61560753, strand = − |
| DLX4 | chr17: 48042426-48042820, strand = − |
| DMRTA2 | chr1: 50884390-50884519, strand = − |
| DNMT3A | chr2: 25499967-25500072, strand = − |
| DOCK2 | chr5: 169064370-169064454, strand = − |
| EMX1 | chr2: 73147685-73147792, strand = + |
| FAM59B | chr2: 26407701-26407828, strand = + |
| FERMT3 | chr11: 63974820-63974959, strand = + |
| FGF14 | chr13: 103046888-103046991, strand = + |
| FU34208 | chr3: 194208249-194208355, strand = + |
| FU45983 | chr10: 8097592-8097699, strand = + |
| GRIN2D | chr19: 48918160-48918300, strand = − |
| HIST1H2BE | chr6: 26184248-26184340, strand = + |
| HOPX | chr4: 57521932-57522261 5′pad = 0 3′pad = 0 strand = − |
| IFFO1 | chr12: 6665277-6665348 strand = + |
| HOXA9 | chr7: 27205002-27205102, strand = − |
| HOXB2 | chr17: 46620545-46620639, strand = − |
| KLHDC7B | chr22: 50987199-50987256, strand = + |
| LOC100129726 | chr2: 43451705-43451810, strand = + |
| MATK | chr19: 3786127-3786197, strand = + |
| MAX.chr10.22541891-22541946 | chr10: 22541881-22541975, strand = + |
| MAX.chr10.22624430-22624544 | chr10: 22624411-22624553, strand = − |
| MAX. chr12.52652268-52652362 | chr12: 52652262-52652377, strand = − |
| MAX.chr16.50875223-50875241 | chr16: 50875167-50875274, strand = − |
| MAX.chr19.16394489-16394575 | chr19: 16394457-16394593, strand = − |
| MAX.chr19.37288426-37288480 | range = chr19: 37288396-37288512, strand = − |
| MAX.chr8.124173236-124173370 | chr8: 124173231-124173386, strand = − |
| MAX.chr8.145105646-145105653 | chr8: 145105572-145105685, strand = − |
| MAX_Chr1.110 | chr1: 110627118-110627224 strand = − |
| NFIX | chr19: 13207426-13207513, strand = + |
| NKX2-6 | chr8: 23564052-23564145, strand = − |
| OPLAH | chr8: 145106777-145106865, strand = − |
| PARP15 | chr3: 122296692-122296805, strand = + |
| PRDM14 | chr8: 70981945-70982039, strand = − |
| PRKAR1B | chr7: 644172-644237, strand = + |
| PRKCB_28 | chr16: 23847607-23847698, strand = − |
| PTGDR | chr14: 52735270-52735400, strand = − |
| PTGDR_9 | chr14: 52735221-52735300, strand = + |
| RASSF1 | chr3: 50378408-50378550, strand = − |
| SHOX2 | chr3: 157821263-157821382, strand = − |
| SHROOM1 | chr5: 132161371-132161425, strand = + |
| SIPR4 | chr19: 3179921-3180068 strand = − |
| SKI | chr1: 2232328-2232423, strand = + |
| SLC12A8 | chr3: 124860704-124860791, strand = + |
| SOBP | chr6: 107956176-107956234, strand = + |
| SP9 | chr2: 175201210-175201341, strand = − |
| SPOCK2 | chr10: 73847236-73847324, strand = − |
| ST8SIA1 | chr12: 22487518-22487630, strand = + |
| ST8SIA1_22 | chr12: 22486873-22487009, strand = − |
| SUCLG2 | chr3: 67706477-677065610, strand = − |
| TBX15 Region 1 | chr1: 119527066-119527655, strand = + |
| TBX15 Region 2 | chr1: 119532813-119532920strand = − |
| TRH | chr3: 129693481-129693580, strand = + |
| TSC22D4 | chr7: 100075328-100075445, strand = − |
| ZDHHC1 | chr16: 67428559-67428628, strand = − |
| ZMIZ1 | chr10: 81002910-81003005, strand = + |
| ZNF132 | chr19: 58951403-58951529, strand = − |
| ZNF329 | chr19: 58661889- 58662028, strand = − |
| ZNF671 | chr19: 58238790-58238906, strand = + |
| ZNF781 | ch19: 38183018-38183137, strand = − |

Analyzing Selected Markers for Cross-Reactivity with Buffy Coat.

### 1) Buffy Coat Screening

Markers from the list above were screened on DNA extracted from buffy coat obtained from 10 mL blood of a healthy patient. DNA was extracted using Promega Maxwell RSC system (Promega Corp., Fitchburg, Wis.) and converted using Zymo EZ DNA Methylation™ Kit (Zymo Research, Irvine, Calif.). Using biplexed reaction with bisulfite-converted P3-actin DNA (“BTACT”), and using approximately 40,000 strands of target genomic DNA, the samples were tested using a QuARTS flap endonuclease assay as described above, to test for cross reactivity. Doing so, the assays for 3 markers showed significant cross reactivity:

| | % Cross |
|---|---|
| Marker | reactivity |
| HIST1H2B | 72.93% |
| chr7_636 | 3495.47% |
| chr5_132 | 0.20% |

### 2) Tissue Screening

264 tissue samples were obtained from various commercial and non-commercial sources (Asuragen, BioServe, ConversantBio, Cureline, Mayo Clinic, M D Anderson, and PrecisionMed), as shown below in Table 2.

| No. of cases | Pathology | Subtype | Details |
|---|---|---|---|
| 82 | Normal | NA | 68 smokers, 34 never |
| 37 | Normal | benign nodule | smokers, 17 smoking |
| 7 | NSCLC | bronchioalveolar | unknown |
| 13 | NSCLC | large cell | |
| 2 | NSCLC | neuroendocrine | |
| 42 | NSCLC | squamous cell | |
| 68 | NSCLC | adenocarcinomas | |
| 4 | SCLC | small cell | |
| 9 | NSCLC | carcinoid | |

Tissue sections were examined by a pathologist, who circled histologically distinct lesions to direct the micro-dissection. Total nucleic acid extraction was performed using the Promega Maxwell RSC system. Formalin-fixed, paraffin-embedded (FFPE) slides were scraped and the DNA was extracted using the Maxwell® RSC DNA FFPE Kit (#AS1450) using the manufacturer's procedure but skipping the RNase treatment step. The same procedure was used for FFPE curls. For frozen punch biopsy samples, a modified procedure using the lysis buffer from the RSC DNA FFPE kit with the Maxwell® RSC Blood DNA kit (#AS1400) was utilized omitting the RNase step. Samples were eluted in 10 mM Tris, 0.1 mM EDTA, pH 8.5 and 10 uL were used to setup 6 multiplex PCR reactions.

The following multiplex PCR primer mixes were made at 10× concentration (10×=2 μM each primer):
Multiplex PCR reaction 1 consisted of each of the following markers: BARX1, LOC100129726, SPOCK2, TSC22D4, PARP15, MAX.chr8.145105646-145105653, ST8SIA1_22, ZDHHC1, BIN2_Z, SKI, DNMT3A, BCL2L11, RASSF1, FERMT3, and BTACT.
Multiplex PCR reaction 2 consisted of each of the following markers: ZNF671, ST8SIA1, NKX6-2, SLC12A8, FAM59B, DIDO1, MAX_Chr1.110, AGRN, PRKCB_28, SOBP, and BTACT.
Multiplex PCR reaction 3 consisted of each of the following markers: MAX.chr10.22624430-22624544, ZMIZ1, MAX.chr8.145105646-145105653, MAX.chr10.22541891-22541946, PRDM14, ANGPT1, MAX.chr16.50875223-50875241, PTGDR_9, ANKRD13B, DOCK2, and BTACT.
Multiplex PCR reaction 4 consisted of each of the following markers: MAX.chr19.16394489-16394575, HOXB2, ZNF132, MAX.chr19.37288426-37288480, MAX.chr12.52652268-52652362, FLJ45983, HOXA9, TRH, SP9, DMRTA2, and BTACT.
Multiplex PCR reaction 5 consisted of each of the following markers: EMX1, ARHGEF4, OPLAH, CYP26C1, ZNF781, DLX4, PTGDR, KLHDC7B, GRIN2D, chr17_737, and BTACT.
Multiplex PCR reaction 6 consisted of each of the following markers: TBX15, MATK, SHOX2, BCAT1, SUCLG2, BIN2, PRKAR1B, SHROOM1, SIPR4, NFIX, and BTACT.

Each multiplex PCR reaction was setup to a final concentration of 0.2 μM reaction buffer, 0.2 μM each primer, 0.05 μM Hotstart Go Taq (5 U/μL), resulting in 40 μL of master mix that was combined with 10 μL of DNA template for a final reaction volume of 50 μL.

The thermal profile for the multiplex PCR entailed a pre-incubation stage of 95° for 5 minutes, 10 cycles of amplification at 95° for 30 seconds, 64° for 30 seconds, 72° for 30 seconds, and a cooling stage of 4° that was held until further processing. Once the multiplex PCR was complete, the PCR product was diluted 1:10 using a diluent of 20ng/μL of fish DNA (e.g., in water or buffer, see U.S. Pat. No. 9,212,392, incorporated herein by reference) and 10 μL of diluted amplified sample were used for each QuARTS assay reaction.

Each QuARTS assay was configured in triplex form, consisting of 2 methylation markers and BTACT as the reference gene.
From multiplex PCR product 1, the following 7 triplex QuARTS assays were run: (1) BARX1, LOC100129726, BTACT; (2) SPOCK2, TSC22D4, BTACT; (3) PARP15, MAXchr8145105646-145105653, BTACT; (4) ST8SIA1_22, ZDHHC1, BTACT; (5) BIN2_Z, SKI, BTACT; (6) DNMT3A, BCL2L11, BTACT; (7) RASSF1, FERMT3, and BTACT.
From multiplex PCR product 2, the following 5 triplex QuARTS assays were run: (1) ZNF671, ST8SIA1, BTACT; (2) NKX6-2, SLC12A8, BTACT; (3) FAM59B, DIDO1, BTACT; (4) MAX_Chr1110, AGRN, BTACT; (5) PRKCB_28, SOBP, and BTACT.
From multiplex PCR product 3, the following 5 triplex QuARTS assays were run: (1) MAXchr1022624430-22624544, ZMIZ1, BTACT; (2) MAXchr8145105646-145105653, MAXchr1022541891-22541946, BTACT; (3) PRDM14, ANGPT1, BTACT; (4) MAXchr1650875223-50875241, PTGDR_9, BTACT; (5) ANKRD13B, DOCK2, and BTACT.
From multiplex PCR product 4, the following 5 triplex QuARTS assays were run: (1) MAXchr1916394489-16394575, HOXB2, BTACT; (2) ZNF132, MAXchr1937288426-37288480, BTACT; (3) MAXchr1252652268-52652362, FLJ45983, BTACT; (4) HOXA9, TRH, BTACT; (5) SP9, DMRTA2, and BTACT.
From multiplex PCR product 5, the following 5 triplex QuARTS assays were run: (1) EMX1, ARHGEF4, BTACT; (2) OPLAH, CYP26C1, BTACT; (3) ZNF781, DLX4, BTACT; (4) PTGDR, KLHDC7B, BTACT; (5) GRIN2D, chr17_737, and BTACT.
From multiplex PCR product 6, the following 5 triplex QuARTS assays were run: (1) TBX15, MATK, BTACT; (2) SHOX2, BCAT1, BTACT; (3) SUCLG2, BIN2, BTACT; (4) PRKAR1B, SHROOM1, BTACT; (5) SIPR4, NFIX, and BTACT.

### 3) Data Analysis:

For tissue data analysis, markers that were selected based on RRBS criteria with <0.5% methylation in normal tissue and >10% methylation in cancer tissue were included. This resulted in 51 markers for further analysis.

To determine marker sensitivities, the following was performed:
1. % methylation for each marker was computed by dividing strand values obtained for that specific marker by the strand values of ACTB (β-actin).
2. The maximum % methylation for each marker was determined on normal tissue. This is defined as 100% specificity.
3. The cancer tissue positivity for each marker was determined as the number of cancer tissues that had greater than the maximum normal tissue % methylation for that marker.
   The sensitivities for the 51 markers are shown below.

TABLE 2

| | Maximum % | | | |
|---|---|---|---|---|
| | methylation | Cancer (N = 136) | | |
| Marker | for normal | # Negative | # Positive | sensitivity |
| BARXI | 1.665 | 66 | 70 | 51% |
| LOC100129726 | 1.847 | 109 | 27 | 20% |
| SPOCK2 | 0.261 | 86 | 50 | 37% |
| TSC22D4 | 0.618 | 70 | 66 | 49% |
| MAX.chr8.124 | 0.293 | 45 | 91 | 67% |
| RASSF1 | 1.605 | 79 | 57 | 42% |
| ZNF671 | 0.441 | 73 | 63 | 46% |
| ST8SIA1 | 1.56 | 119 | 17 | 13% |
| NKX6_2 | 15.58 | 102 | 34 | 25% |
| FAM59B | 0.433 | 85 | 51 | 38% |
| DIDO1 | 2.29 | 93 | 43 | 32% |
| MAX_Chr1.110 | 0.076 | 85 | 51 | 38% |
| AGRN | 2.16 | 66 | 70 | 51% |
| SOBP | 38.5 | 110 | 26 | 19% |
| MAX_chr10.226 | 0.7 | 52 | 84 | 62% |
| ZMIZ1 | 0.025 | 72 | 64 | 47% |
| MAX_chr8.145 | 5.56 | 57 | 79 | 58% |
| MAX_chr10.225 | 0.77 | 72 | 64 | 47% |
| PRDM14 | 0.22 | 35 | 101 | 74% |
| ANGPT1 | 1.6 | 99 | 37 | 27% |
| MAX.chr16.50 | 0.27 | 92 | 44 | 32% |
| PTGDR_9 | 4.62 | 82 | 54 | 40% |
| ANKRD13B | 7.03 | 93 | 43 | 32% |
| DOCK2 | 0.001 | 71 | 65 | 48% |
| MAX_chr19.163 | 0.61 | 56 | 80 | 59% |
| ZNF132 | 1.3 | 83 | 53 | 39% |
| MAX chr19.372 | 0.676 | 79 | 57 | 42% |
| HOXA9 | 16.7 | 53 | 83 | 61% |
| TRH | 2.64 | 61 | 75 | 55% |
| SP9 | 14.99 | 75 | 61 | 45% |
| DMRTA2 | 7.9 | 55 | 81 | 60% |
| ARHGEF4 | 7.41 | 113 | 23 | 17% |
| CYP26C1 | 39.2 | 101 | 35 | 26% |
| ZNF781 | 5.28 | 44 | 92 | 68% |
| PTGDR | 6.13 | 76 | 60 | 44% |
| GRIN2D | 16.1 | 113 | 23 | 17% |
| MATK | 0.04 | 93 | 43 | 32% |
| BCAT1 | 0.64 | 75 | 61 | 45% |
| PRKCB_28 | 1.68 | 57 | 79 | 58% |
| ST8SIA_22 | 1.934 | 55 | 81 | 60% |
| FLI45983 | 8.34 | 39 | 97 | 71% |
| DLX4 | 15.1 | 41 | 95 | 70% |
| SHOX2 | 7.48 | 32 | 104 | 76% |
| EMX1 | 11.34 | 34 | 102 | 75% |
| HOXB2 | 0.114 | 61 | 75 | 55% |
| MAX.chr12.526 | 5.58 | 34 | 102 | 75% |
| BCL2L11 | 10.7 | 44 | 92 | 68% |
| OPLAH | 5.11 | 29 | 107 | 79% |
| PARP15 | 3.077 | 42 | 94 | 69% |
| KLHDC7B | 8.86 | 38 | 98 | 72% |
| SLC12A8 | 0.883 | 34 | 102 | 75% |

Combinations of markers may be used to increase specificity and sensitivity. For example, a combination of the 8 markers SLC12A8, KLHDC7B, PARP15, OPLAH, BCL2L11, MAX.chr12.S26, HOXB2, and EMX1 resulted in 98.5% sensitivity (134/136 cancers) for all of the cancer tissues tested, with 100% specificity.

In some embodiments, markers are selected for sensitive and specific detection associated with a particular type of lung cancer tissue, e.g., adenocarcinoma, large cell carcinoma, squamous cell carcinoma, or small cell carcinoma, e.g., by use of markers that show sensitivity and specificity for particular cancer types or combinations of types.

This panel of methylated DNA markers assayed on tissue achieves extremely high discrimination for all types of lung cancer while remaining negative in normal lung tissue and benign nodules. Assays for this panel of markers can be also be applied to blood or bodily fluid-based testing, and finds applications in, e.g., lung cancer screening and discrimination of malignant from benign nodules.

### Example 3

### Testing a 30-Marker Set on Plasma Samples

From the list of markers in Example 2, 30 markers were selected for use in testing DNA from plasma samples from 295 subjects (64 with lung cancer, 231 normal controls. DNA was extracted from 2 mL of plasma from each subject and treated with bisulfite as described in Example 1. Aliquots of the bisulfite-converted DNA were used in two multiplex QuARTS assays, as described in Example 1. The markers selected for analysis are:
1, BARX1
2, BCL2L11
3. BIN2_Z
4, CYP26C1
5, DLX4
6, DMRTA2
7. DNMT3A
8. EMX1
9. FERMT3
10, FLJ45983
11, HOXA9
12, KLHDC7B
13, MAX.chr10.22624430-22624544
14, MAX.chr12.52652268-52652362
15, MAX.chr8.124173236-124173370
16, MAX.chr8.145105646-145105653
17. NFIX
18. OPLAH
19. PARP15
20. PRKCB_28
21. SIPR4
22, SHOX2
23, SKI
24. SLC12A8
25, SOBP
26. SP9
27. SUCLG2
28, TBX15
29, ZDHHC1
30, ZNF781

The target sequences, bisulfite converted target sequences, and the assay oligonucleotides for these markers were as shown in FIG. 5. The primers and flap oligonucleotides (probes) used for each converted target were as follows:

TABLE 3

| | Oligonucleotide | | | SEQ ID |
|---|---|---|---|---|
| Marker | Name | Component | Sequence (5′-3′) | NO: |
| BARX1 | BARX1_FP | Forward | CGTTAATTTGTTAGATAGAGGGCG | 23 |
| | | Primer | | |
| | BARX1_RP | Reverse | ACGATCGTCCGAACAACC | 24 |
| | | Primer | | |
| | BARX1_PB_A5 | Flap Oligo. | CCACGGACGCGCCTACGAAAA/3C6/ | 25 |
| SLC12A8 | SLC12A8_FP | Forward | TTAGGAGGGTGGGGTTCG | 289 |
| | | Primer | | |
| | SLC12A8_RP | Reverse | CTTTCCTCGCAAAACCGC | 290 |
| | | Primer | | |
| | SLC12A8_Pb_A1 | Flap Oligo. | CCACGGACGGGAGGGCGTAGG/3C6/ | 291 |
| PARP15 | PARP15_FP | Forward | GGTTGAGTTTGGGGTTCG | 236 |
| | | Primer | | |
| | PARP15_RP | Reverse | CGTAACGTAAAATCTCTACGCCC | 237 |
| | | Primer | | |
| | PARP15_Pb_A5 | Flap Oligo. | CCACGGACGCGCTCGAACTAC/3C6/ | 238 |
| MAX.Chr8. | MAX.Chr8.124_ | Forward | GGTTGAGGTTTTCGGGTTTTTAG | 203 |
| 124 | FP | Primer | | |
| | MAX.Chr8.124_ | Reverse | CCTCCCCACGAAATCGC | 204 |
| | RP | Primer | | |
| | MAX.Chr8.124_ | Flap Oligo. | CGCCGAGGGCGGGTTTTCGT/3C6/ | 205 |
| | Pb_A1 | | | |
| SHOX2 | SHOX2_FP | Forward | GTTCGAGTTTAGGGGTAGCG | 269 |
| | | Primer | | |
| | SHOX2_RP | Reverse | CCGCACAAAAAACCGCA | 270 |
| | | Primer | | |
| | SHOX2_Pb_A5 | Flap Oligo. | CCACGGACGATCCGCAAACGC/3C6/ | 271 |
| ZDHHC1 | ZDHHC1FP | Forward | GTCGGGGTCGATAGTTTACG | 348 |
| | | Primer | | |
| | ZDHHC1RP_V3 | Reverse | ACTCGAACTCACGAAAACG | 349 |
| | | Primer | | |
| | ZDHHClProbe_ | Flap Oligo. | CGCCGAGGGACGAACGCACG/3C6/ | 350 |
| | v3_A1 | | | |
| BIN2_Z | BIN2_FP_Z | Forward | GGGTTTATTTTTAGGTAGCGTTCG | 50 |
| | | Primer | | |
| | BIN2_RP_Z | Reverse | CGAAATTTCGAACAAAAATTAAAACTCGA | 51 |
| | | Primer | | |
| | BIN2_Pb_A5_Z | Flap Oligo. | CCACGGACGGTTCGAGGTTAG/3C6/ | 52 |
| SKI | SKI_FP | Forward | ACGGTTTTTTCGTTATTTTTACGGG | 279 |
| | | Primer | | |
| | SKI_RP | Reverse | CAACGCCTAAAAACACGACTC | 280 |
| | | Primer | | |
| | SKI_Pb_A1 | Flap Oligo. | CGCCGAGGGGCGGTTGTTGG/3C6/ | 281 |
| DNMT3A | DNMT3A_FP | Forward | GTTACGAATAAAGCGTTGGCG | 93 |
| | | Primer | | |
| | DNMT3A_RP | Reverse | AACGAAACGTCTTATCGCGA | 94 |
| | | Primer | | |
| | DNMT3A_Pb_A5 | Flap Oligo. | CCACGGACGGAGTGCGCGTTC/3C6/ | 95 |
| BC2L11 | BCL2L11_FP | Forward | CGTAATGTTTCGCGTTTTTCG | 35 |
| | | Primer | | |
| | BCL2L11_RP | Reverse | ACTTTCTTCTACGTAATTCTTTTCCGA | 36 |
| | | Primer | | |
| | BCL2L11_Pb_A1 | Flap Oligo. | CGCCGAGGGCGGGGTCGGGC/3C6/ | 37 |
| TBX15 | TBX15_Reg2_FP | Forward | AGGAAATTGCGGGTTTTCG | 332 |
| | | Primer | | |
| | TBX15_Reg2_RP | Reverse | CCAAAAATCGTCGCTAAAAATCAAC | 334 |
| | | Primer | | |
| | TBX15_Reg2_Pb_ | Flap Oligo. | CCACGGACGCGCGCATTCACT/3C6/ | 335 |
| | A5 | | | |
| FERMT3 | FERMT3_FP | Forward | GTTTTCGGGGATTATATCGATTCG | 118 |
| | | Primer | | |
| | FERMT3_RP | Reverse | CCCAATAACCCGCAAAATAACC | 119 |
| | | Primer | | |
| | FERMT3_Pb_A1 | Flap Oligo. | CGCCGAGGCGACTCGACCTC/3C6/ | 120 |
| PRKCB_28 | PRKCB_28_FP | Forward | GGAAGGTGTTTTGCGCG | 249 |
| | | Primer | | |
| | PRKCB_28_RP | Reverse | CTTCTACAACCACTACACCGA | 250 |
| | | Primer | | |
| | PRKCB_28_Pb_ | Flap Oligo. | CCACGGACGGCGCGCGTTTAT/3C6/ | 251 |
| | A5 | | | |
| SOBP_HM | SOBP_HM_FP | Forward | TTTCGGCGGGTTTCGAG | 294 |
| | | Primer | | |
| | SOBP_HM_RP | Reverse | CGTACCGTTCACGATAACGT | 295 |
| | | Primer | | |
| | SOBP_HM_Pb_ | Flap Oligo. | CGCCGAGGGGCGGTCGCGGT/3C6/ | 296 |
| | A1 | | | |
| MAX.chr8. | MAX.Chr8.145_ | Forward | GCGGTATTAGTTAGAGTTTTAGTCG | 211 |
| 145 | FP | Primer | | |
| | MAX.Chr8.145_ | Reverse | ACAACCCTAAACCCTAAATATCGT | 212 |
| | RP | Primer | | |
| | MAX.Chr8.145_ | Flap Oligo. | CCACGGACGGACGGCGTTTTT/3C6/ | 213 |
| | Pb_A5 | | | |
| MAX.chr10. | MAX.Chr10.226_ | Forward | GGGAAATTTGTATTTCGTAAAATCG | 178 |
| 226 | FP | Primer | | |
| | MAX.Chr10.226_ | Reverse | ACAACTAACTTATCTACGTAACATCGT | 179 |
| | RP | Primer | | |
| | MAX_Chr10.226_ | Flap Oligo. | CGCCGAGGGCGGTTAAGAAA/3C6/ | 180 |
| | Pb_A1 | | | |
| MAX.chr12. | MAX.Chr12.52_ | Forward | TCGTTCGTTTTTGTCGTTATCG | 183 |
| 52 | FP | Primer | | |
| | MAX.Chr12.52_ | Reverse | AACCGAAATACAACTAAAAACGC | 184 |
| | RP | Primer | | |
| | MAX.Chr12. | Flap Oligo. | CCACGGACGCGAACCCCGCAA/3C6/ | 185 |
| | 52PbA1 | | | |
| FLJ45983 | FLJ45983_FP | Forward | GGGCGCGAGTATAGTCG | 133 |
| | | Primer | | |
| | FLJ45983_RP | Reverse | CAACGCGACTAATCCGC | 134 |
| | | Primer | | |
| | FLJ45983_Pb_A1 | Flap Oligo. | CGCCGAGGCCGTCACCTCCA/3C6/ | 135 |
| HOXA9 | HOXA9_FP | Forward | TTGGGTAATTATTACGTGGATTCG | 148 |
| | | Primer | | |
| | HOXA9_RP | Reverse | ACTCATCCGCGACGTC | 149 |
| | | Primer | | |
| | HOXA9_Pb_A5 | Flap Oligo. | CCACGGACGCGACGCCCAACA/3C6/ | 150 |
| EMX1 | EMX1_FP | Forward | GGCGTCGCGTTTTTTAGAGAA | 108 |
| | | Primer | | |
| | EMX1_RP | Reverse | TTCCTTTTCGTTCGTATAAAATTTCGTT | 109 |
| | | Primer | | |
| | EMX1PbA1 | Flap Oligo. | CGCCGAGGATCGGGTTTTAG/3C6/ | 110 |
| SP9 | SP9_FP | Forward | TAGCGTCGAATGGAAGTTCGA | 315 |
| | | Primer | | |
| | SP9_RP | Reverse | GCGCGTAAACATAACGCACC | 317 |
| | | Primer | | |
| | SP9_Pb_A5 | Flap Oligo. | CCACGGACGCCGTACGAATCC/3C6/ | 318 |
| DMRTA2 | DMRTA2_FP | Forward | TGGTGTTTACGTTCGGTTTTCGT | 88 |
| | | Primer | | |
| | DMRTA2_RP | Reverse | CCGCAACAACGACGACC | 89 |
| | | Primer | | |
| | DMRTA2_Pb_A1 | Flap Oligo. | CGCCGAGGCGAACGATCACG/3C6/ | 90 |
| OPLAH | FPrimerOPLAH | Forward | cGTcGcGTTTTTcGGTTATACG | 231 |
| | | Primer | | |
| | RPrimerOPLAH | Reverse | CGCGAAAACTAAAAAACCGCG | 232 |
| | | Primer | | |
| | ProbeA5OPLAH | Flap Oligo. | CCACGGACG-GCACCGTAAAAC/3C6/ | 233 |
| CYP26C1 | CYP26C1_FP | Forward | TGGTTTTTTGGTTATTTCGGAATCGT | 70 |
| | | Primer | | |
| | CYP26C1_RP | Reverse | GCGCGTAATCAACGCTAAC | 71 |
| | | Primer | | |
| | CYP26C1_Pb_A1 | Flap Oligo. | CGCCGAGGCGACGATCTAAC/3C6/ | 72 |
| ZNF781 | ZNF781F.primer | Forward | CGTTTTTTTGTTTTTCGAGTGCG | 373 |
| | | Primer | | |
| | ZNF781R.primer | Reverse | TCAATAACTAAACTCACCGCGTC | 374 |
| | | Primer | | |
| | ZNF781probe.A5 | Flap Oligo. | CCACGGACGGCGGATTTATCG/3C6/ | 375 |
| DLX4 | DLX4_FP | Forward | TGAGTGCGTAGTGTTTTCGG | 80 |
| | | Primer | | |
| | DLX4_RP | Reverse | CTCCTCTACTAAAACGTACGATAAACA | 81 |
| | | Primer | | |
| | DLX4_Pb_A1 | Flap Oligo. | CGCCGAGGATCGTATAAAAC/3C6/ | 82 |
| SUCLG2 | SUCLG2_HM_FP | Forward | TCGTGGGTTTTTAATCGTTTCG | 321 |
| | | Primer | | |
| | SUCLG2_HM_RP | Reverse | TCACGCCATCTTTACCGC | 322 |
| | | Primer | | |
| | SUCLG2_HM_Pb_ | Flap Oligo. | CCACGGACGCGAAAATCTACA/3C6/ | 323 |
| | A5 | | | |
| KLHDC7B | KLHDC7B_FP | Forward | AGTTTTCGGGTTTTGGAGTTCGTTA | 158 |
| | | Primer | | |
| | KLHDC7B_RP | Reverse | CCAAATCCAACCGCCGC | 159 |
| | | Primer | | |
| | KLHDC7B_Pb_A1 | Flap Oligo. | CGCCGAGGACGGCGGTAGTT/3C6/ | 160 |
| S1PR4_HM | S1PR4_HM_FP | Forward | TTATATAGGCGAGGTTGCGT | 284 |
| | | Primer | | |
| | S1PR4_HM_RP | Reverse | CTTACGTATAAATAATACAACCACCGAATA | 285 |
| | | Primer | | |
| | S1PR4_HM_Pb_ | Flap Oligo. | CCACGGACGACGTACCAAACA/3C6/ | 286 |
| | A5 | | | |
| NFIX_HM | NFIX_HM_FP | Forward | TGGTTCGGGCGTGACGCG | 221 |
| | | Primer | | |
| | NFIX_HM_RP | Reverse | TCTAACCCTATTTAACCAACCGA | 222 |
| | | Primer | | |
| | NFIX_HM_Pb_A1 | Flap Oligo. | CGCCGAGGGCGGTTAAAGTG/3C6/ | 223 |
| Reference | Oligonucleotide | | | |
| DNAs | Name | Component | Sequence (5′-3′) | |
| Zebrafish | ZF_RASSF1_FP | BT Forward | TGCGTATGGTGGGCGAG | 394 |
| Synthetic | | Primer | | |
| (RASSF1) | ZF_RASSF1_RP | BT Reverse | CCTAATTTACACGTCAACCAATCGAA | |
| BT | | Primer | | |
| converted) | ZF_RASSF1_Pb_ | BT Flap | CCACGGACGGCGCGTGCGTTT/3C6/ | 395 |
| † | A5 | Oligo. | | |
| B3GALT6* | B3GALT6_FP_V2 | Forward | GGTTTATTTTGGTTTTTTGAGTTTTCGG | 386 |
| | | Primer | | |
| | B3GALT6_RP | Reverse | TCCAACCTACTATATTTACGCGAA | 387 |
| | | Primer | | |
| | B3GALT6_Pb_A1 | Flap Oligo. | CCACGGACGGCGGATTTAGGG/3C6/ | 388 |
| BTACT | ACTB_BT_FP65 | Forward | GTGTTTGTTTTTTTGATTAGGTGTTTAAGA | 381 |
| | | Primer | | |
| | ACTB_BT_RP65 | Reverse | CTTTACACCAACCTCATAACCTTATC | 382 |
| | | Primer | | |
| | ACTBBTPbA3 | Flap Oligo. | GACGCGGAGATAGTGTTGTGG/3C6/ | 383 |

*The B3GALT6 marker is used as both a cancer methylation marker and as a
reference target. See U.S. patent application Ser. No. 62/364,082, filed 07/19/16, which is incorporated herein by reference in its entirety.
*For zebrafish reference DNA see U.S.patent application Ser. No. 62/364,049, filed 07/19/16, which is incorporated herein by reference in its entirety.

The DNA prepared from plasma as described above was amplified in two multiplexed pre-amplification reactions, as described in Example 1. The multiplex pre-amplification reactions comprised reagents to amplify the following marker combinations.

TABLE 4

| Multiplex Mix 1 | Multiplex Mix 2 |
|---|---|
| B3GALT6 (reference) | B3GALT6 (reference) |
| ZF_RASSF1 (reference) | ZF_RASSF1 (reference) |
| BARX1 | CYP26C1 |
| BCL2L11 | DLX4 |
| BCL2L11 | DMRTA2 |
| BIN2_Z | EMX1 |
| DNMT3A | HOXA9 |
| FERMT3 | KLHDC7B |
| PARP15 | MAX.chr8.125 |
| PRKCB_28 | MAX_chr10.226 |
| SHOX2 | NFIX |
| SLC12A8 | OPLAH |
| SOBP | S1PR4 |
| TBX15_Reg2 | SP9 |
| ZDHHC1 | SUCLG2 |
| | ZNF781 |

Following pre-amplification, aliquots of the pre-amplified mixtures were diluted 1:10 in 10 mM Tris HCl, 0.1 mM EDTA, then were assayed in triplex QuARTS PCR-flap assays, as described in Example 1. The Group 1 triplex reactions used pre-amplified material from Multiplex Mix 1, and the Group 2 reactions used the pre-amplified material from Multiplex Mix 2. The triplex combinations were as follows:

Group 1:

| | |
|---|---|
| ZF_RASSF1-B3GALT6-BTACT | (ZBA Triplex) |
| BARX1-SLC12A8-BTACT | (BSA2 Triplex) |
| PARP15-MAX. chr8.124-BTACT | (PMA Triplex) |
| SHOX2-ZDHHC1-BTACT | (SZA2 Triplex) |
| BIN2_Z-SKI-BTACT | (BSA Triplex) |
| DNMT3A-BCL2L11-BTACT | (DBA Triplex) |
| TBX15-FERMT3 -BTACT | (TFA Triplex) |
| PRKCB_28-SOBP-BTACT | (PSA2 Triplex) |

Group 2:

| | |
|---|---|
| ZF_RASSF1-B3GALT6-BTACT | (ZBA Triplex) |
| MAX.chr8.145-MAX_chr10.226-BTACT | (MMA2 Triplex) |
| MAX.chr12.526-FLJ45983-BTACT | (MFA Triplex) |
| HOXA9-EMX1-BTACT | (HEA Triplex) |
| SP9-DMRTA2-BTACT | (SDA Triplex) |
| OPLAH-CYP26C1-BTACT | (OCA Triplex) |
| ZNF781-DLX4-BTACT | (ZDA Triplex) |
| SUCLG2-KLHDC7B-BTACT | (SKA Triplex) |
| S1PR4-NFIX-BTACT | (SNA Triplex) |

Each triplex acronym uses the first letter of each gene name (for example, the combination of HOXA9-EMX1-BTACT=“HEA”). If an acronym is repeated for a different combination of markers or from another experiment, the second grouping having that acronym includes the number 2. The dye reporters used on the FRET cassettes for each member of the triplexes listed above is FAM-HEX-Quasar670, respectively.

Plasmids containing target DNA sequences were used to calibrate the quantitative reactions. For each calibrator plasmid, a series of 10× calibrator dilution stocks, having from 10 to 10⁶ copies of the target strand per μl in fish DNA diluent (20 ng/mL fish DNA in 10 mM Tris-HCl, 0.1 mM EDTA) were prepared. For triplex reactions, a combined stock having plasmids that contain each of the targets of the triplex were used. A mixture having each plasmid at 1×10⁵ copies per μL was prepared and used to create a 1:10 dilution series. Strands in unknown samples were back calculated using standard curves generated by plotting Cp vs Log (strands of plasmid).

Using receiver operating characteristic (ROC) curve analysis, the area under the curve (AUC) for each marker was calculated and is shown in the table below, sorted by Upper 95 Pct Coverage Interval.

TABLE 5

| | | Sensitivity at |
|---|---|---|
| Marker Name | AUC | 90% specificity |
| CYP26C1 | 0.940 | 80% |
| SOBP | 0.929 | 80% |
| SHOX2 | 0.905 | 73% |
| SUCLG2 | 0.905 | 64% |
| NFIX | 0.895 | 63% |
| ZDHHC1 | 0.890 | 69% |
| BIN2_Z | 0.872 | 59% |
| DLX4 | 0.856 | 56% |
| FLI45983 | 0.834 | 67% |
| HOXA9 | 0.824 | 53% |
| TBX15 | 0.813 | 53% |
| ACTB | 0.803 | 50% |
| S1PR4 | 0.802 | 55% |
| SP9 | 0.782 | 38% |
| FERMT3 | 0.773 | 36% |
| ZNF781 | 0.769 | 55% |
| B3GALT6 | 0.746 | 39% |
| BTACT | 0.742 | 44% |
| BCL2L11 | 0.732 | 39% |
| PARP15 | 0.673 | 31% |
| DNMT3A | 0.689 | 20% |
| MAX.chr12.526 | 0.668 | 33% |
| MAX.chr10.226 | 0.671 | 30% |
| SLC12A8 | 0.655 | 19% |
| BARX1 | 0.663 | 25% |
| KLHDC7B | 0.604 | 10% |
| OPLAH | 0.571 | 14% |
| MAX.chr8.145 | 0.572 | 16% |
| SKI | 0.521 | 14% |

The markers worked very well in distinguishing samples from cancer patients from samples from normal subjects (see ROC table, above). Use of the markers in combination improved sensitivity. For example, using a logistic fit of the data and a six-marker fit using markers SHOX2, SOBP, ZNF781, BTACT, CYP26CL, and DLX4, ROC curve analysis gave an area under the curve (AUC) of 0.973. Using this 6-marker fit, sensitivity of 92.2% is obtained at 93% specificity. Using SHOX2, SOBP, ZNF781, CYP26CL, SUCLG2, and SKI gave an ROC curve with an AUC of 0.97982.

### Example 4

Archival plasmas from a second independent study group were tested in blinded fashion. Lung cancer cases and controls (apparently healthy smokers) for each group were balanced on age and sex (23 cases, 80 controls). Using multiplex PCR followed by QuARTS (Quantitative Allele-Specific Real-time Target and Signal amplification) assay as described in Example 1, a post-bisulfite quantification of methylated DNA markers on DNA extracted from plasma was performed. Top individual methylation markers from Example 3 were tested in this experiment to identify optimal marker panels for lung cancer detection (2 ml/patient).

Results: 13 high performance methylated DNA markers were tested (CYP26C1, SOBP, SUCLG2, SHOX2, ZDHHC1, NFIX FLJ45983, HOXA9, B3GALT6, ZNF781, SP9, BARX1, and EMX1). Data were analyzed using two methods: a logistic regression fit and a regression partition tree approach. The logistic fit model identified a 4-marker panel (ZNF781, BARX1, EMX1, and SOBP) with an AUC of 0.96 and an overall sensitivity of 91% and 90% specificity. Analysis of the data using a regression partition tree approach identified 4 markers (ZNF781, BARX1, EMX1, and HOXA9) with AUC of 0.96 and an overall sensitivity of 96% and specificity of 94%. For both approaches, B3GALT6 was used as a standardizing marker of total DNA input. These panels of methylated DNA markers assayed in plasma achieved high sensitivity and specificity for all types of lung cancer.

### Example 5

### Differentiating Lung Cancers

Using the methods described above, methylation markers are selected that exhibit high performance in detecting methylation associated with specific types of lung cancer.

For a subject suspected of having lung cancer, a sample is collected, e.g., a plasma sample, and DNA is isolated from the sample and treated with bisulfite reagent, e.g., as described in Example 1. The converted DNA is analyzed using a multiplex PCR followed by QuARTS flap endonuclease assay as described in Example 1, configured to provide different identifiable signals for different methylation markers or combinations of methylation markers, thereby providing data sets configured to specifically identify the presence of one or more different types of lung carcinoma in the subject (e.g., adenocarcinoma, large cell carcinoma, squamous cell carcinoma, and/or small cell carcinoma). In preferred embodiments, a report is generated indicating the presence or absence of an assay result indicative of the presence of lung carcinoma and, if present, further indicative of the presence of one or more identified types of lung carcinoma. In some embodiments, samples from a subject are collected over the course of a period of time or a course of treatment, and assay results are compared to monitor changes in the cancer pathology.

Marker and marker panels sensitive to different types of lung cancer find use, e.g., in classifying type(s) of cancer present, identifying mixed pathologies, and/or in monitoring cancer progression over time and/or in response to treatment.

### Example 6

Using multiplex PCR followed by QuARTS (Quantitative Allele-Specific Real-time Target and Signal amplification) assay as described in Example 1, a post-bisulfite quantification of methylated DNA markers on DNA extracted from plasma was performed. The target sequences, bisulfite converted target sequences, and the assay oligonucleotides for these markers were as shown in FIG. 5. The primers and flap oligonucleotides (probes) used for each converted target were as follows:

TABLE 6

| | Oligo. | | | SEQ ID | |
|---|---|---|---|---|---|
| Marker | Name | Component | Sequence (5′-3′) | NO: | Arm |
| BARX1 | BARX1_FP | Primer | CGTTAATTTGTTAGATAGAGGGC | 23 | 5-FAM |
| | | | G | | |
| | BARX1_RP_ | Primer | TCCGAACAACCGCCTAC | 26 | |
| | universal | | | | |
| | BARX1_Pb_ | Flap Oligo. | AGGCCACGGACG | 405 | |
| | A5_63_v6 | | CGAAAAATCCCACGC/3C6/ | | |
| FLJ45983 | FLJ45983_ | Primer | CGAGGTTATGGAGGTGACG | 409 | 5-FAM |
| | FP_v4 | | | | |
| | FLJ45983_ | Primer | CGAATACTACCCGTTAAACACG | 410 | |
| | RP_v4 | | | | |
| | FLJ45983_ | Flap Oligo. | AGGCCACGGACG | 411 | |
| | Pb_A5_63_ | | GGCGGATTAGTCGCG/3C6/ | | |
| | v4 | | | | |
| HOXA9 | HOXA9_FP | Primer | TTGGGTAATTATTACGTGGATTC | 148 | 5-FAM |
| | | | G | | |
| | HOXA9_RP_ | Primer | CAACTCATCCGCGACG | 423 | |
| | v2 | | | | |
| | HOXA9_Pb_ | Flap Oligo. | AGGCCACGGACG | 424 | |
| | A5_63 | | GTCGACGCCCAACAA/3C6/ | | |
| HOPX | HOPX_2149_ | Primer | GTAGCGCGTAGGGATTATGTCG | 417 | 5-FAM |
| | FP | | | | |
| | HOPX_2149_ | Primer | TTTCCACCTAATCCTCTATAAAAC | 418 | |
| | RP | | CGC | | |
| | HOPX_2149_ | Flap Oligo. | AGGCCACGGACG | 419 | |
| | Pb_A5 | | CTCGCGATCTCCGC/3C6/ | | |
| ZNF781 | ZNF781F. | Primer | CGTTTTTTTGTTTTTCGAGTGCG | 373 | 5-FAM |
| | primer | | | | |
| | ZNF781R. | Primer | TCAATAACTAAACTCACCGCGTC | 374 | |
| | primer | | | | |
| | ZNF781_Pb_ | Flap Oligo. | AGGCCACGGACG | 435 | |
| | A5_63_v2 | | GCGGATTTATCGGGTTATAGT/3C6/ | | |
| HOXB2 | HOXB2_FP | Primer | GTTAGAAGACGTTTTTTCGGGG | 153 | 1-HEX |
| | HOXB2_RP | Primer | AAAACAAAAATCGACCGCGA | 154 | |
| | HOXB2_Pb_ | Flap Oligo. | CGCGCCGAGG | 425 | |
| | A1_63 | | GCGTTAGGATTTATTTTTTTTTTT | | |
| | | | CGA/3C6/ | | |
| IFFO1 | IFFO1_FP_ | Primer | CGGGATAGAGTCGATTAATTAG | 428 | 1-HEX |
| | HQ_ | | GC | | |
| | corrected | | | | |
| | IFFO1_RP | Primer | TAACTTCCCCTCGACCCG | 429 | |
| | IFF01_Pb_ | Flap Oligo. | CGCGCCGAGG | 430 | |
| | A1_63 | | CGGTTCGGTAGCGG/3C6/ | | |
| SOBP | SOBP HM | Primer | TTTCGGCGGGTTTCGAG | 294 | 1-HEX |
| | FP | | | | |
| | SOBP HM | Primer | CGTACCGTTCACGATAACGT | 295 | |
| | RP | | | | |
| | SOBP HM | Flap Oligo. | CGCGCCGAGG | 431 | |
| | Pb A1 63 | | TTACAAACCGCGACCG/3C6/ | | |
| TRH | TRH_FP | Primer | TTTTCGTTGATTTTATTCGAGTCG | 432 | 1-HEX |
| | | | TC | | |
| | TRH_RP | Primer | GAACCCTCTTCAAATAAACCGC | 433 | |
| | TRH_Pb_A1_ | Flap Oligo. | CGCGCCGAGG | 434 | |
| | 63 | | CGTTTGGCGTAGATATAAGC/3C6/ | | |
| FAM59B | FAM59B_ | Primer | GTCGAGCGTTTGGTGCG | 406 | 1-HEX |
| | FP_V3 | | | | |
| | FAM59B_ | Primer | CTCGTCGAAATCGAAACGC | 407 | |
| | RP_V3 | | | | |
| | FAM59B_ | Flap Oligo. | CGCGCCGAGG | 408 | |
| | Pb_A1_63_ | | GCGATAGCGTTTTTTATTGTCG/3C6/ | | |
| | V3 | | | | |

*All methylation assays were triplexed with an assay for bisulfite-converted B3GALT6 marker, reporting to Quasar:

| | Oligo- | | | SEQ | |
|---|---|---|---|---|---|
| | nucleotide | | | ID | |
| Marker | Name | Component | Sequence (5′-3′) | NO: | |
| B3GALT6 | B3GALT6_ | Primer | GGTTTATTTTGGTTTTTTGAGTTTTCGG | 386 | 3-Quasar |
| (BST) | FP_V2 | | | | |
| | B3GALT6_ | Primer | TCCAACCTACTATATTTACGCGAA | 387 | |
| | RP | | | | |
| | B3GALT6_ | Flap Oligo. | ACGGACGCGGAG | 436 | |
| | Pb_A3_63 | | GCGGATTTAGGGTATTTAAGGAG/3C6/ | | |

The DNA prepared from plasma as described above was amplified in a multiplexed pre-amplification reaction, as described in Example 1. Following pre-amplification, aliquots of the pre-amplified mixtures were diluted 1:10 in 10 mM Tris HCl, 0.1 mM EDTA, then were assayed in triplex QuARTS PCR-flap assays, as described in Example 1. The triplex combinations were as follows:

Triplex Assays

BARX1/HOXB2/B3GALT6 (BHB)
FU45983/IFFO1/B3GALT6 (FIB)
HOXA9/SOBP/B3GALT6 (HSB)
HOPX 2149/TRH/B3GALT6 (HTB)
ZNF781/FAM59B/B3GALT6 (ZFB)

Plasmids containing target DNA sequences were used to calibrate the quantitative reactions. For each calibrator plasmid, a series of 10× calibrator dilution stocks, having from 10 to 10⁶ copies of the target strand per μl in fish DNA diluent (20 ng/mL fish DNA in 10 mM Tris-HCl, 0.1 mM EDTA) were prepared. For triplex reactions, a combined stock having plasmids that contain each of the targets of the triplex were used. A mixture having each plasmid at 1×10⁵ copies per μL was prepared and used to create a 1:10 dilution series. Strands in unknown samples were back calculated using standard curves generated by plotting Cp vs Log (strands of plasmid).

Using receiver operating characteristic (ROC) curve analysis using % methylation relative to B3GALT6 strands, the area under the curve (AUC) for each marker was calculated and is shown in the table below.

| Marker Name | AUC |
|---|---|
| BARX1 | 0.754 |
| FLI45983 | 0.709 |
| HOXA9 | 0.800 |
| HOPX | 0.654 |
| ZNF781 | 0.760 |
| HOXB2 | 0.700 |
| IFFO1 | 0.788 |
| SOBP | 0.717 |
| FAM59B | 0.685 |

Using a 6-marker logistic fit using markers BARX1, FLJ45983, SOBP, HOPX, IFFO1, and ZNF781, ROC curve analysis shows an area under the curve (AUC) of 0.85881. Use of the markers in combination improved sensitivity compared to single markers.

### Example 7

### Combination of mRNA and Methylation Markers to Improve Lung Cancer Detection Sensitivity

Expression level of FPR1 mRNA (Formyl Peptide Receptor 1) has been shown previously to be a lung cancer marker detectable in blood (Morris, S., et al., Int J Cancer., (2018) 142:2355-2362). In some embodiments, the methylation marker assays described above are used in combination with measurement of one or more expression markers. An exemplary combination assay comprises measurement of FPR1 mRNA levels and detection of methylation marker DNA(s) (e.g., as described in Examples 1-6) in a sample or samples from the same subject.

The FPR1 sequence (NM_001193306.1 Homo sapiens formyl peptide receptor 1 (FPR1), transcript variant 1, mRNA, is shown in SEQ ID NO:437. As described by Morris, et al. supra, blood samples are collected in a blood collection tube suitable for subsequent RNA detection (e.g., PAXgene Blood RNA Tube; Qiagen, Inc.) Samples may be assayed immediately or frozen until future analysis. RNA is extracted from a sample by standard methods, e.g., Qiasymphony PAXgene blood RNA kit. Levels of RNA, e.g., an mRNA marker, are determined using a suitable assay for measurement of specific RNAs present in a sample, e.g., RT-PCR. In some embodiments, a QuARTS flap endonuclease assay reaction comprising a reverse transcription step is used. See, e.g., U.S. patent application Ser. No. 15/587,806, which is incorporated herein by reference. In preferred embodiments, assay probes and/or primers for an RT-PCR or an RT-QuARTS assay are designed to span an exon junction(s) so that the assay will specifically detect mRNA targets rather than detecting the corresponding genomic loci.

An exemplary RT-QuARTS reaction contains 20U of MMLV reverse transcriptase (MMLV-RT), 219 ng of Cleavase® 2.0, 1.5 U of GoTaq® DNA Polymerase, 200 nM of each primer, 500 nM each of probe and FRET oligonucleotides, 10 mM MOPS buffer, pH7.5, 7.5 mM MgCl₂, and 250 μM each dNTP. Reactions are typically run on a thermal cycler configured to collect fluorescence data in real time (e.g., continuously, or at the same point in some or all cycles). For example, a Roche LightCycler 480 system may be used under the following conditions: 42° C. for 30 minutes (RT reaction), 95° C. for 3 min, 10 cycles of 95° C. for 20 seconds, 63° C. for 30 sec, 70° C. for 30 sec, followed by 35 cycles of 95° C. for 20 sec, 53° C. for 1 min, 70° C. for 30 sec, and hold at 40° C. for 30 sec.

In some embodiments, RT-QuARTS assays may comprise a step of multiplex pre-amplification, e.g., to pre-amplify 2, 5, 10, 12, or more targets in a sample (or any number of targets greater than 1 target), as described above in Example 1. In preferred embodiments, an RT-pre-amplification is conducted in a reaction mixture containing, e.g., 20U of MMLV reverse transcriptase, 1.5 U of GoTaq® DNA Polymerase, 10 mM MOPS buffer, pH7.5, 7.5 mM MgCl₂, 250 μM each dNTP, and oligonucleotide primers, (e.g., for 12 targets, 12 primer pairs/24 primers, in equimolar amounts (e.g., 200 nM each primer), or with individual primer concentrations adjusted to balance amplification efficiencies of the different targets). Thermal cycling times and temperatures are selected to be appropriate for the volume of the reaction and the amplification vessel. For example, the reactions may be cycled as follows:

| | | # of |
|---|---|---|
| Stage | Temp/Time | Cycles |
| RT | 42° C./30′ | 1 |
| | 95° C./3′ | 1 |
| Amplification 1 | 95° C./20″ | 10 |
| | 63° C./30″ | |
| | 70° C./30″ | |
| Cooling | 4° C./Hold | 1 |

After thermal cycling, aliquots of the pre-amplification reaction (e.g., 10 μL) are diluted to 500 μL in 10 mM Tris, 0.1 mM EDTA, with or without fish DNA. Aliquots of the diluted pre-amplified DNA (e.g., 10 μL) are used in QuARTS PCR-flap assays, as described above.

In some embodiments, DNA targets, e.g., methylated DNA marker genes, mutation marker genes, and/or genes corresponding to the RNA marker, etc., may be amplified and detected along with the reverse-transcribed cDNAs in a QuARTS assay reaction, e.g., as described in Example 1, above. In some embodiments, DNA and cDNA are co-amplified and detected in a single-tube reaction, i.e., without the need to open the reaction vessel at any point between combining the reagents and collecting the output data. In other embodiments, marker DNA from the same sample or from a different sample may be separately isolated, with or without a bisulfite conversion step, and may be combined with sample RNA in an RT-QuARTS assay. In yet other embodiments, RNA and/or DNA samples may be pre-amplified as described above.

In Morris, ROC curve analysis of the FPR1 mRNA ratio relative to a housekeeping gene (HNRNPA1) resulted in a sensitivity of 68% at a specificity of 89%, and ROC curve analysis using methylation markers BARX1, FAM59B, HOXA9, SOBP, and IFFO1 results in a sensitivity of 77.2% at a specificity of 92.3%. Using these assays together results in a theoretical sensitivity of 92.7% at a specificity of 82%.

This analysis shows that a combination assay for levels of FPR1 mRNA along with detection of one or more methylation markers results in an assay having improved sensitivity compared to either method alone. A cancer detection assay that combines different classes of markers has the advantage of being able to detect the biological differences between early and late diseases stages as well as different biological responses or sources of cancer. It will be clear to one skilled in the art that other RNA targets, including mRNA targets other than or in addition to FPR1, such as LunX mRNA (Yu, et al., 2014, Chin J Cancer Res., 26:89-94), can be combined with methylation markers for enhanced sensitivity.

### Example 8

### RT-LQAS Assay of Combinations of mRNA Markers and DNA Markers to Improve Lung Cancer Detection Sensitivity

For RNA, blood was collected in PAXgene Blood RNA tubes for the RNA assays, and in BD Vacutainer PPT plasma preparation tubes (BD Biosciences) for DNA assays, and the samples were stored in accordance with manufacturer's instructions. RNA samples were extracted on the Qiagen QIAsymphony instrument using the QIAsymphony PAXgene Blood RNA Kit (ID: 762635) per manufacturer's instructions. Prior to testing in RT-LQAS, RNA samples were diluted 1:50 in 10 mM TrisHCl, pH 8.0, 0.1 mM EDTA. DNA was extracted as described in Example 1. Samples were as follows:

### RNA Study:

155 samples from subjects with lung cancer

317 samples from healthy, normal subjects

### DNA Study:

102 samples from subjects with lung cancer

142 samples from healthy, normal subjects

Primers and probes were designed for detection of a combination of 8 mRNAs and 3 reference genes, as shown below in Table 3.

TABLE 3

| | Name | Function |
|---|---|---|
| Symbol | | |
| FPR1 | Formyl Peptide Receptor 1 | Protein is important in host |
| | Accession number: NM_001193306 | defense and inflammation |
| S100A12 | S100 Calcium Binding Protein A12 | Plays a role in the regulation of |
| | Accession number: NM_005621 | inflammatory processes and |
| | | immune response |
| TYMP | Thymidine Phosphorylase | Promotes angiogenesis in vivo |
| | Accession number: NM_001113755 | |
| APOBEC3A | Apolipoprotein B MRNA Editing | May play a role in the epigenetic |
| | Enzyme Catalytic Subunit 3A | regulation of gene expression |
| | Accession number: NM_145699 | through the process of active |
| | | DNA demethylation |
| MMP9 | Matrix Metallopeptidase 9 | May play an essential role in |
| | Accession number: NM_004994 | local proteolysis of the |
| | | extracellular matrix and in |
| | | leukocyte migration |
| SELL | Selectin L | Required for binding and |
| | Accession number: NM_000655 | subsequent rolling of leucocytes |
| | | on endothelial cells, facilitating |
| | | their migration into secondary |
| | | lymphoid organs and |
| | | inflammation sites |
| S100A9 | S100 Calcium Binding Protein A9 | Plays a role in the regulation of |
| | Accession number: NM_002965 | inflammatory processes and |
| | | immune response |
| PADI4 | Peptidyl Arginine Deiminase 4 | May play a role in granulocyte |
| | Accession number: NM_012387 | and macrophage development |
| | | leading to inflammation and |
| | | immune response |
| Reference | | |
| Gene | | |
| CASC3 | CASC3 Exon Junction Complex | Protein is a core component of |
| | Subunit | the exon junction complex |
| | Accession number: NM_007359 | (EJC) |
| SKP1 | S-Phase Kinase Associated Protein | Component of the SCF (SKP1- |
| | Accession number: NM_006930 | CUL1-F-box protein) ubiquitin |
| | | ligase complex |
| STK4 | Serine/Threonine Kinase 4 | Stress-activated, pro-apoptotic |
| | Accession number: NM_006282 | kinase |
| HNRNPA1 | Heterogeneous Nuclear | RNA binding protein |
| | Ribonucleoprotein A1 | |
| | Accession number: NM_002136 | |

Primers and flap oligonucleotide probes for the target nucleic acids listed above are shown in FIG. 6. The RT-LQAS assay was conducted as described in Example 1, above. The analysis used % RNA levels calculated by:
Calculating strand values of mRNA levels using RT-LQAS and synthetic RNA targets for calibrators;
Averaging strand levels of the three reference genes (CASC3, SKP1, STK4);
Dividing mRNA strands of measured marker by the average of the strands of the three reference genes;
Performing ROC analysis of % RNA

LQAS Assay performance using these RNA markers individually and analyzed using receiver operating characteristic (ROC) curve analysis, the area under the curve (AUC) for each RNA marker was calculated and is summarized below:

| | | Sensitivity at |
|---|---|---|
| RNA Marker | AUC | 90% specificity |
| S100A9 | 0.76286 | 45.80% |
| SELL | 0.72854 | 43.90% |
| PADI4 | 0.81801 | 57.40% |
| APOBE3CA | 0.72034 | 38.10% |
| S100A12 | 0.76801 | 50.10% |
| MMP9 | 0.76518 | 49.70% |
| FPR1 | 0.66952 | 27.10% |
| TYMP | 0.54448 | 16.80% |

Analysis of both RNA and methylated DNA was conducted using 102 samples from subjects with lung cancer and 142 samples from healthy normal subjects. Using a high-performing mRNA marker pair PADI4 and SELL, the logistical fit of the combined RNA markers had an area under the curve of 0.85626, and showed 63.7% sensitivity at 90% specificity. Using the high-performing DNA methylation marker pair HOXA9 and IFFO1, the logistical fit of the combined DNA methylation assay had an area under the curve of 0.091677, and showed 78.4% sensitivity at 90% specificity. Combining results of these mRNA markers and DNA methylation markers yielded and area under the curve of 0.95070, and showed 90.2% sensitivity at 90% specificity.

### Example 9

### Combination of a Protein (e.g., Autoantibody) and Methylation Markers to Improve Lung Cancer Detection Sensitivity

Tumor-associated antigens in lung and other solid tumors can provoke a humoral immune response in the form of autoantibodies, and these antibodies have been observed to be present very early in the disease course, e.g., prior to the presentation of symptoms. (see Chapman C J, Murray A, McElveen J E. et al. Thorax 2008; 63:228-233, which is incorporated herein by reference in its entirety for all purposes). However, the sensitivity of autoantibody detection for detecting lung carcinomas is relatively low. For example, autoantibodies to tumor antigen NY-ESO-1 (Accession #P78358, sequence shown as SEQ ID NO: 442; also known as CTAG1B) has been shown in the literature to be a good marker for non small-cell lung cancer (NSCLC; Chapman, supra), but it is not sufficiently sensitive to be useful alone. The detection of one or more tumor-associated autoantibodies in combination with the detection of one or more methylation markers provides an assay with greater sensitivity.

Blood samples are collected, and autoantibodies are detected using standard methods, e.g., ELISA detection, as described by Chapman, supra. Detecting methylation and/or mutation markers in DNA isolated the samples is done as described in Example 1, above.

Detection of NY-ESO-1 autoantibody alone results in a sensitivity of 40% at 95% specificity (Türeci, et al., Cancer Letters 236(1):64 (2006). As discussed above, assaying the methylation of the combination of BARX1, FAM59B, HOXA9, SOBP, and IFFO1 markers results in a sensitivity of 77.2% at 92.3% specificity. Combining analysis of this autoantibody marker with the assay for this combination of methylation markers results in a combined theoretical sensitivity of 86.3%, with at specificity of 87.7%.

This analysis shows that combined assays of levels of autoantibodies with analysis of one or more methylation markers results in an assay having improved sensitivity compared to either method alone. A cancer detection assay that combines different classes of markers has the advantage of being able to detect the biological differences between early and late diseases stages as well as different biological responses or sources of cancer.

### Example 10

### Combination of mRNA, Methylation Marker(s), and Protein (e.g., Autoantibody) to Improve Lung Cancer Detection Sensitivity

Analysis of combinations of one or more RNAs, marker DNAs, and autoantibodies in a sample or samples from a subject may be performed for enhanced detection of lung and other cancers in the subject. Methods for sample preparation and DNA, RNA, and protein detection are as discussed above.

As discussed in Example 7, analysis of the FPR1 mRNA ratio relative to a housekeeping gene (HNRNPA1) as reported by Morris, et al. resulted in a sensitivity of 68% at a specificity of 89% (Morris, supra); detection of NY-ESO-1 autoantibody alone as reported by Chapman resulted in a sensitivity of 40% at 95% specificity; and assaying the methylation of the combination of BARX1, FAM59B, HOXA9, SOBP, and IFFO1 markers results in a sensitivity of 77.2% at 92.3% specificity. Combining analysis of the mRNA, the autoantibody marker, and the assay for this combination of methylation markers results in a combined theoretical sensitivity of 95.6%, with a specificity of 77.9%, showing that combined assays of levels of mRNA and levels of autoantibodies with analysis of one or more methylation markers results in an assay having improved sensitivity compared to any one of these methods alone.

Assays as described above may be further enhanced by the addition of an assay to detect one or more antigens. Those of skill in the art will appreciate that detection of an antigen may be added to the detection of any of: RNA(s), methylation marker gene(s), and/or autoantibody(ies), individually or in any combination, and will further enhance overall sensitivity.

### Example 11

### RNA Expression in Samples from Subjects Having Different Stage Cancers

Blood samples were collected from patients known to have stage I, stage II, stage III, and stage IV non-small cell lung cancer (“NSCLC”). For comparison, blood samples were also collected from people without any known lung cancer (putatively “cancer free” individuals), for both non-smokers and tobacco smokers. There was some possibility that people without any known lung cancer may in fact have an otherwise undetected cancer. The presence of these patients would lead to an over-estimation of the false positive rate for this test (because “false positives” from “healthy individuals” may in fact represent the presence of cancer in these individuals). The blood samples were collected in PAXgene Blood RNA Tubes, and shipped to a testing facility at room temperature, or on ice, to minimize sample degradation. After the samples were received in the testing facility, white blood cell RNA from each blood sample was extracted with the QIAamp® RNA Blood Mini Kit.

After RNA was extracted, the Illumina TruSeq Stranded Total RNA Library Prep Human/Mouse/Rat protocol was used to prepare a cDNA library from the RNA of each blood sample. Next, the cDNA library of each blood sample was sequenced in the Illumina NextSeq 550 System to profile the whole transcriptome and to obtain the RNA expression level of each gene. The following results were obtained.

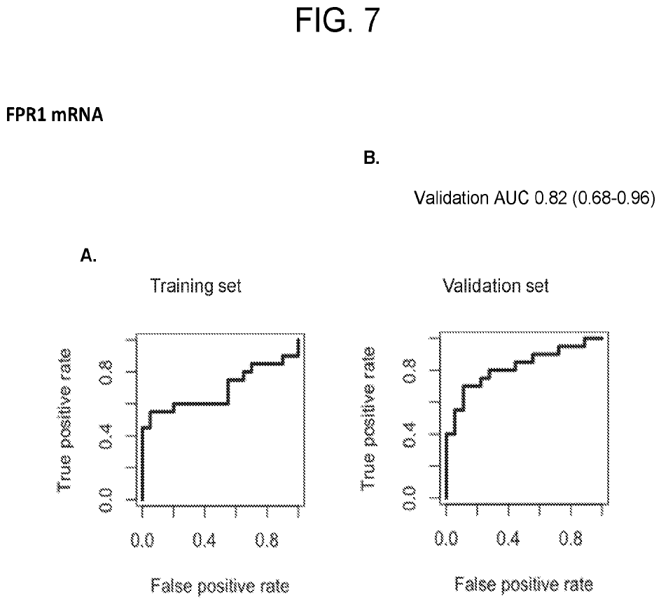

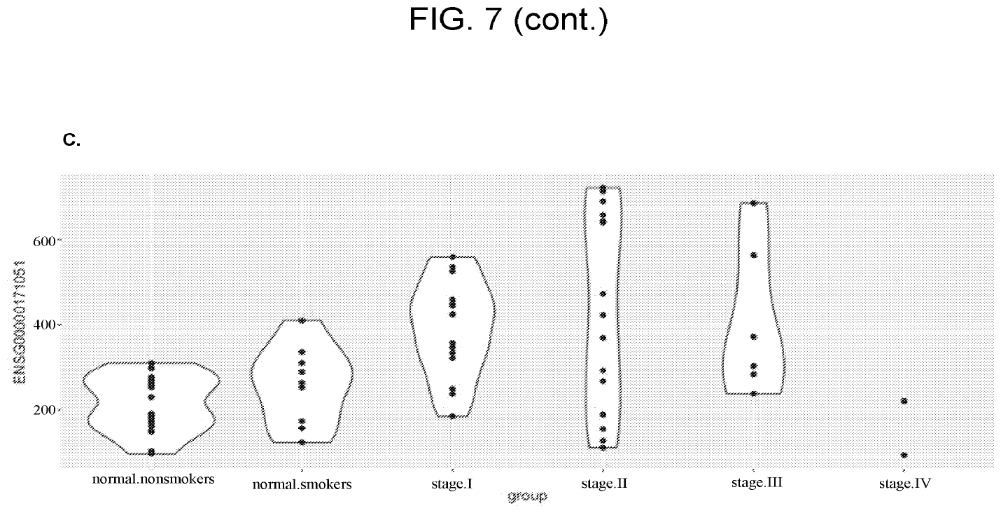

Referring to FIGS. 7-10, from the whole transcriptome analysis on white blood cell RNA, target genes that showed significant gene expression changes between healthy individuals and lung cancer patients were identified. The gene expression changes presumably reflected the immune response of immune cells to tumors in the patients. These results showed that measuring the RNA expression levels of at least the disclosed target genes allows one to predict the presence of lung cancer in a person.

As shown in Panel C of FIG. 7, each data point represented the RNA expression level of the target gene FPR1 (y-axis) from the blood sample of an individual. The x-axis grouped the individuals by healthy non-smokers, healthy tobacco smokers, and stage I-IV NSCLC patients. Compared to healthy individuals, stages I-III NSCLC involved significant increases in FPR1 gene expression levels. In addition, FPR1 gene expression was slightly increased for normal tobacco smokers.

Panels A and B of FIG. 7 showed receiver operating characteristic (ROC) curves for a portion of the data assigned as a training set and a portion of the data assigned as a validation set. At each selected RNA expression threshold level (a slice at a y-value of the Panel C), the true positive rates and the false positive rates were calculated. The percentage of NSCLC patients who were correctly identified as having the particular condition defined the true positive rate (sensitivity), while the percentage of healthy people who were correctly identified as not having the NSCLC defined the specificity. The false positive rate was defined as (1—specificity). For a random guess, the ROC curve would be a diagonal line and the area-under-curve (AUC) would be 0.5. The AUC for the validation set was 0.82, which demonstrated that FPR1 gene expression was predictive of NSCLC risk.

Similarly, in Panel C of FIG. 8, each data point represented the RNA expression level of the target gene S100A12 (y-axis) from a white blood cell sample of an individual. The x-axis grouped the individuals by healthy non-smokers, healthy tobacco smokers, and stage I-IV NSCLC patients. Compared to healthy individuals, stages I-III NSCLC involved significant increases in S100A12 gene expression levels. Panels A and B of FIG. 8 showed the ROC curves for a portion of the data assigned as training set and a portion of the data assigned as validation set. The AUC for the validation set was 0.93, which demonstrated that S100A12 gene expression was predictive of NSCLC risk and was significantly better than using FPR1 as target gene.

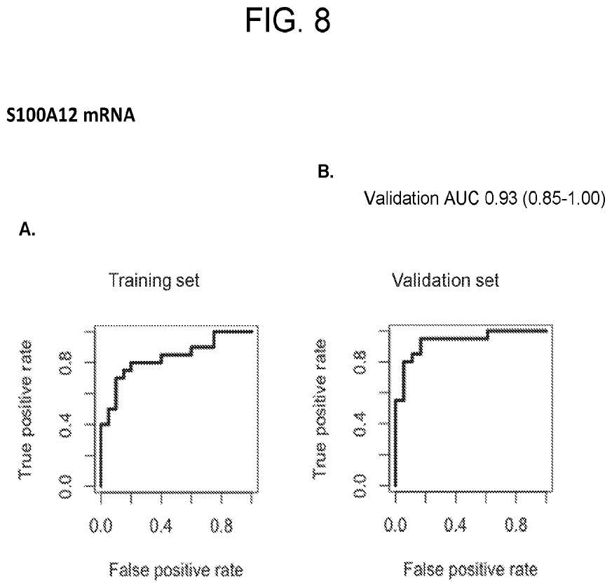

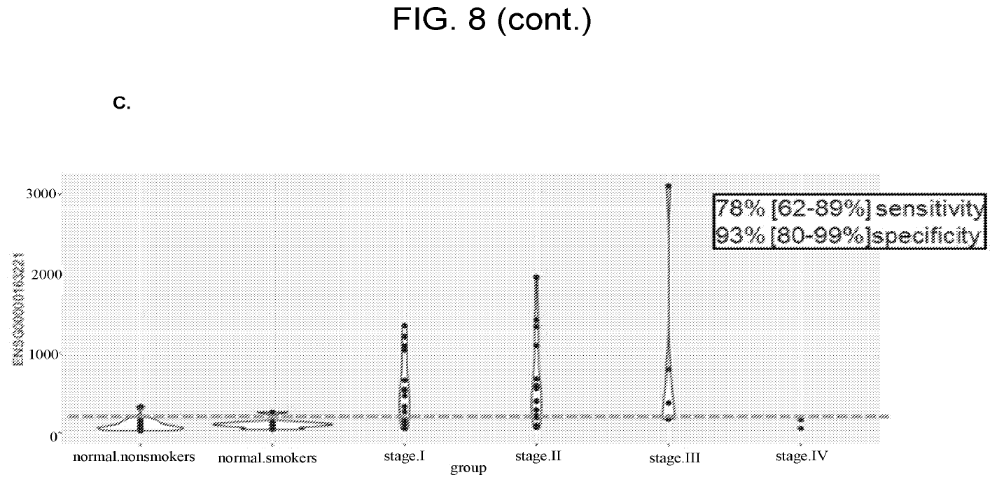

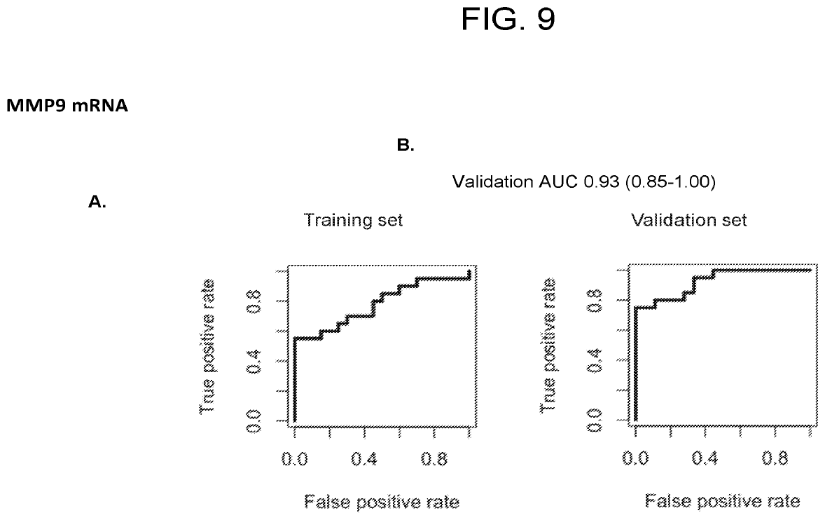

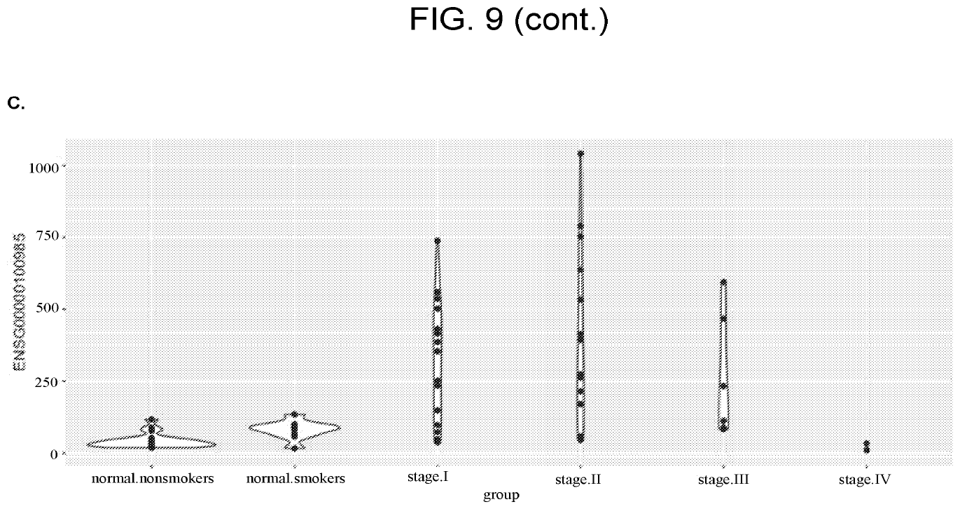

In Panel C of FIG. 9, each data point represented the RNA expression level of the target gene MMP9 (y-axis) from the white blood cell sample of an individual. The x-axis grouped the individuals by healthy non-smokers, healthy tobacco smokers, and stage I-IV NSCLC patients. Compared to healthy individuals, stages I-III NSCLC involved significant increases in MMP9 gene expression levels. In addition, MMP9 gene expression slightly increased for tobacco smokers. Panels A and B of FIG. 9 showed the ROC curves for a portion of the data assigned as training set and a portion of the data assigned as validation set. The AUC for the validation set was 0.93, which demonstrated that MMP9 gene expression was predictive of NSCLC risk and was also significantly better than using FPR1 as target gene.

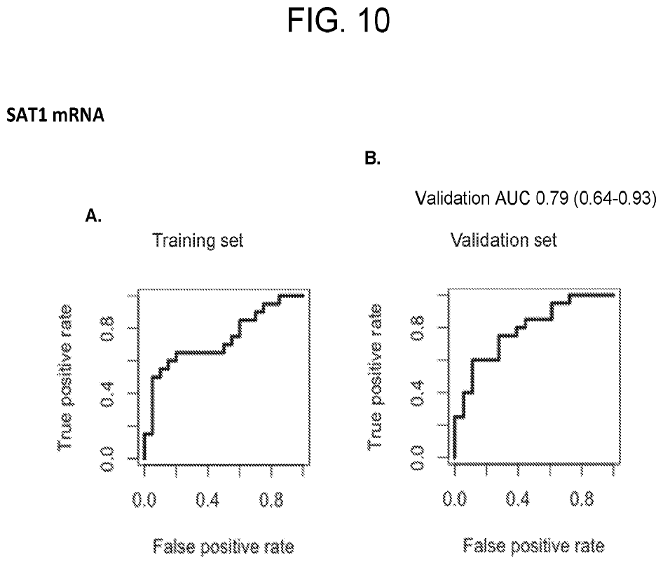

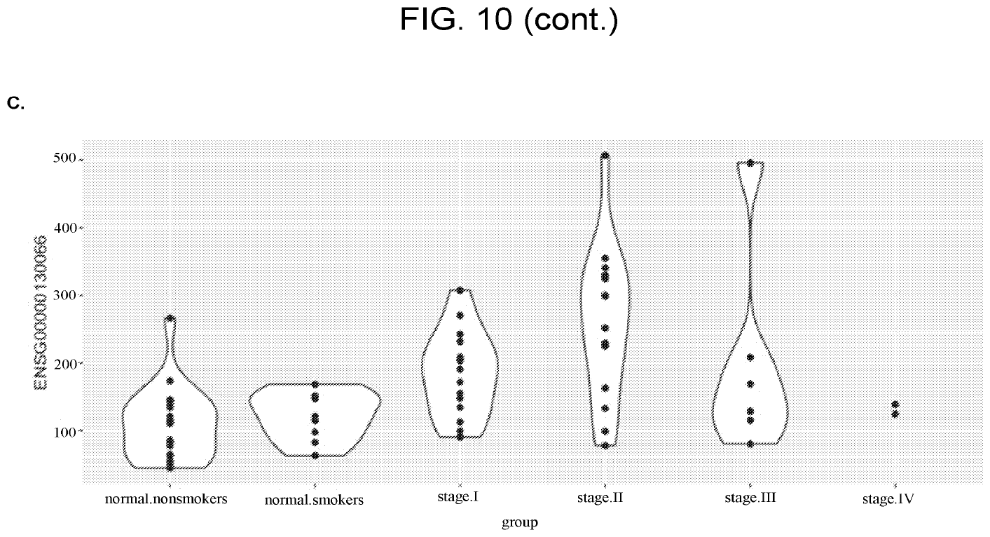

In the Panel C of FIG. 10, each data point represented the RNA expression level of the target gene SAT1 (y-axis) from a white blood cell sample of an individual. The x-axis grouped the individuals by healthy non-smokers, healthy tobacco smokers, and stage I-IV NSCLC patients. Compared to healthy individuals, stages I-III NSCLC involved significant increases in SAT1 gene expression levels. Panels A and B of FIG. 10 showed the ROC curves for a portion of the data assigned as training set and a portion of the data assigned as validation set. The AUC for the validation set was 0.79, which demonstrated that SAT1 gene expression was predictive of NSCLC risk.

These experimental results showed that detecting the RNA expression levels of the disclosed target genes allowed one to predict the presence of lung cancer in a person.

### Example 12

### Comparing RNA Expression Levels to Expression from Reference Genes

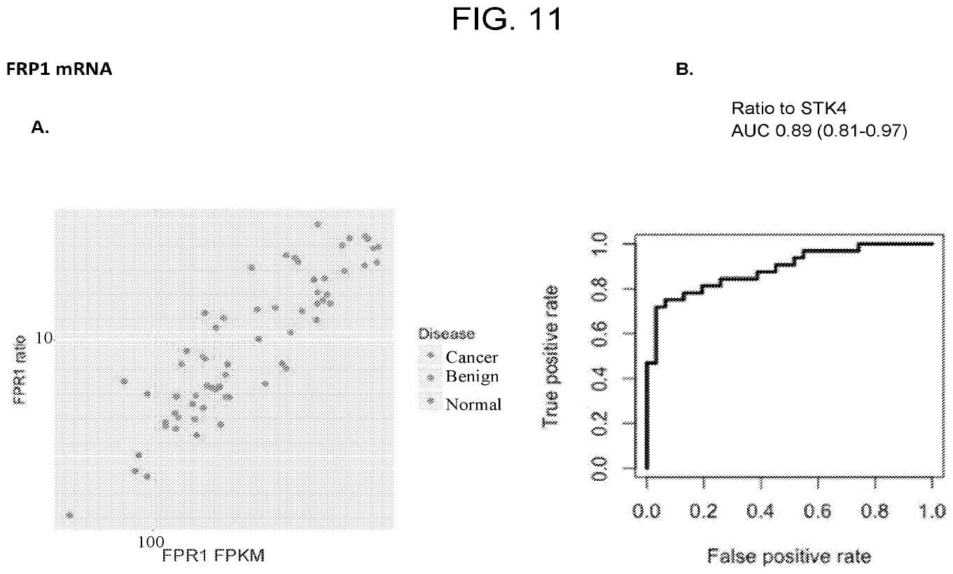

FIGS. 11-13 show that comparing the RNA expression levels of a target gene to a reference gene may allow for a better prediction of the presence of lung cancer in a person.

As shown in Panel A of FIG. 11, each data point represents a white blood sample taken from an individual who was 1) healthy, 2) has a benign lung tumor, or 3) has been diagnosed with lung cancer. The x-axis (FPR1 FPKM) represents the Fragments Per Kilobase Million normalization of the bare FPR1 expression level. The y-axis (FPR1 ratio) represents the ratio of the level of FPR1 expression to the level of reference gene STK4 expression. As shown in Panel B of FIG. 11, a ROC analysis was performed for the FPR1 ratio, and the AUC was found to be 0.89, which improved upon the predictive power of using FPR1 expression alone (FIG. 7).

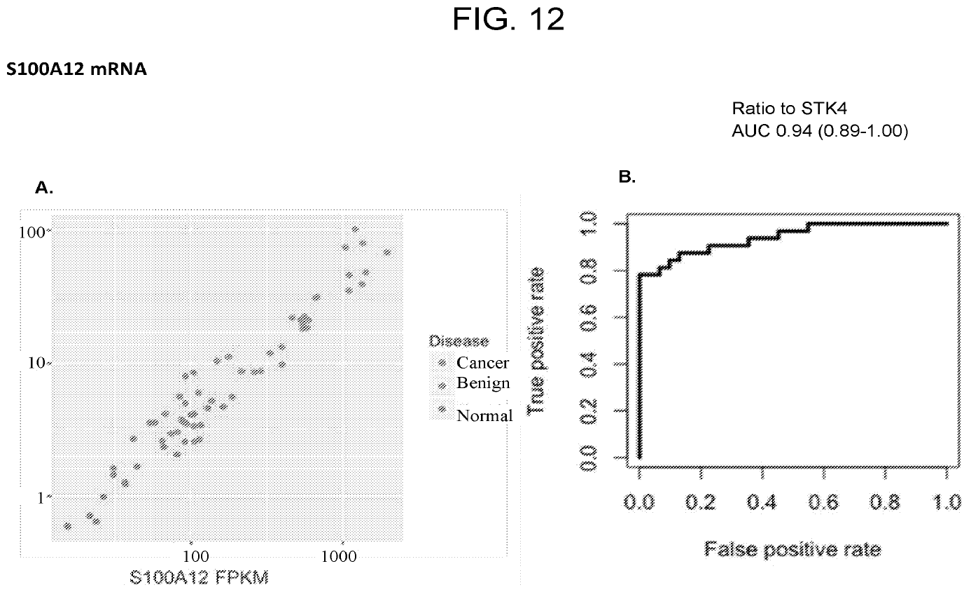

As shown in Panel A of FIG. 12, each data point represents a white blood cell sample from an individual who was 1) healthy, 2) has a benign lung tumor, or 3) has been diagnosed with lung cancer. The x-axis (1 FPKM) represents the Fragments Per Kilobase Million normalization of the bare S100A12 expression level. The y-axis (S100A12 ratio) represented the ratio of S100A12 expression level to the reference gene STK4 expression level. As shown Panel B of FIG. 12, a ROC analysis was performed for the S100A12 ratio, and the AUC was 0.94, which improved upon the predictive power of using S100A12 expression alone (FIG. 8).

As shown in Panel A of FIG. 13, each data point represents a white blood cell sample from an individual who was healthy, having benign lung tumor, or having lung cancer. The x-axis (MMP9 FPKM) represents the Fragments Per Kilobase Million normalization of the bare MMP9 expression level. The y-axis (MMP9 ratio) represented the ratio of MMP9 expression level to the reference gene STK4 expression level. As shown in Panel B of FIG. 13, a ROC analysis was performed for the MMP9 ratio, and the AUC was 0.94, which improved upon the predictive power of using MMP9 expression alone (FIG. 9).

These experimental results showed that comparing the RNA expression levels of the target genes to the disclosed reference gene resulted in a better prediction of the presence of lung cancer in a person.

### Example 13

### RNA Expression Levels from Combinations of Marker Genes

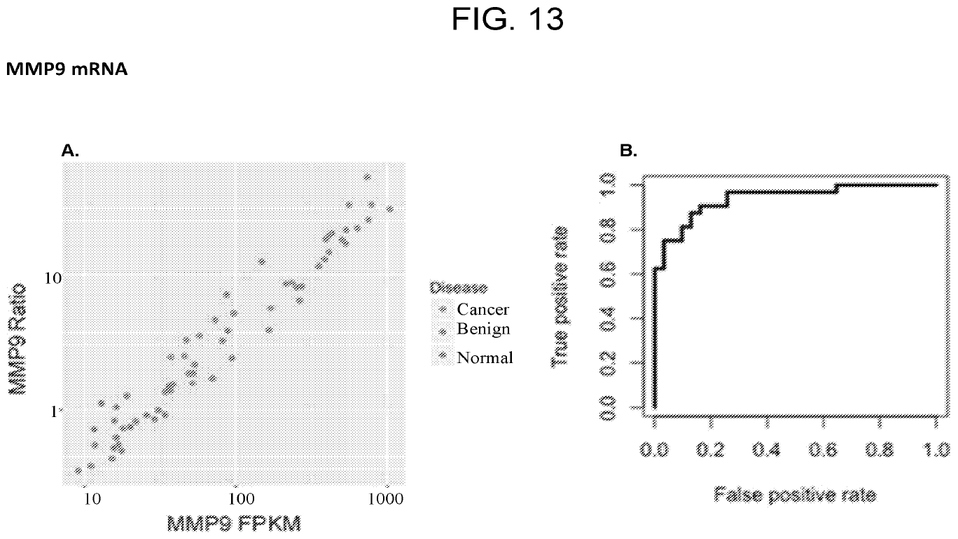

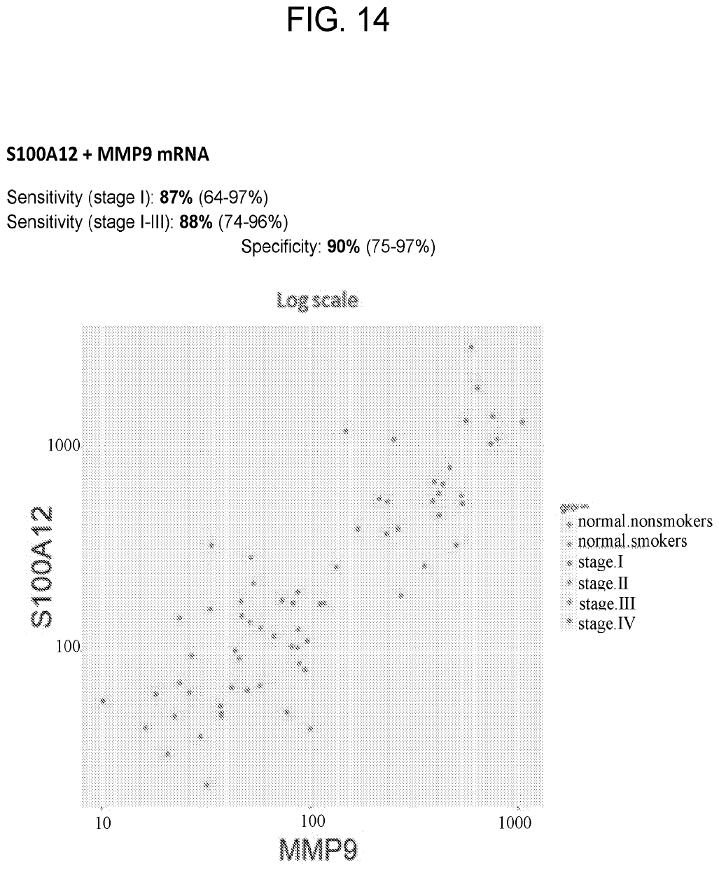

FIGS. 14-16 show that using the RNA expression levels of two target genes together allowed one to predict the presence of lung cancer in a person.

In FIG. 14, using data of the two most predictive target genes from Example 12, e.g., S100A12 and MMP9, a binary classifier (represented by the dashed line) was learned. S100A12 is on the Y-axis and MMP9 is on the X axis. The data shown is FPKM normalized. Each data point represents a blood sample from an individual who was 1) a healthy non-smoker, 2) a healthy tobacco smoker, 3) having stage I NSCLC, 4) having stage II NSCLC, 5) having stage III NSCLC, or 6) having stage IV NSCLC. The classifier had a sensitivity of 0.87 for stage I NSCLC, a sensitivity of 0.88 for stages I-III NSCLC, and a specificity of 0.9. This demonstrates that combining the gene expression data of S100A12 and MMP9 resulted in a good predictive power for lung cancer risk.

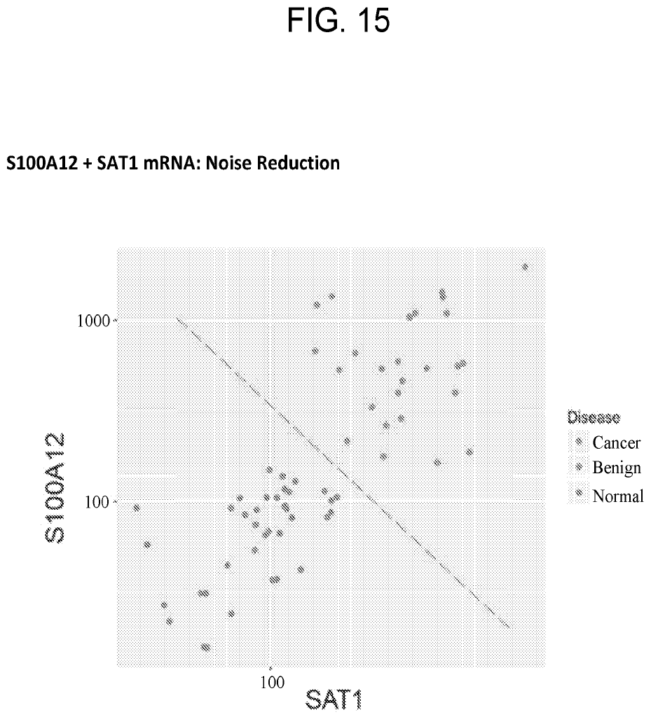

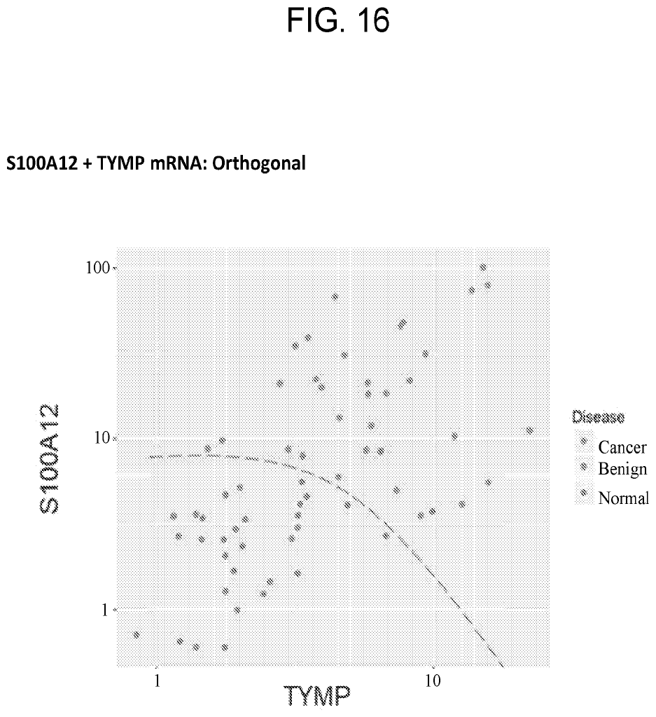

Alternatively, FIG. 15 used the gene expression data of S100A12 and SAT1, and FIG. 16 used the gene expression data of S100A12 and TYMP. Each data point represents a blood sample from an individual who was 1) healthy, 2) has a benign lung tumor, or 3) has been diagnosed with lung cancer. FIG. 15 shows genes selected to maximize the distance between groups. This minimizes the impact of detection error and pre-analytical variables on the data. FIG. 16 attempts to find an orthogonal marker to S100A12. It was found that TYMP was very good for separating benign nodules from cancers, meaning it could be used as part of a good reflex test for nodules discovered in CT scans.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

While certain embodiments of the inventions have been described, these embodiments have been presented by way of example only, and are not intended to limit the scope of the disclosure. Indeed, the novel methods and systems described herein may be embodied in a variety of other forms. Further, various modifications, omissions, substitutions, and variations of the described compositions, methods, systems, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims. The accompanying claims and their equivalents are intended to cover such forms or modifications as would fall within the scope and spirit of the disclosure. Accordingly, the scope of the present inventions is defined only by reference to the appended claims.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

We claim:

1. A method for measuring amounts of one or more gene expression products in blood sampled from a subject, comprising:
a) extracting from blood sampled from a subject:
i) at least one gene expression marker, wherein the at least one gene expression marker is product from expression of a marker gene selected from S100A9, SELL, PADI4, APOBE3CA, S100A12, MMP9, FPR1, TYMP, and SAT1; and
ii) at least one reference marker;
b) measuring an amount of the at least one gene expression marker and an amount of at least one reference marker extracted in a);
c) calculating a value for the amount of the at least one gene expression marker as a percentage of the amount of the at least one reference marker, wherein the value indicates an amount of the at least one gene expression marker in the blood sampled from the subject.

2. The method of claim 1, wherein the extracting comprises extracting markers from a sample selected from whole blood, a blood product comprising white blood cells, and a blood product comprising plasma.

3. The method of claim 1 or claim 2, wherein the at least one gene expression marker comprises protein or RNA.

4. The method of claim 3, wherein RNA extracted from the blood sampled from the subject comprises circulating cell-free RNA.

5. The method of any one of claims 3-4, wherein RNA extracted from the blood sampled from the subject comprises RNA expressed by immune cells.

6. The method of any one of claims 3-5, wherein RNA extracted from the blood sampled from the subject comprises mRNA.

7. The method of any one of claims 1-6, wherein the at least one gene expression marker consists of 2, 3, 4, 5, 6, 7, 8, or 9 gene expression markers.

8. The method any one of claims 1-7, wherein the at least one reference marker comprises RNA or protein expressed from a gene selected from PLGLB2, GABARAP, NACA, EIF1, UBB, UBC, CD81, TMBIM6, MYL12B, HSP90BL, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACTB, HNRNPA1, CASC3, and SKP1.

9. The method of claim 8, wherein the at least one reference marker comprises RNA.

10. The method of any one of claims 1-9, wherein the at least one reference marker comprises RNA selected from U1 snRNA and U6 snRNA.

11. The method of any one of claims 1-10, wherein measuring an amount of the at least one gene expression marker comprises using one or more of reverse transcription, polymerase chain reaction, nucleic acid sequencing, mass spectrometry, mass-based separation, and target capture, quantitative pyrosequencing, flap endonuclease assay, PCR-flap assay, enzyme-linked immunosorbent assay (ELISA) detection and protein immunoprecipitation.

12. The method of claim 11, wherein the measuring comprises multiplex amplification.

13. The method of an one of claims 1-12, further comprising:
d) extracting from blood sampled from the subject at least one methylation marker DNA and at least one reference marker DNA;
e) measuring an amount of at least one methylation marker DNA, wherein the at least one methylation marker DNA comprises a nucleotide sequence associated with at least one of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX.chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329;
f) measuring an amount of at least one reference marker DNA; and
g) calculating a value for the amount of the at least one methylation marker DNA as a percentage of the amount of the reference marker DNA, wherein the value Indicates an amount of the at least one methylation marker DNA In the blood sampled from a subject.

14. The method of claim 13, wherein said at least one methylation marker DNA consists of 2, 3, 4, 5, 6, 7, 8, 9, 10, 11, 12, 13, 14, or 15 methylation marker DNAs.

15. The method of claim 13 or claim 14, wherein DNA extracted from the blood sampled from the subject comprises circulating cell-free DNA.

16. The method of any one of claims 13-15, wherein the at least one reference marker DNA is selected from B3GALT6 DNA and β-actin DNA.

17. The method of any one of claims 13-16, wherein the at least one methylation marker DNA comprises a nucleotide sequence associated with at least one of BARX1, FLJ45983, HOPX, ZNF781, FAM59B, HOXA9, SOBP, and IFFO1.

18. The method of claim 17, wherein the at least one gene expression marker comprises a product from expression of a marker gene selected from FPR1, PADI4 and SELL.

19. The method of any one of claims 13-18, wherein the methylation marker DNA is treated with a reagent that selectively modifies DNA in a manner specific to the methylation status of the DNA.

20. The method of claim 19, wherein the reagent comprises a bisulfite reagent, a methylation-sensitive restriction enzyme, or a methylation-dependent restriction enzyme.

21. The method of claim 20, wherein the bisulfite reagent comprises ammonium bisulfite.

22. The method of any one of claims 13-21, wherein measuring an amount of at least one methylation marker DNA comprises using one or more of polymerase chain reaction, nucleic acid sequencing, mass spectrometry, methylation-specific nuclease, mass-based separation, and target capture.

23. The method of claim 22, wherein the measuring comprises multiplex amplification.

24. The method of any one of claims 13-23, wherein measuring an amount of at least one methylation marker DNA comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

25. A method of characterizing blood sampled from a subject, comprising:
i) treating blood sampled from a subject to produce extracted DNA and extracted RNA;
ii) measuring amounts of two or more marker RNAs in the extracted RNA, wherein the marker RNAs are selected from S100A9, SELL, PADI4, APOBE3CA, S100A12, MMP9, FPR1, TYMP, and SAT1 RNAs;
iii) measuring an amount of at least one reference RNA in the extracted RNA, wherein the reference RNA is selected from CASC3A, SKP1, and STK4;
iv) calculating a values for the amount of each of the two or more marker RNAs as a percentage of the amount of the at least one reference RNA, wherein the value for each marker RNA is Indicative of the amount of the marker RNA in the blood sampled from the subject;
v) treating the extracted DNA with a bisulfite reagent to produce bisulfite-treated DNA;
vi) measuring amounts of two or more methylation marker DNAs in the bisulfite-treated DNA, wherein the methylation marker DNAs are selected from EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST831A1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC128, BHLHE23, CAPN2, FGF14, FLJ34208, BIN2_Z, DNMT3A, FERMT3, NFIX, SIPR4, SKI, SUCLG2, TBX15, and ZNF329 genes;
vii) measuring an amount of at least one reference DNA in the bisulfite-treated DNA wherein the at least one reference DNA is selected from B3GALT6 DNA and β-actin DNA; and
viii) calculating a value for the amount of each of the two or more methylation marker DNAs as a percentage of the amount of a reference DNA measured in the bisulfite-treated DNA, wherein the value for each methylation marker DNA is indicative of the amount of the methylation marker DNA in the blood sampled from the subject.

26. The method of any one of claims 13-25, wherein DNA and RNA are isolated from blood collected in a single blood collection device.

27. The method of any one of claims 1-26, wherein the subject has or is suspected of having a lung neoplasm.

28. The method of any one of claims 1-27, wherein amounts of the at least one gene expression marker in the blood sampled from the subject is indicative of lung cancer risk of the subject.

29. The method of any one of claims 13-28, wherein amounts of the at least one methylation marker DNA in the blood sampled from the subject is indicative of lung cancer risk of the subject.

30. A kit, comprising:
a) set of reagents for measuring an amount of at least one gene expression marker in blood sampled from a subject, wherein the at least one gene expression marker is produced from expression of a marker gene selected from S100A9, SELL, PADI4, APOBE3CA, S100A12, MMP9, FPR1, TYMP, and SAT1;
b) a set of reagents for measuring an amount of at least one reference marker in blood sampled from the subject.

31. The kit of claim 30, further comprising a set of reagents for extracting the at least one gene expression marker and the at least one reference marker from blood.

32. The kit of claim 30 or 31, wherein the at least one gene expression marker comprises one or more of RNA and protein, and wherein the at least one reference marker comprises one or more of RNA, DNA, and protein.

33. The kit of any one of claims 30-32, wherein the kit comprises:
i) at least one first oligonucleotide, wherein at least a portion of the at least one first oligonucleotide specifically hybridizes to a nucleic acid strand comprising a nucleotide sequence associated with a gene expression marker selected from S100A9, SELL, PADI4, APOBE3CA, S100A12, MMP9, FPR1, TYMP, and SAT1;
ii) at least one second oligonucleotide, wherein at least a portion of the at least one second oligonucleotide specifically hybridizes to a reference marker, wherein the reference marker is a reference nucleic acid.

34. The kit of claim 33, wherein the nucleic acid strand comprising a nucleotide sequence associated with a gene expression marker is selected from RNA, cDNA, or amplified DNA.

35. The kit of claim 33 or 34, wherein the reference nucleic acid comprises RNA or DNA.

36. The kit of any one of claims 30-35, wherein the reference marker comprises RNA or protein expressed from a gene selected from PLGLB2, GABARAP, NACA, EIF1, UBB, UBC, CD81, TMBIM6, MYL12B, HSP90BL, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACT, HNRNPA1, CASC3, and SKP1.

37. The kit of any one of claims 33-36, further comprising:
c) a set of reagents for measuring an amount at least one methylation marker DNA in blood sampled from the subject, wherein the at least one methylation marker DNA comprises a nucleotide sequence associated with at least one of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOD, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX1, and ZNF329.

38. The kit of claim 37, wherein the set of reagents for measuring an amount at least one methylation marker DNA comprises:
iii) at least one third oligonucleotide, wherein at least a portion of the at least one third oligonucleotide specifically hybridizes to a nucleic acid strand comprising a nucleotide sequence associated with a methylation maker gene of EMX1, GRIN2D, ANKRD13B, ZNF781, ZNF671, IFFO1, HOPX, BARX1, HOXA9, LOC100129726, SPOCK2, TSC22D4, MAX.chr8.124, RASSF1, ST8SIA1, NKX6_2, FAM39B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARNGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329.

39. The kit of claim 38, further comprising at least one fourth oligonucleotide, wherein at least a portion of the at least one fourth oligonucleotide specifically hybridizes to a reference marker DNA, preferably a reference marker DNA selected from B3GALT6 DNA and β-actin DNA.

40. The kit of claim 38 or 39, wherein at least one of the nucleic acid strand comprising a nucleotide sequence associated with a methylation maker gene and the reference marker DNA comprises bisulfite-treated DNA.

41. The kit of any one of claims 38-40, further comprising a reagent that selectively modifies DNA in a manner specific to the methylation status of the DNA.

42. The kit of claim 41, wherein the reagent that selectively modifies DNA in a manner specific to the methylation status of the DNA comprises a bisulfite reagent, a methylation-sensitive restriction enzyme, or a methylation-dependent restriction enzyme.

43. The kit of claim 42, wherein the bisulfite reagent comprises ammonium bisulfite.

44. The kit of any one of claims 33-43, wherein one or more of the at least one first, second, third, and fourth oligonucleotides are selected from a capture oligonucleotide, a pair of nucleic acid primers, a nucleic acid probe, and an invasive oligonucleotide.

45. The kit of claim 44, wherein the capture oligonucleotide is attached to a solid support.

46. The kit of claim 45, wherein the solid support is a magnetic bead.

47. The kit of any one of claims 33-46, comprising
i) a first primer pair for producing a first amplified DNA from a gene expression marker product of expression of a marker gene selected from S100A9, SELL, PADI4, APOBE3CA, S100A12, MMP9, FPR1, TYMP, and SAT1;
ii) a first probe comprising a sequence complementary to a region of said first amplified DNA;
iii) a second primer pair for producing a second amplified DNA;
iv) a second probe comprising a sequence complementary to a region of said second amplified DNA;
v) reverse transcriptase; and
vi) a thermostable DNA polymerase.

48. The kit of claim 47, wherein the second amplified DNA is produced from a methylation marker gene or a reference marker nucleic acid.

49. The kit of claim 47 or 48, wherein the first probe further comprises a flap portion having a first flap sequence that is not substantially complementary to said first amplified DNA.

50. The kit of any one of claims 47-49, wherein the second probe further comprises a flap portion having a second flap sequence that is not substantially complementary to said second amplified DNA.

51. The kit of any one of claims 49-50, further comprising one or more of:
vii) a FRET cassette comprising a sequence complementary to said first flap sequence;
viii) a FRET cassette comprising a sequence complementary to said second flap sequence.

52. The kit of any one of claims 49-51, further comprising a flap endonuclease, preferably a FEN-1 endonuclease.

53. A composition, comprising:
i) a first primer pair for producing a first amplified DNA from a gene expression marker product from expression of a gene selected from S100A9, SELL, PADI4, APOBE3CA, S100A12, MMP9, FPR1, TYMP, and SAT1;
ii) a first probe comprising a sequence complementary to a region of said first amplified DNA;
iii) a second primer pair for producing a second amplified DNA;
iv) a second probe comprising a sequence complementary to a region of said second amplified DNA;
v) reverse transcriptase; and
vi) a thermostable DNA polymerase.

54. The composition of claim 53, further comprising nucleic acid extracted from blood sampled from a subject, wherein the subject preferably has or is suspected of having a lung neoplasm.

55. The composition of claim 54, wherein the nucleic acid comprises one or more of:
cellular RNA;
circulating cell-free RNA;
cellular DNA;
circulating cell-free DNA.

56. The composition of any one of claims 53-55, wherein the second primer pair produces a second amplified DNA from a methylation marker gene or a reference marker nucleic acid.

57. The composition of claim 56, wherein the second primer pair produces a second amplified DNA from a reference nucleic acid selected from:
RNA expressed from a gene selected from PLGLB2, GABARAP, NACA, EIF1, UBB, UBC, CD81, TMBIM6, MYL12B, HSP90B, CLDN18, RAMP2, MFAP4, FABP4, MARCO, RGL1, ZBTB16, C10orf116, GRK5, AGER, SCGB1A1, HBB, TCF21, GMFG, HYAL1, TEK, GNG11, ADH1A, TGFBR3, INPP1, ADH1B, STK4, ACTB, HNRNPA1, CASC3, and SKP1;
RNA selected from U1 snRNA and U6 snRNA;
DNA selected from B3GALT6 DNA and β-actin DNA.

58. The composition of claim 56, wherein the second primer pair produces a second amplified DNA from a methylation marker gene selected from EMX1, GRIN2D, ANKRD13B, ZNF781, ZNF671, IFFO1, HOPX, BARX1, HOXA9, LC100129726, SPOCK2, TSC22D4, MAX.chr8124, RASSF1, ST8SIA1, NKX6_2, FAM59B, DIDO1, MAX_Chr1.110, AGRN, SOBP, MAX_chr10.226, ZMIZ1, MAX_chr8.145, MAX_chr10.225, PRDM14, ANGPT1, MAX.chr16.50, PTGDR_9, DOCK2, MAX_chr19.163, ZNF132, MAX chr19.372, TRH, SP9, DMRTA2, ARHGEF4, CYP26C1, PTGDR, MATK, BCAT1, PRKCB_28, ST8SIA_22, FLJ45983, DLX4, SHOX2, HOXB2, MAX.chr12.526, BCL2L11, OPLAH, PARP15, KLHDC7B, SLC12A8, BHLHE23, CAPN2, FGF14, FLJ34208, BIN2_Z, DNMT3A, FERMT3, NFIX, SIPR4, SKI, SUCLG2, TBX15, and ZNF329.

59. The composition of any one of claims 53-58, wherein the first probe and/or the second probe comprises a detection moiety comprising a fluorophore.

60. The composition of any one of claims 53-58, wherein the first probe further comprises a flap portion having a first flap sequence that is not substantially complementary to said first amplified DNA, and/or wherein the second probe further comprises a flap portion having a second flap sequence that is not substantially complementary to said second amplified DNA.

61. The composition of claim 60, further comprising one or more of:
vii) a FRET cassette comprising a sequence complementary to the first flap sequence;
viii) a FRET cassette comprising a sequence complementary to the second flap sequence.

62. The composition of any one of claims 53-61, further comprising a flap endonuclease, preferably a FEN-1 endonuclease.

63. The composition of any one of claims 53-62, further comprising a buffer comprising 6-10 mM Mg⁺⁺.

64. A reaction mixture comprising a composition of any one of claims 53-63.
